SEQUENCE LISTING (25 sequences)

SEQ ID NO: 1 - DNA, 8879 bp, synthetic construct
  source 1..8879: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgggcgcgggcggccggatcatggtgaccccctcctccaagaagtccgagaccgaggccctgaagcgcggcccctgcgagaagccccccttcaccgtgaaggacctgaagaaggccatcccccagcactgcttcaagcgctccatcccccgctccttctcctacctgctgaccgacatcaccctggtgtcctgcttctactacgtggccaccaactacttctccctgctgccccagcccctgtcgacctacctggcctggcccctgtactgggtgtgccagggctgcgtgctgacgggcatctgggtgctgggccacgagtgcggccaccacgccttctccgactaccagtggctggacgacaccgtgggcttcatcttccactccctgctgctggtgccctacttctcctggaagtactcccaccgccgccaccactccaacaacggctccctggagaaggacgaggtgttcgtgccccccaagaaggccgccgtgaagtggtacgtgaagtacctgaacaaccccctgggccgcatcctggtgctgaccgtgcagttcatcctgggctggcccctgtacctggccttcaacgtgtccggccgcccctacgacggcttcgcctcccacttcttcccccacgcccccatcttcaaggaccgcgagcgcctccagatctacatctccgacgcgggcatcctggccgtgtgctacggcctgtaccgctacgccgcctcccagggcctgaccgccatgatctgcgtgtacggcgtgcccctgctgatcgtcaacttcttcctggtgctggtgaccttcctccagcacacccacccctccctgccccactacgactccaccgagtgggagtggatcaggggcgccctggtgaccgtggaccgcgactacggcatcctgaacaaggtgttccacaacatcaccgacacccacgtggcccaccacctgttcgccaccatcccccactacaacgccatggaggccaccgaggccatcaagcccatcctgggcgactactaccacttcgacggcaccccctggtacgtggccatgtaccgcgaggccaaggagtgcctgtacgtggagcccgacaccgagcgcggcaagaagggcgtgtactactacaacaacaagctgtgaacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaagcttgcctgctcaagcgggcgctcaacatgcagagcgtcagcgagacgggctgtggagatcgggagacggacgaggccgcctctgccctgtttgaactgagcgtcagcgctggctaaggggagggagactcatccccacgcccgcgccagggctctgatcccgtctcgggcggtgatcggcgcgcatgactacgacccaacgacgtacgagactgatgtcggtcccgacgaggagcgccgcgaggcactccagggctgccgaccatgtttacaccgaccaaaagcactcgctcgcatccattccgtgcgcccgcacatgcatcatcttttggtaccgatttcggtcttgttttacccctacgacctgccttccaaggtgtgagcaactcgcccggacatgaccgagggtgatcatccggatccccaggccccagcagcccctgccagaatggctcgcgctttccagcctgcaggcccgtctcccaggtcgacgcaacctacatgaccaccccaatctgtcccagaccccaaacaccctcctgccctgcttctctgtgatcgctgatcagcaacacccgggatggccaccgcctccaccttctccgccttcaacgcccgctgcggcgacctgcgccgctccgccggctccggcccccgccgccccgcccgccccctgcccgtgcgcgccgccatcggcaacgcccagacctccctgaagatgatcgacggcaccaagttctcctacaccgagtccctgaagcgcctgcccgactggtccaagctggacgaccgcttcggcctgcacggcctggtgttccgccgcaccttcgccatccgctcctacgaggtgggccccgaccgctccgcctccatcctggccgtgctgaaccacctgcaggaggccaccctgaaccacgccgagtccgtgggcatcctgggcgaccgcttcggcgagaccctggagatgtccaagcgcgacctgatgtgggtggtgcgccgcacctacgtggccgtggagcgctaccccgcctggggcgacaccgtggagatcgagtcctggatcggcgcctccggcaacaacggcatgcgccgcgagttcctggtgcgcgacttcaagaccggcgagatcctgacccgctgcacctccctgtccgtgatgatgaacacccgcacccgccgcctgtccaagatccccgaggaggtgcgcggcgagatcggccccgtgttcatcgacaacgtggccgtgaaggacgaggagatcaagaagctgcagaagctgaacgactccaccgccgactacatccagggcggcctgatcccccgctggaacgacctggacctgaaccagcacgtgaacaacatcaagtacgtgtcctggatcctggagaccgtgcccgactccatcctggagtcctaccacatgtcctccatcaccctggagtaccgccgcgagtgcacccgcgactccgtgctgcagtccctgaccaccgtgtccggcggctcctccgaggccggcctggtgtgcgagcactccctgctgctggagggcggctccgaggtgctgcgcgcccgcaccgagtggcgccccaagctgaccgactccttccgcggcatctccgtgatccccgccgagcagtccgtgatggactacaaggaccacgacggcgactacaaggaccacgacatcgactacaaggacgacgacgacaagtgaatcgatgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggagagctcagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttc

SEQ ID NO: 2 - protein, 384 aa, Physaria fendleri
MGAGGRIMVTPSSKKSETEALKRGPCEKPPFTVKDLKKAIPQHCFKRSIPRSFSYLLTDITLVSCFYYVATNYFSLLPQPLSTYLAWPLYWVCQGCVLTGIWVLGHECGHHAFSDYQWLDDTVGFIFHSLLLVPYFSWKYSHRRHHSNNGSLEKDEVFVPPKKAAVKWYVKYLNNPLGRILVLTVQFILGWPLYLAFNVSGRPYDGFASHFFPHAPIFKDRERLQIYISDAGILAVCYGLYRYAASQGLTAMICVYGVPLLIVNFFLVLVTFLQHTHPSLPHYDSTEWEWIRGALVTVDRDYGILNKVFHNITDTHVAHHLFATIPHYNAMEATEAIKPILGDYYHFDGTPWYVAMYREAKECLYVEPDTERGKKGVYYYNNKL

SEQ ID NO: 3 - protein, 378 aa, synthetic construct
MATASTFSAFNARCGDLRRSAGSGPRRPARPLPVRAAIGNAQTSLKMIDGTKFSYTESLKRLPDWSKLDDRFGLHGLVFRRTFAIRSYEVGPDRSASILAVLNHLQEATLNHAESVGILGDRFGETLEMSKRDLMWVVRRTYVAVERYPAWGDTVEIESWIGASGNNGMRREFLVRDFKTGEILTRCTSLSVMMNTRTRRLSKIPEEVRGEIGPVFIDNVAVKDEEIKKLQKLNDSTADYIQGGLIPRWNDLDLNQHVNNIKYVSWILETVPDSILESYHMSSITLEYRRECTRDSVLQSLTTVSGGSSEAGLVCEHSLLLEGGSEVLRARTEWRPKLTDSFRGISVIPAEQSVMDYKDHDGDYKDHDIDYFENTQHV

SEQ ID NO: 4 - DNA, 6770 bp, synthetic construct
  source 1..6770: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgggcgcgggcggccggatcatggtgaccccctcctccaagaagtccgagaccgaggccctgaagcgcggcccctgcgagaagccccccttcaccgtgaaggacctgaagaaggccatcccccagcactgcttcaagcgctccatcccccgctccttctcctacctgctgaccgacatcaccctggtgtcctgcttctactacgtggccaccaactacttctccctgctgccccagcccctgtcgacctacctggcctggcccctgtactgggtgtgccagggctgcgtgctgacgggcatctgggtgctgggccacgagtgcggccaccacgccttctccgactaccagtggctggacgacaccgtgggcttcatcttccactccctgctgctggtgccctacttctcctggaagtactcccaccgccgccaccactccaacaacggctccctggagaaggacgaggtgttcgtgccccccaagaaggccgccgtgaagtggtacgtgaagtacctgaacaaccccctgggccgcatcctggtgctgaccgtgcagttcatcctgggctggcccctgtacctggccttcaacgtgtccggccgcccctacgacggcttcgcctcccacttcttcccccacgcccccatcttcaaggaccgcgagcgcctccagatctacatctccgacgcgggcatcctggccgtgtgctacggcctgtaccgctacgccgcctcccagggcctgaccgccatgatctgcgtgtacggcgtgcccctgctgatcgtcaacttcttcctggtgctggtgaccttcctccagcacacccacccctccctgccccactacgactccaccgagtgggagtggatcaggggcgccctggtgaccgtggaccgcgactacggcatcctgaacaaggtgttccacaacatcaccgacacccacgtggcccaccacctgttcgccaccatcccccactacaacgccatggaggccaccgaggccatcaagcccatcctgggcgactactaccacttcgacggcaccccctggtacgtggccatgtaccgcgaggccaaggagtgcctgtacgtggagcccgacaccgagcgcggcaagaagggcgtgtactactacaacaacaagctgtgagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggagagctcagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 5 - protein, 384 aa, Physaria fendleri; identical to SEQ ID NO: 2

SEQ ID NO: 6 - DNA, 6779 bp, synthetic construct
  source 1..6779: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgggcggcggcggccggatgtccaccgtgatcacctccaacaactccgagaagaagggcggctcctcccacctgaagcgcgccccccacaccaagccccccttcaccctgggcgacctgaagcgcgccatccccccccactgcttcgagcgctccttcgtgcgctccttctcctacgtggcctacgacgtgtgcctgtccttcctgttctactccatcgccaccaacttcttcccctacatctcctcccccctgtcctacgtggcctggctggtgtactggctgttccagggctgcatcctgacgggcctgtgggtgatcggccacgagtgcggccaccacgccttctccgagtaccagctcgccgacgacatcgtgggcctgatcgtgcactccgccctgctggtgccctacttctcctggaagtactcccaccgccgccaccactccaacatcggctccctggagcgcgacgaggtgttcgtgcccaagtccaagtccaagatctcctggtactccaagtactcgaacaacccccccggccgcgtgctgaccctggccgccaccctgctgctgggctggcccctgtacctggccttcaacgtgtccggccgcccctacgaccgcttcgcctgccactacgacccctacggccccatcttctccgagcgcgagcgcctccagatctacatcgccgacctgggcatcttcgccaccaccttcgtgctgtaccaggccaccatggcgaagggcctggcctgggtgatgcgcatctacggcgtgcccctgctgatcgtcaactgcttcctggtgatgatcacctacctccagcacacccaccccgccatcccccgctacggctcctccgagtgggactggctgcgcggcgccatggtgaccgtggaccgcgactacggcgtgctgaacaaggtgttccacaacatcgccgacacccacgtggcccaccacctgttcgccaccgtgccccactaccacgccatggaggccaccaaggccatcaagcccatcatgggcgagtactaccgctacgacggcacccccttctacaaggccctgtggcgcgaggccaaggagtgcctgttcgtggagcccgacgagggcgcccccacccagggcgtgttctggtacaggaacaagtactgagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggagagctcagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 7 - protein, 387 aa, Ricinus communis
MGGGGRMSTVITSNNSEKKGGSSHLKRAPHTKPPFTLGDLKRAIPPHCFERSFVRSFSYVAYDVCLSFLFYSIATNFFPYISSPLSYVAWLVYWLFQGCILTGLWVIGHECGHHAFSEYQLADDIVGLIVHSALLVPYFSWKYSHRRHHSNIGSLERDEVFVPKSKSKISWYSKYSNNPPGRVLTLAATLLLGWPLYLAFNVSGRPYDRFACHYDPYGPIFSERERLQIYIADLGIFATTFVLYQATMAKGLAWVMRIYGVPLLIVNCFLVMITYLQHTHPAIPRYGSSEWDWLRGAMVTVDRDYGVLNKVFHNIADTHVAHHLFATVPHYHAMEATKAIKPIMGEYYRYDGTPFYKALWREAKECLFVEPDEGAPTQGVFWYRNKY

SEQ ID NO: 8 - DNA, 7049 bp, synthetic construct
  source 1..7049: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatggcctccgccacccccgccatgtccgagaacgccgtgctgcgccacaaggccgcctccaccacgggcatcgactacgagtcctccgccgccgtgtcccccgccgagtccccccgcacctccgcctcctccacctccctgtcctccctgtcctccctggacgccaacgagaagaaggacgagtacgcgggcctgctggacacctacggcaacgccttcaccccccccgacttctccatcaaggacatccgcgccgccatccccaagcactgctacgagcgctccaccatcaagtcctacgcctacgtgctgcgcgacctgctgtgcctgtccaccaccttctacctgttccacaacttcgtgacccccgagaacatcccctccaaccccctgcgcttcgtgctgtggtccatctacaccgtgctccagggcctgttcgccacgggcctgtgggtgatcggccacgagtgcggccactgcgccttctccccctcccccttcatctccgacctgacgggctgggtgatccactccgccctgctggtgccctacttctcctggaagttctcccactccgcccaccacaagggcatcggcaacatggagcgcgacatggtgttcctgccccgcacccgcgagcagcaggcgacccgcctgggccgcgccgtggaggagctgggcgacctgtgcgaggagacccccatctacaccgccctgcacctggtgggcaagcagctcatcggctggccctcctacctgatgaccaacgccacgggccacaacttccacgagcgccagcgcgagggccgcggcaagggcaagaagaacggctttggcggcggcgtcaaccacttcgacccccgctcccccatcttcgaggcccgccaggccaagtacatcgtgctgtccgacatcggcctgggcctggccatcgccgccctggtgtacctgggcaaccgcttcggctgggccaacatgaccgtgtggtacttcctgccctacctgtgggtgaaccactggctggtggccatcaccttcctccagcacaccgaccccaccctgccccactacaaccgcgaggagtggaacttcgtgcgcggcggcgcctgcaccatcgaccgcgacctgggcttcatcggccgccacctgttccacggcatcgccgacacccacgtggtgcaccactacgtgtcccgcatccccttctacaacgccgacgaggcctccgaggccatcaagcccatcatgggcaagcactaccgctccgacaccgcccacggccccgtgggcttcctgcacgccctgtggaagaccgcccgctggtgccagtgggtggagccctccgccgacgcccagggcgcgggcaagggcatcctgttctaccgcaaccgcaacaagctgggcaccaagcccatctccatgaagacccagtgagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggagagctcagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 9 - protein, 477 aa, unidentified
MASATPAMSENAVLRHKAASTTGIDYESSAAVSPAESPRTSASSTSLSSLSSLDANEKKDEYAGLLDTYGNAFTPPDFSIKDIRAAIPKHCYERSTIKSYAYVLRDLLCLSTTFYLFHNFVTPENIPSNPLRFVLWSIYTVLQGLFATGLWVIGHECGHCAFSPSPFISDLTGWVIHSALLVPYFSWKFSHSAHHKGIGNMERDMVFLPRTREQQATRLGRAVEELGDLCEETPIYTALHLVGKQLIGWPSYLMTNATGHNFHERQREGRGKGKKNGFGGGVNHFDPRSPIFEARQAKYIVLSDIGLGLAIAALVYLGNRFGWANMTVWYFLPYLWVNHWLVAITFLQHTDPTLPHYNREEWNFVRGGACTIDRDLGFIGRHLFHGIADTHVVHHYVSRIPFYNADEASEAIKPIMGKHYRSDTAHGPVGFLHALWKTARWCQWVEPSADAQGAGKGILFYRNRNKLGTKPISMKTQ

SEQ ID NO: 10 - DNA, 7788 bp, synthetic construct
  source 1..7788: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcactgaagaatgggaggcaggtgctgctgattacgagtgtggaaaagaaagggggcagagagccgtcctcagatccgaccactatgcaggtagccgctcgcccgtgcccgcctggctgaatattgacacatgcccatcaaggcaggcaggcatttctgtgcatgcaccaaacccataatcttcgaaaacacacagcatgtaccaacgcacgcgtaaaagttggggtgctgccagtgcgtcatgccaggcatgatgtgctcctgcacatcggccatgatctcctccatcgtctcgggcgtctccggcgcctggtccgggagccgttccgccagatacccagacgccacctccgacctcacggggtacttttcgagcgactgtcggtagtcgacgatcgcgtccaccatggagtagccgaggcgccggaactggcgtgacggagggagaacagggggggttgatgatcacacgccagtctcacaacgcatgtaagacccgttggattatgagtataatcatgcattactagttgggtgagcgccaggcataaggcacaccgacgtcgatggcacgagcaactcccgcatcatatttcctattgtcctaacgccaagccggtcaccacccgcatgctcgtactacagcgcacgcaccgcttcgtgatccaccgggtgaacgtagtcctcgacggaaacatctggttcgggcctcctgcttgcactcccgcccatgccgacaacctttctgctgttaccacgacccacaatgcaacgcgacacgaccgtgtgggactgatcggttcactgcacctgcatgcaattgtcacaagcgcttactccaattgtattcgtttgttttctgggagcagttgctcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacacatatgatgcccgtgatccgccgcaagaagcccacctccgagcccaacaagaactccgcctccgactccaagaccccctccgaggaggaggagcacgagcaggagcaggagcaggaggaggacaagaacaacaagaagaagtaccccaagaagaagtcctccgagatcaacgccaagaagtggtcctgcatcgactcctgctgctggttcgtgggctgcatctgcgtgacctggtgggtgctgctgttcctgtacaacgccgtgcccgcctccctgccccagtacgtgaccgaggccatcacgggccccctgcccgacccccccggcgtgaagctgaagaaggagggcctgaccgccaagcaccccgtggtgttcgtgcccggcatcgtgaccgcgggcctggagctgtgggagggccaccagtgcgccgacggcctgttccgcaagcgcctgtggggcggcaccttcggcgaggtgtacaagcgccccctgtgctgggtggagcacatgtccctggacaacgagacgggcctggacccccccggcatccgcgtgcgccccgtgtccggcctggtggccgccgactacttcgcccccggctacttcgtgtgggccgtgctgatcgccaacctggcccgcatcggctacgaggagaagacgatgttcatggcctcctacgactggcgcctgtccttccagaacaccgaggtgcgcgaccagaccctgtcccgcatgaagtccaacatcgagctgatggtgtccatcaacggcggcaacaaggccgtgatcgtgccccactccatgggcgtgctgtacttcctgcacttcatgaagtgggtggaggcccccgcccccatgggcggcggcggcggccccgactggtgcgccaagcacatcaaggccgtgatgaacatcggcggccccttcctgggcgtgcccaaggccgtcgcgggcctgttctccgccgaggcccgcgacatcgccgtggcccgcgccatcgcccccggcttcctggacaacgacatgttccgcctccagaccctccagcacatgatgcgcatgtcccgcacctgggactccaccatgtccatgatcccccgcggcggcgacaccatctggggcgacctggactggtcccccgaggagggctacatcccccgcaagaagcgccagcgcaacaacgccaccgacaacgtgaacgagggcggcgccgagtccgagatctcccagcgcaagatcgtgcgctacggccgcatgatctccttcggcaagaacatcgccgaggccccctcctacgacatcgagcgcatcgacttccgcgacgccgtgaagggccgctccgtggccaacaacacctgcctggacgtgtggaccgagtaccacgagatgggcttcggcggcatcaaggccgtggccgagtacaaggtgtacaccgcgggctccaccatcgagctgctccagttcgtggccccgaagatgatggagcgcggctccgcccacttctcctacgagatcgccgacaacctggaggaccccaagtacgagcactacaagtactggtcgaaccccctggagaccaagctgcccaacgcccccgagatggagatcttctccatgtacggcgtgggcatccccaccgagcgcgcctacgtgtacgagttctcccccgccgccgagtgctacatccccttccagatcgacacctccgccaacgacggcgacgaggacggctgcctgaaggacggcgtgtacaccgtggacggcgacgagaccgtgcccgtgctgtccgcgggcttcatgtgcgccaaggcctggcgcggcaagacccgcttcaacccctccggctcccgcacctacatccgcgagtacgaccactccccccccgcgaacctgctggagggccgcggcacccagtccggcgcccacgtggacatcatgggcaacttcgccctgatcgaggacatcatgcgcgtggccgcgggcgccacgggcgaggacctgggcggcgaccaggtgtactccgacatcttcaagtggtcccagaagatcaagctgcccctgtaattaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 11 - protein, 685 aa, Ricinus communis
MPVIRRKKPTSEPNKNSASDSKTPSEEEEHEQEQEQEEDKNNKKKYPKKKSSEINAKKWSCIDSCCWFVGCICVTWWVLLFLYNAVPASLPQYVTEAITGPLPDPPGVKLKKEGLTAKHPVVFVPGIVTAGLELWEGHQCADGLFRKRLWGGTFGEVYKRPLCWVEHMSLDNETGLDPPGIRVRPVSGLVAADYFAPGYFVWAVLIANLARIGYEEKTMFMASYDWRLSFQNTEVRDQTLSRMKSNIELMVSINGGNKAVIVPHSMGVLYFLHFMKWVEAPAPMGGGGGPDWCAKHIKAVMNIGGPFLGVPKAVAGLFSAEARDIAVARAIAPGFLDNDMFRLQTLQHMMRMSRTWDSTMSMIPRGGDTIWGDLDWSPEEGYIPRKKRQRNNATDNVNEGGAESEISQRKIVRYGRMISFGKNIAEAPSYDIERIDFRDAVKGRSVANNTCLDVWTEYHEMGFGGIKAVAEYKVYTAGSTIELLQFVAPKMMERGSAHFSYEIADNLEDPKYEHYKYWSNPLETKLPNAPEMEIFSMYGVGIPTERAYVYEFSPAAECYIPFQIDTSANDGDEDGCLKDGVYTVDGDETVPVLSAGFMCAKAWRGKTRFNPSGSRTYIREYDHSPPANLLEGRGTQSGAHVDIMGNFALIEDIMRVAAGATGEDLGGDQVYSDIFKWSQKIKLPL

SEQ ID NO: 12 - DNA, 7560 bp, synthetic construct
  source 1..7560: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcactgaagaatgggaggcaggtgctgctgattacgagtgtggaaaagaaagggggcagagagccgtcctcagatccgaccactatgcaggtagccgctcgcccgtgcccgcctggctgaatattgacacatgcccatcaaggcaggcaggcatttctgtgcatgcaccaaacccataatcttcgaaaacacacagcatgtaccaacgcacgcgtaaaagttggggtgctgccagtgcgtcatgccaggcatgatgtgctcctgcacatcggccatgatctcctccatcgtctcgggcgtctccggcgcctggtccgggagccgttccgccagatacccagacgccacctccgacctcacggggtacttttcgagcgactgtcggtagtcgacgatcgcgtccaccatggagtagccgaggcgccggaactggcgtgacggagggagaacagggggggttgatgatcacacgccagtctcacaacgcatgtaagacccgttggattatgagtataatcatgcattactagttgggtgagcgccaggcataaggcacaccgacgtcgatggcacgagcaactcccgcatcatatttcctattgtcctaacgccaagccggtcaccacccgcatgctcgtactacagcgcacgcaccgcttcgtgatccaccgggtgaacgtagtcctcgacggaaacatctggttcgggcctcctgcttgcactcccgcccatgccgacaacctttctgctgttaccacgacccacaatgcaacgcgacacgaccgtgtgggactgatcggttcactgcacctgcatgcaattgtcacaagcgcttactccaattgtattcgtttgttttctgggagcagttgctcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacacatatgatgatcggctacctgtgcaccgcctggtggctgctgctgttcctgtaccactgcctgcccgccaccctgcccggcttccaggtgcccgagtcccccggcgcccgcctgaagcgcgagggcctgatcgcccagcaccccgtggtgctggtgcccggcatcatcacgggcgccctggagctgtgggagggcaagccctgcgccgagggcctgttccgcaagcgcctgtggggcggctccttctccgagatcctgaagcgccccctgtgctggctggaccacctggccctgcacaacgagacgggcctggacccccccggcatccgcgtgcgcgccgtgacgggcctggtggccgccgactacttcgcccccggctacttcgtgtgggccgtgctgatcgagaacctggccaagatcggctacgagggcaagaacctgcacatggccgcctacgactggcgcctgtccttccagaacaccgagatccgcgaccaggccctgacccgcctgaagtccaagatcgagttcatgtacgtgaccaacggctacaagaaggtggtggtggtgccccactccatgggcgtgatctacttcctgcacttcctgaagtgggtggagacccccccccccatgggcggcggcggcggccccggctggtgcaacaagcacatcaaggccatcatgaacatcggcccgaccttcctgggcgtgcccaagaccgtgtccaacatcctgtccgccgaggccaaggacaccgccttcatccgcgccatcctgcccggcatcctggactccgagatcctgggcgtgcaggccatcgagcacgtgctgcgcatgacccgcacctgggactccaccatgtccctgctgcccaagggcggcgagaccatctggggcaacctggactgggcccccgaggagcgcgaggcctgcgactcctccaagaagcgctacctgcgctccatcaacgactccaactccgacgtgaagcgcggcttccaggtgaaggagtccgtgaactacggccgcatcgtgtccttctccaaggccgccgcccagctcccctcctccctgctgccctccttcgacctcaaggagttcttcggcgagcacaccaacacctcctccggctcctgcggcaagatctggaccgagtacgacgagatcaaccgcgagtccatccgcaagttcgccgagaacaaggccttcaccgcctccaccttccacgacctgctgcgcttcgtggcccccaagatggtgcagcgcgcgggcgcccacttctcccacggcatcgccgacaacctggacgaccccaagtacgagcactacaagtactggtcgaaccccctggagacccgcctgcccgacgcccccgacatggagatctactgctcctacggcgtgggcatccccaccgagcgctcctacgccttcaagctgtccccctccgaccgctgcaagtccatccccctgcgcatcgacacctccgtgggcgactccggcttcaccaacggcgtgtccttcgtggacggcgacgtgtccgtgcccgtgctgtccgcgggcttcatgtgcgccaaggtgtggcggggccgcacccgcttcaacccctccggcatccgcacctacgtgcgcgagttccagcacaagccccccggctccctgctggacggccgcggcaccgagtccggctcccacgtggacatcatgggcaacaacgccctgatcgaggacgtgctgcgcgtggccgcgggcgccacgggcaccgagatgggcggcgaccgcatccactccgacatcctgcgcatggccgagaagatcaacatccagctctaattaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 13 - protein, 609 aa, Ricinus communis
MIGYLCTAWWLLLFLYHCLPATLPGFQVPESPGARLKREGLIAQHPVVLVPGIITGALELWEGKPCAEGLFRKRLWGGSFSEILKRPLCWLDHLALHNETGLDPPGIRVRAVTGLVAADYFAPGYFVWAVLIENLAKIGYEGKNLHMAAYDWRLSFQNTEIRDQALTRLKSKIEFMYVTNGYKKVVVVPHSMGVIYFLHFLKWVETPPPMGGGGGPGWCNKHIKAIMNIGPTFLGVPKTVSNILSAEAKDTAFIRAILPGILDSEILGVQAIEHVLRMTRTWDSTMSLLPKGGETIWGNLDWAPEEREACDSSKKRYLRSINDSNSDVKRGFQVKESVNYGRIVSFSKAAAQLPSSLLPSFDLKEFFGEHTNTSSGSCGKIWTEYDEINRESIRKFAENKAFTASTFHDLLRFVAPKMVQRAGAHFSHGIADNLDDPKYEHYKYWSNPLETRLPDAPDMEIYCSYGVGIPTERSYAFKLSPSDRCKSIPLRIDTSVGDSGFTNGVSFVDGDVSVPVLSAGFMCAKVWRGRTRFNPSGIRTYVREFQHKPPGSLLDGRGTESGSHVDIMGNNALIEDVLRVAAGATGTEMGGDRIHSDILRMAEKINIQL

SEQ ID NO: 14 - DNA, 6588 bp, synthetic construct
  source 1..6588: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcactgaagaatgggaggcaggtgctgctgattacgagtgtggaaaagaaagggggcagagagccgtcctcagatccgaccactatgcaggtagccgctcgcccgtgcccgcctggctgaatattgacacatgcccatcaaggcaggcaggcatttctgtgcatgcaccaaacccataatcttcgaaaacacacagcatgtaccaacgcacgcgtaaaagttggggtgctgccagtgcgtcatgccaggcatgatgtgctcctgcacatcggccatgatctcctccatcgtctcgggcgtctccggcgcctggtccgggagccgttccgccagatacccagacgccacctccgacctcacggggtacttttcgagcgactgtcggtagtcgacgatcgcgtccaccatggagtagccgaggcgccggaactggcgtgacggagggagaacagggggggttgatgatcacacgccagtctcacaacgcatgtaagacccgttggattatgagtataatcatgcattactagttgggtgagcgccaggcataaggcacaccgacgtcgatggcacgagcaactcccgcatcatatttcctattgtcctaacgccaagccggtcaccacccgcatgctcgtactacagcgcacgcaccgcttcgtgatccaccgggtgaacgtagtcctcgacggaaacatctggttcgggcctcctgcttgcactcccgcccatgccgacaacctttctgctgttaccacgacccacaatgcaacgcgacacgaccgtgtgggactgatcggttcactgcacctgcatgcaattgtcacaagcgcttactccaattgtattcgtttgttttctgggagcagttgctcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacacatatgatgaagtccaccgtgccccccaccaccaccaccaccaccaccaccaccctgtacaagcgcaagaaggacatcaacctgacctccgtgaacgactccgtggacatggtgtccaacaagaacttcgccaacggcaacgtgaacggcggcggcggctacaccgccttcaaccgcttcgacccctccttcatgaagtggaccacccacgacgtggtgaacgtggtgaagttccactggctgccctgcgtgttcggcctgggcctgctgttcttcatggccgtggagtacaccctgcgcatggtgcccgcctcctccccccccttcgacctgggcttcctggtgacccgccacctgcacctgctgctgtcctcctggcccgccctgaacaccctgctggccttcctgaacaccgtgttcgtgctgatgcagaccgcctacatcctgtggacctggctgatcgagggccgcccccgcgccaccatctccgccctgttcatgttcacctgccgcggcatcctgggctactccacccagctccccctgcccgagggcttcctgggctccggcgtggacttccccgtgggcaacgtgtccttcttcctgttcttctccggccacgtggcgggctccgtgatcgcctccctggacatgcgccgcatgcagcgctgggagctggcctggacctacgacgtgctgaacgtgctccaggccgtgcgcctgctgggcacccgcggccactacacgatcgacctggccacgggcgtcggcgcgggcatcctgttcgactccctggcgggcaagtacgaggagtccaagcgcaagcaggccgtggtggccaaggagtcctccctgttctcctaattaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 15 - protein, 285 aa, Ricinus communis
MKSTVPPTTTTTTTTTLYKRKKDINLTSVNDSVDMVSNKNFANGNVNGGGGYTAFNRFDPSFMKWTTHDVVNVVKFHWLPCVFGLGLLFFMAVEYTLRMVPASSPPFDLGFLVTRHLHLLLSSWPALNTLLAFLNTVFVLMQTAYILWTWLIEGRPRATISALFMFTCRGILGYSTQLPLPEGFLGSGVDFPVGNVSFFLFFSGHVAGSVIASLDMRRMQRWELAWTYDVLNVLQAVRLLGTRGHYTIDLATGVGAGILFDSLAGKYEESKRKQAVVAKESSLFS

SEQ ID NO: 16 - DNA, 6753 bp, synthetic construct
  source 1..6753: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcactgaagaatgggaggcaggtgctgctgattacgagtgtggaaaagaaagggggcagagagccgtcctcagatccgaccactatgcaggtagccgctcgcccgtgcccgcctggctgaatattgacacatgcccatcaaggcaggcaggcatttctgtgcatgcaccaaacccataatcttcgaaaacacacagcatgtaccaacgcacgcgtaaaagttggggtgctgccagtgcgtcatgccaggcatgatgtgctcctgcacatcggccatgatctcctccatcgtctcgggcgtctccggcgcctggtccgggagccgttccgccagatacccagacgccacctccgacctcacggggtacttttcgagcgactgtcggtagtcgacgatcgcgtccaccatggagtagccgaggcgccggaactggcgtgacggagggagaacagggggggttgatgatcacacgccagtctcacaacgcatgtaagacccgttggattatgagtataatcatgcattactagttgggtgagcgccaggcataaggcacaccgacgtcgatggcacgagcaactcccgcatcatatttcctattgtcctaacgccaagccggtcaccacccgcatgctcgtactacagcgcacgcaccgcttcgtgatccaccgggtgaacgtagtcctcgacggaaacatctggttcgggcctcctgcttgcactcccgcccatgccgacaacctttctgctgttaccacgacccacaatgcaacgcgacacgaccgtgtgggactgatcggttcactgcacctgcatgcaattgtcacaagcgcttactccaattgtattcgtttgttttctgggagcagttgctcgaccgcccgcgtcccgcaggcagcgatgacgtgtgcgtggcctgggtgtttcgtcgaaaggccagcaaccctaaatcgcaggcgatccggagattgggatctgatccgagtttggaccagatccgccccgatgcggcacgggaactgcatcgactcggcgcggaacccagctttcgtaaatgccagattggtgtccgatacctggatttgccatcagcgaaacaagacttcagcagcgagcgtatttggcgggcgtgctaccagggttgcatacattgcccatttctgtctggaccgctttactggcgcagagggtgagttgatggggttggcaggcatcgaaacgcgcgtgcatggtgtgcgtgtctgttttcggctgcacgaattcaatagtcggatgggcgacggtagaattgggtgtggcgctcgcgtgcatgcctcgccccgtcgggtgtcatgaccgggactggaatcccccctcgcgaccatcttgctaacgctcccgactctcccgaccgcgcgcaggatagactcttgttcaaccaatcgacacatatgatgggcgaggaggccaaccacaacaacaacaacaacaacatcaactccaacgacgagaagaacgaggagaagtccaactacaccgtggtgaactcccgcgagctgtaccccaccaacatcttccacgccctgctggccctgtccatctggatcggctccatccacttcaacctgttcctgctgttcatctcctacctgttcctgtccttccccaccttcctgctgatcgtgggcttcttcgtggtgctgatgttcatccccatcgacgagcactccaagctgggccgccgcctgtgccgctacgtgtgccgccacgcctgctcccacttccccgtgaccctgcacgtggaggacatgaacgcctttcactccgaccgcgcctacgtgttcggctacgagccccactccgtgttccccctgggcgtgtccgtgctgtccgaccacttcgccgtgctgcccctgcccaagatgaaggtgctggcctccaacgccgtgttccgcacccccgtgctgcgccacatctggacctggtgcggcctgacctccgccaccaagaagaactttaccgccctgctggcctccggctactcctgcatcgtgatccccggcggcgtgcaggagaccttctacatgaagcacggctccgagatcgccttcctgaaggcccgccgcggcttcgtgcgcgtggcgatggagatgggcaagcccctggtgcccgtgttctgcttcggccagtccaacgtgtacaagtggtggaagcccgacggcgagctgttcatgaagatcgcccgcgccatcaagttctcccccatcgtgttctggggcgtgctgggctcccacctgcccctccagcgccccatgcacgtcgtggtgggcaagcccatcgaggtgaagcagaacccccagcccaccgtggaggaggtgtccgaggtgcagggccagttcgtggccgccctgaaggacctgttcgagcgccacaaggcccgcgtgggctacgccgacctgaccctggagatcctgtaattaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaggccggcccgcgtctcgaacagagcgcggcggccgcatcgatacctgcaggacagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 17 - protein, 340 aa, Ricinus communis
MGEEANHNNNNNNINSNDEKNEEKSNYTVVNSRELYPTNIFHALLALSIWIGSIHFNLFLLFISYLFLSFPTFLLIVGFFVVLMFIPIDEHSKLGRRLCRYVCRHACSHFPVTLHVEDMNAFHSDRAYVFGYEPHSVFPLGVSVLSDHFAVLPLPKMKVLASNAVFRTPVLRHIWTWCGLTSATKKNFTALLASGYSCIVIPGGVQETFYMKHGSEIAFLKARRGFVRVAMEMGKPLVPVFCFGQSNVYKWWKPDGELFMKIARAIKFSPIVFWGVLGSHLPLQRPMHVVVGKPIEVKQNPQPTVEEVSEVQGQFVAALKDLFERHKARVGYADLTLEIL

SEQ ID NO: 18 - DNA, 7219 bp, synthetic construct
  source 1..7219: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaataacaatcaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgggcgcgggcggccggatcatggtgaccccctcctccaagaagtccgagaccgaggccctgaagcgcggcccctgcgagaagccccccttcaccgtgaaggacctgaagaaggccatcccccagcactgcttcaagcgctccatcccccgctccttctcctacctgctgaccgacatcaccctggtgtcctgcttctactacgtggccaccaactacttctccctgctgccccagcccctgtcgacctacctggcctggcccctgtactgggtgtgccagggctgcgtgctgacgggcatctgggtgctgggccacgagtgcggccaccacgccttctccgactaccagtggctggacgacaccgtgggcttcatcttccactccctgctgctggtgccctacttctcctggaagtactcccaccgccgccaccactccaacaacggctccctggagaaggacgaggtgttcgtgccccccaagaaggccgccgtgaagtggtacgtgaagtacctgaacaaccccctgggccgcatcctggtgctgaccgtgcagttcatcctgggctggcccctgtacctggccttcaacgtgtccggccgcccctacgacggcttcgcctcccacttcttcccccacgcccccatcttcaaggaccgcgagcgcctccagatctacatctccgacgcgggcatcctggccgtgtgctacggcctgtaccgctacgccgcctcccagggcctgaccgccatgatctgcgtgtacggcgtgcccctgctgatcgtcaacttcttcctggtgctggtgaccttcctccagcacacccacccctccctgccccactacgactccaccgagtgggagtggatcaggggcgccctggtgaccgtggaccgcgactacggcatcctgaacaaggtgttccacaacatcaccgacacccacgtggcccaccacctgttcgccaccatcccccactacaacgccatggaggccaccgaggccatcaagcccatcctgggcgactactaccacttcgacggcaccccctggtacgtggccatgtaccgcgaggccaaggagtgcctgtacgtggagcccgacaccgagcgcggcaagaagggcgtgtactactacaacaacaagctgtgattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggagagctcagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 19 - DNA, 6818 bp, synthetic construct
  source 1..6818: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaatcaaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgggcgcgggcggccggatcatggtgaccccctcctccaagaagtccgagaccgaggccctgaagcgcggcccctgcgagaagccccccttcaccgtgaaggacctgaagaaggccatcccccagcactgcttcaagcgctccatcccccgctccttctcctacctgctgaccgacatcaccctggtgtcctgcttctactacgtggccaccaactacttctccctgctgccccagcccctgtcgacctacctggcctggcccctgtactgggtgtgccagggctgcgtgctgacgggcatctgggtgctgggccacgagtgcggccaccacgccttctccgactaccagtggctggacgacaccgtgggcttcatcttccactccctgctgctggtgccctacttctcctggaagtactcccaccgccgccaccactccaacaacggctccctggagaaggacgaggtgttcgtgccccccaagaaggccgccgtgaagtggtacgtgaagtacctgaacaaccccctgggccgcatcctggtgctgaccgtgcagttcatcctgggctggcccctgtacctggccttcaacgtgtccggccgcccctacgacggcttcgcctcccacttcttcccccacgcccccatcttcaaggaccgcgagcgcctccagatctacatctccgacgcgggcatcctggccgtgtgctacggcctgtaccgctacgccgcctcccagggcctgaccgccatgatctgcgtgtacggcgtgcccctgctgatcgtcaacttcttcctggtgctggtgaccttcctccagcacacccacccctccctgccccactacgactccaccgagtgggagtggatcaggggcgccctggtgaccgtggaccgcgactacggcatcctgaacaaggtgttccacaacatcaccgacacccacgtggcccaccacctgttcgccaccatcccccactacaacgccatggaggccaccgaggccatcaagcccatcctgggcgactactaccacttcgacggcaccccctggtacgtggccatgtaccgcgaggccaaggagtgcctgtacgtggagcccgacaccgagcgcggcaagaagggcgtgtactactacaacaacaagctgtgaacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 20 - DNA, 7165 bp, synthetic construct
  source 1..7165: mol_type = other DNA
gctcttccgcttgcccgcaccctcgttgatctgggagccctgcgcagccccttaaatcatctcagtcaggtttctatgttcaactgagcctaaagggctttcgtcacgcgcacgagcacacgtatatcggctacgcagtctctcagaagcggtagaacagttcgcaagccctcgtcggtcgaaaacttgcgccagtactattgaattaaattaaatgatcgaatgagacgcgaaacttttgcagaatgccactgagtttgcccagagaatgggagtggcgccattcaccatccgcctgtgcacggcctgattcgccgagacgatgaatggcgagaccagggagcggcttgcgagccccgagccggtagcaggaataacggtcgacaatcttcctgtccaattactggcaaccattagaaagagccggagcgcgttgaaattctgcaatcgagtaatttttcgatgcggcgggcctgctgaaccctaaggctccggcctatgtttaaggcgatccaagatgcacgcggccccaggcacgtgtctcaagcacaaaccccagccttagtttcgagactttgggagatagcggcagatatctagtttggcattttgtatattaattaagtctcgcaatggagcgctctgatgcggtgcagcgtcggctgcagcacctggcagtggcgctggggtcgccctatcgctcggcgcctggtcagctggctcccgcctcctgcggcgcgccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactgtacaatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgacttaagacgcccgcgcggcgcacctgacctgttctctcgagggcgcctgttctgccttgcgaaacaagcccctggagcatgcgtgcatgatcgtctctggcgccccgccgcgcggtttgtcgccctcgcgggcgccgcggccgcgggggcgcattgaaattgttgcaaaccccacctgacagattgagggcccaggcaggaaggcgttgagatggaggtacaggagtcaagtaactgaaagtttttatgataactaacaacaaagggtcgtttctggccagcgaatgacaagaacaagattccacatttccgtgtagaggcttgccatcgaatgtgagcgggcgggccgcggacccgacaaaacccttacgacgtggtaagaaaaacgtggcgggcactgtccctgtagcctgaagaccagcaggagacgatcggaagcatcacagcacaaccggtgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctaggaaataacaaatcaagcttggccgacaggacgcgcgtcaaaggtgctggtcgtgtatgccctggccggcaggtcgttgctgctgctggttagtgattccgcaaccctgattttggcgtcttattttggcgtggcaaacgctggcgcccgcgagccgggccggcggcgatgcggtgccccacggctgccggaatccaagggaggcaagagcgcccgggtcagttgaagggctttacgcgcaaggtacagccgctcctgcaaggctgcgtggtggaattggacgtgcaggtcctgctgaagttcctccaccgcctcaccagcggacaaagcaccggtgtatcaggtccgtgtcatccactctaaagagctcgactacgacctactgatggccctagattcttcatcaaaaacgcctgagacacttgcccaggattgaaactccctgaagggaccaccaggggccctgagttgttccttccccccgtggcgagctgccagccaggctgtacctgtgatcgaggctggcgggaaaataggcttcgtgtgctcaggtcatgggaggtgcaggacagctcatgaaacgccaacaatcgcacaattcatgtcaagctaatcagctatttcctcttcacgagctgtaattgtcccaaaattctggtctaccgggggtgatccttcgtgtacgggcccttccctcaaccctaggtatgcgcgcatgcggtcgccgcgcaactcgcgcgagggccgagggtttgggacgggccgtcccgaaatgcagttgcacccggatgcgtggcaccttttttgcgataatttatgcaatggactgctctgcaaaattctggctctgtcgccaaccctaggatcagcggcgtaggatttcgtaatcattcgtcctgatggggagctaccgactaccctaatatcagcccgactgcctgacgccagcgtccacttttgtgcacacattccattcgtgcccaagacatttcattgtggtgcgaagcgtccccagttacgctcacctgtttcccgacctccttactgttctgtcgacagagcgggcccacaggccggtcgcagccatgggcgcgggcggccggatcatggtgaccccctcctccaagaagtccgagaccgaggccctgaagcgcggcccctgcgagaagccccccttcaccgtgaaggacctgaagaaggccatcccccagcactgcttcaagcgctccatcccccgctccttctcctacctgctgaccgacatcaccctggtgtcctgcttctactacgtggccaccaactacttctccctgctgccccagcccctgtcgacctacctggcctggcccctgtactgggtgtgccagggctgcgtgctgacgggcatctgggtgctgggccacgagtgcggccaccacgccttctccgactaccagtggctggacgacaccgtgggcttcatcttccactccctgctgctggtgccctacttctcctggaagtactcccaccgccgccaccactccaacaacggctccctggagaaggacgaggtgttcgtgccccccaagaaggccgccgtgaagtggtacgtgaagtacctgaacaaccccctgggccgcatcctggtgctgaccgtgcagttcatcctgggctggcccctgtacctggccttcaacgtgtccggccgcccctacgacggcttcgcctcccacttcttcccccacgcccccatcttcaaggaccgcgagcgcctccagatctacatctccgacgcgggcatcctggccgtgtgctacggcctgtaccgctacgccgcctcccagggcctgaccgccatgatctgcgtgtacggcgtgcccctgctgatcgtcaacttcttcctggtgctggtgaccttcctccagcacacccacccctccctgccccactacgactccaccgagtgggagtggatcaggggcgccctggtgaccgtggaccgcgactacggcatcctgaacaaggtgttccacaacatcaccgacacccacgtggcccaccacctgttcgccaccatcccccactacaacgccatggaggccaccgaggccatcaagcccatcctgggcgactactaccacttcgacggcaccccctggtacgtggccatgtaccgcgaggccaaggagtgcctgtacgtggagcccgacaccgagcgcggcaagaagggcgtgtactactacaacaacaagctgtgacagacgaccttggcaggcgtcgggtagggaggtggtggtgatggcgtctcgatgccatcgcacgcatccaacgaccgtatacgcatcgtccaatgaccgtcggtgtcctctctgcctccgttttgtgagatgtctcaggcttggtgcatcctcgggtggccagccacgttgcgcgtcgtgctgcttgcctctcttgcgcctctgtggtactggaaaatatcatcgaggcccgtttttttgctcccatttcctttccgctacatcttgaaagcaaacgacaaacgaagcagcaagcaaagagcacgaggacggtgaacaagtctgtcacctgtatacatctatttccccgcgggtgcacctactctctctcctgccccggcagagtcagcgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggagagctcagcgtctgcgtgttgggagctggagtcgtgggcttgacgacggcgctgcagctcttgcaggatgtgcctggcgtgcgcgttcacgtcgtggctgagaaatatggcgacgaaacgttgacggctggggccggcgggctgtggatgccatacgcattgggtacgcggccattggatgggattggtaggcttatggagggataatagagtttttgtcggatccaacgcatgttgatgcagtagcccggtgtgctgaaagtatggaaggatggtgcattggctattcacatgcgctgcccaccccttttcgcaggaaatgtgccggcatcgttggtgcaccgatggggaaaatcgacgttcgaccactacatgaaaatgtatacctctgaagatgcagcaactgcgggtgcgaaacggataacggtttggtaggtgtatgtcgcagcatgtgctggattttgcgggcaactccccctgccacggcccattgcaggtgtcatgttgactggaggatacgaactttcggccgtcaaattcccagaggaggaccccctctgggccgacattgtgcccactgctcttccgcttgctgtttcctgtgtgaaattgt

SEQ ID NO: 21 - DNA, 3499 bp, synthetic construct
  source 1..3499: mol_type = other DNA
gctcttccgcttggagtcactgtgccactgagttcgactggtagctgaatggagtcgctgctccactaaacgaattgtcagcaccgccagccggccgaggacccgagtcatagcgagggtagtagcgcgccatggcaccgaccagcctgcttgccagtactggcgtctcttccgcttctctgtggtcctctgcgcgctccagcgcgtgcgcttttccggtggatcatgcggtccgtggcgcaccgcagcggccgctgcccatgcagcgccgctgcttccgaacagtggcggtcagggccgcacccgcggtagccgtccgtccggaacccgcccaagagttttgggagcagcttgagccctgcaagatggcggaggacaagcgcatcttcctggaggagcaccggtgcgtggaggtccggggctgaccggccgtcgcattcaacgtaatcaatcgcatgatgatcagaggacacgaagtcttggtggcggtggccagaaacactgtccattgcaagggcatagggatgcgttccttcacctctcatttctcatttctgaatccctccctgctcactctttctcctcctccttcccgttcacgcagcattcggggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcagcggccgcgacagggtggttggctggatggggaaacgctggtcgcgggattcgatcctgctgcttatatcctccctggaagcacacccacgactctgaagaagaaaacgtgcacacacacaacccaaccggccgaatatttgcttccttatcccgggtccaagagagactgcgatgcccccctcaatcagcatcctcctccctgccgcttcaatcttccctgcttgcctgcgcccgcggtgcgccgtctgcccgcccagtcagtcactcctgcacaggccccttgtgcgcagtgctcctgtaccctttaccgctccttccattctgcgaggccccctattgaatgtattcgttgcctgtgtggccaagcgggctgctgggcgcgccgccgtcgggcagtgctcggcgactttggcggaagccgattgttcttctgtaagccacgcgcttgctgctttgggaagagaagggggggggtactgaatggatgaggaggagaaggaggggtattggtattatctgagttgggtgctcttccgct

SEQ ID NO: 22 - DNA, 3352 bp, synthetic construct
  source 1..3352: mol_type = other DNA
cttaatggagtcgctgctccactaatcgaattgtcagcaccgccagccggccgaggacccgagtcatagcgagggtagtagcgcgccatggcaccgaccagcctgcttgcccgtactggcgtctcttccgcttctctgtgctcctctacgcgctccggcgcgtgcgcttttccggtggatcatgcggtccgtggcgcaccgcagcggccgctgcccatgcagcgccgctgcttccgaacagtggctgtcagggccgcacccgcagtagccgtccgtccggaacccgcccaagagttttgggagcagcttgagccctgcaagatggcggaggacaagcgcatcttcctggaggagcaccggtgcgcggaggtccggggctgaccggccgtcgcattcaacgtaatcaatcgcatgatgatcacaggacgcgacgtcttggtggcggtggccagggacactgcccattgcacaggcataggaatgcgttccttctcatttctcagttttctgagcccctccctcttcactctttctcctcctcctcccctctcacgcagcattcgtggggtaccctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacactagtatgctgctgcaggccttcctgttcctgctggccggcttcgccgccaagatcagcgcctccatgacgaacgagacgtccgaccgccccctggtgcacttcacccccaacaagggctggatgaacgaccccaacggcctgtggtacgacgagaaggacgccaagtggcacctgtacttccagtacaacccgaacgacaccgtctgggggacgcccttgttctggggccacgccacgtccgacgacctgaccaactgggaggaccagcccatcgccatcgccccgaagcgcaacgactccggcgccttctccggctccatggtggtggactacaacaacacctccggcttcttcaacgacaccatcgacccgcgccagcgctgcgtggccatctggacctacaacaccccggagtccgaggagcagtacatctcctacagcctggacggcggctacaccttcaccgagtaccagaagaaccccgtgctggccgccaactccacccagttccgcgacccgaaggtcttctggtacgagccctcccagaagtggatcatgaccgcggccaagtcccaggactacaagatcgagatctactcctccgacgacctgaagtcctggaagctggagtccgcgttcgccaacgagggcttcctcggctaccagtacgagtgccccggcctgatcgaggtccccaccgagcaggaccccagcaagtcctactgggtgatgttcatctccatcaaccccggcgccccggccggcggctccttcaaccagtacttcgtcggcagcttcaacggcacccacttcgaggccttcgacaaccagtcccgcgtggtggacttcggcaaggactactacgccctgcagaccttcttcaacaccgacccgacctacgggagcgccctgggcatcgcgtgggcctccaactgggagtactccgccttcgtgcccaccaacccctggcgctcctccatgtccctcgtgcgcaagttctccctcaacaccgagtaccaggccaacccggagacggagctgatcaacctgaaggccgagccgatcctgaacatcagcaacgccggcccctggagccggttcgccaccaacaccacgttgacgaaggccaacagctacaacgtcgacctgtccaacagcaccggcaccctggagttcgagctggtgtacgccgtcaacaccacccagacgatctccaagtccgtgttcgcggacctctccctctggttcaagggcctggaggaccccgaggagtacctccgcatgggcttcgaggtgtccgcgtcctccttcttcctggaccgcgggaacagcaaggtgaagttcgtgaaggagaacccctacttcaccaaccgcatgagcgtgaacaaccagcccttcaagagcgagaacgacctgtcctactacaaggtgtacggcttgctggaccagaacatcctggagctgtacttcaacgacggcgacgtcgtgtccaccaacacctacttcatgaccaccgggaacgccctgggctccgtgaacatgacgacgggggtggacaacctgttctacatcgacaagttccaggtgcgcgaggtcaagtgattaattaagcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctgcagggcggccgcagaggagagggtggctggtagttgcgggatggctggtcgcccgtcgatcctgctgctgctattgtctcctcctgcacaagcccacccacgactccgaagaagaagaagaaaacgcgcacacacacaacccaaccggccgaatatttgcttccttatcccgggtccaagagagacggcgatgcccccctcaatcagcctcctcctccctgccgctccaatcttccctgcttgcatgcgcccgcgagaggctgtctgcgcgccccgtcagtcactccccgtgcagacgcctcgtgctcggtgctcctgtatcctttaccgctcctttcattctgcgaggccccctgttgaatgtattcgttgcctgtgtggccaagcgcgctgctgggcgcgccgccgtcgggcggtgctcggcgactctggcggaagccggttgttct

SEQ ID NO: 23 - DNA, 3344 bp, synthetic construct
  source 1..3344: mol_type = other DNA
cttaatggagtcgctgctccactaatcgaattgtcagcaccgccagccggccgaggacccgagtcatagcgagggtagtagcgcgccatggcaccgaccagcctgcttgcccgtactggcgtctcttccgcttctctgtgctcctctacgcgctccggcgcgtgcgcttttccggtggatcatgcggtccgtggcgcaccgcagcggccgctgcccatgcagcgccgctgcttccgaacagtggctgtcagggccgcacccgcagtagccgtccgtccggaacccgcccaagagttttgggagcagcttgagccctgcaagatggcggaggacaagcgcatcttcctggaggagcaccggtgcgcggaggtccggggctgaccggccgtcgcattcaacgtaatcaatcgcatgatgatcacaggacgcgacgtcttggtggcggtggccagggacactgcccattgcacaggcataggaatgcgttccttctcatttctcagttttctgagcccctccctcttcactctttctcctcctcctcccctctcacgcagcattcgtggccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcagaggagagggtggctggtagttgcgggatggctggtcgcccgtcgatcctgctgctgctattgtctcctcctgcacaagcccacccacgactccgaagaagaagaagaaaacgcgcacacacacaacccaaccggccgaatatttgcttccttatcccgggtccaagagagacggcgatgcccccctcaatcagcctcctcctccctgccgctccaatcttccctgcttgcatgcgcccgcgagaggctgtctgcgcgccccgtcagtcactccccgtgcagacgcctcgtgctcggtgctcctgtatcctttaccgctcctttcattctgcgaggccccctgttgaatgtattcgttgcctgtgtggccaagcgcgctgctgggcgcgccgccgtcgggcggtgctcggcgactctggcggaagccggttgttct

SEQ ID NO: 24 - DNA, 5583 bp, synthetic construct
  source 1..5583: mol_type = other DNA
ccctcaactgcgacgctgggaaccttctccgggcaggcgatgtgcgtgggtttgcctccttggcacggctctacaccgtcgagtacgccatgaggcggtgatggctgtgtcggttgccacttcgtccagagacggcaagtcgtccatcctctgcgtgtgtggcgcgacgctgcagcagtccctctgcagcagatgagcgtgactttggccatttcacgcactcgagtgtacacaatccatttttcttaaagcaaatgactgctgattgaccagatactgtaacgctgatttcgctccagatcgcacagatagcgaccatgttgctgcgtctgaaaatctggattccgaattcgaccctggcgctccatccatgcaacagatggcgacacttgttacaattcctgtcacccatcggcatggagcaggtccacttagattcccgatcacccacgcacatctcgctaatagtcattcgttcgtgtcttcgatcaatctcaagtgagtgtgcatggatcttggttgacgatgcggtatgggtttgcgccgctggctgcagggtctgcccaaggcaagctaacccagctcctctccccgacaatactctcgcaggcaaagccggtcacttgccttccagattgccaataaactcaattatggcctctgtcatgccatccatgggtctgatgaatggtcacgctcgtgtcctgaccgttccccagcctctggcgtcccctgccccgcccaccagcccacgccgcgcggcagtcgctgccaaggctgtctcggaggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaatcgatgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaatcgatctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatggctatcaagacgaacaggcagcctgtggagaagcctccgttcacgatcgggacgctgcgcaaggccatccccgcgcactgtttcgagcgctcggcgcttcgtagcagcatgtacctggcctttgacatcgcggtcatgtccctgctctacgtcgcgtcgacgtacatcgaccctgcaccggtgcctacgtgggtcaagtacggcatcatgtggccgctctactggttcttccaggtgtgtttgagggttttggttgcccgtattgaggtcctggtggcgcgcatggaggagaaggcgcctgtcccgctgacccccccggctaccctcccggcaccttccagggcgcgtacgggaagaaccagtagagcggccacatgatgccgtacttgacccacgtaggcaccggtgcagggtcgatgtacgtcgacgcgacgtagagcagggacatgaccgcgatgtcaaaggccaggtacatgctgctacgaagcgccgagcgctcgaaacagtgcgcggggatggccttgcgcagcgtcccgatcgtgaacggaggcttctccacaggctgcctgttcgtcttgatagccatcctagggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctgcagggcggccgccagcgccatgccacgccctttgatggcttcaagtacgattacggtgttggattgtgtgtttgttgcgtagtgtgcatggtttagaataatacacttgatttcttgctcacggcaatctcggcttgtccgcaggttcaaccccatttcggagtctcaggtcagccgcgcaatgaccagccgctacttcaaggacttgcacgacaacgccgaggtgagctatgtttaggacttgattggaaattgtcgtcgacgcatattcgcgctccgcgacagcacccaagcaaaatgtcaagtgcgttccgatttgcgtccgcaggtcgatgttgtgatcgtcggcgccggatccgccggtctgtcctgcgcttacgagctgaccaagcaccctgacgtccgggtacgcgagctgagattcgattagacataaattgaagattaaacccgtagaaaaatttgatggtcgcgaaactgtgctcgattgcaagaaattgatcgtcctccactccgcaggtcgccatcatcgagcagggcgttgctcccggcggcggcgcctggctggggggacagctgttctcggccatgtgtgtacgtagaaggatgaatttcagctggttttcgttgcacagctgtttgtgcatgatttgtttcagactattgttgaatgtttttagatttcttaggatgcatgatttgtctgcatgcgact

SEQ ID NO: 25 - DNA, 5092 bp, synthetic construct
  source 1..5092: mol_type = other DNA
cttaatggagtcgctgctccactaatcgaattgtcagcaccgccagccggccgaggacccgagtcatagcgagggtagtagcgcgccatggcaccgaccagcctgcttgcccgtactggcgtctcttccgcttctctgtgctcctctacgcgctccggcgcgtgcgcttttccggtggatcatgcggtccgtggcgcaccgcagcggccgctgcccatgcagcgccgctgcttccgaacagtggctgtcagggccgcacccgcagtagccgtccgtccggaacccgcccaagagttttgggagcagcttgagccctgcaagatggcggaggacaagcgcatcttcctggaggagcaccggtgcgcggaggtccggggctgaccggccgtcgcattcaacgtaatcaatcgcatgatgatcacaggacgcgacgtcttggtggcggtggccagggacactgcccattgcacaggcataggaatgcgttccttctcatttctcagttttctgagcccctccctcttcactctttctcctcctcctcccctctcacgcagcattcgtggggtaccccgctcccgtctggtcctcacgttcgtgtacggcctggatcccggaaagggcggatgcacgtggtgttgccccgccattggcgcccacgtttcaaagtccccggccagaaatgcacaggaccggcccggctcgcacaggccatgacgaatgcccagatttcgacagcaaaacaatctggaataatcgcaaccattcgcgttttgaacgaaacgaaaagacgctgtttagcacgtttccgatatcgtgggggccgaagcatgattggggggaggaaagcgtggccccaaggtagcccattctgtgccacacgccgacgaggaccaatccccggcatcagccttcatcgacggctgcgccgcacatataaagccggacgccttcccgacacgttcaaacagttttatttcctccacttcctgaatcaaacaaatcttcaaggaagatcctgctcttgagcaactcgtatgttcgcgttctacttcctgacggcctgcatctccctgaagggcgtgttcggcgtctccccctcctacaacggcctgggcctgacgccccagatgggctgggacaactggaacacgttcgcctgcgacgtctccgagcagctgctgctggacacggccgaccgcatctccgacctgggcctgaaggacatgggctacaagtacatcatcctggacgactgctggtcctccggccgcgactccgacggcttcctggtcgccgacgagcagaagttccccaacggcatgggccacgtcgccgaccacctgcacaacaactccttcctgttcggcatgtactcctccgcgggcgagtacacgtgcgccggctaccccggctccctgggccgcgaggaggaggacgcccagttcttcgcgaacaaccgcgtggactacctgaagtacgacaactgctacaacaagggccagttcggcacgcccgagatctcctaccaccgctacaaggccatgtccgacgccctgaacaagacgggccgccccatcttctactccctgtgcaactggggccaggacctgaccttctactggggctccggcatcgcgaactcctggcgcatgtccggcgacgtcacggcggagttcacgcgccccgactcccgctgcccctgcgacggcgacgagtacgactgcaagtacgccggcttccactgctccatcatgaacatcctgaacaaggccgcccccatgggccagaacgcgggcgtcggcggctggaacgacctggacaacctggaggtcggcgtcggcaacctgacggacgacgaggagaaggcgcacttctccatgtgggccatggtgaagtcccccctgatcatcggcgcgaacgtgaacaacctgaaggcctcctcctactccatctactcccaggcgtccgtcatcgccatcaaccaggactccaacggcatccccgccacgcgcgtctggcgctactacgtgtccgacacggacgagtacggccagggcgagatccagatgtggtccggccccctggacaacggcgaccaggtcgtggcgctgctgaacggcggctccgtgtcccgccccatgaacacgaccctggaggagatcttcttcgactccaacctgggctccaagaagctgacctccacctgggacatctacgacctgtgggcgaaccgcgtcgacaactccacggcgtccgccatcctgggccgcaacaagaccgccaccggcatcctgtacaacgccaccgagcagtcctacaaggacggcctgtccaagaacgacacccgcctgttcggccagaagatcggctccctgtcccccaacgcgatcctgaacacgaccgtccccgcccacggcatcgcgttctaccgcctgcgcccctcctcctgatacaacttattacgtattctgaccggcgctgatgtggcgcggacgccgtcgtactctttcagactttactcttgaggaattgaacctttctcgcttgctggcatgtaaacattggcgcaattaattgtgtgatgaagaaagggtggcacaagatggatcgcgaatgtacgagatcgacaacgatggtgattgttatgaggggccaaacctggctcaatcttgtcgcatgtccggcgcaatgtgatccagcggcgtgactctcgcaacctggtagtgtgtgcgcaccgggtcgctttgattaaaactgatcgcattgccatcccgtcaactcacaagcctactctagctcccattgcgcactcgggcgcccggctcgatcaatgttctgagcggagggcgaagcgtcaggaaatcgtctcggcagctggaagcgcatggaatgcggagcggagatcgaatcaatcgatgcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggaatctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaactctagaatggctatcaagacgaacaggcagcctgtggagaagcctccgttcacgatcgggacgctgcgcaaggccatccccgcgcactgtttcgagcgctcggcgcttcgtagcagcatgtacctggcctttgacatcgcggtcatgtccctgctctacgtcgcgtcgacgtacatcgaccctgcaccggtgcctacgtgggtcaagtacggcatcatgtggccgctctactggttcttccaggtgtgtttgagggttttggttgcccgtattgaggtcctggtggcgcgcatggaggagaaggcgcctgtcccgctgacccccccggctaccctcccggcaccttccagggcgcgtacgggaagaaccagtagagcggccacatgatgccgtacttgacccacgtaggcaccggtgcagggtcgatgtacgtcgacgcgacgtagagcagggacatgaccgcgatgtcaaaggccaggtacatgctgctacgaagcgccgagcgctcgaaacagtgcgcggggatggccttgcgcagcgtcccgatcgtgaacggaggcttctccacaggctgcctgttcgtcttgatagccatcctagggcagcagcagctcggatagtatcgacacactctggacgctggtcgtgtgatggactgttgccgccacacttgctgccttgacctgtgaatatccctgccgcttttatcaaacagcctcagtgtgtttgatcttgtgtgtacgcgcttttgcgagttgctagctgcttgtgctatttgcgaataccacccccagcatccccttccctcgtttcatatcgcttgcatcccaaccgcaacttatctacgctgtcctgctatccctcagcgctgctcctgctcctgctcactgcccctcgcacagccttggtttgggctccgcctgtattctcctggtactgcaacctgtaaaccagcactgcaatgctgatgcacgggaagtagtgggatgggaacacaaatggacctgcagggcggccgcagaggagagggtggctggtagttgcgggatggctggtcgcccgtcgatcctgctgctgctattgtctcctcctgcacaagcccacccacgactccgaagaagaagaagaaaacgcgcacacacacaacccaaccggccgaatatttgcttccttatcccgggtccaagagagacggcgatgcccccctcaatcagcctcctcctccctgccgctccaatcttccctgcttgcatgcgcccgcgagaggctgtctgcgcgccccgtcagtcactccccgtgcagacgcctcgtgctcggtgctcctgtatcctttaccgctcctttcattctgcgaggccccctgttgaatgtattcgttgcctgtgtggccaagcgcgctgctgggcgcgccgccgtcgggcggtgctcggcgactctggcggaagccggttgttct

(19) United States
(12) Patent Application Publication
(10) Pub. No.: US 2025/0248921 A1
(43) Pub. Date: Aug. 7, 2025

(54) Ricinoleate Oil Production and Uses Thereof

(71) Applicant: Checkerspot, Inc., Alameda, CA (US)

(72) Inventors: Xinhua Zhao, Dublin, CA (US); Xiaoying Zhou, Dublin, CA (US); Jesus Barajas, Oakland, CA (US); Estelle Schaefer, San Pablo, CA (US); Leon H. Parker, IV, San Francisco, CA (US); Scott Franklin, Woodside, CA (US)

(73) Assignee: Checkerspot, Inc., Alameda, CA (US)

(21) Appl. No.: 18/854,722

(22) PCT Filed: Apr. 6, 2023
(86) PCT No.: PCT/US2023/065466

§ 371 Date: Oct. 7, 2024

Related U.S. Application Data
(60) Provisional application No. 63/328,458, filed on Apr. 7, 2022.

(51) Int. Cl. A61K 8/92; A61Q 17/04; C12N 9/02; C12N 9/06; C12N 9/10; C12N 9/16; C12N 15/82; C12P 7/6409

(52) U.S. Cl. CPC A61K 8/922; A61Q 17/04; C12N 9/0044; C12N 9/0071; C12N 9/1029; C12N 9/16; C12N 15/82; C12P 7/6409; C12Y 107/01001; C12Y 203/01158; C12Y 301/02014; C12N 2800/10

(57) ABSTRACT

Provided herein are triglyceride oil compositions enriched in ricinoleic acid. Further provided herein are methods of producing non-naturally occurring triglyceride oil compositions enriched in ricinoleic acid from non-naturally occurring microorganisms and applications thereof in a variety of end products, including, for example, polyols, polyurethane products, lubricants, personal care products, and food products.

### CROSS-REFERENCE

This application claims the benefit of U.S. Provisional Application No. 63/328,458, filed on Apr. 7, 2022, which is incorporated herein by reference in its entirety.

### SEQUENCE LISTING

The instant application contains a Sequence Listing which has been submitted electronically in XML file format and is hereby incorporated by reference in its entirety. Said XML copy, created on Mar. 28, 2023, is named 50727-728_601_SL, and is 126,499 bytes in size.

### BACKGROUND

Oleaginous microorganisms have the ability to convert carbon substrates into oils, including triacylglycerides (TAGs) or lipids, and accumulate these oils intracellularly. Some microorganisms can have capacity to accumulate lipids in amounts of up to 80% dry weight. Thus, oleaginous microorganisms, including microalgae, bacteria, fungi, and yeasts, can serve as an ideal source for biobased oil production. Genetic and non-genetic modification techniques can allow for the production of non-naturally occurring oils having particular fatty acid profiles.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a flow diagram for the preparation and analysis of fatty acids produced by P. moriformis strain CHK64.

FIG. 2 shows ricinoleate levels in untransformed P. moriformis strain CHK22 (strain A), CHK22 transformed with LFAH12 (strain B), CHK22 transformed with RcFAH12 (strain C), and CHK22 transformed with CpFAH12 (strain D).

FIG. 3 depicts an overview of de novo triacylglycerol biosynthesis in microalgae and higher plant oilseeds. Abbreviations for lipid classes (gray boxes): G3P, glycerol-3-phosphate; LPA, lysophosphatidic acid; PA, phosphatidic acid; DAG, diacylglycerol; PC, phosphatidylcholine; TAG, triacylglycerol. Abbreviations for enzymes (no boxes): GPAT, G3P acyltransferase; LPAT, lysophosphatidylacyltransferase; PAP, phosphatidate phosphatase; PDCT, PC:DAG cholinephosphotransferase; DGAT, acyl-CoA:DAG acyltransferase; PDAT, phospholipid:DAG acyltransferase.

FIG. 4 shows ricinoleate levels in untransformed P. moriformis strain CHK22 (strain A), CHK22 with expression of LFAH12 (strain B), strain B with expression of RcPDAT1 (strain E), strain B with expression of RcPDAT2 (strain F), strain B with expression of RcPDCT1 (strain G), and strain B with expression of RcDGAT2 (strain H).

FIG. 5 shows comparisons of oleate and ricinoleate levels in strains disclosed herein. FIG. 5, Panel A shows oleate levels in CHK22 (strain A) as compared to CHK48 (strain I). FIG. 5, Panel B shows ricinoleate levels in CHK22 with expression of LFAH12 (strain B) as compared to CHK48 with expression of LFAH12 (strain J).

FIG. 6 shows a diagram of ricinoleate TAG formation. Delta-12 fatty acid desaturase (PmFAD2) and oleate 12-hydroxylase (LFAH12) both use oleic acid as a substrate, while acyl-ACP thioesterases, FATA-1 and FATA-2, cleave fatty acyl groups from acyl carrier protein (ACP), which terminates fatty acid elongation at 18 carbons.

FIG. 7 shows ricinoleate levels in a high oleic base strain (strain K), a high oleic base strain with expression of optimized LFAH12 constructs (strains L and M), strain L with down-regulation of FATA-2 and PmFAD2 (strain N) and strain M with down-regulation of FATA-2 and PmFAD2 (strain O). Biomass was collected after incubation in M22 pH 7.0 media for FAME analysis.

### SUMMARY

In some embodiments, provided herein is a non-naturally occurring oil (e.g., a TAG oil) having a fatty acid profile comprising:
at least 10% C18:1; and
at least 1% of a hydroxylated C18:1.

In some embodiments, at least 10% of the fatty acids identified in the fatty acid profile are C18:1; and at least 1% of the fatty acids identified in the fatty acid profile are hydroxylated C18:1.

In some embodiments, the fatty acid profile is determined by gas chromatography-flame ionization detection (GC/FID).

In some embodiments, the fatty acid profile comprises at least 30% of the hydroxylated C18:1. In some embodiments, at least 30% of the fatty acids identified in the fatty acid profile are hydroxylated C18:1.

In some embodiments, the fatty acid profile comprises at least 50% of the hydroxylated C18:1. In some embodiments, at least 50% of the fatty acids identified in the fatty acid profile are hydroxylated C18:1.

In some embodiments, the fatty acid profile comprises at least 60% of the hydroxylated C18:1. In some embodiments, at least 60% of the fatty acids identified in the fatty acid profile are hydroxylated C18:1.

In some embodiments, the fatty acid profile comprises 30-40% of the hydroxylated C18:1. In some embodiments, 30-40% of the fatty acids identified in the fatty acid profile are hydroxylated C18:1.

In some embodiments, the fatty acid profile comprises 50-60% of the hydroxylated C18:1. In some embodiments, 50-60% of the fatty acids identified in the fatty acid profile are hydroxylated C18:1.

In some embodiments, the fatty acid profile comprises 60-70% of the hydroxylated C18:1. In some embodiments, 60-70% of the fatty acids identified in the fatty acid profile are hydroxylated C18:1.

In some embodiments, the hydroxylated C18:1 is 12-OH-C18:1.

In some embodiments, the hydroxylated C18:1 is ricinoleic acid.

In some embodiments, the fatty acid profile comprises at least 20% C18:1. In some embodiments, at least 20% of the fatty acids identified in the fatty acid profile are C18:1.

In some embodiments, the fatty acid profile comprises at least 30% C18:1. In some embodiments, at least 30% of the fatty acids identified in the fatty acid profile are C18:1.

In some embodiments, the fatty acid profile comprises 20-30% C18:1. In some embodiments, 20-30% of the fatty acids identified in the fatty acid profile are C18:1.

In some embodiments, the fatty acid profile comprises 30-40% C18:1. In some embodiments, 30-40% of the fatty acids identified in the fatty acid profile are C18:1.

In some embodiments, the C18:1 is oleic acid.

In some embodiments, the fatty acid profile further comprises less than 5% C16:0. In some embodiments, less than 5% of the fatty acids identified in the fatty acid profile are C16:0.

In some embodiments, the fatty acid profile further comprises at least 20% C16:0. In some embodiments, less than 20% of the fatty acids identified in the fatty acid profile are C16:0.

In some embodiments, the fatty acid profile further comprises at least 30% C16:0. In some embodiments, less than 30% of the fatty acids identified in the fatty acid profile are C16:0.

In some embodiments, the fatty acid profile further comprises 1-5% C16:0. In some embodiments, 1-5% of the fatty acids identified in the fatty acid profile are C16:0.

In some embodiments, the fatty acid profile further comprises 20-30% C16:0. In some embodiments, 20-30% of the fatty acids identified in the fatty acid profile are C16:0.

In some embodiments, the fatty acid profile further comprises 30-40% C16:0. In some embodiments, 30-40% of the fatty acids identified in the fatty acid profile are C16:0.

In some embodiments, the fatty acid profile further comprises less than 20% C18:2. In some embodiments, less than 20% of the fatty acids identified in the fatty acid profile are C18:2.

In some embodiments, the fatty acid profile further comprises at least 20% C18:2. In some embodiments, at least 20% of the fatty acids identified in the fatty acid profile are C18:2.

In some embodiments, the fatty acid profile further comprises at least 30% C18:2. In some embodiments, at least 30% of the fatty acids identified in the fatty acid profile are C18:2.

In some embodiments, the fatty acid profile further comprises 20-30% C18:2. In some embodiments, 20-30% of the fatty acids identified in the fatty acid profile are C18:2.

In some embodiments, the fatty acid profile further comprises 30-40% C18:2. In some embodiments, 30-40% of the fatty acids identified in the fatty acid profile are C18:2.

In some embodiments, the C18:2 is linoleic acid.

In some embodiments, the fatty acid profile further comprises less than 20% of saturated fatty acids. In some embodiments, less than 20% of the fatty acids identified in the fatty acid profile are saturated fatty acids.

In some embodiments, the fatty acid profile further comprises at least 5% of saturated fatty acids. In some embodiments, at least 5% of the fatty acids identified in the fatty acid profile are saturated fatty acids.

In some embodiments, the fatty acid profile further comprises at least 30% of saturated fatty acids. In some embodiments, at least 30% of the fatty acids identified in the fatty acid profile are saturated fatty acids.

In some embodiments, the fatty acid profile further comprises 5-10% of saturated fatty acids. In some embodiments, 5-10% of the fatty acids identified in the fatty acid profile are saturated fatty acids.

In some embodiments, the fatty acid profile further comprises 10-20% of saturated fatty acids. In some embodiments, 10-20% of the fatty acids identified in the fatty acid profile are saturated fatty acids.

In some embodiments, the fatty acid profile comprises 20-30% of saturated fatty acids. In some embodiments, 20-30% of the fatty acids identified in the fatty acid profile are saturated fatty acids.

In some embodiments, the saturated fatty acids are midchain saturated fatty acids.

In some embodiments, the midchain fatty acids comprise C10 and C12 fatty acids.

In some embodiments, the fatty acid profile comprises at least 20% of one or more midchain fatty acids. In some embodiments, at least 20% of the fatty acids identified in the fatty acid profile are midchain fatty acids.

In some embodiments, the fatty acid profile comprises at least 30% of one or more midchain fatty acids. In some embodiments, at least 30% of the fatty acids identified in the fatty acid profile are midchain fatty acids.

In some embodiments, the fatty acid profile comprises 20-30% of one or more midchain fatty acids. In some embodiments, 20-30% of the fatty acids identified in the fatty acid profile are midchain fatty acids.

In some embodiments, the fatty acid profile comprises 30-40% of one or more midchain fatty acids. In some embodiments, 30-40% of the fatty acids identified in the fatty acid profile are midchain fatty acids.

In some embodiments, the fatty acid profile further comprises 0.1-5% C10:0. In some embodiments, 0.1-5% of the fatty acids identified in the fatty acid profile are C10:0.

In some embodiments, the fatty acid profile further comprises 10-30% C12:0. In some embodiments, 10-30% of the fatty acids identified in the fatty acid profile are C12:0.

In some embodiments, the non-naturally occurring oil is a microalgal oil.

In some embodiments, provided herein is a non-naturally occurring oil (e.g., a TAG oil) having a fatty acid profile comprising:
at least 5% of saturated fatty acids; and
at least 1% of a hydroxylated C18:1.

In some embodiments, the fatty acid profile is determined by gas chromatography-flame ionization detection (GC/FID).

In some embodiments, the saturated fatty acids are midchain fatty acids.

In some embodiments, the fatty acid profile comprises at least 20% of one or more midchain fatty acids. In some embodiments, at least 20% of the fatty acids identified in the fatty acid profile are midchain fatty acids.

In some embodiments, the fatty acid profile comprises at least 30% of one or more midchain fatty acids. In some embodiments, at least 30% of the fatty acids identified in the fatty acid profile are midchain fatty acids.

In some embodiments, the fatty acid profile comprises 20-30% of one or more midchain fatty acids. In some embodiments, 20-30% of the fatty acids identified in the fatty acid profile are midchain fatty acids.

In some embodiments, the fatty acid profile comprises 30-40% of one or more midchain fatty acids. In some embodiments, 30-40% of the fatty acids identified in the fatty acid profile are midchain fatty acids.

In some embodiments, the midchain fatty acids comprise C10 and C12 fatty acids.

In some embodiments, the fatty acid profile further comprises 0.1-5% C10:0. In some embodiments, 0.1-5% of the fatty acids identified in the fatty acid profile are C10:0.

In some embodiments, the fatty acid profile further comprises 10-30% C12:0. In some embodiments, 10-30% of the fatty acids identified in the fatty acid profile are C12:0.

In some embodiments, the fatty acid profile comprises at least 5%, at least 10%, at least 15%, at least 20%, or at least 30% of the hydroxylated C18:1. In some embodiments, at least 5%, at least 10%, at least 15%, at least 20%, or at least 30% of the fatty acids identified in the fatty acid profile are hydroxylated C18:1.

In some embodiments, the fatty acid profile comprises at least 50% of the hydroxylated C18:1. In some embodiments, at least 50% of the fatty acids identified in the fatty acid profile are hydroxylated C18:1.

In some embodiments, the fatty acid profile comprises at least 60% of the hydroxylated C18:1. In some embodiments, at least 60% of the fatty acids identified in the fatty acid profile are hydroxylated C18:1.

In some embodiments, the fatty acid profile comprises 30-40% of the hydroxylated C18:1. In some embodiments, 30-40% of the fatty acids identified in the fatty acid profile are hydroxylated C18:1.

In some embodiments, the fatty acid profile comprises 50-60% of the hydroxylated C18:1. In some embodiments, 50-60% of the fatty acids identified in the fatty acid profile are hydroxylated C18:1.

In some embodiments, the fatty acid profile comprises 60-70% of the hydroxylated C18:1. In some embodiments, 60-70% of the fatty acids identified in the fatty acid profile are hydroxylated C18:1.

In some embodiments, the hydroxylated C18:1 is 12-OH-C18:1.

In some embodiments, the hydroxylated C18:1 is ricinoleic acid.

In some embodiments, the non-naturally occurring oil is a microalgal oil.

In some embodiments, provided herein is a composition comprising a non-naturally occurring oil (e.g., a TAG oil) described herein.

In some embodiments, the composition further comprises a UV filtering agent.

In some embodiments, the composition is a sunscreen.

In some embodiments, provided herein is a composition comprising a non-naturally occurring oil (e.g., a TAG oil) described herein having a fatty acid profile comprising:
at least 10% C18:1; and
at least 1% of a hydroxylated C18:1.

In some embodiments, at least 10% of the fatty acids identified in the fatty acid profile are C18:1 and at least 1% of the fatty acids identified in the fatty acid profile are hydroxylated C18:1.

In some embodiments, the composition further comprises a UV filtering agent.

In some embodiments, the composition is a sunscreen.

In some embodiments, provided herein is a composition comprising a non-naturally occurring oil (e.g., a TAG oil) described herein having a fatty acid profile comprising:
at least 5% of saturated fatty acids; and
at least 1% of a hydroxylated C18:1.

In some embodiments, at least 5% of the fatty acids identified in the fatty acid profile are saturated fatty acids and at least 1% of the fatty acids identified in the fatty acid profile are hydroxylated C18:1.

In some embodiments, the composition further comprises a UV filtering agent.

In some embodiments, the composition is a sunscreen.

In some embodiments, provided herein is a microalgal cell comprising an exogenous oleate 12-hydroxylase, wherein the cell produces an oil (e.g., a TAG oil) described herein.

In some embodiments, provided herein is a microalgal cell comprising an exogenous oleate 12-hydroxylase, wherein the cell produces an oil (e.g., a TAG oil) having a fatty acid profile comprising at least 1% of a hydroxylated C18:1.

In some embodiments, at least 1% of the fatty acids identified in the fatty acid profile are hydroxylated C18:1.

In some embodiments, the cell produces the oil described herein.

In some embodiments, the fatty acid profile is determined by gas chromatography-flame ionization detection (GC/FID).

In some embodiments, the exogenous oleate 12-hydroxylase is codon-optimized for expression in a Prototheca base strain.

In some embodiments, the cell is from a Prototheca base strain.

In some embodiments, the cell is from a non-genetically modified Prototheca base strain that produces an oil having a fatty acid profile of at least 50%, at least 60%, or at least 70% oleic acid. In some embodiments, at least 50%, at least 60%, or at least 70% of the fatty acids identified in the fatty acid profile are oleic fatty acids.

In some embodiments, the Prototheca base strain is Prototheca wickerhamii.

In some embodiments, the Prototheca base strain is Prototheca moriformis.

In some embodiments, the cell produces at least 60% lipid by dry cell weight.

In some embodiments, the exogenous oleate 12-hydroxylase is Lesquerella fendleri oleate 12-hydroxylase (LFAH12).

In some embodiments, the exogenous oleate 12-hydroxylase has at least 80%, at least 85%, at least 90%, at least 95%, or at least 99% sequence identity to the amino acid sequence of SEQ ID NO: 2 or SEQ ID NO: 5.

In some embodiments, the cell comprises a DNA sequence having at least 80%, at least 85%, at least 90%, at least 95%, or at least 99% sequence identity to the amino acid sequence of SEQ ID NO: 4.

In some embodiments, the exogenous oleate 12-hydroxylase is Ricinus communis oleate 12-hydroxylase (RcFAH12).

In some embodiments, the exogenous oleate 12-hydroxylase has at least 80%, at least 85%, at least 90%, at least 95%, or at least 99% sequence identity to the amino acid sequence of SEQ ID NO: 7.

In some embodiments, the exogenous oleate 12-hydroxylase is Claviceps purpurea oleate 12-hydroxylase (CpFAH12).

In some embodiments, the exogenous oleate 12-hydroxylase has at least 80%, at least 85%, at least 90%, at least 95%, or at least 99% sequence identity to the amino acid sequence of SEQ ID NO: 9.

In some embodiments, the cell further comprises an exogenous acyl-ACP thioesterase.

In some embodiments, the exogenous acyl-ACP thioesterase is a Lindera benzoin acyl-ACP thioesterase (FATB).

In some embodiments, the exogenous acyl-ACP thioesterase is L. benzoin FATB1.

In some embodiments, the exogenous thioesterase has at least 80%, at least 85%, at least 90%, at least 95%, or at least 99% sequence identity to the amino acid sequence of SEQ ID NO: 3.

In some embodiments, the cell comprises a DNA sequence having at least 80%, at least 85%, at least 90%, at least 95%, or at least 99% sequence identity to the amino acid sequence of SEQ ID NO: 1.

In some embodiments, the cell further comprises an exogenous acyltransferase.

In some embodiments, the exogenous acyltransferase is a phospholipid:diacylglycerol acyltransferase (PDAT).

In some embodiments, the exogenous acyltransferase is a R. communis PDAT.

In some embodiments, the exogenous acyltransferase is R. communis PDAT1.

In some embodiments, the exogenous acyltransferase has at least 80%, at least 85%, at least 90%, at least 95%, or at least 99% sequence identity to the amino acid sequence of SEQ ID NO: 11.

In some embodiments, the cell comprises a DNA sequence having at least 80%, at least 85%, at least 90%, at least 95%, or at least 99% sequence identity to the amino acid sequence of SEQ ID NO: 10.

In some embodiments, the exogenous acyltransferase is R. communis PDAT2.

In some embodiments, the exogenous acyltransferase has at least 80%, at least 85%, at least 90%, at least 95%, or at least 99% sequence identity to the amino acid sequence of SEQ ID NO: 13.

In some embodiments, the cell comprises a DNA sequence having at least 80%, at least 85%, at least 90%, at least 95%, or at least 99% sequence identity to the amino acid sequence of SEQ ID NO: 12.

In some embodiments, the exogenous acyltransferase is a phosphatidylcholine:diacylglycerol cholinephosphotransferase (PDCT).

In some embodiments, the exogenous acyltransferase is a R. communis PDCT.

In some embodiments, the exogenous acyltransferase is R. communis PDCT1.

In some embodiments, the exogenous acyltransferase has at least 80%, at least 85%, at least 90%, at least 95%, or at least 99% sequence identity to the amino acid sequence of SEQ ID NO: 15

In some embodiments, the cell comprises a DNA sequence having at least 80%, at least 85%, at least 90%, at least 95%, or at least 99% sequence identity to the amino acid sequence of SEQ ID NO: 14.

In some embodiments, the exogenous acyltransferase is an acyl-CoA:diacylglycerol acyltransferase (DGAT).

In some embodiments, the exogenous acyltransferase is a R. communis DGAT.

In some embodiments, the exogenous acyltransferase is R. communis DGAT2.

In some embodiments, the exogenous acyltransferase has at least 80%, at least 85%, at least 90%, at least 95%, or at least 99% sequence identity to the amino acid sequence of SEQ ID NO: 17.

In some embodiments, the cell comprises a DNA sequence having at least 80%, at least 85%, at least 90%, at least 95%, or at least 99% sequence identity to the amino acid sequence of SEQ ID NO: 16.

In some embodiments, the cell further comprises an exogenous three prime untranslated region (3′-UTR) downstream of the exogenous oleate 12-hydroxylase.

In some embodiments, the exogenous 3′-UTR is Chlorella vulgaris nitrate reductase (CvNR) 3′-UTR.

In some embodiments, the cell comprises a DNA sequence having at least 80%, at least 85%, at least 90%, at least 95%, or at least 99% sequence identity to the amino acid sequence of SEQ ID NO: 4.

In some embodiments, the exogenous 3′-UTR is P. moriformis phosphoglucokinase (PmPGK) 3′-UTR.

In some embodiments, the cell comprises a DNA sequence having at least 80%, at least 85%, at least 90%, at least 95%, or at least 99% sequence identity to the amino acid sequence of SEQ ID NO: 18.

In some embodiments, the exogenous 3′-UTR is P. moriformis phosphoglycerate dehydrogenase (PmPGH) 3′-UTR.

In some embodiments, the cell comprises a DNA sequence having at least 80%, at least 85%, at least 90%, at least 95%, or at least 99% sequence identity to the amino acid sequence of SEQ ID NO: 19.

In some embodiments, the exogenous 3′-UTR is P. moriformis heat shock protein 90 (PmHSP90) 3′-UTR.

In some embodiments, the cell comprises a DNA sequence having at least 80%, at least 85%, at least 90%, at least 95%, or at least 99% sequence identity to the amino acid sequence of SEQ ID NO: 20.

In some embodiments, the cell is genetically modified to decrease expression of an endogenous acyl-ACP thioesterase.

In some embodiments, the cell does not comprise an endogenous acyl-ACP thioesterase.

In some embodiments, the endogenous acyl-ACP thioesterase is FATA-1.

In some embodiments, the cell comprises a DNA sequence having at least 80%, at least 85%, at least 90%, at least 95%, or at least 99% sequence identity to the amino acid sequence of SEQ ID NO: 21.

In some embodiments, the endogenous acyl-ACP thioesterase is FATA-2.

In some embodiments, the cell comprises a DNA sequence having at least 80%, at least 85%, at least 90%, at least 95%, or at least 99% sequence identity to the amino acid sequence of SEQ ID NO: 22.

In some embodiments, the endogenous acyl-ACP thioesterase has at least 80%, at least 85%, at least 90%, at least 95%, or at least 99% sequence identity to the amino acid sequence of SEQ ID NO: 23.

In some embodiments, the cell is genetically modified to decrease expression of an endogenous delta-12 fatty acid desaturase.

In some embodiments, the cell does not comprise the endogenous delta-12 fatty acid desaturase.

In some embodiments, the endogenous delta-12 fatty acid desaturase is FAD-2.

In some embodiments, the cell further comprises an exogenous PmFAD2 hairpin sequence.

In some embodiments, the cell comprises a DNA sequence having at least 80%, at least 85%, at least 90%, at least 95%, or at least 99% sequence identity to the amino acid sequence of SEQ ID NO: 24.

In some embodiments, the cell comprises a DNA sequence having at least 80%, at least 85%, at least 90%, at least 95%, or at least 99% sequence identity to the amino acid sequence of SEQ ID NO: 25.

In some embodiments, provided herein is a method of producing an oil (e.g., a TAG oil) composition described herein, the method comprising cultivating a microalgal cell described herein in a culture medium.

In some embodiments, the cell produces an oil described herein.

In some embodiments, the cell is a cell described herein.

In some embodiments, the method further comprises isolating the oil composition from the culture medium.

In some embodiments, provided herein is a method of producing an oil (e.g., a TAG oil) composition, the method comprising cultivating a microalgal cell in a culture medium, wherein the oil composition has a fatty acid profile comprising at least 1% of a hydroxylated C18:1.

In some embodiments, the cell produces an oil described herein.

In some embodiments, the cell is a cell described herein.

In some embodiments, the method further comprises isolating the oil composition from the culture medium.

In some embodiments, provided herein is a method of producing an oil (e.g., a TAG oil) composition, the method comprising cultivating a microalgal cell in a culture medium, wherein the oil composition has a fatty acid profile comprising at least 5% of saturated fatty acids and at least 1% of a hydroxylated C18:1.

In some embodiments, the cell produces an oil described herein.

In some embodiments, the cell is a cell described herein.

In some embodiments, the method further comprises isolating the oil composition from the culture medium.

### DETAILED DESCRIPTION

Provided herein are oil compositions comprising TAGs enriched in ricinoleate, methods of making thereof, and formulations and applications thereof. Further provided herein are TAGs enriched in ricinoleate and midchain fatty acids, methods of making thereof, and formulations and applications thereof. Oil compositions (e.g., TAG oil compositions) provided herein can be produced by a microorganism that is genetically modified or non-genetically modified. Non-genetically modified microorganisms can be produced by classical strain improvement strategies such as those described herein. In turn, these non-naturally occurring microorganism can produce non-naturally occurring oils (e.g., TAG oils) provided herein. Genetically modified microorganisms can be derived from a base strain that is a non-genetically modified microorganism produced by classical strain improvement strategies.

Genetic and non-genetic modification techniques can allow for the production of non-naturally occurring oils (e.g., TAG oils) having particular phenotypes. While genetic engineering techniques can be targeted to phenotypes elicited in a host oleaginous microbe, classical strain improvement or other non-genetic engineering techniques can be employed to further enhance these phenotypes. Enhancement of phenotypes can include elaboration of a particular fatty acid profile, yield on carbon, volumetric oil accumulation (e.g., g oil/L culture), oil productivity (e.g., g oil/L culture day), and oil as a percent dry cell weight (DCW) as a measure of strain performance.

### Definitions

As used herein, the term “microbial oil” refers to an oil produced or extracted from a microorganism (microbe), e.g., an oleaginous, single-celled, eukaryotic, or prokaryotic microorganism, including but not limited to, microalgae, yeast, bacteria, and fungi.

As used herein, the term “natural oil,” “natural triglyceride oil,” or “naturally occurring oil” refers to an oil derived from a plant, animal, fungi, algae, or bacterium that has not undergone additional chemical or enzymatic manipulation. In some embodiments, the term can exclude refining processes, for example, degumming, refining, bleaching, or deodorization.

As used herein, the term “triacylglycerol”, “triglyceride”, or “TAG” refers to esters between glycerol and three saturated and/or unsaturated fatty acids. TAG can also refer to an oil composed of three saturated and/or unsaturated fatty acids held together by a glycerol backbone. Generally. Fatty acids of TAGs have chain lengths of 6 carbon atoms or more. Unless indicated otherwise, the oils and TAG oils described herein comprise TAG species.

As used herein, the term “TAG purity”, “molecular purity”, or “oil purity” refers to the number of molecular species that make up an oil composition, on an absolute basis or present in amounts above a certain threshold. The fewer the number of TAG species in an oil, the greater the “purity” of the oil.

As used herein, the term “polyol” refers to triglycerols or fatty acid alcohols comprising hydroxyl functional groups. As used herein, the term “polyol derived from a TAG oil” generally refers to a polyol obtained from chemical conversion of a TAG oil, e.g., via epoxidation and ring opening, ozonolysis, and reduction, or hydroformylation and reduction.

As used herein, the term “natural oil polyol” refers to a polyol produced in situ by a plant, animal, fungi, algae, or bacterium, or through chemical modifications of a TAG oil derived from a plant, animal, fungi, algae, or bacterium.

As used herein, the term “polyurethane”, “PU”, or “urethane” refers to a class of polymers comprised of carbamate (urethane) linkages formed between a polyol and an isocyanate moiety.

As used herein, the term “oleic content”, “oleate content”, or “olein content” refers the percentage amount of oleic acid in the fatty acid profile of a substance (e.g., a TAG). As used herein, the term “C18:1 content” refers the percentage amount of a C18:1 fatty acid (e.g., oleic acid) in the fatty acid profile of a substance (e.g., a microbial oil). Unless indicated otherwise, a C18:1 fatty acid described herein does not comprise a hydroxyl functional group.

As used herein, the term “high oleic” can refer to greater than 60% oleic acid, greater than 70% oleic acid, greater than 80% oleic acid, or greater than 90% oleic acid.

As used herein, the term “ricinoleic content”, “ricinoleate content”, or “ricinolein content” refers the percentage amount of ricinoleic acid in the fatty acid profile of a substance (e.g., a TAG).

As used herein, the term “high ricinoleic” can refer to greater than 30% ricinoleic acid, greater than 40% ricinoleic acid, greater than 50% ricinoleic acid, greater than 60% ricinoleic acid, greater than 70% ricinoleic acid, greater than 80% ricinoleic acid, or greater than 90% ricinoleic acid.

As used herein, the term “hydroxylated C18:1” or “C18:1-OH” refers to a C: 18:1 fatty acid having a hydroxyl functional group attached to the fatty acid chain. In some embodiments, a hydroxylated C18:1 is ricinoleic acid.

As used herein, the term “polyunsaturated fatty acid” or “PUFA” refers to a fatty acid having more than one double bond in the backbone. For example, C18:2 is a polyunsaturated fatty acid.

As used herein, the term “medium chain fatty acids,” “midchain fatty acids,” or “MCFA” refers to C8, C10, or C12 fatty acids. A “midchain TAG oil” or “MCT oil” refers to a TAG oil containing C8, C10, or C12 fatty acids.

As used herein, unless otherwise stated, a percent (%) of a fatty acid species in an oil herein refers to a normalized area percent (area %) of a peak corresponding to the fatty acid species from a GC-FID chromatogram as determined by FAMEs analysis of the oil using GC-FID.

As used herein, unless otherwise stated, a percent (%) of a TAG species in an oil herein refers to a normalized area percent (area %) of a peak corresponding to the TAG species from a mass spectrum as determined by TAG analysis of the oil using Liquid Chromatography/Time of Flight-Mass Spectrometry (LC/MS).

As used herein, the term “about” refers to +10% from the value provided.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are described herein.

### Microbial Oils

In some embodiments, an oil (e.g., a TAG oil) provided herein is obtained from a genetically modified microorganism, for example, microalgae, oleaginous yeast, or oleaginous bacteria. In some embodiments, the genetically modified microorganism is a genetically modified Prototheca sp. strain. In some embodiments, a genetically modified microorganism comprises an exogenous gene or exogenous nucleotides. Alternatively, or additionally, a genetically modified microorganism does not comprise an endogenous gene or endogenous nucleotides.

In other embodiments, an oil (e.g., a TAG oil) provided herein is obtained from a non-genetically modified microorganism, for example, microalgae, oleaginous yeast, or oleaginous bacteria. In some embodiments, the non-genetically modified microorganism is a non-genetically modified Prototheca sp. strain. The non-genetically modified Prototheca sp. strain can be produced by classical strain improvement strategies. For example, a non-genetically modified microorganism does not comprise an exogenous gene or exogenous nucleotides. In some embodiments, the microorganism does not comprise an exogenous gene encoding an active ketoacyl-ACP synthase.

In some embodiments, an oil (e.g., a TAG oil) provided herein is produced by microalgae. In some embodiments, the microalgae is a species of a genus selected from the group consisting of: Chlorella sp., Pseudochlorella sp., Heterochlorella sp., Prototheca sp., Arthrospira sp., Euglena sp., Nannochloropsis sp., Phaeodactylum sp., Chlamydomonas sp., Scenedesmus sp., Ostreococcus sp., Selenastrum sp., Haematococcus sp., Nitzschia, Dunaliella, Navicula sp., Trebouxia sp., Pseudotrebouxia sp., Vavicula sp., Bracteococcus sp., Gomphonema sp., Watanabea, sp., Botryococcus sp., Tetraselmis sp., and Isochrysis sp. In some embodiments, the microalgae is Prototheca sp. In some embodiments, the microalgae is P. moriformis. In some embodiments, the microalgae is P. wickerhamii.

In some embodiments, an oil (e.g., a TAG oil) provided herein is produced by oleaginous yeast. In some embodiments, the oleaginous yeast is a species of a genus selected from the group consisting of: Candida sp., Cryptococcus sp., Debaromyces sp., Endomycopsis sp., Geotrichum sp., Hyphopichia sp., Lipomyces sp., Pichia, sp., Rodosporidium sp., Rhodotorula sp., Sporobolomyces sp., Starmerella sp., Torulaspora sp., Trichosporon sp., Wickerhamomyces sp., Yarrowia sp., and Zygoascus sp.

In some embodiments, an oil (e.g., a TAG oil) provided herein is obtained from or produced by oleaginous bacteria. In some embodiments, the oleaginous bacteria is a species selected from the group consisting of: Flavimonas oryzihabitans, Pseudomonas aeruginosa, Morococcus sp., Rhodobacter sphaeroides, Rhodococcus opacus, Rhodococcus erythropolis, Streptomyces jeddahensis, Ochrobactrum sp., Arthrobacter sp., Nocardia sp., Mycobacteria sp., Gordonia sp., Catenisphaera sp., and Dietzia sp.

Further provided herein are bioreactors comprising a non-naturally occurring microorganism provided herein. For example, these bioreactors comprise an oleaginous, non-naturally occurring microorganism.

### Classical Strain Improvement

Classical strain improvement strategies can be used to select for organisms having desired phenotypes, e.g., high oleic oil production. Classical strain improvement (also called “mutation breeding”) involves exposing organisms to chemicals or radiation to generate mutants with desirable traits. Ultraviolet (UV) light can be used to introduce random mutations within a microorganism's nuclear genome. Chemical mutagens include compounds which inhibit or disrupt biosynthetic processes of a microorganism, e.g., antibiotics, antifungals, or carcinogens. Non-limiting examples of chemical mutagens include ICR-191, L-canavanine, cerulenin, ethyl methanesulfonate (EMS), triparanol, phenethyl alcohol, 4-nitroquinoline-1-oxide (4-NQO), clomiphene citrate, terfenadine, fluphenazine, AZD-8055, BASF 13-338, cafenstrole, clomiphene, and PF-042110. Combinations of chemical mutagens can also be used simultaneously to induce mutagenesis.

Methods provided herein include classical strain improvement methods to improve strain productivity, carbon yield, and oleic acid content. Glucose consumption rate can be highly predictive indicator of lipid titer. As such, glucose consumption rate can be used as an enrichment tool in the mutant selection process.

Methods provided herein also include the genetic modification of organisms produced from classical strain improvement methods.

### High Ricinoleic Oils of the Disclosure

Ricinoleic acid (12-hydroxy-9-cis-octadecenoic acid) is a high-value hydroxy fatty acid with broad industrial applications. Ricinoleic acid is an unsaturated fatty acid with a double bond and hydroxyl group at positions C9 and C12, respectively. Ricinoleic acid has unusual polarity due to the position of its hydroxyl group, and is soluble in alcohol, acetone, ether, and chloroform. Ricinoleic acid is a major component of the seed oil obtained from mature castor plant (Ricinus communis) seeds or in the sclerotium of ergot (Claviceps purpurea). About 90% of the fatty acid content in castor oil is the triglyceride formed from ricinoleic acid.

Ricinoleic acid is used in the oleochemical industry and can undergo a wide range of reactions to form multiple derivatives. Ricinoleic acid can be used in pigments, inks, detergents, lubricants, polyesters, biodiesel, and textiles. Derivatives of ricinoleic acid can be used as plasticizers, emulsifiers, antistatic, or softening agents. Ricinoleic acid can also be used as the starting material for various surfactants such as betaine and imidazoline.

Ricinoleic acid can be produced by hydrolysis of castor oil or other oils. Reaction conditions can be mild (e.g., 70-100° C.). Hydrolysis can be catalyzed by an enzyme (e.g., a lipase; a fatty acid hydroxylase). Oleate 12-hydroxylase (FAH) synthesizes ricinoleic acid from oleic acid through hydrolysis at the C12 position. Ricinoleic acid can also be produced by saponification or fractional distillation of hydrolyzed castor oil or other oils.

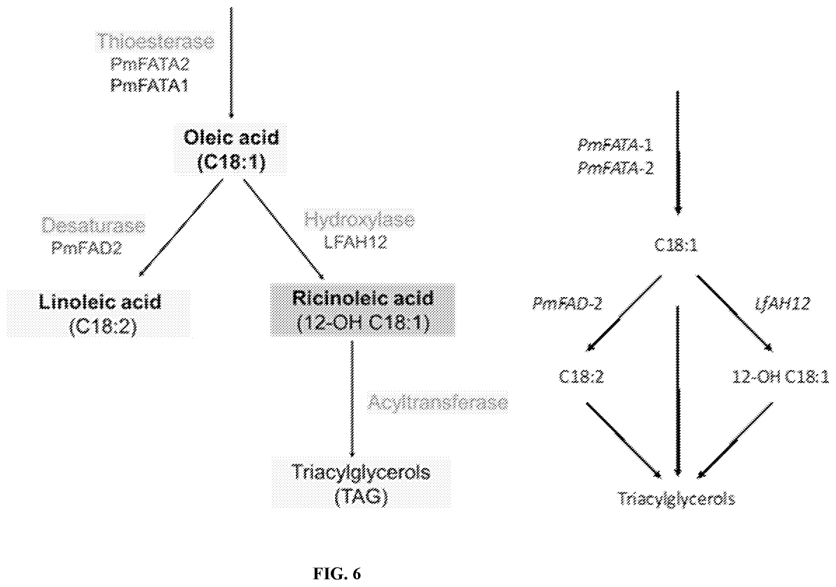

FIG. 6 shows biosynthesis pathways of ricinoleic acid. Recombinant genetic engineering techniques can be used to modify microbes to enrich for production of ricinoleic acid, i.e., ricinoleic acid enriched TAGs. For example, a microbe can be modified to express one or more exogenous genes that enhance the ability of the microbe to produce ricinoleic acid. A microbe can also be modified to remove endogenous genes that limit production of ricinoleic acid, thereby enhancing the ability of the microbe to produce of ricinoleic acid. These genes can encode for enzymes involved in ricinoleic acid and TAG biosynthesis. Non-limiting examples of such enzymes include oleate 12-hydroxylase, acyltransferases, acyl-ACP thioesterases, and delta-12 fatty acid desaturase.

Oleate 12-hydroxylase (FAH12) is a fatty acid hydroxylase that catalyzes the hydroxylation of oleate to ricinoleate. FAH12 can act on both oleic acid and eicosenoic acid, and uses cytochrome B5 as an electron donor in reactions. Upregulation of FAH12 expression can increase ricinoleic acid production, thereby increasing levels of ricinoleic acid incorporated into TAGs. Nonlimiting examples of oleate 12-hydroxylase include Lesquerella fendleri FAH12, R. communis FAH12, C. purpurea FAH12.

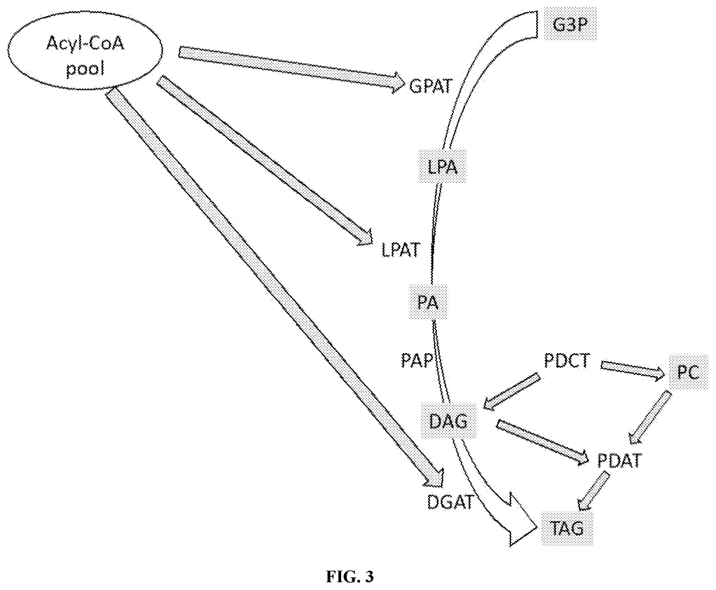

Acyltransferases are transferases that act upon acyl groups in TAG biosynthesis. Non-limiting examples of acyltransferases include phospholipid:diacylglycerol acyltransferase (PDAT), phosphatidylcholine:diacylglycerol cholinephosphotransferase (PDCT), and acyl-CoA:diacylglycerol acyltransferase (DGAT). FIG. 3 shows an overview of de novo TAG biosynthesis.

PDAT plays a significant role in channeling fatty acyl groups from phosphatidylocholine (PC) to form TAG. PDAT catalyzes the reaction of phospholipid and diacylglycerol (DAG) to form lysophospholipid and TAG. The enzyme preferentially transfers acyl groups from the sn-2 position of a phospholipid to DAG, thus forming an sn-1-lysophospholipid. Upregulation of PDAT expression can increase TAG production. Nonlimiting examples of PDAT include R. communis PDAT, e.g., RcPDAT1 and RcPDAT2.

PDCT catalyzes the interconversion of DAG and PC. PDCT preferentially transfers acyl groups from the sn-2 position of a phospholipid to DAG to form an sn-1-lysophospholipid. Upregulation of PDCT expression can increase TAG production. Nonlimiting examples of PDCT include R. communis PDCT, e.g., RcPDCT1.

DGAT catalyzes the acylation of DAG from acyl-CoA to form TAG, the final step in TAG biosynthesis. DGAT catalyzes the esterification of DAG with fatty acids by forming an ester linkage between a fatty acyl CoA and the free hydroxyl group of DAG. Upregulation of DGAT expression can increase TAG production. Nonlimiting examples of DGAT include R. communis DGAT, e.g., RcDGAT2, which catalyzes the acylation of the sn-3 hydroxy group of sn-1,2-diricinolein using ricinoleoyl-CoA.

Acyl-ACP thioesterase catalyzes the hydrolysis of thioester bonds, an essential step in chain termination in fatty acid synthesis. Acyl-ACP thioesterases can act on acyl-carrier-protein (ACP) thioesters of fatty acids from C12 to C18. Downregulation of acyl-ACP thioesterase expression can increase ricinoleic acid production, thereby increasing levels of ricinoleic acid incorporated into TAGs. In some embodiments, downregulation of acyl-ACP thioesterase expression is achieved through knockout of one or more endogenous acyl-ACP thioesterase genes.

In some embodiments, a microbe described herein is modified to remove an endogenous acyl-ACP thioesterase to increase C18 production, e.g., ricinoleic acid. Nonlimiting examples of these acyl-ACP thioesterases include P. moriformis FATA-1 and P. moriformis FATA-2.

In some embodiments, a microbe described herein is modified to express an exogenous acyl-ACP thioesterase to increase C12 production. Nonlimiting examples of such acyl-ACP thioesterases include Lindera benzoin acyl-ACP thioesterase, e.g., LbeFATB1.

Delta-12 fatty acid desaturase catalyzes the desaturation of oleic acid (C18:1) to linoleic acid (C18:2 cis-9,12). Oleic acid is a key substrate for delta-12 fatty acid desaturases. Thus, increasing the levels of oleic acid substrate can positively impact ricinoleate accumulation. For example, the endogenous FAD2 enzyme in P. moriformis is an important regulator of oleate levels. FAD2 requires oleic acid as substrate for the formation of linoleic acid, and hence, competes with the delta-12 fatty acid desaturase. Downregulation of delta-12 fatty acid desaturase expression can increase ricinoleic acid production, thereby increasing levels of ricinoleic acid incorporated into TAGs. In some embodiments, downregulation of delta-12 fatty acid desaturase expression is achieved through knockout of one or more endogenous acyl-ACP thioesterase genes.

Gene expression of an enzyme described herein can be upregulated or downregulated to enrich for ricinoleic acid. Expression of a gene described herein can be modulated singly or in combination with expression of one or more enzymes described herein, to affect enrichment of ricinoleic acid. In some embodiments, a cell described herein is modified to express an exogenous oleate 12-hydroxylase. In some embodiments, a cell described herein is modified to express an exogenous acyltransferase, such as PDAT, PDCT, DGAT, or a combination thereof. In some embodiments, a cell described herein is modified to decrease expression of an endogenous acyl-ACP thioesterase. In some embodiments, a cell described herein is modified to decrease expression of endogenous delta-12 fatty acid desaturase.

Provided herein are methods of genetically modifying and cultivating a cell to enrich for production of an oil (e.g., a TAG oil) described herein. Further provided herein are cells that are genetically modified to enrich for production of an oil (e.g., a TAG oil) described herein. A cell described herein can contain recombinant nucleic acids operable to increase expression of an enzyme described herein. Further, a cell described herein can exclude endogenous nucleic acids operable to decrease expression of an enzyme described herein. In some embodiments, the enzyme is in the ricinoleic acid biosynthetic pathway. In some embodiments, a microalgal cell is modified to enrich for production of a TAG described herein.

In some embodiments, an oil (e.g., a TAG oil) provided herein has a high ricinoleic content. For example, the fatty acid content of the TAG component of an oil provided herein can be enriched in ricinoleic acid.

In some embodiments, an oil (e.g., a TAG oil) provided herein has a fatty acid content having at least 1%, at least 5%, at least 10%, at least 15%, at least 20%, at least 30%, at least 40%, at least 50%, at least 60%, at least 70%, at least 80%, or at least 90% of hydroxylated C18:1 fatty acids. In some embodiments, an oil (e.g., a TAG oil) provided herein has a fatty acid content having 1-50%, 1-40%, 1-30%, 1-20%, 1-10%, 1-5%, 30-40%, 50-60%, or 60-70% hydroxylated C18:1 fatty acids. In some embodiments, the hydroxylated C18:1 is 12-OH-C18:1. In some embodiments, the hydroxylated C18:1 is ricinoleic acid.

In some embodiments, an oil (e.g., a TAG oil) provided herein has a fatty acid content having at least 30%, at least 40%, at least 50%, at least 60%, at least 70%, at least 80%, or at least 90% of C18:1-OH fatty acids. In some embodiments, the C18:1-OH fatty acids have ricinoleic acid. In some embodiments, the C18:1 fatty acids have at least 90% ricinoleic acid.

In some embodiments, an oil (e.g., a TAG oil) provided herein has a fatty acid profile having at least 30% ricinoleic acid and at least 30% palmitic acid. In some embodiments, an oil (e.g., a TAG oil) provided herein has a fatty acid profile having at least 40% ricinoleic acid and at least 10% palmitic acid. In some embodiments, an oil (e.g., a TAG oil) provided herein has a fatty acid profile having at least 50% ricinoleic acid and at least 20% palmitic acid. In some embodiments, an oil (e.g., a TAG oil) provided herein has a fatty acid profile having at least 60% ricinoleic acid and at least 30% palmitic acid. In some embodiments, an oil (e.g., a TAG oil) provided herein has a fatty acid profile having at least 67% ricinoleic acid and at least 33% palmitic acid.

In some embodiments, an oil (e.g., a TAG oil) provided herein has a high midchain fatty acid content. For example, the fatty acid content of the TAG component of an oil (e.g., a TAG oil) provided herein can be high in one or more midchain fatty acids. In some embodiments, the one or more midchain fatty acids are C10:0 and C12:0.

Gene expression of an enzyme described herein can be upregulated or downregulated to enrich for having ricinoleic acid and midchain fatty acids. Expression of a gene described herein can be modulated singly or in combination with expression of one or more enzymes described herein, to affect enrichment of ricinoleic acid and midchain fatty acids.

In some embodiments, an oil (e.g., a TAG oil) provided herein has a fatty acid content having at least 10%, at least 20%, or at least 30% of one or more midchain fatty acids. In some embodiments, the midchain fatty acids are C10 and C12 fatty acids.

In some embodiments, an oil (e.g., a TAG oil) provided herein has a fatty acid profile having at least 1% ricinoleic acid and at least 30% of one or more midchain fatty acids. In some embodiments, an oil (e.g., a TAG oil) provided herein has a fatty acid profile having at least 40% ricinoleic acid and at least 10% of one or more midchain fatty acids. In some embodiments, an oil (e.g., a TAG oil) provided herein has a fatty acid profile having at least 50% ricinoleic acid and at least 20% of one or more midchain fatty acids. In some embodiments, an oil provided herein has a fatty acid profile having at least 60% ricinoleic acid and at least 30% of one or more midchain fatty acids. In some embodiments, an oil (e.g., a TAG oil) provided herein has a fatty acid profile having at least 67% ricinoleic acid and at least 33% of one or more midchain fatty acids. In some embodiments, the one or more midchain fatty acids are C10:0 and C12:0.

In some embodiments, an oil (e.g., a TAG oil) provided herein has at least 60%, at least 70%, at least 80%, or at least 90% of a TAG species. In some embodiments, the TAG species is ricinolein-palmitin-ricinolein (RPR) or a regioisomer thereof. In some embodiments, the TAG species is ricinolein-midchain fatty acid-ricinolein (RMR) or a regioisomer thereof.

An oil herein (e.g., a TAG oil) can be evaluated by the fatty acid profile of the TAG component of the oil. The fatty acid profile is a measure of fatty acid composition, and can be determined by subjecting an oil to transesterification to generate fatty acid methyl esters (FAMEs) and subsequently quantitating fatty acid type by Gas Chromatography equipped with a Flame Ionization Detector (GC/FID) as a normalized percentage of total FAMEs. In some embodiments, the normalized percentage is an area % of a peak from the GC-FID chromatogram. Accordingly, fatty acid content can be determined by GC/FID. Since TAGs are comprised of three fatty acids arrayed along the glycerol backbone in the triglyceride molecule, the number of possible distinct regioisomers of TAGs can be defined by the number of fatty acid species in the oil raised to the third power.

An oil herein (e.g., a TAG oil) can also be evaluated by the TAG profile of the TAG component of the oil. The TAG profile provides relative amounts of various TAG species in an oil, which can be determined by subjecting the oil to TAG fractionation using Liquid Chromatography/Time of Flight-Mass Spectrometry (LC/TOF-MS) equipped with an Atmospheric Pressure Chemical Ionization (APCI) source.

### Polyol Applications and End Products

The oils described herein can be used or formulated with one or more excipients for a variety of applications, including but not limited to, process oils (e.g., for tires), waxes, lubricants, polyols, macrodiols, polyesterdiols, and polyurethane products, e.g., hard foams, soft foams, cast polyurethanes, thermoplastic polyurethanes (TPUs), elastomers, adhesives, coatings, laminates, films, and dispersions. Polyurethane products can be used to construct aerospace, automotive, medical, electronic, building and construction goods; sporting goods or recreational equipment, e.g., skis, snowboards, sidewalls, boating equipment, kayaks; and other consumer goods, e.g., industrial containers, coolers, mattresses, leather goods, apparel, footwear, mannequins, and phone cases. These polyurethane applications can serve as sustainable alternatives to petroleum-based, non-renewable materials, such as acrylonitrile butadiene styrene (ABS), ultra-high molecular weight polyethylene (UHMWPE), or high density polyethylene (HDPE).

Oils provided herein can have improved production efficiency and a TAG composition that is enhanced for improved control of urethane chemistry. These characteristics of microbial oil can result in a greater degree of hydroxyl group (—OH) uniformity relative to oils with greater TAG heterogeneity (hence, lower purity) and/or diversity (e.g., oilseed or plant derived oils). Polyols derived from oils (e.g., TAG oils) provided herein highly enriched in single, hydroxylated TAG species, can be preferable in generating polymers, including in instances where physical properties of a polymer can be compromised by molecular impurities, such as non-hydroxylated fatty acids or randomness in the regioselective insertion of fatty acid moieties on the glycerol backbone that may be present in oils having a more diverse or heterogeneous TAG profile.

In some embodiments, a polyol composition can be prepared by chemical conversion of an oil described herein (e.g., an oil having a fatty acid profile having at least 10% C18:1 and at least 1% of a hydroxylated C18:1). Such a polyol can be generated by conversion of an acyl moiety of a fatty acid in a TAG containing an olefinic group to a hydroxyl group. Various methods can be used to prepare a polyol composition from an oil described herein including, for example:
i) Epoxidation in the presence of hydrogen peroxide and acid catalyst, followed by ring opening with reagents, such as water, hydrogen, methanol, ethanol, or other polyols. These chemistries result in secondary hydroxyl moieties, and are therefore less reactive, for example, with isocyanate or methyl esters.
ii) Ozonolysis by molecular oxygen results in the formation of ozonides, which upon further oxidation results in scission at the double bond and formation of di-acids, carboxylic acids, and, upon reduction with hydrogen, aldehydes. Ozonolysis and reduction of oleic acid, for example, produces azaleic acid, pelargonic acid, and pelargonaldehyde, respectively.
iii) Hydroformylation with synthesis gas (syngas), using rhodium or cobalt catalysts to form the aldehyde at the olefinic group, followed by reduction of the aldehyde to alcohol in the presence of hydrogen.

While typically carried out in organic solvent, processes that utilize aqueous systems have been developed to improve sustainability of these chemistries. Of the chemistries described above, only hydroformylation results in the preservation of fatty acid length and formation of primary hydroxyl moieties. Primary hydroxyl functionalities are highly desirable due to increased reactivity compared to secondary hydroxyl moieties. Furthermore, only olefinic fatty acids with a double bond that is converted into a site possessing hydroxyl functionality, through epoxidation and ring opening, ozonolysis, or hydroformylation/reduction, can participate in subsequent downstream chemistries, i.e., reaction with an isocyanate moiety to form a urethane linkage or reaction with methyl esters to form polyesters. All other fatty acids, namely, fully saturated fatty acids that do not contain carbon-carbon double bonds, cannot participate in crosslinking reactions with isocyanates. Hence, saturated fatty acids can compromise the structural integrity and degrade performance of the polymer produced therefrom.

In some embodiments, a polyurethane composition can be prepared by reacting a polyol produced from an oil described herein (e.g., an oil having a fatty acid profile comprising at least 10% C18:1; and at least 1% of a hydroxylated C18:1) with an isocyanate.

Polyols described herein can be particularly useful for producing polyurethane materials. For example, oils provided herein can have relatively low TAG diversity, low fatty acid diversity, and the fatty acids present in the oils may be hydroxylated fatty acids. A higher ratio of hydroxylated fatty acids to non-hydroxylated fatty acids can allow for increased chemical reactivity. Oils having low TAG diversity and a high proportion of hydroxylated fatty acids can be especially desirable in production of polyurethanes because hydroxylated fatty acids that can participate in crosslinking reactions with isocyanates. Thus, polyols generated from oil having enriched in hydroxylated fatty acids can yield polyurethane materials having superior properties.

### Personal Care Product Applications

The oils (e.g., TAG oils) described herein can be used or formulated with one or more excipients for a variety of personal care product applications, including but not limited to, cosmetics, creams, face creams, hand creams, sunscreens, sunblocks, balms, lip balms, serums, face serums, body oils, hair oils, soaps, shampoos, and conditioners.

### Ultraviolet Filter Applications

UV (ultraviolet) filters are sunscreen agents that absorb UV rays. Many personal care products contain UV filters to provide UV protection against sun exposure. Solid organic UV filters are compounds generally having aromatic structures conjugated to carbonyl groups or to carbon-carbon double bonds. The UV protective effect of such filters can be optimal when the filters are evenly distributed on the surface to protect from UV rays. Furthermore, a UV filter is more effective for absorbing UV rays when the filter is adequately solubilized. Thus, optimum absorption of UV radiation requires improvement of solubility. For example, a substance can improve solubility of a UV filter by reducing the likelihood of recrystallization of an organic UV filter in a formulation.

In some embodiments, an oil (e.g., a TAG oil) described herein can be used for as a solubilizer of a UV filter. In some embodiments, an oil (e.g., a TAG oil) described herein can be used for as a solubilizer of a solid organic UV filter. In some embodiments, provided herein are UV filter compositions comprising an oil (e.g., a TAG oil) described herein.

### EXAMPLES

### Example 1: Engineering Prototheca moriformis for Midchain Fatty Acid and Ricinoleate Production

Naturally occurring oils generally do not contain significant amounts of both ricinoleic acid (12-hydroxy-9-cis-octadecenoic acid) and midchain fatty acids (e.g., C8-C12). Recombinant genetic engineering techniques can be used to modify microbes to produce TAG oils enriched in ricinoleic acid and midchain fatty acids. In this experiment, oleate hydroxylase and acyl-ACP thioesterase were co-expressed in a P. moriformis strain CHK64. Oleate 12-hydroxylase from Lesquerella fendleri (LFAH12, Genbank Accession No. AAC32755.1) was co-expressed with acyl-ACP thioesterase B from Lindera benzoin (FATB or LbeFATB1) in the algal host P. moriformis strain CHK64. CHK64 is a non-genetically engineered strain of P. moriformis that can produce TAG oils having 78% oleic acid content under lipid production conditions.

Strains described herein were grown in 96-well block formats with shaking at 200 rpm at 28° C. for 72 hrs. Culture supernatants were harvested via centrifugation and the resulting cellular pellet lyophilized to dryness, and subjected to direct transesterification to generate FAMEs for subsequent quantitation and characterization by GC/FID.

SEQ ID NO: 1 shows the integrative portion of the P. moriformis expression construct, pCHK523. The construct can be written as DA01b::PmHXT1-ScarMEL1-PmPGK-CvNR:Amt 03-LFAH12-PGH:PmACP1-CpSADtpTrimmed-LbeFATB1a-CvNR::DA01b. Relevant restriction sites in the construct pCHK523 are 5′-3′ BspQI, KpnI, AgeI, HindIII, ClaI, SacI, and BspQI, which are indicated in lowercase, bold, underlining in SEQ ID NO: 1. The expression construct contains 5′ and 3′ homology arms that permit targeted integration of the construct into the P. moriformis genome. Proceeding from the 5′ to 3′ direction, bold, lowercase sequences represent genomic DNA from P. moriformis that permit targeted integration at the 5′ end of the DAO1b locus via homologous recombination. This locus is followed by the P. moriformis hexose transporter 1 (PmHXT1) promoter that drives expression of the yeast melibiose α-galactosidase gene, which confers the ability of CHK64 to metabolize melibiose). The initiator ATG and terminator TGA for the α-galactosidase cassette are indicated by uppercase, bold italics, while the coding region is indicated in lowercase italics. The P. moriformis phosphoglucokinase (PmPGK) three prime untranslated region (3′-UTR) is indicated by uppercase underlining followed by the Chlorella vulgaris nitrate reductase 3′-UTR, indicated by lowercase, bold italics. An ammonium transporter (Amt03) promoter from P. moriformis, indicated by boxed italicized text, was used to drive the expression of the Lesquerella fendleri oleate hydroxylase (LFAH12) gene. The initiator ATG and terminator TGA codons of LFAH12 are indicated by uppercase, bold italics. The remainder of the gene is indicated by bold italics. The C. vulgaris nitrate reductase 3′-UTR is indicated by lowercase underlined text. An acyl carrier protein (ACP1) promoter from P. moriformis, indicated by boxed italicized text, drives the expression of the L. benzoin acyl-ACP thioesterase (LbeFATB1) gene. The initiator ATG and terminator TGA codons of LbeFATB1 are indicated by uppercase, bold italics. The remainder of the gene is indicated by bold italics. The LbeFATB1 was fused to the Chlorella protothecoides S106 stearoyl-ACP desaturase transit peptide, which is located between the initiator ATG and the underlined lowercase atc. The LbeFATB1 also includes a C-terminal 3×FLAG epitope tag indicated in bold, underlined, lowercase italics. The C. vulgaris nitrate reductase 3′-UTR is indicated by lowercase underlined text followed by the 3′ end of the P. moriformis DAO1b genomic region that is indicated by bold, lowercase text.

TABLE 1 shows sequences of constructs. SEQ ID NO: 2 is the primary amino acid sequence of the L. fendleri oleate 12-hydroxylase gene LFAH12. SEQ ID NO: 3 is the primary amino acid sequence of the L. benzoin acyl-ACP thioesterase gene FATB1 (LbeFATB1). The LbeFATB1 polypeptide also contains the Chlorella protothecoides stearoyl-ACP desaturase transit peptide at its N-terminus and the 3×FLAG tag at its C-terminus, both of which are underlined.

TABLE 1
Sequences

| SEQ | |
|---|---|
| ID NO | Sequence |
| 1 | gctcttccgcttgcccgcaccctcgttgatctgggagccctgcgcagccccttaaatcatctcagtcaggtttctatgttca |
| | actgagcctaaagggctttcgtcacgcgcacgagcacacgtatatcggctacgcagtctctcagaagcggtagaacagt |
| | tcgcaagccctcgtcggtcgaaaacttgcgccagtactattgaattaaattaaatgatcgaatgagacgcgaaacttttg |
| | cagaatgccactgagtttgcccagagaatgggagtggcgccattcaccatccgcctgtgcacggcctgattcgccgaga |
| | cgatgaatggcgagaccagggagcggcttgcgagccccgagccggtagcaggaataacggtcgacaatcttcctgtcc |
| | aattactggcaaccattagaaagagccggagcgcgttgaaattctgcaatcgagtaatttttcgatgcggcgggcctgct |
| | gaaccctaaggctccggcctatgtttaaggcgatccaagatgcacgcggccccaggcacgtgtctcaagcacaaaccc |
| | cagccttagtttcgagactttgggagatagcggcagatatctagtttggcattttgtatattaattaagtctcgcaatggag |
| | cgctctgatgcggtgcagcgtcggctgcagcacctggcagtggcgctggggtcgccctatcgctcggcgcctggtcagct |
| | 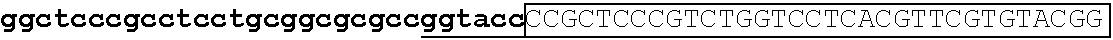 |
| | 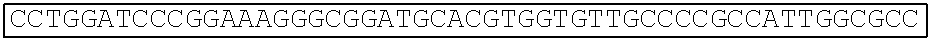 |
| | 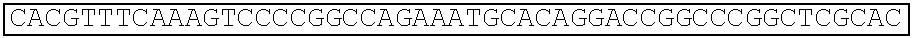 |
| | 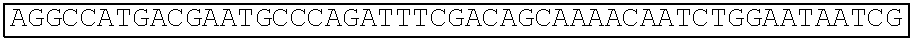 |
| | 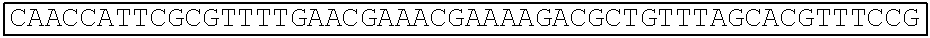 |
| | 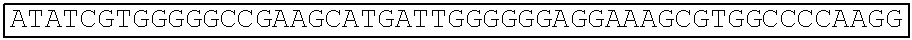 |
| | 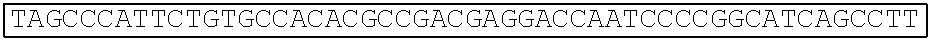 |
| | 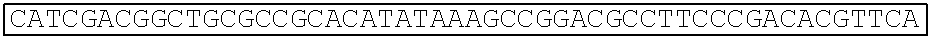 |
| | 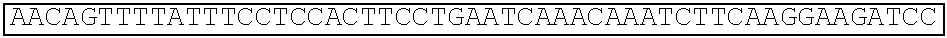 |
| | 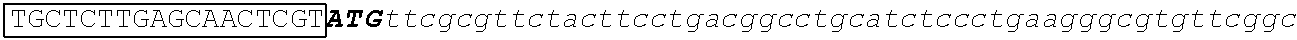 |
| | gtctccccctcctacaacggcctgggcctgacgccccagatgggctgggacaactggaacacgttcgcctgcgacgtctcc |
| | gagcagctgctgctggacacggccgaccgcatctccgacctgggcctgaaggacatgggctacaagtacatcatcctgga |
| | cgactgctggtcctccggccgcgactccgacggcttcctggtcgccgacgagcagaagttccccaacggcatgggccacg |
| | tcgccgaccacctgcacaacaactccttcctgttcggcatgtactcctccgcgggcgagtacacgtgcgccggctaccccgg |
| | ctccctgggccgcgaggaggaggacgcccagttcttcgcgaacaaccgcgtggactacctgaagtacgacaactgctac |
| | aacaagggccagttcggcacgcccgagatctcctaccaccgctacaaggccatgtccgacgccctgaacaagacgggcc |
| | gccccatcttctactccctgtgcaactggggccaggacctgaccttctactggggctccggcatcgcgaactcctggcgcatg |
| | tccggcgacgtcacggcggagttcacgcgccccgactcccgctgcccctgcgacggcgacgagtacgactgcaagtacg |
| | ccggcttccactgctccatcatgaacatcctgaacaaggccgcccccatgggccagaacgcgggcgtcggcggctggaa |
| | cgacctggacaacctggaggtcggcgtcggcaacctgacggacgacgaggagaaggcgcacttctccatgtgggccatg |
| | gtgaagtcccccctgatcatcggcgcgaacgtgaacaacctgaaggcctcctcctactccatctactcccaggcgtccgtca |
| | tcgccatcaaccaggactccaacggcatccccgccacgcgcgtctggcgctactacgtgtccgacacggacgagtacggc |
| | cagggcgagatccagatgtggtccggccccctggacaacggcgaccaggtcgtggcgctgctgaacggcggctccgtgt |
| | cccgccccatgaacacgaccctggaggagatcttcttcgactccaacctgggctccaagaagctgacctccacctgggac |
| | atctacgacctgtgggcgaaccgcgtcgacaactccacggcgtccgccatcctgggccgcaacaagaccgccaccggca |
| | tcctgtacaacgccaccgagcagtcctacaaggacggcctgtccaagaacgacacccgcctgttcggccagaagatcgg |
| | ctccctgtcccccaacgcgatcctgaacacgaccgtccccgcccacggcatcgcgttctaccgcctgcgcccctcctccTG |
| | ATACAACTTATTACGTATTCTGACCGGCGCTGATGTGGCGCGGACGCCGTCG |
| | TACTCTTTCAGACTTTACTCTTGAGGAATTGAACCTTTCTCGCTTGCTGGCAT |
| | GTAAACATTGGCGCAATTAATTGTGTGATGAAGAAAGGGTGGCACAAGATG |
| | GATCGCGAATGTACGAGATCGACAACGATGGTGATTGTTATGAGGGGCCAA |
| | ACCTGGCTCAATCTTGTCGCATGTCCGGCGCAATGTGATCCAGCGGCGTGAC |
| | TCTCGCAACCTGGTAGTGTGTGCGCACCGGGTCGCTTTGATTAAAACTGATC |
| | GCATTGCCATCCCGTCAACTCACAAGCCTACTCTAGCTCCCATTGCGCACTCG |
| | GGCGCCCGGCTCGATCAATGTTCTGAGCGGAGGGCGAAGCGTCAGGAAATC |
| | GTCTCGGCAGCTGGAAGCGCATGGAATGCGGAGCGGAGATCGAATCAaccggt |
| | gcagcagcagctcggatagtatcgacacactctggacgctggtcgtgtgatggactgttgccgccacacttgctgccttgac |
| | ctgtgaatatccctgccgcttttatcaaacagcctcagtgtgtttgatcttgtgtgtacgcgcttttgcgagttgctagctgcttgtg |
| | ctatttgcgaataccacccccagcatccccttccctcgtttcatatcgcttgcatcccaaccgcaacttatctacgctgtcctgct |
| | atccctcagcgctgctcctgctcctgctcactgcccctcgcacagccttggtttgggctccgcctgtattctcctggtactgcaac |
| | 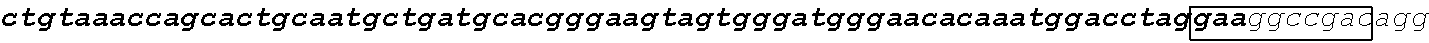 |
| | 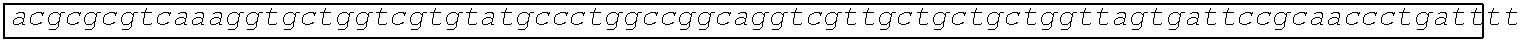 |
| | 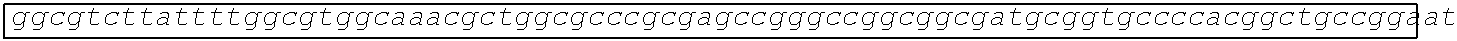 |
| | 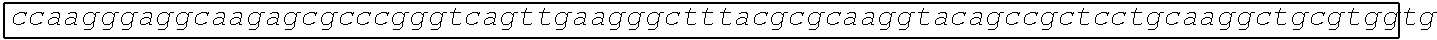 |
| | 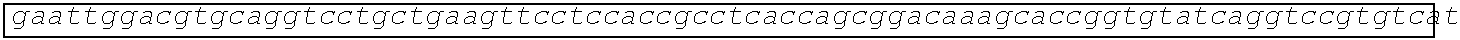 |
| | 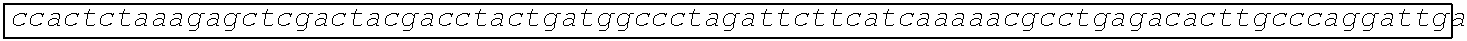 |
| | 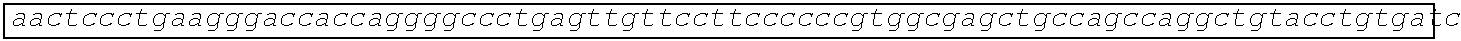 |
| | 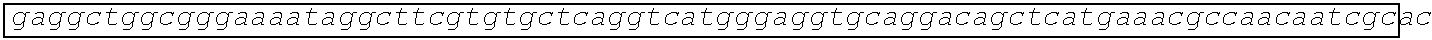 |
| | 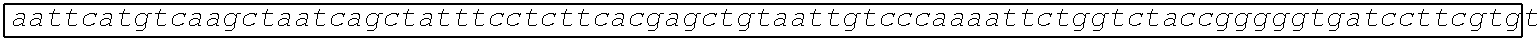 |
| | 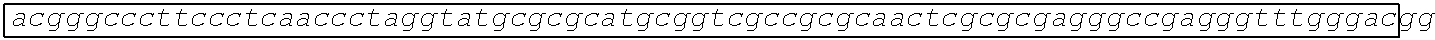 |
| | 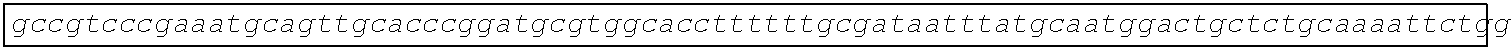 |
| | 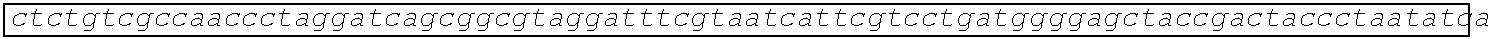 |
| | 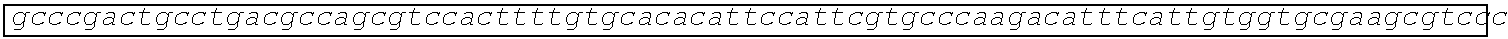 |
| | 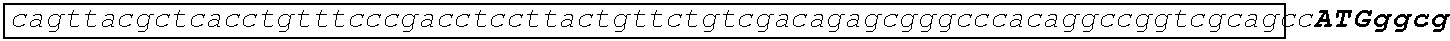 |
| | cgggcggccggatcatggtgaccccctcctccaagaagtccgagaccgaggccctgaagcgcggcccctgcgagaagc |
| | cccccttcaccgtgaaggacctgaagaaggccatcccccagcactgcttcaagcgctccatcccccgctccttctcctacctg |
| | ctgaccgacatcaccctggtgtcctgcttctactacgtggccaccaactacttctccctgctgccccagcccctgtcgacctac |
| | ctggcctggcccctgtactgggtgtgccagggctgcgtgctgacgggcatctgggtgctgggccacgagtgcggccaccac |
| | gccttctccgactaccagtggctggacgacaccgtgggcttcatcttccactccctgctgctggtgccctacttctcctggaagt |
| | actcccaccgccgccaccactccaacaacggctccctggagaaggacgaggtgttcgtgccccccaagaaggccgccgt |
| | gaagtggtacgtgaagtacctgaacaaccccctgggccgcatcctggtgctgaccgtgcagttcatcctgggctggcccctg |
| | tacctggccttcaacgtgtccggccgcccctacgacggcttcgcctcccacttcttcccccacgcccccatcttcaaggaccg |
| | cgagcgcctccagatctacatctccgacgcgggcatcctggccgtgtgctacggcctgtaccgctacgccgcctcccaggg |
| | cctgaccgccatgatctgcgtgtacggcgtgcccctgctgatcgtcaacttcttcctggtgctggtgaccttcctccagcacacc |
| | cacccctccctgccccactacgactccaccgagtgggagtggatcaggggcgccctggtgaccgtggaccgcgactacgg |
| | catcctgaacaaggtgttccacaacatcaccgacacccacgtggcccaccacctgttcgccaccatcccccactacaacgc |
| | catggaggccaccgaggccatcaagcccatcctgggcgactactaccacttcgacggcaccccctggtacgtggccatgt |
| | accgcgaggccaaggagtgcctgtacgtggagcccgacaccgagcgcggcaagaagggcgtgtactactacaacaac |
| | aagctgTGAacgcccgcgcggcgcacctgacctgttctctcgagggcgcctgttctgccttgcgaaacaagcccctggagc |
| | atgcgtgcatgatcgtctctggcgccccgccgcgcggtttgtcgccctcgcgggcgccgcggccgcgggggcgcattgaaat |
| | tgttgcaaaccccacctgacagattgagggcccaggcaggaaggcgttgagatggaggtacaggagtcaagtaactgaaagt |
| | ttttatgataactaacaacaaagggtcgtttctggccagcgaatgacaagaacaagattccacatttccgtgtagaggcttgccatc |
| | gaatgtgagcggggggccgcggacccgacaaaacccttacgacgtggtaagaaaaacgtgggggcactgtccctgtagc |
| | 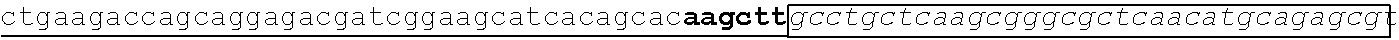 |
| | 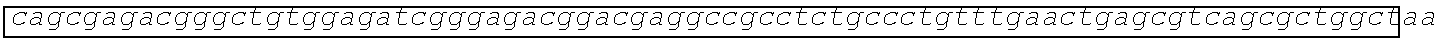 |
| | 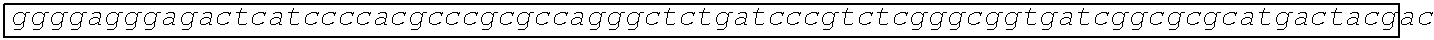 |
| | 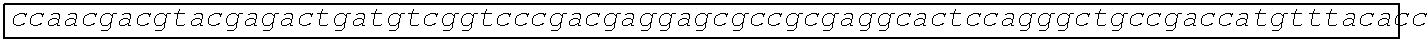 |
| | 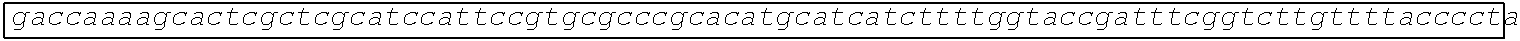 |
| | 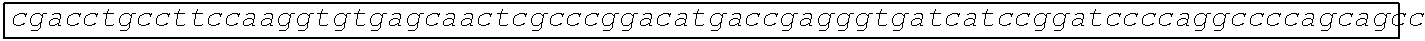 |
| | 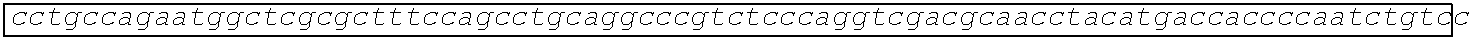 |
| | 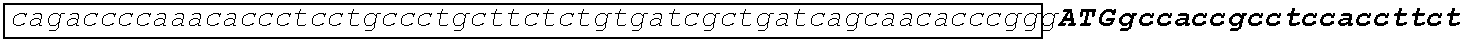 |
| | ccgccttcaacgcccgctgcggcgacctgcgccgctccgccggctccggcccccgccgccccgcccgccccctgcccgtg |
| | cgcgccgccatcggcaacgcccagacctccctgaagatgatcgacggcaccaagttctcctacaccgagtccctgaagcg |
| | cctgcccgactggtccaagctggacgaccgcttcggcctgcacggcctggtgttccgccgcaccttcgccatccgctcctac |
| | gaggtgggccccgaccgctccgcctccatcctggccgtgctgaaccacctgcaggaggccaccctgaaccacgccgagt |
| | ccgtgggcatcctgggcgaccgcttcggcgagaccctggagatgtccaagcgcgacctgatgtgggtggtgcgccgcacc |
| | tacgtggccgtggagcgctaccccgcctggggcgacaccgtggagatcgagtcctggatcggcgcctccggcaacaacg |
| | gcatgcgccgcgagttcctggtgcgcgacttcaagaccggcgagatcctgacccgctgcacctccctgtccgtgatgatga |
| | acacccgcacccgccgcctgtccaagatccccgaggaggtgcgcggcgagatcggccccgtgttcatcgacaacgtggc |
| | cgtgaaggacgaggagatcaagaagctgcagaagctgaacgactccaccgccgactacatccagggcggcctgatccc |
| | ccgctggaacgacctggacctgaaccagcacgtgaacaacatcaagtacgtgtcctggatcctggagaccgtgcccgact |
| | ccatcctggagtcctaccacatgtcctccatcaccctggagtaccgccgcgagtgcacccgcgactccgtgctgcagtccct |
| | gaccaccgtgtccggcggctcctccgaggccggcctggtgtgcgagcactccctgctgctggagggcggctccgaggtgct |
| | gcgcgcccgcaccgagtggcgccccaagctgaccgactccttccgcggcatctccgtgatccccgccgagcagtccgtga |
| | tggactacaaggaccacgacggcgactacaaggaccacgacatcgactacaaggacgacgacgacaagTGAatcg |
| | atgcagcagcagctcggatagtatcgacacactctggacgctggtcgtgtgatggactgttgccgccacacttgctgccttgac |
| | ctgtgaatatccctgccgcttttatcaaacagcctcagtgtgtttgatcttgtgtgtacgcgcttttgcgagttgctagctgcttgtgct |
| | atttgcgaataccacccccagcatccccttccctcgtttcatatcgcttgcatcccaaccgcaacttatctacgctgtcctgctatcc |
| | ctcagcgctgctcctgctcctgctcactgcccctcgcacagccttggtttgggctccgcctgtattctcctggtactgcaacctgta |
| | aaccagcactgcaatgctgatgcacgggaagtagtgggatgggaacacaaatggagagctcagcgtctgcgtgttgggagc |
| | tggagtcgtgggcttgacgacggcgctgcagctcttgcaggatgtgcctggcgtgcgcgttcacgtcgtggctgagaaat |
| | atggcgacgaaacgttgacggctggggccgggggctgtggatgccatacgcattgggtacgcggccattggatggga |
| | ttggtaggcttatggagggataatagagtttttgtcggatccaacgcatgttgatgcagtagcccggtgtgctgaaagtat |
| | ggaaggatggtgcattggctattcacatgcgctgcccaccccttttcgcaggaaatgtgccggcatcgttggtgcaccga |
| | tggggaaaatcgacgttcgaccactacatgaaaatgtatacctctgaagatgcagcaactgcgggtgcgaaacggata |
| | acggtttggtaggtgtatgtcgcagcatgtgctggattttgcgggcaactccccctgccacggcccattgcaggtgtcatg |
| | ttgactggaggatacgaactttcggccgtcaaattcccagaggaggaccccctctgggccgacattgtgcccactgctct |
| | tc |
| 2 | MGAGGRIMVTPSSKKSETEALKRGPCEKPPFTVKDLKKAIPQHCFKRSIPRSFSYL |
| | LTDITLVSCFYYVATNYFSLLPQPLSTYLAWPLYWVCQGCVLTGIWVLGHECGH |
| | HAFSDYQWLDDTVGFIFHSLLLVPYFSWKYSHRRHHSNNGSLEKDEVFVPPKKA |
| | AVKWYVKYLNNPLGRILVLTVQFILGWPLYLAFNVSGRPYDGFASHFFPHAPIF |
| | KDRERLQIYISDAGILAVCYGLYRYAASQGLTAMICVYGVPLLIVNFFLVLVTFL |
| | QHTHPSLPHYDSTEWEWIRGALVTVDRDYGILNKVFHNITDTHVAHHLFATIPH |
| | YNAMEATEAIKPILGDYYHFDGTPWYVAMYREAKECLYVEPDTERGKKGVYY |
| | YNNKL |
| 3 | MATASTFSAFNARCGDLRRSAGSGPRRPARPLPVRAAIGNAQTSLKMIDGTKFS |
| | YTESLKRLPDWSKLDDRFGLHGLVFRRTFAIRSYEVGPDRSASILAVLNHLQEAT |
| | LNHAESVGILGDRFGETLEMSKRDLMWVVRRTYVAVERYPAWGDTVEIESWIG |
| | ASGNNGMRREFLVRDFKTGEILTRCTSLSVMMNTRTRRLSKIPEEVRGEIGPVFI |
| | DNVAVKDEEIKKLQKLNDSTADYIQGGLIPRWNDLDLNQHVNNIKYVSWILETV |
| | PDSILESYHMSSITLEYRRECTRDSVLQSLTTVSGGSSEAGLVCEHSLLLEGGSEV |
| | LRARTEWRPKLTDSFRGISVIPAEQSVMDYKDHDGDYKDHDIDYFENTQHV |

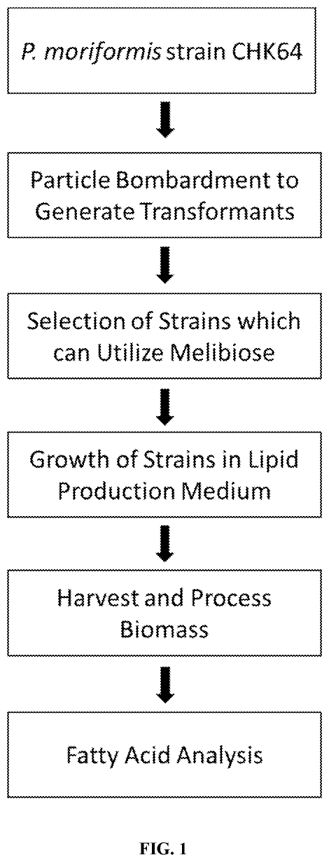

FIG. 1 illustrates a flow diagram for the preparation and analysis of fatty acids produced by P. moriformis strain CHK64. Transformants of CHK64 were generated via particle bombardment using gold nanoparticles. Primary transformants were selected for the ability to grow on plates containing melibiose as the sole carbon source. Transformants were grown in lipid production medium. The resulting biomass of the transformants was processed as previously described for fatty acid analysis.

TABLE 2 shows the fatty acid profiles of ten primary transformants of CHK64 with PCHK523 and two controls. The four bolded strains (Sample 1.0, Sample 2.0, Sample 4.0, and Sample 5.0) were selected for further evaluation in 10-mL bioreactor tubes using the same lipid production medium used in the 96-well block format but in a slightly larger volume and run for 120 hours instead of 72 hours. P. moriformis base strain CHK22 and classically improved strain CHK64 were used as the non-transgenic controls. CHK22 is a Prototheca wickerhamii strain (UTEX1533) obtained from the University of Texas at Austin Culture Collection of Algae (UTEX). Analysis of the 23S ribosomal RNA (rRNA) sequence of UTEX1533 suggests that UTEX1533 is closely related to a P. moriformis strain, UTEX1435. Strains were grown for 72 hrs in 96-well blocks at 200 rpm at 28° C. Biomass was harvested, lyophilized to dryness, and subjected to direct transesterification to generate FAMEs for subsequent quantitation and characterization by GC/FID

Results of samples selected for further evaluation are shown in TABLE 3. P. moriformis base strain CHK22 and classically improved strain CHK64 are shown as controls. Base levels of total oil are measured as percent of dry cell weight (DCW). Samples were evaluated along with non-transgenic control strains in 10-mL bioreactor tubes in lipid production medium. Strains were grown for 120 hours at 200 rpm at 28° C. Culture supernatants were harvested via centrifugation. The resulting cellular pellet was lyophilized to dryness, and subjected to direct transesterification to generate FAMEs for subsequent quantitation and characterization by GC/FID.

TABLE 2
Screen of primary transformants of CHK64 with pCHK523

| | | | | | | | | | | | | | 12-OH, |
|---|---|---|---|---|---|---|---|---|---|---|---|---|---|
| Sample | | | | | | | | | C18:3 | | | | 9c- |
| Name | C10:0 | C12:0 | C14:0 | C16:0 | C16:1 | C18:0 | C18:1 | C18:2 | alpha | C20:0 | C20:1 | C22:0 | C18:1 |
| Sample | 3.73 | 31.21 | 4.36 | 4.56 | 0.15 | 1.30 | 40.53 | 6.24 | 0.31 | 0.14 | 0.37 | 0.04 | 6.69 |
| 1.0 | | | | | | | | | | | | | |
| Sample | 2.31 | 21.91 | 3.31 | 5.11 | 0.20 | 1.78 | 52.36 | 6.62 | 0.27 | 0.16 | 0.41 | 0.06 | 5.14 |
| 2.0 | | | | | | | | | | | | | |
| Sample | 1.44 | 14.74 | 2.49 | 6.39 | 0.28 | 2.02 | 59.45 | 7.19 | 0.34 | 0.17 | 0.36 | 0.05 | 4.74 |
| 3.0 | | | | | | | | | | | | | |
| Sample | 2.35 | 22.42 | 3.41 | 5.43 | 0.21 | 1.59 | 51.25 | 7.42 | 0.33 | 0.16 | 0.37 | 0.06 | 4.52 |
| 4.0 | | | | | | | | | | | | | |
| Sample | 2.44 | 23.37 | 3.54 | 5.12 | 0.19 | 1.58 | 51.47 | 6.54 | 0.30 | 0.15 | 0.39 | 0.05 | 4.46 |
| 5.0 | | | | | | | | | | | | | |
| Sample | 1.93 | 19.28 | 3.10 | 5.83 | 0.23 | 1.69 | 54.25 | 7.97 | 0.34 | 0.16 | 0.36 | 0.05 | 4.44 |
| 6.0 | | | | | | | | | | | | | |
| Sample | 2.47 | 22.61 | 3.40 | 5.31 | 0.22 | 1.57 | 52.88 | 6.61 | 0.28 | 0.14 | 0.36 | 0.05 | 3.72 |
| 7.0 | | | | | | | | | | | | | |
| Sample | 1.83 | 18.14 | 2.92 | 5.73 | 0.23 | 1.78 | 56.92 | 7.50 | 0.33 | 0.16 | 0.38 | 0.04 | 3.64 |
| 8.0 | | | | | | | | | | | | | |
| Sample | 1.26 | 15.67 | 2.79 | 6.52 | 0.27 | 1.85 | 57.38 | 9.27 | 0.48 | 0.22 | 0.35 | 0.07 | 3.49 |
| 9.0 | | | | | | | | | | | | | |
| Sample | 1.47 | 14.10 | 2.64 | 6.34 | 0.26 | 2.03 | 60.07 | 8.28 | 0.36 | 0.18 | 0.38 | 0.04 | 3.45 |
| 10.0 | | | | | | | | | | | | | |
| CHK22 | 0.00 | 0.05 | 1.56 | 22.53 | 1.04 | 2.04 | 59.82 | 11.69 | 0.35 | 0.03 | 0.00 | 0.12 | 0.08 |
| Control | | | | | | | | | | | | | |
| CHK64 | 0.04 | 0.02 | 0.50 | 8.30 | 0.49 | 2.04 | 79.76 | 7.64 | 0.22 | 0.08 | 0.00 | 0.07 | 0.08 |
| Control | | | | | | | | | | | | | |

TABLE 3
10-mL bioreactor screens of lead primary transformants of CHK64 with PCHK523

| | | | | | | | | | | | | | 12-OH, |
|---|---|---|---|---|---|---|---|---|---|---|---|---|---|
| Sample | Oil as % | | | | | | | | | C18:3 | | | 9c- |
| Name | DCW | C10:0 | C12:0 | C14:0 | C16:0 | C16:1 | C18:0 | C18:1 | C18:2 | alpha | C20:0 | C20:1 | C18:1 |
| Sample | 69.5 | 3.23 | 26.99 | 3.69 | 3.67 | 0.14 | 1.70 | 47.69 | 5.03 | 0.26 | 0.18 | 0.62 | 6.42 |
| 1.1 | | | | | | | | | | | | | |
| Sample | 68.8 | 3.22 | 27.03 | 3.72 | 3.68 | 0.14 | 1.71 | 47.67 | 5.02 | 0.26 | 0.17 | 0.60 | 6.44 |
| 1.2 | | | | | | | | | | | | | |
| Sample | 69.0 | 2.77 | 24.36 | 3.31 | 3.99 | 0.15 | 1.82 | 51.44 | 5.39 | 0.29 | 0.16 | 0.56 | 5.41 |
| 5.1 | | | | | | | | | | | | | |
| Sample | 70.3 | 2.79 | 24.45 | 3.30 | 3.96 | 0.15 | 1.81 | 51.37 | 5.37 | 0.29 | 0.16 | 0.58 | 5.40 |
| 5.2 | | | | | | | | | | | | | |
| Sample | 64.3 | 2.24 | 19.52 | 2.68 | 4.20 | 0.16 | 2.13 | 56.97 | 5.43 | 0.24 | 0.17 | 0.64 | 5.24 |
| 2.1 | | | | | | | | | | | | | |
| Sample | 68.3 | 2.06 | 18.33 | 2.56 | 4.32 | 0.18 | 2.13 | 59.41 | 5.23 | 0.23 | 0.16 | 0.64 | 4.39 |
| 2.2 | | | | | | | | | | | | | |
| Sample | 70.0 | 1.57 | 14.85 | 2.21 | 4.77 | 0.22 | 1.98 | 65.36 | 5.42 | 0.27 | 0.12 | 0.51 | 2.41 |
| 4.1 | | | | | | | | | | | | | |
| Sample | 67.0 | 2.57 | 22.54 | 3.12 | 4.16 | 0.17 | 1.84 | 54.19 | 5.40 | 0.28 | 0.16 | 0.57 | 4.66 |
| 4.2 | | | | | | | | | | | | | |
| CHK22 | 69.1 | 0.01 | 0.04 | 1.36 | 25.35 | 0.79 | 3.16 | 60.27 | 7.53 | 0.45 | 0.31 | 0.09 | 0.08 |
| Control 1 | | | | | | | | | | | | | |
| CHK22 | 72.4 | 0.02 | 0.04 | 1.36 | 25.21 | 0.79 | 3.15 | 60.54 | 7.43 | 0.44 | 0.31 | 0.09 | 0.08 |
| Control 2 | | | | | | | | | | | | | |
| CHK64 | 69.9 | 0.02 | 0.02 | 0.38 | 6.92 | 0.34 | 2.67 | 81.94 | 6.40 | 0.32 | 0.13 | 0.47 | 0.09 |
| Control 1 | | | | | | | | | | | | | |
| CHK64 | 64.7 | 0.03 | 0.02 | 0.41 | 6.93 | 0.32 | 2.80 | 81.29 | 6.82 | 0.33 | 0.13 | 0.46 | 0.10 |
| Control 2 | | | | | | | | | | | | | |

### Example 2: Expressing Oleate Hydroxylases in P. moriformis Strain CHK22

Recombinant genetic engineering techniques can be used to modify microbes to produce TAG oils enriched in ricinoleic acid. In this experiment, oleate 12-hydroxylase was expressed in a P. moriformis strain CHK22. In this example, oleate 12-hydroylase from R. communis (RcFAH12, GenBank Accession No. AAC49010.1), Lesquerella fendleri (LFAH12, Genbank Accession No. AAC32755.1), and C. purpurea (CpFAH12, GenBank Accession No. B4YQU1.1) were expressed in the algal host P. moriformis strain CHK22.

Expressing LFAH12 Oleate 12-Hydroxylase in P. moriformis Strain CHK22

The L. fendleri oleate 12-hydroxylase (LFAH12) was introduced into a P. moriformis strain CHK22. The expression construct pCHK277 contains 5′ and 3′ homology arms to permit its targeted integration into the P. moriformis genome, as shown in SEQ ID NO: 4.

TABLE 4 shows sequences of constructs. SEQ ID NO: 4 shows the integrative sequences for the transformation of P. moriformis with pCHK277; the construct can be written as 5′DAO1b::CrTUB2:ScSUC2:PmPGH:CvNR:PmAMT03:LFAH12:CvNR::3′DAO1b. Proceeding in the 5′ to 3′ direction, bold, lowercase sequences represent genomic DNA from CHK22 that permit targeted integration at the DAO1b locus via homologous recombination. Proceeding in the 5′ to 3′ direction, the Chlamydomonas reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics which contains a Chlorella vulgaris nitrate reductase 3′-UTR. An AMT03 promoter from P. moriformis, indicated by boxed italicized text, drives expression of the LFAH12 gene. The initiator ATG and terminator TGA codons of LFAH12 are indicated by uppercase, bold italics, while the remainder of the gene is indicated by bold italics. The C. vulgaris nitrate reductase 3′-UTR is indicated by lowercase underlined text followed by the CHK22 DAO1b genomic region indicated by bold, lowercase text. SEQ ID NO: 5 is the amino acid sequence of the LFAH12 polypeptide.

TABLE 4
Sequences

| SEQ | |
|---|---|
| ID NO | Sequence |
| 4 | gctcttccgcttgcccgcaccctcgttgatctgggagccctgcgcagccccttaaatcatctcagtcaggtttctatgttca |
| | actgagcctaaagggctttcgtcacgcgcacgagcacacgtatatcggctacgcagtctctcagaagcggtagaacagt |
| | tcgcaagccctcgtcggtcgaaaacttgcgccagtactattgaattaaattaaatgatcgaatgagacgcgaaacttttg |
| | cagaatgccactgagtttgcccagagaatgggagtggcgccattcaccatccgcctgtgcacggcctgattcgccgaga |
| | cgatgaatggcgagaccagggagcggcttgcgagccccgagccggtagcaggaataacggtcgacaatcttcctgtcc |
| | aattactggcaaccattagaaagagccggagcgcgttgaaattctgcaatcgagtaatttttcgatgcggcgggcctgct |
| | gaaccctaaggctccggcctatgtttaaggcgatccaagatgcacgcggccccaggcacgtgtctcaagcacaaaccc |
| | cagccttagtttcgagactttgggagatagcggcagatatctagtttggcattttgtatattaattaagtctcgcaatggag |
| | cgctctgatgcggtgcagcgtcggctgcagcacctggcagtggcgctggggtcgccctatcgctcggcgcctggtcagct |
| | 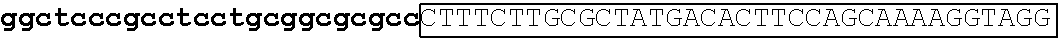 |
| | 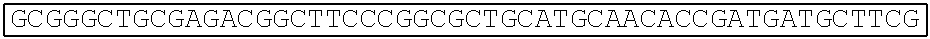 |
| | 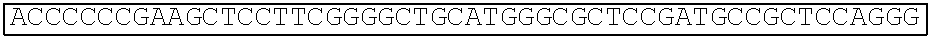 |
| | 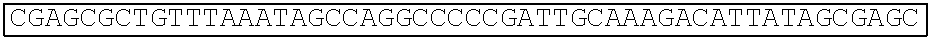 |
| | 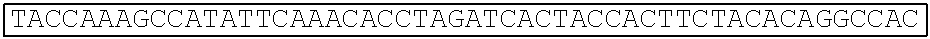 |
| | 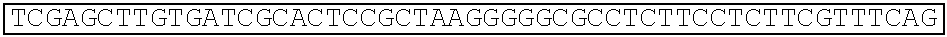 |
| | 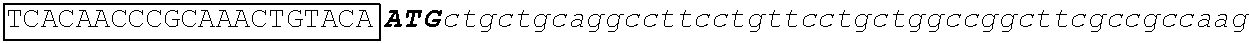 |
| | atcagcgcctccatgacgaacgagacgtccgaccgccccctggtgcacttcacccccaacaagggctggatgaacgacc |
| | ccaacggcctgtggtacgacgagaaggacgccaagtggcacctgtacttccagtacaacccgaacgacaccgtctgggg |
| | gacgcccttgttctggggccacgccacgtccgacgacctgaccaactgggaggaccagcccatcgccatcgccccgaag |
| | cgcaacgactccggcgccttctccggctccatggtggtggactacaacaacacctccggcttcttcaacgacaccatcgacc |
| | cgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctg |
| | gtacgagccctcccagaagtggatcatgaccgcggccaagtcccaggactacaagatcgagatctactcctccgacgacc |
| | tgaagtcctggaagctggagtccgcgttcgccaacgagggcttcctcggctaccagtacgagtgccccggcctgatcgagg |
| | tccccaccgagcaggaccccagcaagtcctactgggtgatgttcatctccatcaaccccggcgccccggccggcggctcct |
| | tcaaccagtacttcgtcggcagcttcaacggcacccacttcgaggccttcgacaaccagtcccgcgtggtggacttcggcaa |
| | ggactactacgccctgcagaccttcttcaacaccgacccgacctacgggagcgccctgggcatcgcgtgggcctccaactg |
| | ggagtactccgccttcgtgcccaccaacccctggcgctcctccatgtccctcgtgcgcaagttctccctcaacaccgagtacc |
| | aggccaacccggagacggagctgatcaacctgaaggccgagccgatcctgaacatcagcaacgccggcccctggagc |
| | cggttcgccaccaacaccacgttgacgaaggccaacagctacaacgtcgacctgtccaacagcaccggcaccctggagt |
| | tcgagctggtgtacgccgtcaacaccacccagacgatctccaagtccgtgttcgcggacctctccctctggttcaagggcctg |
| | gaggaccccgaggagtacctccgcatgggcttcgaggtgtccgcgtcctccttcttcctggaccgcgggaacagcaaggtg |
| | aagttcgtgaaggagaacccctacttcaccaaccgcatgagcgtgaacaaccagcccttcaagagcgagaacgacctgt |
| | cctactacaaggtgtacggcttgctggaccagaacatcctggagctgtacttcaacgacggcgacgtcgtgtccaccaaca |
| | cctacttcatgaccaccgggaacgccctgggctccgtgaacatgacgacgggggtggacaacctgttctacatcgacaagt |
| | tccaggtgcgcgaggtcaagtgactTAAGACGCCCGCGCGGCGCACCTGACCTGTTCTCTC |
| | GAGGGCGCCTGTTCTGCCTTGCGAAACAAGCCCCTGGAGCATGCGTGCATGA |
| | TCGTCTCTGGCGCCCCGCCGCGCGGTTTGTCGCCCTCGCGGGCGCCGCGGCC |
| | GCGGGGGCGCATTGAAATTGTTGCAAACCCCACCTGACAGATTGAGGGCCCA |
| | GGCAGGAAGGCGTTGAGATGGAGGTACAGGAGTCAAGTAACTGAAAGTTTT |
| | TATGATAACTAACAACAAAGGGTCGTTTCTGGCCAGCGAATGACAAGAACA |
| | AGATTCCACATTTCCGTGTAGAGGCTTGCCATCGAATGTGAGCGGGCGGGCC |
| | GCGGACCCGACAAAACCCTTACGACGTGGTAAGAAAAACGTGGCGGGCACT |
| | GTCCCTGTAGCCTGAAGACCAGCAGGAGACGATCGGAAGCATCACAGCACA |
| | ACCGGTgcagcagcagctcggatagtatcgacacactctggacgctggtcgtgtgatggactgttgccgccacacttg |
| | ctgccttgacctgtgaatatccctgccgcttttatcaaacagcctcagtgtgtttgatcttgtgtgtacgcgcttttgcgagttgcta |
| | gctgcttgtgctatttgcgaataccacccccagcatccccttccctcgtttcatatcgcttgcatcccaaccgcaacttatctacg |
| | ctgtcctgctatccctcagcgctgctcctgctcctgctcactgcccctcgcacagccttggtttgggctccgcctgtattctcctgg |
| | tactgcaacctgtaaaccagcactgcaatgctgatgcacgggaagtagtgggatgggaacacaaatggacctaggaaat |
| | 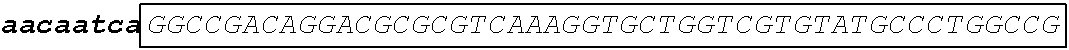 |
| | 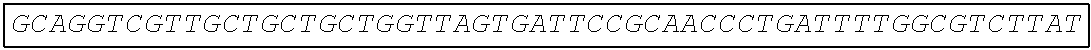 |
| | 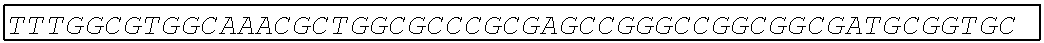 |
| | 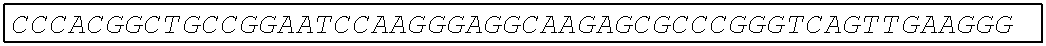 |
| | 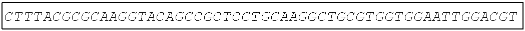 |
| | 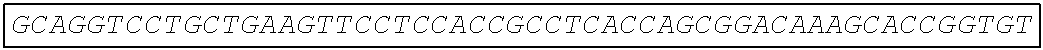 |
| | 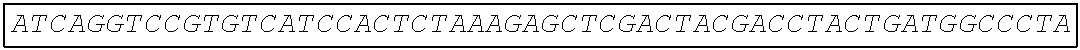 |
| | 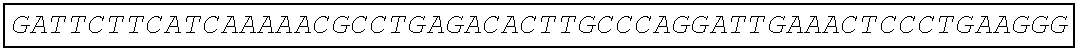 |
| | 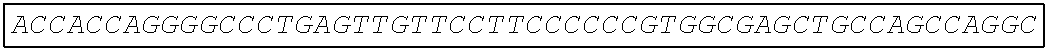 |
| | 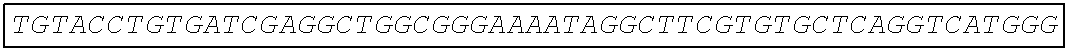 |
| | 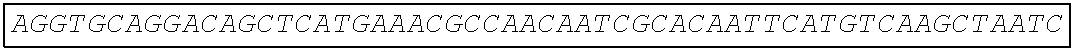 |
| | 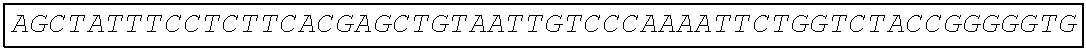 |
| | 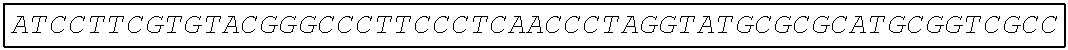 |
| | 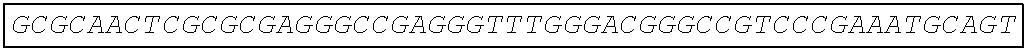 |
| | 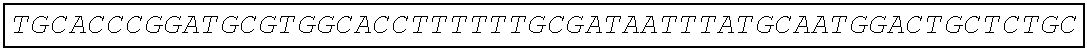 |
| | 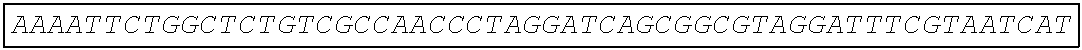 |
| | 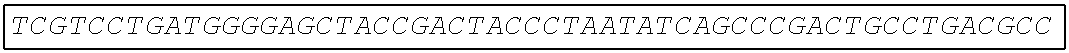 |
| | 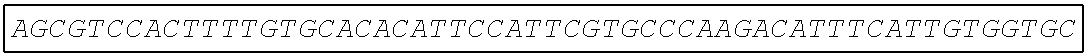 |
| | 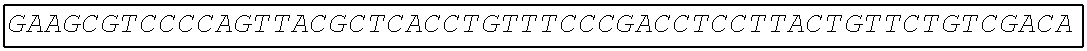 |
| | 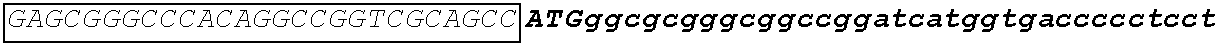 |
| | ccaagaagtccgagaccgaggccctgaagcgcggcccctgcgagaagccccccttcaccgtgaaggacctgaagaag |
| | gccatcccccagcactgcttcaagcgctccatcccccgctccttctcctacctgctgaccgacatcaccctggtgtcctgcttct |
| | actacgtggccaccaactacttctccctgctgccccagcccctgtcgacctacctggcctggcccctgtactgggtgtgccag |
| | ggctgcgtgctgacgggcatctgggtgctgggccacgagtgcggccaccacgccttctccgactaccagtggctggacga |
| | caccgtgggcttcatcttccactccctgctgctggtgccctacttctcctggaagtactcccaccgccgccaccactccaacaa |
| | cggctccctggagaaggacgaggtgttcgtgccccccaagaaggccgccgtgaagtggtacgtgaagtacctgaacaac |
| | cccctgggccgcatcctggtgctgaccgtgcagttcatcctgggctggcccctgtacctggccttcaacgtgtccggccgccc |
| | ctacgacggcttcgcctcccacttcttcccccacgcccccatcttcaaggaccgcgagcgcctccagatctacatctccgacg |
| | cgggcatcctggccgtgtgctacggcctgtaccgctacgccgcctcccagggcctgaccgccatgatctgcgtgtacggcgt |
| | gcccctgctgatcgtcaacttcttcctggtgctggtgaccttcctccagcacacccacccctccctgccccactacgactccac |
| | cgagtgggagtggatcaggggcgccctggtgaccgtggaccgcgactacggcatcctgaacaaggtgttccacaacatc |
| | accgacacccacgtggcccaccacctgttcgccaccatcccccactacaacgccatggaggccaccgaggccatcaagc |
| | ccatcctgggcgactactaccacttcgacggcaccccctggtacgtggccatgtaccgcgaggccaaggagtgcctgtacg |
| | tggagcccgacaccgagcgcggcaagaagggcgtgtactactacaacaacaagctgTGAgcagcagcagctcggata |
| | gtatcgacacactctggacgctggtcgtgtgatggactgttgccgccacacttgctgccttgacctgtgaatatccctgccgctttt |
| | atcaaacagcctcagtgtgtttgatcttgtgtgtacgcgcttttgcgagttgctagctgcttgtgctatttgcgaataccacccccag |
| | catccccttccctcgtttcatatcgcttgcatcccaaccgcaacttatctacgctgtcctgctatccctcagcgctgctcctgctcctg |
| | ctcactgcccctcgcacagccttggtttgggctccgcctgtattctcctggtactgcaacctgtaaaccagcactgcaatgctgat |
| | gcacgggaagtagtgggatgggaacacaaatggagagctcagcgtctgcgtgttgggagctggagtcgtgggcttgacga |
| | cggcgctgcagctcttgcaggatgtgcctggcgtgcgcgttcacgtcgtggctgagaaatatggcgacgaaacgttgac |
| | ggctggggccggcgggctgtggatgccatacgcattgggtacgcggccattggatgggattggtaggcttatggaggg |
| | ataatagagtttttgtcggatccaacgcatgttgatgcagtagcccggtgtgctgaaagtatggaaggatggtgcattgg |
| | ctattcacatgcgctgcccaccccttttcgcaggaaatgtgccggcatcgttggtgcaccgatggggaaaatcgacgttc |
| | gaccactacatgaaaatgtatacctctgaagatgcagcaactgcgggtgcgaaacggataacggtttggtaggtgtatg |
| | tcgcagcatgtgctggattttgcgggcaactccccctgccacggcccattgcaggtgtcatgttgactggaggatacgaa |
| | ctttcggccgtcaaattcccagaggaggaccccctctgggccgacattgtgcccactgctcttccgcttgctgtttcctgtg |
| | tgaaattgt |
| 5 | MGAGGRIMVTPSSKKSETEALKRGPCEKPPFTVKDLKKAIPQHCFKRSIPRSFSYL |
| | LTDITLVSCFYYVATNYFSLLPQPLSTYLAWPLYWVCQGCVLTGIWVLGHECGH |
| | HAFSDYQWLDDTVGFIFHSLLLVPYFSWKYSHRRHHSNNGSLEKDEVFVPPKKA |
| | AVKWYVKYLNNPLGRILVLTVQFILGWPLYLAFNVSGRPYDGFASHFFPHAPIF |
| | KDRERLQIYISDAGILAVCYGLYRYAASQGLTAMICVYGVPLLIVNFFLVL VTFL |
| | QHTHPSLPHYDSTEWEWIRGALVTVDRDYGILNKVFHNITDTHVAHHLFATIPH |
| | YNAMEATEAIKPILGDYYHFDGTPWYVAMYREAKECLYVEPDTERGKKGVYY |
| | YNNKL |

Unless indicated otherwise, strains described herein were grown in 96-well block formats with shaking at 200 rpm at 28° C. for 72 hours for lipid production. Culture supernatants were harvested via centrifugation and washed once with MilliQ water. The resulting cellular pellet was lyophilized to dryness, and subjected to direct transesterification to generate FAMEs for subsequent quantitation and characterization by GC/FID. The process of strain engineering and subsequent fatty acid analysis is shown in FIG. 1.

Strains utilized for transformation were grown in vegetative growth medium for 24 hours prior to transformation. Cells were pelleted by centrifugation, resuspended in culture medium, and re-centrifuged. The resulting cell pellet was resuspended in culture medium. 5E+07 cells were plated to the appropriate selection medium and allowed to dry in a sterile biosafety cabinet. Transformants of CHK22 were generated via particle bombardment of previously plated cells using gold nanoparticles, and primary transformants were selected for ability to grow on plates containing sucrose as the sole carbon source. Transformants were grown in lipid production medium and the resulting biomass was processed as previously described for fatty acid analysis. TABLE 5 shows the fatty acid profiles of ten primary transformants of CHK22 transformed with pCHK277. P. moriformis base strain CHK22 is shown as a non-transgenic control.

TABLE 5
Screen of primary transformants of CHK22 with pCHK277

| | | | | | | | | | | 12-OH, |
|---|---|---|---|---|---|---|---|---|---|---|
| | | | | | | | C18:3 | | | 9c- |
| Sample Name | C12:0 | C14:0 | C16:0 | C18:0 | C18:1 | C18:2 | alpha | C20:0 | C20:1 | C18:1 |
| Sample 1 | 0.06 | 1.58 | 23.98 | 2.62 | 57.41 | 9.35 | 0.68 | 0.29 | 0.00 | 2.56 |
| Sample 2 | 0.04 | 1.49 | 23.68 | 2.63 | 58.12 | 9.06 | 0.62 | 0.30 | 0.03 | 2.55 |
| Sample 3 | 0.05 | 1.47 | 22.10 | 2.40 | 58.70 | 11.71 | 0.67 | 0.27 | 0.03 | 1.12 |
| Sample 4 | 0.05 | 1.57 | 22.90 | 2.45 | 57.64 | 11.92 | 0.70 | 0.29 | 0.03 | 1.03 |
| Sample 5 | 0.05 | 1.48 | 22.73 | 2.35 | 59.36 | 11.52 | 0.66 | 0.25 | 0.03 | 0.11 |
| Sample 6 | 0.05 | 1.48 | 23.01 | 2.39 | 58.74 | 11.82 | 0.69 | 0.25 | 0.03 | 0.08 |
| CHK22 | 0.04 | 1.56 | 28.91 | 3.18 | 56.89 | 7.27 | 0.55 | 0.25 | 0.02 | 0.07 |
| Control 1 | | | | | | | | | | |

Expressing RcFAH12 Oleate Hydroxylases in P. moriformis Strain CHK22

The R. communis oleate 12-hydroxylase (RcFAH12) was introduced into a P. moriformis strain CHK22. The expression construct contains 5′ and 3′ homology arms to permit its targeted integration into the P. moriformis genome, as shown in SEQ ID NO: 6.

TABLE 6 shows sequences of constructs. SEQ ID NO: 6 shows integrative sequences for the transformation of P. moriformis with PCHK170 encoding the RcFAH12. The construct can be written as 5′DAO1b::CrTUB2:ScSUC2:PmPGH:CvNR:PmAMT03:RcFAH12:CvNR::3′DAO1b. Proceeding in the 5′ to 3′ direction, bold, lowercase sequences represent genomic DNA from strain A that permit targeted integration at the DAO1b locus via homologous recombination. Proceeding in the 5′ to 3′ direction, the Chlamydomonas reinhardtii β-tubulin promoter driving expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics, in which contains a Chlorella vulgaris nitrate reductase 3′-UTR. An AMT03 promoter from P. moriformis, indicated by boxed italicized text, drives the expression of the RcFAH12. The Initiator ATG and terminator TGA codons of the RcFAH12 are indicated by uppercase, bold italics, while the remainder of the gene is indicated by bold italics. The C. vulgaris nitrate reductase 3′-UTR is indicated by lowercase underlined text followed by the CHK22 DAO1b genomic region indicated by bold, lowercase text. SEQ ID NO: 7 is the amino acid sequence of RcFAH12.

TABLE 6
Sequences

| SEQ | |
|---|---|
| ID NO | Sequence |
| 6 | gctcttccgcttgcccgcaccctcgttgatctgggagccctgcgcagccccttaaatcatctcagtcaggtttctatgttca |
| | actgagcctaaagggctttcgtcacgcgcacgagcacacgtatatcggctacgcagtctctcagaagcggtagaacagt |
| | tcgcaagccctcgtcggtcgaaaacttgcgccagtactattgaattaaattaaatgatcgaatgagacgcgaaacttttg |
| | cagaatgccactgagtttgcccagagaatgggagtggcgccattcaccatccgcctgtgcacggcctgattcgccgaga |
| | cgatgaatggcgagaccagggagcggcttgcgagccccgagccggtagcaggaataacggtcgacaatcttcctgtcc |
| | aattactggcaaccattagaaagagccggagcgcgttgaaattctgcaatcgagtaatttttcgatgcggcgggcctgct |
| | gaaccctaaggctccggcctatgtttaaggcgatccaagatgcacgcggccccaggcacgtgtctcaagcacaaaccc |
| | cagccttagtttcgagactttgggagatagcggcagatatctagtttggcattttgtatattaattaagtctcgcaatggag |
| | cgctctgatgcggtgcagcgtcggctgcagcacctggcagtggcgctggggtcgccctatcgctcggcgcctggtcagct |
| | 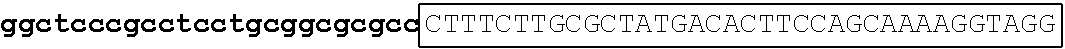 |
| | 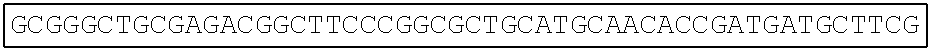 |
| | 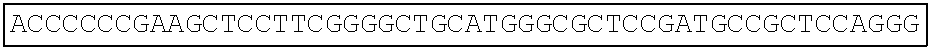 |
| | 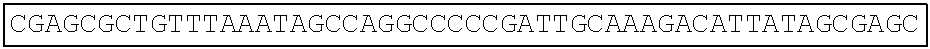 |
| | 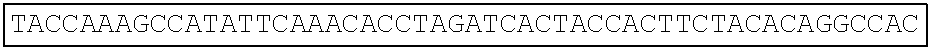 |
| | 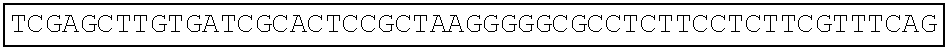 |
| | 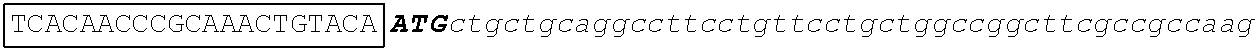 |
| | atcagcgcctccatgacgaacgagacgtccgaccgccccctggtgcacttcacccccaacaagggctggatgaacgacc |
| | ccaacggcctgtggtacgacgagaaggacgccaagtggcacctgtacttccagtacaacccgaacgacaccgtctgggg |
| | gacgcccttgttctggggccacgccacgtccgacgacctgaccaactgggaggaccagcccatcgccatcgccccgaag |
| | cgcaacgactccggcgccttctccggctccatggtggtggactacaacaacacctccggcttcttcaacgacaccatcgacc |
| | cgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctg |
| | gtacgagccctcccagaagtggatcatgaccgcggccaagtcccaggactacaagatcgagatctactcctccgacgacc |
| | tgaagtcctggaagctggagtccgcgttcgccaacgagggcttcctcggctaccagtacgagtgccccggcctgatcgagg |
| | tccccaccgagcaggaccccagcaagtcctactgggtgatgttcatctccatcaaccccggcgccccggccggcggctcct |
| | tcaaccagtacttcgtcggcagcttcaacggcacccacttcgaggccttcgacaaccagtcccgcgtggtggacttcggcaa |
| | ggactactacgccctgcagaccttcttcaacaccgacccgacctacgggagcgccctgggcatcgcgtgggcctccaactg |
| | ggagtactccgccttcgtgcccaccaacccctggcgctcctccatgtccctcgtgcgcaagttctccctcaacaccgagtacc |
| | aggccaacccggagacggagctgatcaacctgaaggccgagccgatcctgaacatcagcaacgccggcccctggagc |
| | cggttcgccaccaacaccacgttgacgaaggccaacagctacaacgtcgacctgtccaacagcaccggcaccctggagt |
| | tcgagctggtgtacgccgtcaacaccacccagacgatctccaagtccgtgttcgcggacctctccctctggttcaagggcctg |
| | gaggaccccgaggagtacctccgcatgggcttcgaggtgtccgcgtcctccttcttcctggaccgcgggaacagcaaggtg |
| | aagttcgtgaaggagaacccctacttcaccaaccgcatgagcgtgaacaaccagcccttcaagagcgagaacgacctgt |
| | cctactacaaggtgtacggcttgctggaccagaacatcctggagctgtacttcaacgacggcgacgtcgtgtccaccaaca |
| | cctacttcatgaccaccgggaacgccctgggctccgtgaacatgacgacgggggtggacaacctgttctacatcgacaagt |
| | tccaggtgcgcgaggtcaagtgactTAAGACGCCCGCGCGGCGCACCTGACCTGTTCTCTC |
| | GAGGGCGCCTGTTCTGCCTTGCGAAACAAGCCCCTGGAGCATGCGTGCATGA |
| | TCGTCTCTGGCGCCCCGCCGCGCGGTTTGTCGCCCTCGCGGGCGCCGCGGCC |
| | GCGGGGGCGCATTGAAATTGTTGCAAACCCCACCTGACAGATTGAGGGCCCA |
| | GGCAGGAAGGCGTTGAGATGGAGGTACAGGAGTCAAGTAACTGAAAGTTTT |
| | TATGATAACTAACAACAAAGGGTCGTTTCTGGCCAGCGAATGACAAGAACA |
| | AGATTCCACATTTCCGTGTAGAGGCTTGCCATCGAATGTGAGCGGGCGGGCC |
| | GCGGACCCGACAAAACCCTTACGACGTGGTAAGAAAAACGTGGCGGGCACT |
| | GTCCCTGTAGCCTGAAGACCAGCAGGAGACGATCGGAAGCATCACAGCACA |
| | ACCGGTgcagcagcagctcggatagtatcgacacactctggacgctggtcgtgtgatggactgttgccgccacacttg |
| | ctgccttgacctgtgaatatccctgccgcttttatcaaacagcctcagtgtgtttgatcttgtgtgtacgcgcttttgcgagttgcta |
| | gctgcttgtgctatttgcgaataccacccccagcatccccttccctcgtttcatatcgcttgcatcccaaccgcaacttatctacg |
| | ctgtcctgctatccctcagcgctgctcctgctcctgctcactgcccctcgcacagccttggtttgggctccgcctgtattctcctgg |
| | tactgcaacctgtaaaccagcactgcaatgctgatgcacgggaagtagtgggatgggaacacaaatggacctaggaaat |
| | 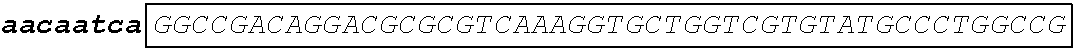 |
| | 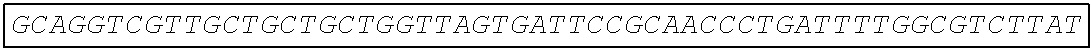 |
| | 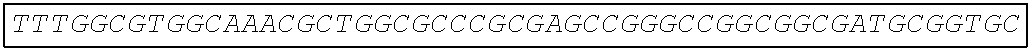 |
| | 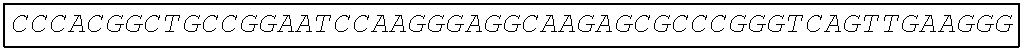 |
| | 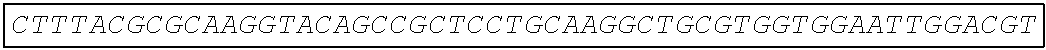 |
| | 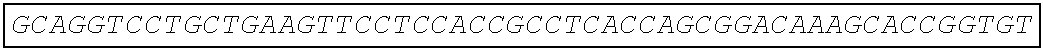 |
| | 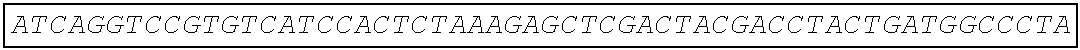 |
| | 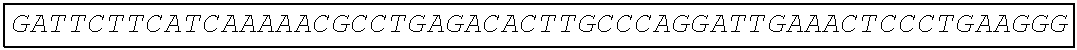 |
| | 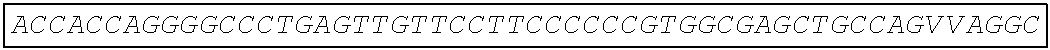 |
| | 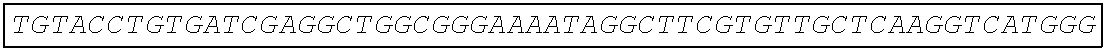 |
| | 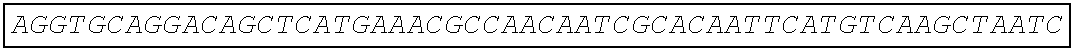 |
| | 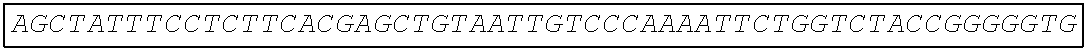 |
| | 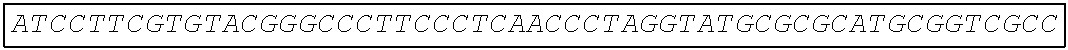 |
| | 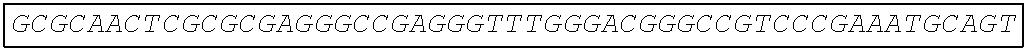 |
| | 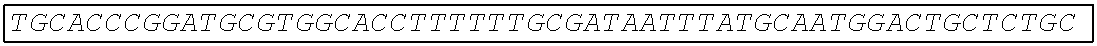 |
| | 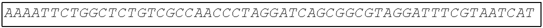 |
| | 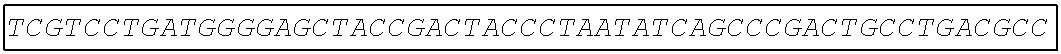 |
| | 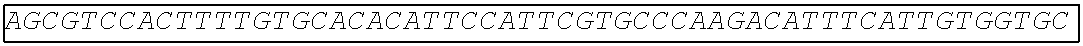 |
| | 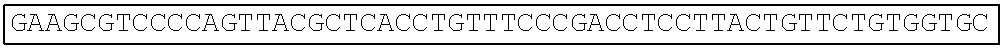 |
| | 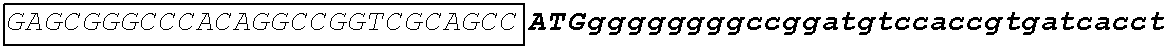 |
| | ccaacaactccgagaagaagggcggctcctcccacctgaagcgcgccccccacaccaagccccccttcaccctgggcg |
| | acctgaagcgcgccatccccccccactgcttcgagcgctccttcgtgcgctccttctcctacgtggcctacgacgtgtgcctgt |
| | ccttcctgttctactccatcgccaccaacttcttcccctacatctcctcccccctgtcctacgtggcctggctggtgtactggctgtt |
| | ccagggctgcatcctgacgggcctgtgggtgatcggccacgagtgcggccaccacgccttctccgagtaccagctcgccg |
| | acgacatcgtgggcctgatcgtgcactccgccctgctggtgccctacttctcctggaagtactcccaccgccgccaccactcc |
| | aacatcggctccctggagcgcgacgaggtgttcgtgcccaagtccaagtccaagatctcctggtactccaagtactcgaac |
| | aacccccccggccgcgtgctgaccctggccgccaccctgctgctgggctggcccctgtacctggccttcaacgtgtccggcc |
| | gcccctacgaccgcttcgcctgccactacgacccctacggccccatcttctccgagcgcgagcgcctccagatctacatcgc |
| | cgacctgggcatcttcgccaccaccttcgtgctgtaccaggccaccatggcgaagggcctggcctgggtgatgcgcatctac |
| | ggcgtgcccctgctgatcgtcaactgcttcctggtgatgatcacctacctccagcacacccaccccgccatcccccgctacg |
| | gctcctccgagtgggactggctgcgcggcgccatggtgaccgtggaccgcgactacggcgtgctgaacaaggtgttccac |
| | aacatcgccgacacccacgtggcccaccacctgttcgccaccgtgccccactaccacgccatggaggccaccaaggcca |
| | tcaagcccatcatgggcgagtactaccgctacgacggcacccccttctacaaggccctgtggcgcgaggccaaggagtgc |
| | ctgttcgtggagcccgacgagggcgcccccacccagggcgtgttctggtacaggaacaagtacTGAgcagcagcagct |
| | cggatagtatcgacacactctggacgctggtcgtgtgatggactgttgccgccacacttgctgccttgacctgtgaatatccctgc |
| | cgcttttatcaaacagcctcagtgtgtttgatcttgtgtgtacgcgcttttgcgagttgctagctgcttgtgctatttgcgaataccacc |
| | cccagcatccccttccctcgtttcatatcgcttgcatcccaaccgcaacttatctacgctgtcctgctatccctcagcgctgctcctg |
| | ctcctgctcactgcccctcgcacagccttggtttgggctccgcctgtattctcctggtactgcaacctgtaaaccagcactgcaatg |
| | ctgatgcacgggaagtagtgggatgggaacacaaatggagagctcagcgtctgcgtgttgggagctggagtcgtgggcttg |
| | acgacggcgctgcagctcttgcaggatgtgcctggcgtgcgcgttcacgtcgtggctgagaaatatggcgacgaaacgt |
| | tgacggctggggccggcgggctgtggatgccatacgcattgggtacgcggccattggatgggattggtaggcttatgga |
| | gggataatagagtttttgtcggatccaacgcatgttgatgcagtagcccggtgtgctgaaagtatggaaggatggtgcat |
| | tggctattcacatgcgctgcccaccccttttcgcaggaaatgtgccggcatcgttggtgcaccgatggggaaaatcgacg |
| | ttcgaccactacatgaaaatgtatacctctgaagatgcagcaactgcgggtgcgaaacggataacggtttggtaggtgt |
| | atgtcgcagcatgtgctggattttgcgggcaactccccctgccacggcccattgcaggtgtcatgttgactggaggatac |
| | gaactttcggccgtcaaattcccagaggaggaccccctctgggccgacattgtgcccactgctcttccgcttgctgtttcct |
| | gtgtgaaattgt |
| 7 | MGGGGRMSTVITSNNSEKKGGSSHLKRAPHTKPPFTLGDLKRAIPPHCFERSFVR |
| | SFSYVAYDVCLSFLFYSIATNFFPYISSPLSYVAWLVYWLFQGCILTGLWVIGHEC |
| | GHHAFSEYQLADDIVGLIVHSALLVPYFSWKYSHRRHHSNIGSLERDEVFVPKSK |
| | SKISWYSKYSNNPPGRVLTLAATLLLGWPLYLAFNVSGRPYDRFACHYDPYGPI |
| | FSERERLQIYIADLGIFATTFVLYQATMAKGLAWVMRIYGVPLLIVNCFLVMITY |
| | LQHTHPAIPRYGSSEWDWLRGAMVTVDRDYGVLNKVFHNIADTHVAHHLFAT |
| | VPHYHAMEATKAIKPIMGEYYRYDGTPFYKALWREAKECLFVEPDEGAPTQGV |
| | FWYRNKY |

TABLE 7 shows the fatty acid profiles of ten primary transformants of CHK22 transformed with PCHK170 and grown as described for oil production and resulting FAMEs analyzed by GC-FID as described. P. moriformis base strain CHK22 is shown as a non-transgenic control. Strains were grown for 72 hours in 96-well blocks at 200 rpm at 28° C. Biomass was harvested, lyophilized to dryness, and subjected to direct transesterification to generate FAMEs for subsequent quantitation and characterization by GC/FID.

TABLE 7
Screen of primary transformants of CHK22 with PCHK170

| | | | | | | | C18:3 | | | 12-OH- |
|---|---|---|---|---|---|---|---|---|---|---|
| Sample Name | C12:0 | C14:0 | C16:0 | C18:0 | C18:1 | C18:2 | alpha | C20:0 | C20:1 | C18:1 |
| Sample 8 | 0.05 | 1.56 | 24.93 | 2.38 | 57.92 | 10.58 | 0.71 | 0.20 | 0.09 | 0.21 |
| Sample 9 | 0.05 | 1.61 | 26.75 | 2.47 | 57.39 | 9.21 | 0.70 | 0.19 | 0.08 | 0.19 |
| Sample 10 | 0.05 | 1.61 | 24.10 | 2.36 | 57.62 | 11.69 | 0.72 | 0.20 | 0.09 | 0.18 |
| Sample 11 | 0.05 | 1.75 | 27.10 | 2.61 | 56.27 | 9.69 | 0.74 | 0.22 | 0.08 | 0.17 |
| Sample 12 | 0.05 | 1.55 | 23.52 | 2.43 | 58.24 | 11.56 | 0.70 | 0.20 | 0.09 | 0.12 |
| Sample 13 | 0.04 | 1.43 | 23.41 | 2.67 | 59.09 | 10.78 | 0.63 | 0.25 | 0.11 | 0.10 |
| Sample 14 | 0.05 | 1.60 | 23.56 | 2.44 | 57.81 | 11.84 | 0.70 | 0.21 | 0.10 | 0.10 |
| Sample 15 | 0.05 | 1.58 | 25.63 | 2.53 | 57.62 | 9.94 | 0.67 | 0.21 | 0.09 | 0.09 |
| CHK22 | 0.05 | 1.60 | 24.39 | 2.37 | 58.00 | 11.14 | 0.70 | 0.20 | 0.10 | 0.05 |
| Control 2 | | | | | | | | | | |

Expressing CpFAH12 Oleate Hydroxylases in P. moriformis Strain CHK22

The C. purpurea oleate 12-hydroxylase (CpFAH12) was introduced into a P. moriformis strain CHK22. The expression construct contains 5′ and 3′ homology arms to permit its targeted integration into the P. moriformis genome, as seen in SEQ ID NO: 8.

TABLE 8 shows sequences of constructs. SEQ ID NO: 8 shows integrative sequences for the transformation of P. moriformis with pCHK271 encoding the CpFAH12. The construct can be written as 5′DAO1b::CrTUB2:ScSUC2:PmPGH:CvNR:PmAMT03:CpFAH12:CvNR::3′DAO1b. Proceeding in the 5′ to 3′ direction, bold, lowercase sequences represent genomic DNA from strain A that permit targeted integration at the DAO1b locus via homologous recombination. Proceeding in the 5′ to 3′ direction, the C. reinhardtii β-tubulin promoter driving expression of the yeast sucrose invertase gene (conferring the ability of strain A to metabolize sucrose) is indicated by boxed uppercase text. The initiator ATG and terminator TAA for invertase are indicated by uppercase, bold italics while the coding region is indicated in lowercase italics. The P. moriformis PmPGH 3′UTR is indicated by uppercase underlined text followed by a linker, indicated by lowercase, bold italics, which contains a C. vulgaris nitrate reductase 3′-UTR that enables amplification of the oleate 12-hydroxylase. An AMT03 promoter from P. moriformis, indicated by boxed italicized text, drives the expression of the CpFAH12. The Initiator ATG and terminator TGA codons of CpFAH12 are indicated by uppercase, bold italics, while the remainder of the gene is indicated by bold italics. The C. vulgaris nitrate reductase 3′-UTR is indicated by lowercase underlined text followed by CHK22 DAO1b genomic region indicated by bold, lowercase text. SEQ ID NO: 9 shows the amino acid sequence of CpFAH12.

TABLE 8
Sequences

| SEQ | |
|---|---|
| ID NO | Sequence |
| 8 | gctcttccgcttgcccgcaccctcgttgatctgggagccctgcgcagccccttaaatcatctcagtcaggtttctatgttca |
| | actgagcctaaagggctttcgtcacgcgcacgagcacacgtatatcggctacgcagtctctcagaagcggtagaacagt |
| | tcgcaagccctcgtcggtcgaaaacttgcgccagtactattgaattaaattaaatgatcgaatgagacgcgaaacttttg |
| | cagaatgccactgagtttgcccagagaatgggagtggcgccattcaccatccgcctgtgcacggcctgattcgccgaga |
| | cgatgaatggcgagaccagggagcggcttgcgagccccgagccggtagcaggaataacggtcgacaatcttcctgtcc |
| | aattactggcaaccattagaaagagccggagcgcgttgaaattctgcaatcgagtaatttttcgatgcggcgggcctgct |
| | gaaccctaaggctccggcctatgtttaaggcgatccaagatgcacgcggccccaggcacgtgtctcaagcacaaaccc |
| | cagccttagtttcgagactttgggagatagcggcagatatctagtttggcattttgtatattaattaagtctcgcaatggag |
| | cgctctgatgcggtgcagcgtcggctgcagcacctggcagtggcgctggggtcgccctatcgctcggcgcctggtcagct |
| | 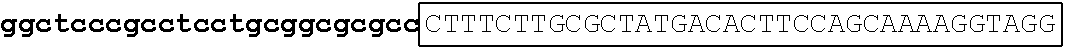 |
| | 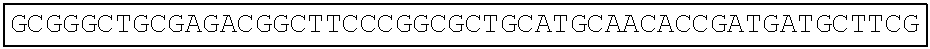 |
| | 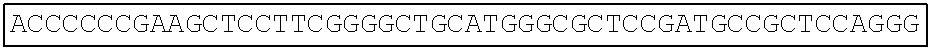 |
| | 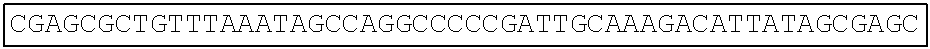 |
| | 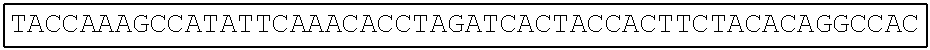 |
| | 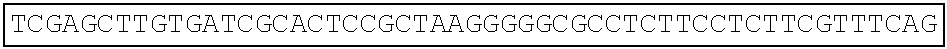 |
| | 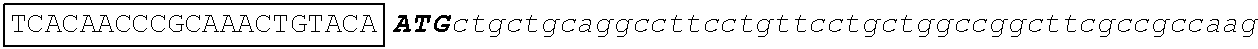 |
| | atcagcgcctccatgacgaacgagacgtccgaccgccccctggtgcacttcacccccaacaagggctggatgaacgacc |
| | ccaacggcctgtggtacgacgagaaggacgccaagtggcacctgtacttccagtacaacccgaacgacaccgtctgggg |
| | gacgcccttgttctggggccacgccacgtccgacgacctgaccaactgggaggaccagcccatcgccatcgccccgaag |
| | cgcaacgactccggcgccttctccggctccatggtggtggactacaacaacacctccggcttcttcaacgacaccatcgacc |
| | cgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctg |
| | gtacgagccctcccagaagtggatcatgaccgcggccaagtcccaggactacaagatcgagatctactcctccgacgacc |
| | tgaagtcctggaagctggagtccgcgttcgccaacgagggcttcctcggctaccagtacgagtgccccggcctgatcgagg |
| | tccccaccgagcaggaccccagcaagtcctactgggtgatgttcatctccatcaaccccggcgccccggccggcggctcct |
| | tcaaccagtacttcgtcggcagcttcaacggcacccacttcgaggccttcgacaaccagtcccgcgtggtggacttcggcaa |
| | ggactactacgccctgcagaccttcttcaacaccgacccgacctacgggagcgccctgggcatcgcgtgggcctccaactg |
| | ggagtactccgccttcgtgcccaccaacccctggcgctcctccatgtccctcgtgcgcaagttctccctcaacaccgagtacc |
| | aggccaacccggagacggagctgatcaacctgaaggccgagccgatcctgaacatcagcaacgccggcccctggagc |
| | cggttcgccaccaacaccacgttgacgaaggccaacagctacaacgtcgacctgtccaacagcaccggcaccctggagt |
| | tcgagctggtgtacgccgtcaacaccacccagacgatctccaagtccgtgttcgcggacctctccctctggttcaagggcctg |
| | gaggaccccgaggagtacctccgcatgggcttcgaggtgtccgcgtcctccttcttcctggaccgcgggaacagcaaggtg |
| | aagttcgtgaaggagaacccctacttcaccaaccgcatgagcgtgaacaaccagcccttcaagagcgagaacgacctgt |
| | cctactacaaggtgtacggcttgctggaccagaacatcctggagctgtacttcaacgacggcgacgtcgtgtccaccaaca |
| | cctacttcatgaccaccgggaacgccctgggctccgtgaacatgacgacgggggtggacaacctgttctacatcgacaagt |
| | tccaggtgcgcgaggtcaagtgactTAAGACGCCCGCGCGGCGCACCTGACCTGTTCTCTC |
| | GAGGGCGCCTGTTCTGCCTTGCGAAACAAGCCCCTGGAGCATGCGTGCATGA |
| | TCGTCTCTGGCGCCCCGCCGCGCGGTTTGTCGCCCTCGCGGGCGCCGCGGCC |
| | GCGGGGGCGCATTGAAATTGTTGCAAACCCCACCTGACAGATTGAGGGCCCA |
| | GGCAGGAAGGCGTTGAGATGGAGGTACAGGAGTCAAGTAACTGAAAGTTTT |
| | TATGATAACTAACAACAAAGGGTCGTTTCTGGCCAGCGAATGACAAGAACA |
| | AGATTCCACATTTCCGTGTAGAGGCTTGCCATCGAATGTGAGCGGGCGGGCC |
| | GCGGACCCGACAAAACCCTTACGACGTGGTAAGAAAAACGTGGCGGGCACT |
| | GTCCCTGTAGCCTGAAGACCAGCAGGAGACGATCGGAAGCATCACAGCACA |
| | ACCGGTgcagcagcagctcggatagtatcgacacactctggacgctggtcgtgtgatggactgttgccgccacacttg |
| | ctgccttgacctgtgaatatccctgccgcttttatcaaacagcctcagtgtgtttgatcttgtgtgtacgcgcttttgcgagttgcta |
| | gctgcttgtgctatttgcgaataccacccccagcatccccttccctcgtttcatatcgcttgcatcccaaccgcaacttatctacg |
| | ctgtcctgctatccctcagcgctgctcctgctcctgctcactgcccctcgcacagccttggtttgggctccgcctgtattctcctgg |
| | tactgcaacctgtaaaccagcactgcaatgctgatgcacgggaagtagtgggatgggaacacaaatggacctaggaaat |
| | 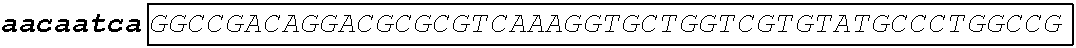 |
| | 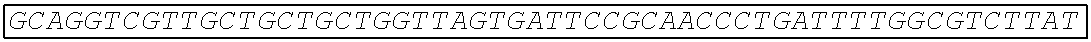 |
| | 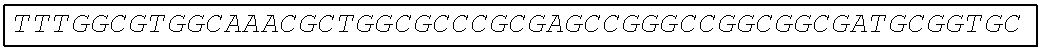 |
| | 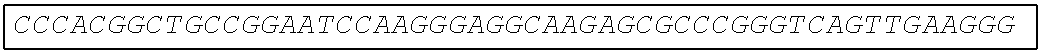 |
| | 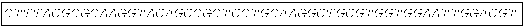 |
| | 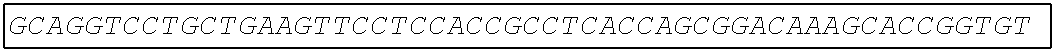 |
| | 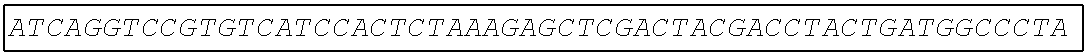 |
| | 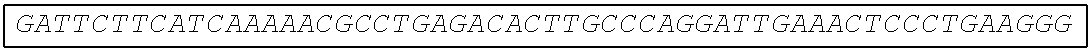 |
| | 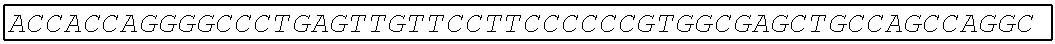 |
| | 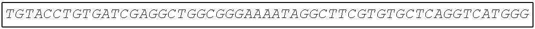 |
| | 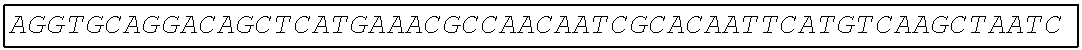 |
| | 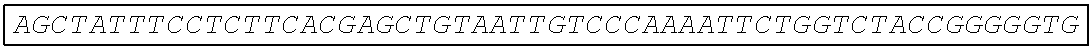 |
| | 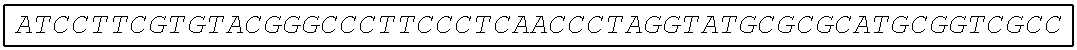 |
| | 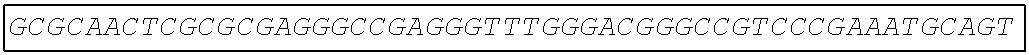 |
| | 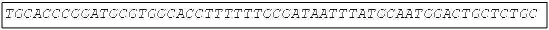 |
| | 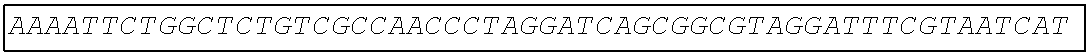 |
| | 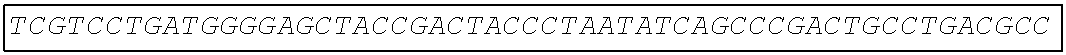 |
| | 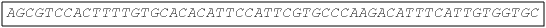 |
| | 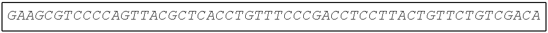 |
| | 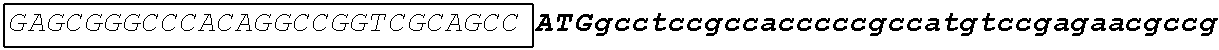 |
| | tgctgcgccacaaggccgcctccaccacgggcatcgactacgagtcctccgccgccgtgtcccccgccgagtccccccgc |
| | acctccgcctcctccacctccctgtcctccctgtcctccctggacgccaacgagaagaaggacgagtacgcgggcctgctg |
| | gacacctacggcaacgccttcaccccccccgacttctccatcaaggacatccgcgccgccatccccaagcactgctacga |
| | gcgctccaccatcaagtcctacgcctacgtgctgcgcgacctgctgtgcctgtccaccaccttctacctgttccacaacttcgt |
| | gacccccgagaacatcccctccaaccccctgcgcttcgtgctgtggtccatctacaccgtgctccagggcctgttcgccacg |
| | ggcctgtgggtgatcggccacgagtgcggccactgcgccttctccccctcccccttcatctccgacctgacgggctgggtgat |
| | ccactccgccctgctggtgccctacttctcctggaagttctcccactccgcccaccacaagggcatcggcaacatggagcgc |
| | gacatggtgttcctgccccgcacccgcgagcagcaggcgacccgcctgggccgcgccgtggaggagctgggcgacctgt |
| | gcgaggagacccccatctacaccgccctgcacctggtgggcaagcagctcatcggctggccctcctacctgatgaccaac |
| | gccacgggccacaacttccacgagcgccagcgcgagggccgcggcaagggcaagaagaacggctttggcggcggcgt |
| | caaccacttcgacccccgctcccccatcttcgaggcccgccaggccaagtacatcgtgctgtccgacatcggcctgggcct |
| | ggccatcgccgccctggtgtacctgggcaaccgcttcggctgggccaacatgaccgtgtggtacttcctgccctacctgtgg |
| | gtgaaccactggctggtggccatcaccttcctccagcacaccgaccccaccctgccccactacaaccgcgaggagtggaa |
| | cttcgtgcgcggcggcgcctgcaccatcgaccgcgacctgggcttcatcggccgccacctgttccacggcatcgccgacac |
| | ccacgtggtgcaccactacgtgtcccgcatccccttctacaacgccgacgaggcctccgaggccatcaagcccatcatggg |
| | caagcactaccgctccgacaccgcccacggccccgtgggcttcctgcacgccctgtggaagaccgcccgctggtgccagt |
| | gggtggagccctccgccgacgcccagggcgcgggcaagggcatcctgttctaccgcaaccgcaacaagctgggcacca |
| | agcccatctccatgaagacccagTGAgcagcagcagctcggatagtatcgacacactctggacgctggtcgtgtgatgga |
| | ctgttgccgccacacttgctgccttgacctgtgaatatccctgccgcttttatcaaacagcctcagtgtgtttgatcttgtgtgtacgc |
| | gcttttgcgagttgctagctgcttgtgctatttgcgaataccacccccagcatccccttccctcgtttcatatcgcttgcatcccaacc |
| | gcaacttatctacgctgtcctgctatccctcagcgctgctcctgctcctgctcactgcccctcgcacagccttggtttgggctccgc |
| | ctgtattctcctggtactgcaacctgtaaaccagcactgcaatgctgatgcacgggaagtagtgggatgggaacacaaatggag |
| | agctcagcgtctgcgtgttgggagctggagtcgtgggcttgacgacggcgctgcagctcttgcaggatgtgcctggcgtg |
| | cgcgttcacgtcgtggctgagaaatatggcgacgaaacgttgacggctggggccggcgggctgtggatgccatacgca |
| | ttgggtacgcggccattggatgggattggtaggcttatggagggataatagagtttttgtcggatccaacgcatgttgatg |
| | cagtagcccggtgtgctgaaagtatggaaggatggtgcattggctattcacatgcgctgcccaccccttttcgcaggaaa |
| | tgtgccggcatcgttggtgcaccgatggggaaaatcgacgttcgaccactacatgaaaatgtatacctctgaagatgca |
| | gcaactgcgggtgcgaaacggataacggtttggtaggtgtatgtcgcagcatgtgctggattttgcgggcaactccccct |
| | gccacggcccattgcaggtgtcatgttgactggaggatacgaactttcggccgtcaaattcccagaggaggaccccctct |
| | gggccgacattgtgcccactgctcttccgcttgctgtttcctgtgtgaaattgt |
| 9 | MASATPAMSENAVLRHKAASTTGIDYESSAAVSPAESPRTSASSTSLSSLSSLDA |
| | NEKKDEYAGLLDTYGNAFTPPDFSIKDIRAAIPKHCYERSTIKSYAYVLRDLLCLS |
| | TTFYLFHNFVTPENIPSNPLRFVLWSIYTVLQGLFATGLWVIGHECGHCAFSPSPFI |
| | SDLTGWVIHSALLVPYFSWKFSHSAHHKGIGNMERDMVFLPRTREQQATRLGR |
| | AVEELGDLCEETPIYTALHLVGKQLIGWPSYLMTNATGHNFHERQREGRGKGK |
| | KNGFGGGVNHFDPRSPIFEARQAKYIVLSDIGLGLAIAALVYLGNRFGWANMTV |
| | WYFLPYLWVNHWLVAITFLQHTDPTLPHYNREEWNFVRGGACTIDRDLGFIGR |
| | HLFHGIADTHVVHHYVSRIPFYNADEASEAIKPIMGKHYRSDTAHGPVGFLHAL |
| | WKTARWCQWVEPSADAQGAGKGILFYRNRNKLGTKPISMKTQ |

TABLE 9 shows the fatty acid profiles of ten primary transformants of CHK22 transformed with pCHK271 and grown as described for oil production and resulting FAMEs analyzed by GC-FID as described. P. moriformis base strain CHK22 is shown as non-transgenic controls. Strains were grown for 72 hours in 96-well blocks at 200 rpm at 28° C. Biomass was harvested, lyophilized to dryness, and subjected to direct transesterification to generate FAMEs for subsequent quantitation and characterization by GC/FID.

TABLE 9
Screen of primary transformants of CHK22 with pCHK271

| | | | | | | | C18:3 | | | 12-OH- |
|---|---|---|---|---|---|---|---|---|---|---|
| Sample Name | C12:0 | C14:0 | C16:0 | C18:0 | C18:1 | C18:2 | alpha | C20:0 | C20:1 | C18:1 |
| Sample 16 | 0.05 | 1.67 | 25.01 | 2.32 | 57.18 | 11.11 | 0.70 | 0.20 | 0.09 | 0.06 |
| Sample 17 | 0.05 | 1.62 | 24.61 | 2.39 | 57.72 | 11.03 | 0.69 | 0.21 | 0.09 | 0.05 |
| Sample 18 | 0.05 | 1.64 | 24.33 | 2.43 | 57.50 | 11.43 | 0.72 | 0.21 | 0.10 | 0.06 |
| Sample 19 | 0.05 | 1.65 | 24.84 | 2.34 | 57.34 | 11.11 | 0.71 | 0.20 | 0.10 | 0.05 |
| Sample 20 | 0.04 | 1.55 | 24.38 | 2.52 | 58.34 | 10.64 | 0.64 | 0.23 | 0.10 | 0.04 |
| Sample 21 | 0.04 | 1.48 | 23.81 | 2.55 | 58.88 | 10.70 | 0.64 | 0.24 | 0.10 | 0.04 |
| Sample 22 | 0.05 | 1.67 | 25.77 | 2.50 | 56.99 | 10.36 | 0.69 | 0.21 | 0.09 | 0.08 |
| Sample 23 | 0.05 | 1.69 | 25.35 | 2.34 | 56.99 | 10.85 | 0.70 | 0.20 | 0.09 | 0.06 |
| Sample 24 | 0.05 | 1.70 | 25.00 | 2.33 | 57.00 | 11.24 | 0.71 | 0.20 | 0.09 | 0.05 |
| Sample 25 | 0.05 | 1.71 | 26.65 | 2.32 | 56.96 | 9.67 | 0.71 | 0.19 | 0.09 | 0.04 |
| CHK22 | 0.04 | 1.66 | 28.92 | 2.72 | 56.36 | 7.67 | 0.60 | 0.19 | 0.08 | 0.06 |
| Control 3 | | | | | | | | | | |

Transformants of CHK22 were generated via particle bombardment using gold nanoparticles, upon transformation of plasmid constructs (expressing oleate hydroxylases) into strain CHK22, positive clones were selected on plates containing sucrose as the sole carbon source. Primary transformants were clonally purified and grown under standard lipid production conditions. Lipid samples were prepared from dried biomass from each transformant. Fatty acid profiles were determined using direct transesterification methods. Fatty acid profiles (expressed as Area % of total fatty acid) of several positive transformants, compared to those of untransformed strain CHK22 controls.

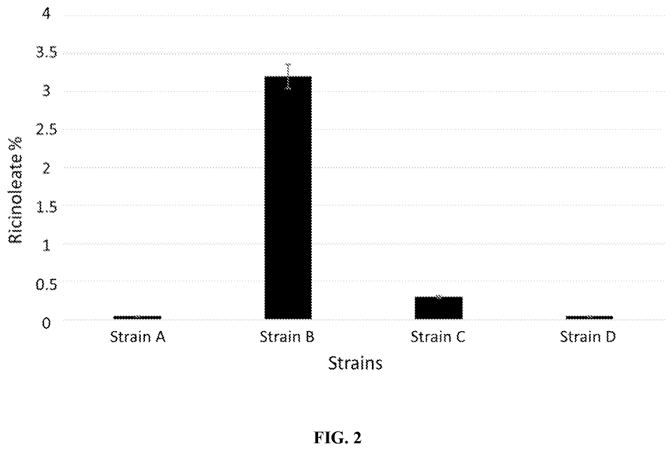

The untransformed P. moriformis (UTEX 1533) strain CHK22 exhibits a fatty acid profile comprising less than 0.1% ricinoleate. In contrast, fatty acid profiles of strain CHK22 expressing RcFAH12 and LFAH12 genes showed increased composition of 12-OH C18:1 fatty acids, ranging from about 0.2% to 2.6% ricinoleate, respectively (TABLE 2 and 3), while strains expressing CpFAH12 showed no increased accumulation of ricinoleate. One of the transformants of CHK22, transformed with pCHK277, was banked as CHK83 for subsequent studies. FIG. 2 shows a comparison of ricinoleate levels in P. moriformis strains.

### Example 3: Expressing R. communis Acyltransferases in Strain CHK83

Studies of transgenic plants have demonstrated that expressing R. communis acyltransferases can enhance accumulation of hydroxy fatty acids in transgenic seeds. In an effort to increase ricinoleate production in microalgae, this experiment assessed the impact of various heterologous acyltransferases from R. communis when introduced into CHK83, a strain expressing LFAH12. In this example, either the R. communis phospholipid:diacylglycerol acyltransferase (RcPDAT1, GenBank Accession No. AEW99982.1), the R. communis putative phospholipid:diacylglycerol acyltransferase 2 precursor (RcPDAT2, GenBank accession No. NP 001310647.1), the R. communis phosphatidylcholine:diacylglycerol cholinephosphotransferase 1 (RcPDCT1, GenBank Accession No. XP 002517643.1), or the R. communis diacylglycerol O-acyltransferase 2 (RcDGAT2, GenBank Accession No. NP 001310616.1) were independently expressed in strain CHK83.

FIG. 3 shows an overview of de novo TAG biosynthesis in microalgae and the oilseeds of higher plants. Abbreviations for lipids (grey boxed letters) are as follows: G3P, glycerol-3-phosphate; LPA, lysophosphatidic acid; PA, phosphatidic acid; DAG, diacylglycerol; PC, phosphatidylcholine; TAG, triacylglycerol. Abbreviations for enzymes (no box): GPAT, G3P acyltransferase; LPAT, lysophosphatidylacyltransferase; PAP, phosphatidate phosphatase; PDCT, PC:DAG cholinephosphotransferase; DGAT, acyl-CoA:DAG acyltransferase; and PDAT, phospholipid:DAG acyltransferase.

Expressing RcPDAT1 in P moriformis Strain CHK83

The R. communis PDAT1 (RcPDAT1) was introduced into a derivative of P. moriformis UTEX1533, strain CHK83. The expression construct contained 5′ and 3′ homology arms to permit its targeted integration into the P. moriformis genome, as seen in SEQ ID NO: 10.

TABLE 10 shows sequences of constructs. SEQ ID NO: 10 shows integrative sequences for the transformation of P. moriformis strain CHK83 with PCHK350 encoding the RcPDAT1. The construct can be written as 5′Thi4::PmHXT1v2:ScMEL1:PmPGK:CvNR:PmSAD2-2:RcPDAT1:CvNR::3′Thi4. Proceeding in the 5′ to 3′ direction, bold, lowercase sequences represent genomic DNA from strain CHK22 that permit targeted integration at the Thi4 locus via homologous recombination. Proceeding in the 5′ to 3′ direction, the P. moriformis hexose transporter 1 (HXT1 v2) promoter driving expression of the S. carlbergensis melibiase (ScMEL1) is indicated by boxed uppercase text. The initiator ATG and terminator TAA for ScMEL1 are indicated by uppercase, bold italics while the coding region is indicated in lowercase italics. P. moriformis phosphoglucokinase (PGK) 3′-UTR is indicated by uppercase underlined text followed by a stearoyl ACP desaturase-2 (SAD2-2) promoter from P. moriformis, indicated by boxed italicized text, driving the expression of RcPDAT1. The Initiator ATG and terminator TGA codons of RcPDAT1 are indicated by uppercase, bold italics, while the remainder of the gene is indicated by bold italics. The C. vulgaris nitrate reductase 3′-UTR is indicated by lowercase underlined text followed by the P. moriformis Thi4 3′ flanking region indicated by bold, lowercase text. SEQ ID NO: 11 shows the amino acid sequence of RcPDAT1.

TABLE 10
Sequences

| SEQ | |
|---|---|
| ID NO | Sequence |
| 10 | ccctcaactgcgacgctgggaaccttctccgggcaggcgatgtgcgtgggtttgcctccttggcacggctctacaccgtc |
| | gagtacgccatgaggcggtgatggctgtgtcggttgccacttcgtccagagacggcaagtcgtccatcctctgcgtgtgt |
| | ggcgcgacgctgcagcagtccctctgcagcagatgagcgtgactttggccatttcacgcactegagtgtacacaatccat |
| | ttttcttaaagcaaatgactgctgattgaccagatactgtaacgctgatttcgctccagatcgcacagatagcgaccatgt |
| | tgctgcgtctgaaaatctggattccgaattcgaccctggcgctccatccatgcaacagatggcgacacttgttacaattcc |
| | tgtcacccatcggcatggagcaggtccacttagattcccgatcacccacgcacatctcgctaatagtcattcgttcgtgtct |
| | tcgatcaatctcaagtgagtgtgcatggatcttggttgacgatgcggtatgggtttgcgccgctggctgcagggtctgccc |
| | aaggcaagctaacccagctcctctccccgacaatactctcgcaggcaaagccggtcacttgccttccagattgccaata |
| | aactcaattatggcctctgtcatgccatccatgggtctgatgaatggtcacgctcgtgtcctgaccgttccccagcctctgg |
| | 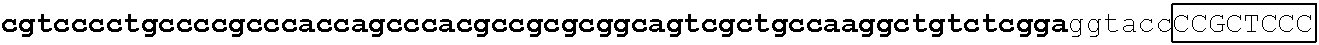 |
| | 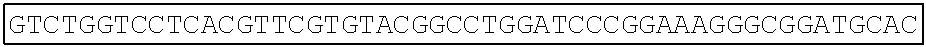 |
| | 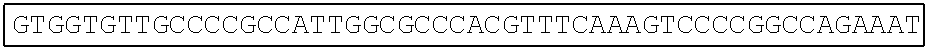 |
| | 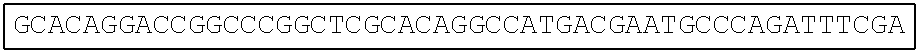 |
| | 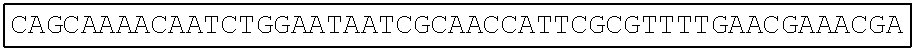 |
| | 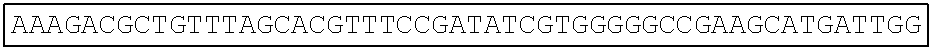 |
| | 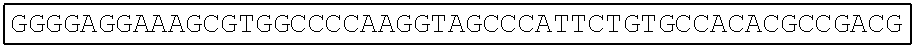 |
| | 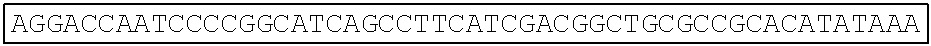 |
| | 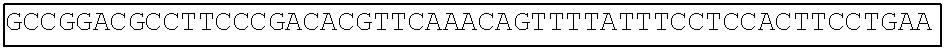 |
| | 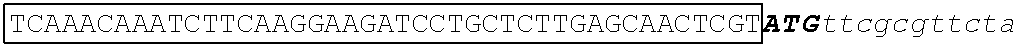 |
| | cttcctgacggcctgcatctccctgaagggcgtgtteggegtctccccctcctacaacggcctgggcctgacgccccagatg |
| | ggctgggacaactggaacacgttcgcctgcgacgtctccgagcagctgctgctggacacggccgaccgcatctccgacct |
| | gggcctgaaggacatgggctacaagtacatcatcctggacgactgctggtcctccggccgcgactccgacggcttcctggt |
| | cgccgacgagcagaagttccccaacggcatgggccacgtcgccgaccacctgcacaacaactccttcctgttcggcatgt |
| | actcctccgcgggcgagtacacgtgcgccggctaccccggctccctgggccgcgaggaggaggacgcccagttcttcgcg |
| | aacaaccgcgtggactacctgaagtacgacaactgctacaacaagggccagttcggcacgcccgagatctcctaccacc |
| | gctacaaggccatgtccgacgccctgaacaagacgggccgccccatcttctactccctgtgcaactggggccaggacctg |
| | accttctactggggctccggcatcgcgaactcctggcgcatgtccggcgacgtcacggeggagttcacgegccccgactcc |
| | cgctgcccctgcgacggcgacgagtacgactgcaagtacgccggcttccactgctccatcatgaacatcctgaacaaggc |
| | cgcccccatgggccagaacgcgggcgtcggcggctggaacgacctggacaacctggaggtcggcgtcggcaacctgac |
| | ggacgacgaggagaaggcgcacttctccatgtgggccatggtgaagtcccccctgatcatcggcgcgaacgtgaacaac |
| | ctgaaggcctcctcctactccatctactcccaggegtccgtcategccatcaaccaggactccaacggcatccccgccacgc |
| | gcgtctggcgctactacgtgtccgacacggacgagtacggccagggcgagatccagatgtggtccggccccctggacaa |
| | cggcgaccaggtcgtggcgctgctgaacggcggctccgtgtcccgccccatgaacacgaccctggaggagatcttcttcga |
| | ctccaacctgggctccaagaagctgacctccacctgggacatctacgacctgtgggcgaaccgcgtcgacaactccacgg |
| | cgtccgccatcctgggccgcaacaagaccgccaccggcatcctgtacaacgccaccgagcagtcctacaaggacggcct |
| | gtccaagaacgacacccgcctgttcggccagaagatcggctccctgtcccccaacgcgatcctgaacacgaccgtccccg |
| | cccacggcatcgcgttctaccgcctgcgcccctcctccTGATACAACTTATTACGTATTCTGACCGG |
| | CGCTGATGTGGCGCGGACGCCGTCGTACTCTTTCAGACTTTACTCTTGAGGA |
| | ATTGAACCTTTCTCGCTTGCTGGCATGTAAACATTGGCGCAATTAATTGTGTG |
| | ATGAAGAAAGGGTGGCACAAGATGGATCGCGAATGTACGAGATCGACAACG |
| | ATGGTGATTGTTATGAGGGGCCAAACCTGGCTCAATCTTGTCGCATGTCCGG |
| | CGCAATGTGATCCAGCGGCGTGACTCTCGCAACCTGGTAGTGTGTGCGCACC |
| | GGGTCGCTTTGATTAAAACTGATCGCATTGCCATCCCGTCAACTCACAAGCC |
| | TACTCTAGCTCCCATTGCGCACTCGGGCGCCCGGCTCGATCAATGTTCTGAGC |
| | GGAGGGCGAAGCGTCAGGAAATCGTCTCGGCAGCTGGAAGCGCATGGAATG |
| | 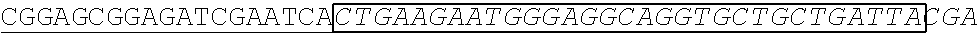 |
| | 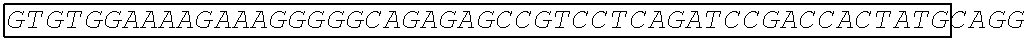 |
| | 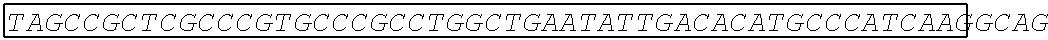 |
| | 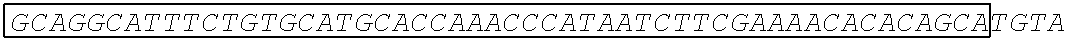 |
| | 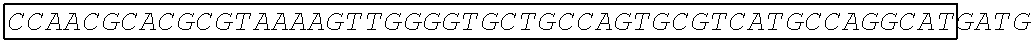 |
| | 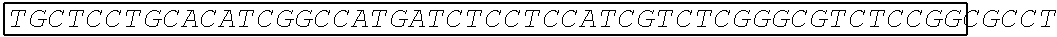 |
| | 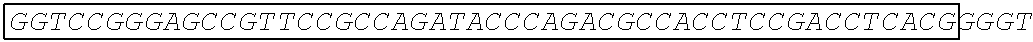 |
| | 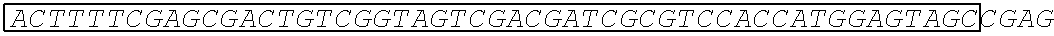 |
| | 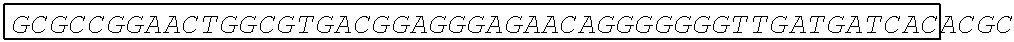 |
| | 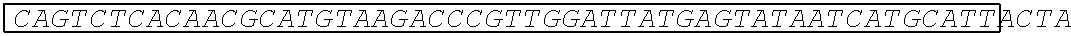 |
| | 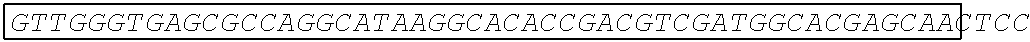 |
| | 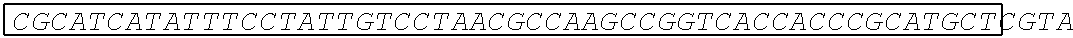 |
| | 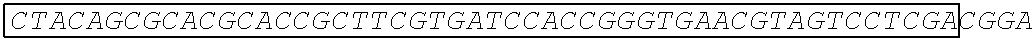 |
| | 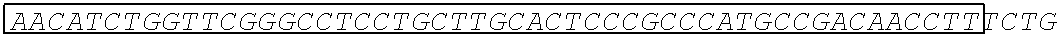 |
| | 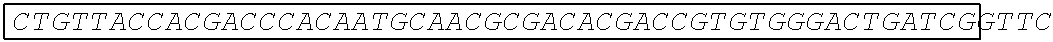 |
| | 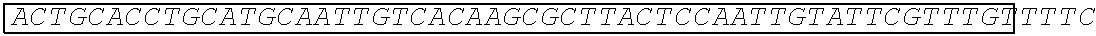 |
| | 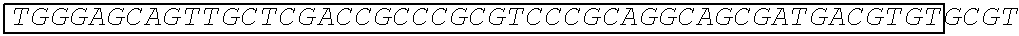 |
| | 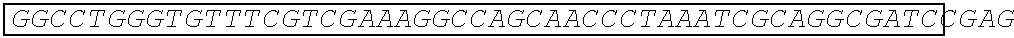 |
| | 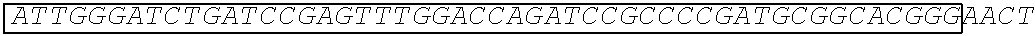 |
| | 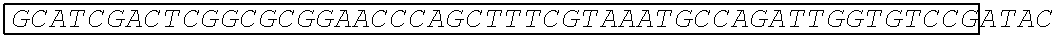 |
| | 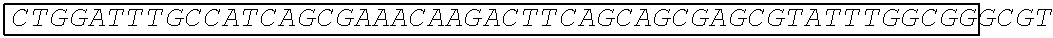 |
| | 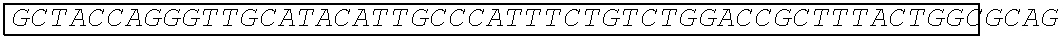 |
| | 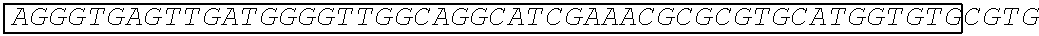 |
| | 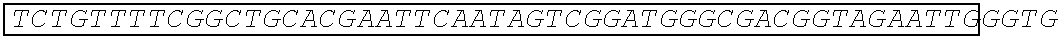 |
| | 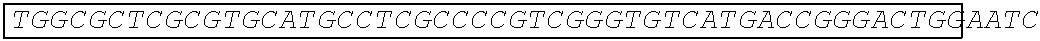 |
| | 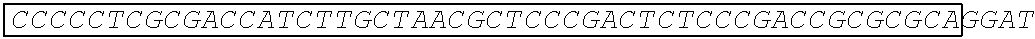 |
| | 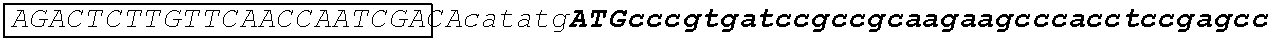 |
| | caacaagaactccgcctccgactccaagaccccctccgaggaggaggagcacgagcaggagcaggagcaggaggag |
| | gacaagaacaacaagaagaagtaccccaagaagaagtcctccgagatcaacgccaagaagtggtcctgcatcgactcc |
| | tgctgctggttcgtgggctgcatctgcgtgacctggtgggtgctgctgttcctgtacaacgccgtgcccgcctccctgccccagt |
| | acgtgaccgaggccatcacgggccccctgcccgacccccccggcgtgaagctgaagaaggagggcctgaccgccaag |
| | caccccgtggtgttcgtgcccggcatcgtgaccgcgggcctggagctgtgggagggccaccagtgcgccgacggcctgttc |
| | cgcaagcgcctgtggggcggcaccttcggcgaggtgtacaagcgccccctgtgctgggtggagcacatgtccctggacaa |
| | cgagacgggcctggacccccccggcatccgcgtgcgccccgtgtccggcctggtggccgccgactacttcgcccccggct |
| | acttcgtgtgggccgtgctgatcgccaacctggcccgcatcggctacgaggagaagacgatgttcatggcctcctacgactg |
| | gcgcctgtccttccagaacaccgaggtgcgcgaccagaccctgtcccgcatgaagtccaacatcgagctgatggtgtccat |
| | caacggcggcaacaaggccgtgatcgtgccccactccatgggcgtgctgtacttcctgcacttcatgaagtgggtggaggc |
| | ccccgcccccatgggcggcggcggcggccccgactggtgcgccaagcacatcaaggccgtgatgaacatcggcggccc |
| | cttcctgggcgtgcccaaggccgtcgcgggcctgttctccgccgaggcccgcgacategccgtggcccgcgccategcccc |
| | cggcttcctggacaacgacatgttccgcctccagaccctccagcacatgatgcgcatgtcccgcacctgggactccaccatg |
| | tccatgatcccccgcggcggcgacaccatctggggcgacctggactggtcccccgaggagggctacatcccccgcaaga |
| | agcgccagcgcaacaacgccaccgacaacgtgaacgagggcggcgccgagtccgagatctcccagcgcaagatcgtg |
| | cgctacggccgcatgatctccttcggcaagaacategccgaggccccctcctacgacatcgagcgcatcgacttccgcgac |
| | gccgtgaagggccgctccgtggccaacaacacctgcctggacgtgtggaccgagtaccacgagatgggcttcggcggca |
| | tcaaggccgtggccgagtacaaggtgtacaccgcgggctccaccatcgagctgctccagttcgtggccccgaagatgatg |
| | gagcgcggctccgcccacttctcctacgagatcgccgacaacctggaggaccccaagtacgagcactacaagtactggtc |
| | gaaccccctggagaccaagctgcccaacgcccccgagatggagatcttctccatgtacggcgtgggcatccccaccgagc |
| | gcgcctacgtgtacgagttctcccccgccgccgagtgctacatccccttccagatcgacacctccgccaacgacggcgacg |
| | ggacggctgcctgaaggacggcgtgtacaccgtggacggcgacgagaccgtgcccgtgctgtccgcgggcttcatgtgc |
| | gccaaggcctggcgcggcaagacccgcttcaacccctceggctcccgcacctacatccgegagtacgaccactccccccc |
| | cgcgaacctgctggagggccgcggcacccagtccggcgcccacgtggacatcatgggcaacttcgccctgatcgaggac |
| | atcatgcgcgtggccgcgggcgccacgggcgaggacctgggggcgaccaggtgtactccgacatcttcaagtggtccc |
| | agaagatcaagctgcccctgTAAttaattaagcagcagcagctcggatagtatcgacacactctggacgctggtcgtgtgat |
| | ggactgttgccgccacacttgctgccttgacctgtgaatatccctgccgcttttatcaaacagcctcagtgtgtttgatcttgtgtgta |
| | cgcgcttttgcgagttgctagctgcttgtgctatttgcgaataccacccccagcatccccttccctcgtttcatatcgcttgcatccca |
| | accgcaacttatctacgctgtcctgctatccctcagcgctgctcctgctcctgctcactgcccctcgcacagccttggtttgggctc |
| | cgcctgtattctcctggtactgcaacctgtaaaccagcactgcaatgctgatgcacgggaagtagtgggatgggaacacaaatg |
| | gaggccggcccgcgtctegaacagagcgcggggccgcategatacctgcaggacagcgccatgccacgccctttgatg |
| | gcttcaagtacgattacggtgttggattgtgtgtttgttgcgtagtgtgcatggtttagaataatacacttgatttcttgctca |
| | cggcaatctcggcttgtccgcaggttcaaccccatttcggagtctcaggtcagccgcgcaatgaccagccgctacttcaa |
| | ggacttgcacgacaacgccgaggtgagctatgtttaggacttgattggaaattgtcgtcgacgcatattcgcgctccgcg |
| | acagcacccaagcaaaatgtcaagtgcgttccgatttgcgtccgcaggtcgatgttgtgatcgtcggcgccggatccgcc |
| | ggtctgtcctgcgcttacgagctgaccaagcaccctgacgtccgggtacgcgagctgagattcgattagacataaattga |
| | agattaaacccgtagaaaaatttgatggtcgcgaaactgtgctcgattgcaagaaattgatcgtcctccactccgcaggt |
| | cgccatcatcgagcagggcgttgctcccggcggcggcgcctggctggggggacagctgttctcggccatgtgtgtacgt |
| | agaaggatgaatttcagctggttttcgttgcacagctgtttgtgcatgatttgtttcagactattgttgaatgtttttagatttc |
| | ttaggatgcatgatttgtctgcatgcgact |
| 11 | MPVIRRKKPTSEPNKNSASDSKTPSEEEEHEQEQEQEEDKNNKKKYPKKKSSEIN |
| | AKKWSCIDSCCWFVGCICVTWWVLLFLYNAVPASLPQYVTEAITGPLPDPPGVK |
| | LKKEGLTAKHPVVFVPGIVTAGLELWEGHQCADGLFRKRLWGGTFGEVYKRPL |
| | CWVEHMSLDNETGLDPPGIRVRPVSGLVAADYFAPGYFVWAVLIANLARIGYEE |
| | KTMFMASYDWRLSFQNTEVRDQTLSRMKSNIELMVSINGGNKAVIVPHSMGVL |
| | YFLHFMKWVEAPAPMGGGGGPDWCAKHIKAVMNIGGPFLGVPKAVAGLFSAE |
| | ARDIAVARAIAPGFLDNDMFRLQTLQHMMRMSRTWDSTMSMIPRGGDTIWGDL |
| | DWSPEEGYIPRKKRQRNNATDNVNEGGAESEISQRKIVRYGRMISFGKNIAEAPS |
| | YDIERIDFRDAVKGRSVANNTCLDVWTEYHEMGFGGIKAVAEYKVYTAGSTIEL |
| | LQFVAPKMMERGSAHFSYEIADNLEDPKYEHYKYWSNPLETKLPNAPEMEIFSM |
| | YGVGIPTERAYVYEFSPAAECYIPFQIDTSANDGDEDGCLKDGVYTVDGDETVP |
| | VLSAGFMCAKAWRGKTRFNPSGSRTYIREYDHSPPANLLEGRGTQSGAHVDIM |
| | GNFALIEDIMRVAAGATGEDLGGDQVYSDIFKWSQKIKLPL |

TABLE 11 shows the fatty acid profiles of seven primary transformants of CHK83 transformed with PCHK350 and grown as described for oil production and resulting FAMEs analyzed by GC-FID as described. P. moriformis base strain CHK22 is shown as non-transgenic control and CHK83 is shown as the parental strain control. Strains were grown for 96 hours in 96-well blocks at 200 rpm at 28° C. . . . Biomass was harvested, lyophilized to dryness, and subjected to direct transesterification to generate FAMEs for subsequent quantitation and characterization by GC/FID.

TABLE 11
Screen of primary transformants of CHK83 with PCHK350

| | | | | | | | | | | 12-OH, |
|---|---|---|---|---|---|---|---|---|---|---|
| | | | | | | | C18:3 | | | 9c- |
| Sample Name | C12:0 | C14:0 | C16:0 | C18:0 | C18:1 | C18:2 | alpha | C20:0 | C20:1 | C18:1 |
| Sample 26 | 0.05 | 1.75 | 24.66 | 3.55 | 53.76 | 8.30 | 0.62 | 0.43 | 0.09 | 5.27 |
| Sample 27 | 0.05 | 1.76 | 24.21 | 3.42 | 54.12 | 8.89 | 0.64 | 0.48 | 0.09 | 4.89 |
| Sample 28 | 0.05 | 1.72 | 24.75 | 3.47 | 54.71 | 8.12 | 0.59 | 0.40 | 0.09 | 4.63 |
| Sample 29 | 0.05 | 1.79 | 25.30 | 3.35 | 53.68 | 8.78 | 0.63 | 0.40 | 0.08 | 4.36 |
| Sample 30 | 0.05 | 1.77 | 24.78 | 3.41 | 54.19 | 8.75 | 0.62 | 0.39 | 0.09 | 4.36 |
| Sample 31 | 0.05 | 1.80 | 24.81 | 3.46 | 54.06 | 8.96 | 0.63 | 0.40 | 0.09 | 4.15 |
| Sample 32 | 0.05 | 1.72 | 25.15 | 3.32 | 54.52 | 8.56 | 0.62 | 0.39 | 0.09 | 4.00 |
| CHK83 | 0.05 | 1.62 | 24.16 | 3.19 | 56.08 | 8.31 | 0.58 | 0.35 | 0.09 | 3.97 |
| Control 1 | | | | | | | | | | |
| CHK22 | 0.04 | 1.58 | 28.88 | 3.67 | 56.14 | 7.50 | 0.52 | 0.29 | 0.07 | 0.07 |
| Control 4 | | | | | | | | | | |

Expressing RcPDAT2 in P moriformis Strain CHK83

The R. communis PDAT2 (RcPDAT2) was introduced into a derivative of P. moriformis UTEX1533, strain CHK83.

TABLE 12 shows sequences of constructs. SEQ ID NO: 12 shows integrative sequences for the transformation of P. moriformis strain CHK83 with pCHK348 encoding the RcPDAT2. The construct can be written as 5′Thi4::PmHXT1v2:ScMEL1:PmPGK:CvNR:PmSAD2-2:RcPDAT2:CvNR::3′Thi4. The expression construct contained 5′ and 3′ homology arms to permit its targeted integration into the P. moriformis genome. Proceeding in the 5′ to 3′ direction, bold, lowercase sequences represent genomic DNA from strain CHK83 that permit targeted integration at the Thi4 locus via homologous recombination. Proceeding in the 5′ to 3′ direction, the P. moriformis hexose transporter 1 (HXT1 v2) promoter driving expression of the S. carlbergensis melibiase (ScMEL1) is indicated by boxed uppercase text. The initiator ATG and terminator TAA for ScMEL1 are indicated by uppercase, bold italics while the coding region is indicated in lowercase italics. P. moriformis phosphoglucokinase (PGK) 3′-UTR is indicated by uppercase underlined text followed by a stearoyl ACP desaturase-2 (SAD2-2) promoter from P. moriformis, indicated by boxed italicized text, drive the expression of the RcPDAT2. The initiator ATG and terminator TGA codons of the RcPDAT2 are indicated by uppercase, bold italics, while the remainder of the gene is indicated by bold italics. The C. vulgaris nitrate reductase 3′-UTR is indicated by lowercase underlined text followed by the CHK83 This genomic region indicated by bold, lowercase text. SEQ ID NO: 13 shows the amino acid sequence of RcPDAT2.

TABLE 12
Sequences

| SEQ | |
|---|---|
| ID NO | Sequence |
| 12 | ccctcaactgcgacgctgggaaccttctccgggcaggcgatgtgcgtgggtttgcctccttggcacggctctacaccgtc |
| | gagtacgccatgaggcggtgatggctgtgtcggttgccacttcgtccagagacggcaagtcgtccatcctctgcgtgtgt |
| | ggcgcgacgctgcagcagtccctctgcagcagatgagcgtgactttggccatttcacgcactegagtgtacacaatccat |
| | ttttcttaaagcaaatgactgctgattgaccagatactgtaacgctgatttcgctccagatcgcacagatagcgaccatgt |
| | tgctgcgtctgaaaatctggattccgaattcgaccctggcgctccatccatgcaacagatggcgacacttgttacaattcc |
| | tgtcacccatcggcatggagcaggtccacttagattcccgatcacccacgcacatctcgctaatagtcattcgttcgtgtct |
| | tcgatcaatctcaagtgagtgtgcatggatcttggttgacgatgcggtatgggtttgcgccgctggctgcagggtctgccc |
| | aaggcaagctaacccagctcctctccccgacaatactctcgcaggcaaagccggtcacttgccttccagattgccaata |
| | aactcaattatggcctctgtcatgccatccatgggtctgatgaatggtcacgctcgtgtcctgaccgttccccagcctctgg |
| | 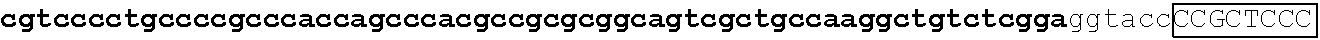 |
| | 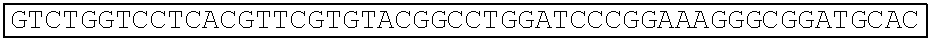 |
| | 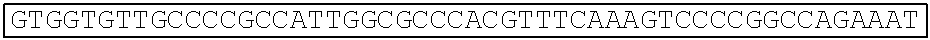 |
| | 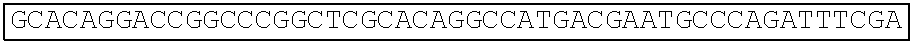 |
| | 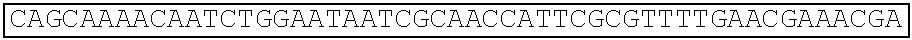 |
| | 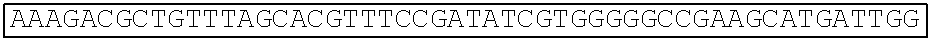 |
| | 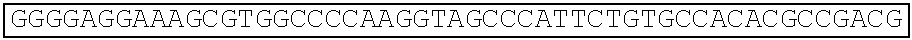 |
| | 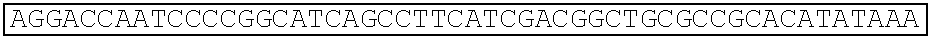 |
| | 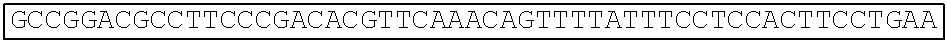 |
| | 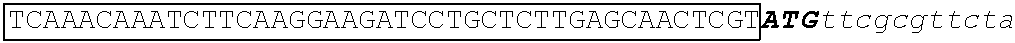 |
| | cttcctgacggcctgcatctccctgaagggcgtgtteggegtctccccctcctacaacggcctgggcctgacgccccagatg |
| | ggctgggacaactggaacacgttcgcctgcgacgtctccgagcagctgctgctggacacggccgaccgcatctccgacct |
| | gggcctgaaggacatgggctacaagtacatcatcctggacgactgctggtcctccggccgcgactccgacggcttcctggt |
| | cgccgacgagcagaagttccccaacggcatgggccacgtcgccgaccacctgcacaacaactccttcctgttcggcatgt |
| | actcctccgcgggcgagtacacgtgcgccggctaccccggctccctgggccgcgaggaggaggacgcccagttcttcgcg |
| | aacaaccgcgtggactacctgaagtacgacaactgctacaacaagggccagttcggcacgcccgagatctcctaccacc |
| | gctacaaggccatgtccgacgccctgaacaagacgggccgccccatcttctactccctgtgcaactggggccaggacctg |
| | accttctactggggctccggcatcgcgaactcctggcgcatgtccggcgacgtcacggeggagttcacgegccccgactcc |
| | cgctgcccctgcgacggcgacgagtacgactgcaagtacgccggcttccactgctccatcatgaacatcctgaacaaggc |
| | cgcccccatgggccagaacgcgggcgtcggcggctggaacgacctggacaacctggaggtcggcgteggcaacctgac |
| | ggacgacgaggagaaggcgcacttctccatgtgggccatggtgaagtcccccctgatcatcggcgcgaacgtgaacaac |
| | ctgaaggcctcctcctactccatctactcccaggegtccgtcategccatcaaccaggactccaacggcatccccgccacgc |
| | gcgtctggcgctactacgtgtccgacacggacgagtacggccagggcgagatccagatgtggtccggccccctggacaa |
| | cggcgaccaggtcgtggcgctgctgaacggcggctccgtgtcccgccccatgaacacgaccctggaggagatcttcttcga |
| | ctccaacctgggctccaagaagctgacctccacctgggacatctacgacctgtgggcgaaccgcgtcgacaactccacgg |
| | cgtccgccatcctgggccgcaacaagaccgccaccggcatcctgtacaacgccaccgagcagtcctacaaggacggcct |
| | gtccaagaacgacacccgcctgttcggccagaagatcggctccctgtcccccaacgcgatcctgaacacgaccgtccccg |
| | cccacggcatcgcgttctaccgcctgegcccctcctccTGATACAACTTATTACGTATTCTGACCGG |
| | CGCTGATGTGGCGCGGACGCCGTCGTACTCTTTCAGACTTTACTCTTGAGGA |
| | ATTGAACCTTTCTCGCTTGCTGGCATGTAAACATTGGCGCAATTAATTGTGTG |
| | ATGAAGAAAGGGTGGCACAAGATGGATCGCGAATGTACGAGATCGACAACG |
| | ATGGTGATTGTTATGAGGGGCCAAACCTGGCTCAATCTTGTCGCATGTCCGG |
| | CGCAATGTGATCCAGCGGCGTGACTCTCGCAACCTGGTAGTGTGTGCGCACC |
| | GGGTCGCTTTGATTAAAACTGATCGCATTGCCATCCCGTCAACTCACAAGCC |
| | TACTCTAGCTCCCATTGCGCACTCGGGCGCCCGGCTCGATCAATGTTCTGAGC |
| | GGAGGGCGAAGCGTCAGGAAATCGTCTCGGCAGCTGGAAGCGCATGGAATG |
| | 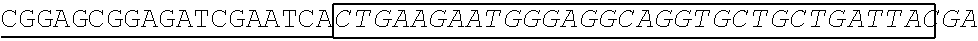 |
| | 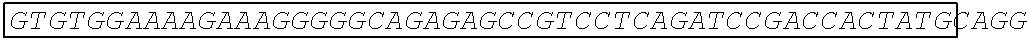 |
| | 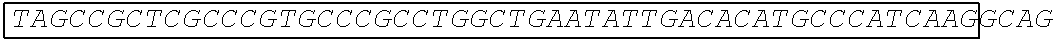 |
| | 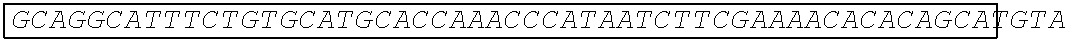 |
| | 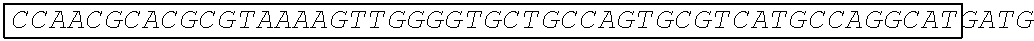 |
| | 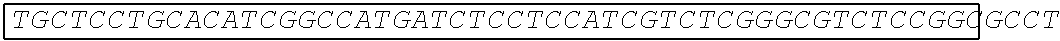 |
| | 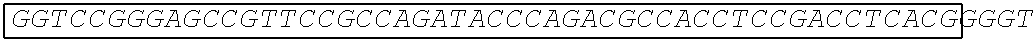 |
| | 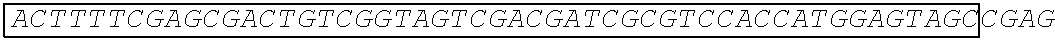 |
| | 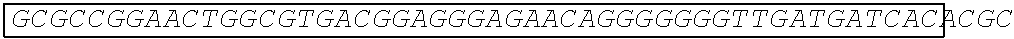 |
| | 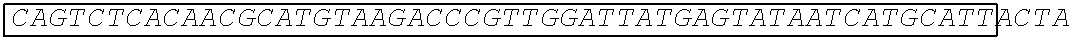 |
| | 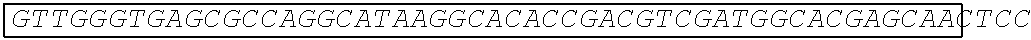 |
| | 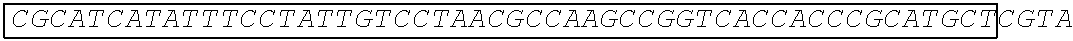 |
| | 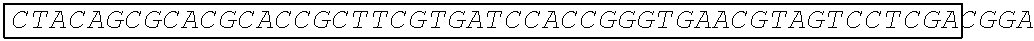 |
| | 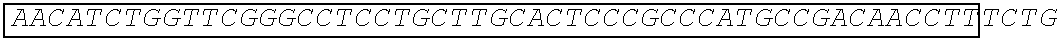 |
| | 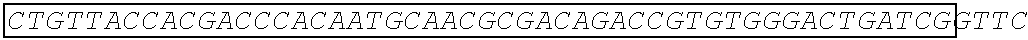 |
| | 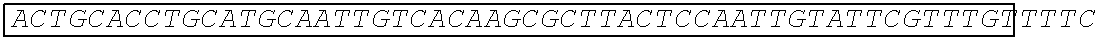 |
| | 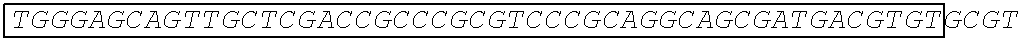 |
| | 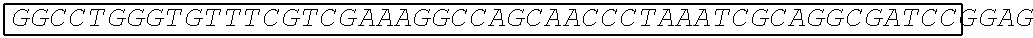 |
| | 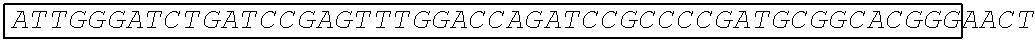 |
| | 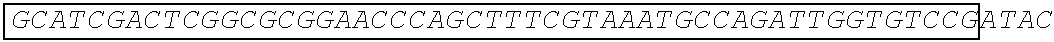 |
| | 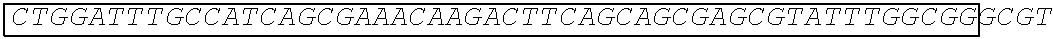 |
| | 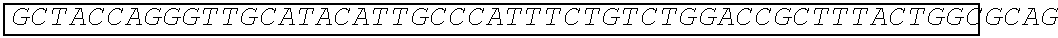 |
| | 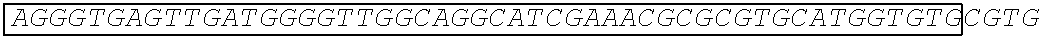 |
| | 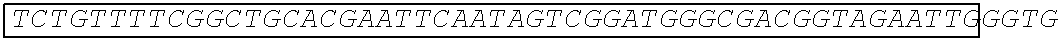 |
| | 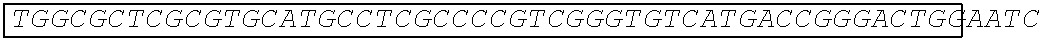 |
| | 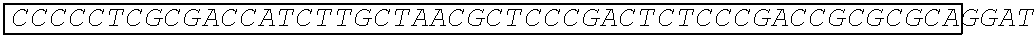 |
| | 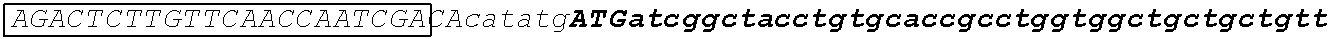 |
| | cctgtaccactgcctgcccgccaccctgcccggcttccaggtgcccgagtcccccggcgcccgcctgaagcgcgagggcc |
| | tgatcgcccagcaccccgtggtgctggtgcccggcatcatcacgggcgccctggagctgtgggagggcaagccctgcgcc |
| | gagggcctgttccgcaagcgcctgtggggcggctccttctccgagatcctgaagcgccccctgtgctggctggaccacctgg |
| | ccctgcacaacgagacgggcctggacccccccggcatccgcgtgcgcgccgtgacgggcctggtggccgccgactactt |
| | cgcccccggctacttcgtgtgggccgtgctgatcgagaacctggccaagatcggctacgagggcaagaacctgcacatgg |
| | ccgcctacgactggcgcctgtccttccagaacaccgagatccgcgaccaggccctgacccgcctgaagtccaagatcgag |
| | ttcatgtacgtgaccaacggctacaagaaggtggtggtggtgccccactccatgggcgtgatctacttcctgcacttcctgaa |
| | gtgggtggagacccccccccccatgggcggcggcggcggccccggctggtgcaacaagcacatcaaggccatcatgaa |
| | catcggcccgaccttcctgggcgtgcccaagaccgtgtccaacatcctgtccgccgaggccaaggacaccgccttcatccg |
| | cgccatcctgcccggcatcctggactccgagatcctgggcgtgcaggccatcgagcacgtgctgcgcatgacccgcacctg |
| | ggactccaccatgtccctgctgcccaagggcggcgagaccatctggggcaacctggactgggcccccgaggagcgcga |
| | ggcctgcgactcctccaagaagcgctacctgcgctccatcaacgactccaactccgacgtgaagcgcggcttccaggtga |
| | aggagtccgtgaactacggccgcatcgtgtccttctccaaggccgccgcccagctcccctectccctgctgccctccttcgac |
| | ctcaaggagttcttcggcgagcacaccaacacctcctccggctcctgcggcaagatctggaccgagtacgacgagatcaa |
| | ccgcgagtccatccgcaagttcgccgagaacaaggccttcaccgcctccaccttccacgacctgctgcgcttegtggccccc |
| | aagatggtgcagcgcgcgggcgcccacttctcccacggcatcgccgacaacctggacgaccccaagtacgagcactaca |
| | agtactggtcgaaccccctggagacccgcctgcccgacgcccccgacatggagatctactgctcctacggcgtgggcatcc |
| | ccaccgagcgctcctacgccttcaagctgtccccctccgaccgctgcaagtccatccccctgcgcatcgacacctccgtggg |
| | cgactccggcttcaccaacggcgtgtccttcgtggacggcgacgtgtccgtgcccgtgctgtccgcgggcttcatgtgcgcca |
| | aggtgtggcggggccgcacccgcttcaacccctccggcatccgcacctacgtgcgcgagttccagcacaagccccccggc |
| | tccctgctggacggccgcggcaccgagtccggctcccacgtggacatcatgggcaacaacgccctgatcgaggacgtgct |
| | gcgcgtggccgcgggcgccacgggcaccgagatgggcggcgaccgcatccactccgacatcctgcgcatggccgagaa |
| | gatcaacatccagctcTAAttaattaagcagcagcagctcggatagtatcgacacactctggacgctggtcgtgtgatggact |
| | gttgccgccacacttgctgccttgacctgtgaatatccctgccgcttttatcaaacagcctcagtgtgtttgatcttgtgtgtacgcgc |
| | ttttgcgagttgctagctgcttgtgctatttgcgaataccacccccagcatccccttccctcgtttcatatcgcttgcatcccaaccgc |
| | aacttatctacgctgtcctgctatccctcagcgctgctcctgctcctgctcactgcccctcgcacagccttggtttgggctccgcct |
| | gtattctcctggtactgcaacctgtaaaccagcactgcaatgctgatgcacgggaagtagtgggatgggaacacaaatggagg |
| | ccggcccgcgtctegaacagagcgcggcggccgcatcgatacctgcaggacagegccatgccacgccctttgatggcttc |
| | aagtacgattacggtgttggattgtgtgtttgttgcgtagtgtgcatggtttagaataatacacttgatttcttgctcacggc |
| | aatctcggcttgtccgcaggttcaaccccatttcggagtctcaggtcagccgcgcaatgaccagccgctacttcaaggac |
| | ttgcacgacaacgccgaggtgagctatgtttaggacttgattggaaattgtcgtcgacgcatattcgcgctccgcgacag |
| | cacccaagcaaaatgtcaagtgcgttccgatttgcgtccgcaggtcgatgttgtgatcgtcggcgccggatccgccggtc |
| | tgtcctgcgcttacgagctgaccaagcaccctgacgtccgggtacgcgagctgagattcgattagacataaattgaaga |
| | ttaaacccgtagaaaaatttgatggtcgcgaaactgtgctcgattgcaagaaattgategtcctccactccgcaggtcgc |
| | catcatcgagcagggcgttgctcccggcggcggcgcctggctggggggacagctgttctcggccatgtgtgtacgtaga |
| | aggatgaatttcagctggttttcgttgcacagctgtttgtgcatgatttgtttcagactattgttgaatgtttttagatttctta |
| | ggatgcatgatttgtctgcatgcgact |
| 13 | MIGYLCTAWWLLLFLYHCLPATLPGFQVPESPGARLKREGLIAQHPVVLVPGIIT |
| | GALELWEGKPCAEGLFRKRLWGGSFSEILKRPLCWLDHLALHNETGLDPPGIRV |
| | RAVTGLVAADYFAPGYFVWAVLIENLAKIGYEGKNLHMAAYDWRLSFQNTEIR |
| | DQALTRLKSKIEFMYVTNGYKKVVVVPHSMGVIYFLHFLKWVETPPPMGGGGG |
| | PGWCNKHIKAIMNIGPTFLGVPKTVSNILSAEAKDTAFIRAILPGILDSEILGVQAI |
| | EHVLRMTRTWDSTMSLLPKGGETIWGNLDWAPEEREACDSSKKRYLRSINDSN |
| | SDVKRGFQVKESVNYGRIVSFSKAAAQLPSSLLPSFDLKEFFGEHTNTSSGSCGKI |
| | WTEYDEINRESIRKFAENKAFTASTFHDLLRFVAPKMVQRAGAHFSHGIADNLD |
| | DPKYEHYKYWSNPLETRLPDAPDMEIYCSYGVGIPTERSYAFKLSPSDRCKSIPL |
| | RIDTSVGDSGFTNGVSFVDGDVSVPVLSAGFMCAKVWRGRTRFNPSGIRTYVRE |
| | FQHKPPGSLLDGRGTESGSHVDIMGNNALIEDVLRVAAGATGTEMGGDRIHSDI |
| | LRMAEKINIQL |

TABLE 13 shows the fatty acid profiles of seven primary transformants of CHK83 transformed with pCHK348 and grown as described for oil production and resulting FAMEs analyzed by GC-FID as described. P. moriformis base strain CHK22 is shown as non-transgenic control and CHK83 is shown as the parental strain control. Strains were grown for 96 hours in 96-well blocks at 200 rpm at 28° C. Biomass was harvested, lyophilized to dryness, and subjected to direct transesterification to generate FAMEs for subsequent quantitation and characterization by GC/FID.

TABLE 13
Screen of primary transformants of CHK83 with pCHK348

| | | | | | | | C18:3 | | | 12-OH- |
|---|---|---|---|---|---|---|---|---|---|---|
| Sample Name | C12:0 | C14:0 | C16:0 | C18:0 | C18:1 | C18:2 | alpha | C20:0 | C20:1 | C18:1 |
| Sample 33 | 0.05 | 1.77 | 24.67 | 3.52 | 53.95 | 8.71 | 0.63 | 0.41 | 0.09 | 4.64 |
| Sample 34 | 0.05 | 1.80 | 24.07 | 3.41 | 54.36 | 9.13 | 0.63 | 0.40 | 0.09 | 4.51 |
| Sample 35 | 0.05 | 1.82 | 25.07 | 3.19 | 53.93 | 8.94 | 0.62 | 0.37 | 0.09 | 4.25 |
| Sample 36 | 0.05 | 1.75 | 25.44 | 3.47 | 54.38 | 8.33 | 0.59 | 0.39 | 0.09 | 4.00 |
| CHK83 | 0.05 | 1.62 | 24.16 | 3.19 | 56.08 | 8.31 | 0.58 | 0.35 | 0.09 | 3.97 |
| Control 1 | | | | | | | | | | |
| Sample 37 | 0.05 | 1.67 | 25.76 | 3.09 | 55.03 | 8.22 | 0.61 | 0.35 | 0.09 | 3.58 |
| Sample 38 | 0.04 | 1.62 | 25.68 | 2.95 | 55.40 | 8.14 | 0.58 | 0.33 | 0.08 | 3.54 |
| Sample 39 | 0.05 | 1.61 | 25.68 | 2.98 | 55.41 | 8.26 | 0.60 | 0.35 | 0.08 | 3.41 |
| CHK22 | 0.04 | 1.58 | 28.88 | 3.67 | 56.14 | 7.50 | 0.52 | 0.29 | 0.07 | 0.07 |
| Control 4 | | | | | | | | | | |

Expressing RcPDCT1 in P. moriformis Strain CHK83

The R. communis gene PDCT1 (RcPDCT1) was introduced into a derivative of P. moriformis UTEX1533, CHK83. The expression construct contained 5′ and 3′ homology arms to permit its targeted integration into the P. moriformis genome.

TABLE 14 shows sequences of constructs. SEQ ID NO: 14 shows integrative sequences for the transformation of P. moriformis with PCHK351 encoding the RcPDCT1. The construct can be written as 5′Thi4::PmHXT1v2:ScMEL1:PmPGK:CvNR:PmSAD2-2:RcPDCT1:CvNR::3′Thi4. Proceeding in the 5′ to 3′ direction, bold, lowercase sequences represent genomic DNA from strain CHK22 that permit targeted integration at the Thi4 locus via homologous recombination. Proceeding in the 5′ to 3′ direction, the P. moriformis hexose transporter 1 (HXT1 v2) promoter driving expression of the S. carlbergensis melibiase (ScMEL1) is indicated by boxed uppercase text. The initiator ATG and terminator TAA for ScMEL1 are indicated by uppercase, bold italics while the coding region is indicated in lowercase italics. P. moriformis phosphoglucokinase (PGK) 3′-UTR is indicated by uppercase underlined text followed by a stearoyl ACP desaturase-2 (SAD2-2) promoter from P. moriformis, indicated by boxed italicized text, drive the expression of the RcPDCT1. The Initiator ATG and terminator TGA codons of the RcPDCT1 are indicated by uppercase, bold italics, while the remainder of the gene is indicated by bold italics. The C. vulgaris nitrate reductase 3′-UTR is indicated by lowercase underlined text followed by the CHK83 Thi4 genomic region indicated by bold, lowercase text. SEQ ID NO: 15 shows the amino acid sequence of RcPDCT1.

TABLE 14
Sequences

| SEQ | |
|---|---|
| ID NO | Sequence |
| 14 | ccctcaactgcgacgctgggaaccttctccgggcaggcgatgtgcgtgggtttgcctccttggcacggctctacaccgtc |
| | gagtacgccatgaggcggtgatggctgtgtcggttgccacttcgtccagagacggcaagtcgtccatcctctgcgtgtgt |
| | ggcgcgacgctgcagcagtccctctgcagcagatgagcgtgactttggccatttcacgcactegagtgtacacaatccat |
| | ttttcttaaagcaaatgactgctgattgaccagatactgtaacgctgatttcgctccagatcgcacagatagcgaccatgt |
| | tgctgcgtctgaaaatctggattccgaattcgaccctggcgctccatccatgcaacagatggcgacacttgttacaattcc |
| | tgtcacccatcggcatggagcaggtccacttagattcccgatcacccacgcacatctcgctaatagtcattegttcgtgtct |
| | tcgatcaatctcaagtgagtgtgcatggatcttggttgacgatgcggtatgggtttgcgccgctggctgcagggtctgccc |
| | aaggcaagctaacccagctcctctccccgacaatactctcgcaggcaaagccggtcacttgccttccagattgccaata |
| | aactcaattatggcctctgtcatgccatccatgggtctgatgaatggtcacgctcgtgtcctgaccgttccccagcctctgg |
| | 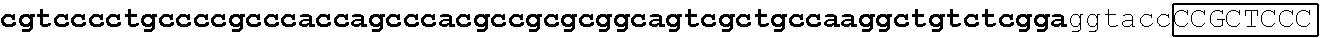 |
| | 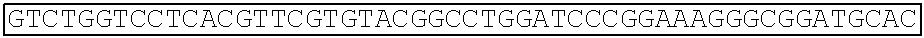 |
| | 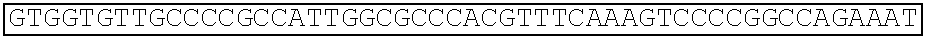 |
| | 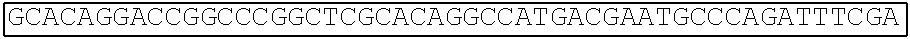 |
| | 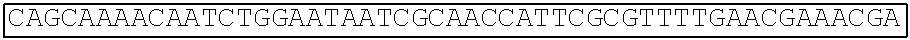 |
| | 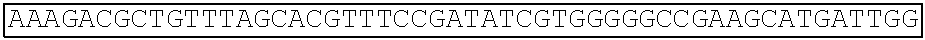 |
| | 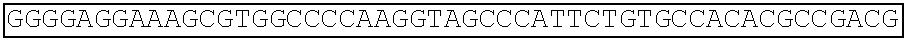 |
| | 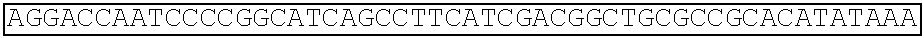 |
| | 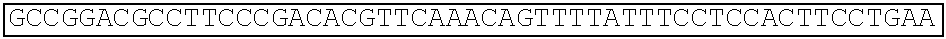 |
| | 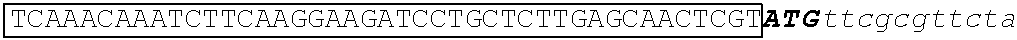 |
| | cttcctgacggcctgcatctccctgaagggcgtgttcggcgtctccccctcctacaacggcctgggcctgacgccccagatg |
| | ggctgggacaactggaacacgttcgcctgcgacgtctccgagcagctgctgctggacacggccgaccgcatctccgacct |
| | gggcctgaaggacatgggctacaagtacatcatcctggacgactgctggtcctccggccgcgactccgacggcttcctggt |
| | cgccgacgagcagaagttccccaacggcatgggccacgtcgccgaccacctgcacaacaactccttcctgttcggcatgt |
| | actcctccgcgggcgagtacacgtgcgccggctaccccggctccctgggccgcgaggaggaggacgcccagttcttcgcg |
| | aacaaccgcgtggactacctgaagtacgacaactgctacaacaagggccagttcggcacgcccgagatctcctaccacc |
| | gctacaaggccatgtccgacgccctgaacaagacgggccgccccatcttctactccctgtgcaactggggccaggacctg |
| | accttctactggggctccggcatcgcgaactcctggcgcatgtccggcgacgtcacggeggagttcacgegccccgactcc |
| | cgctgcccctgcgacggcgacgagtacgactgcaagtacgccggcttccactgctccatcatgaacatcctgaacaaggc |
| | cgcccccatgggccagaacgcgggcgtcggcggctggaacgacctggacaacctggaggtcggcgtcggcaacctgac |
| | ggacgacgaggagaaggcgcacttctccatgtgggccatggtgaagtcccccctgatcatcggcgcgaacgtgaacaac |
| | ctgaaggcctcctcctactccatctactcccaggcgtccgtcatcgccatcaaccaggactccaacggcatccccgccacgc |
| | gcgtctggcgctactacgtgtccgacacggacgagtacggccagggcgagatccagatgtggtccggccccctggacaa |
| | cggcgaccaggtcgtggcgctgctgaacggcggctccgtgtcccgccccatgaacacgaccctggaggagatcttcttcga |
| | ctccaacctgggctccaagaagctgacctccacctgggacatctacgacctgtgggcgaaccgcgtcgacaactccacgg |
| | cgtccgccatcctgggccgcaacaagaccgccaccggcatcctgtacaacgccaccgagcagtcctacaaggacggcct |
| | gtccaagaacgacacccgcctgttcggccagaagatcggctccctgtcccccaacgcgatcctgaacacgaccgtccccg |
| | cccacggcatcgcgttctaccgcctgcgcccctcctccTGATACAACTTATTACGTATTCTGACCGG |
| | CGCTGATGTGGCGCGGACGCCGTCGTACTCTTTCAGACTTTACTCTTGAGGA |
| | ATTGAACCTTTCTCGCTTGCTGGCATGTAAACATTGGCGCAATTAATTGTGTG |
| | ATGAAGAAAGGGTGGCACAAGATGGATCGCGAATGTACGAGATCGACAACG |
| | ATGGTGATTGTTATGAGGGGCCAAACCTGGCTCAATCTTGTCGCATGTCCGG |
| | CGCAATGTGATCCAGCGGCGTGACTCTCGCAACCTGGTAGTGTGTGCGCACC |
| | GGGTCGCTTTGATTAAAACTGATCGCATTGCCATCCCGTCAACTCACAAGCC |
| | TACTCTAGCTCCCATTGCGCACTCGGGCGCCCGGCTCGATCAATGTTCTGAGC |
| | GGAGGGCGAAGCGTCAGGAAATCGTCTCGGCAGCTGGAAGCGCATGGAATG |
| | 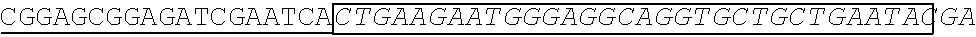 |
| | 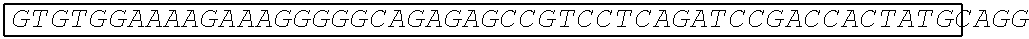 |
| | 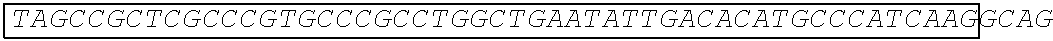 |
| | 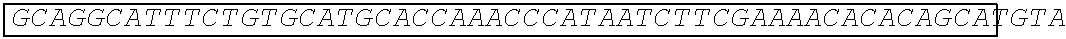 |
| | 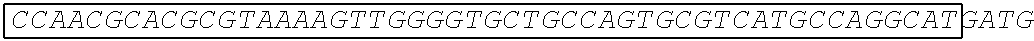 |
| | 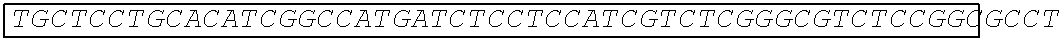 |
| | 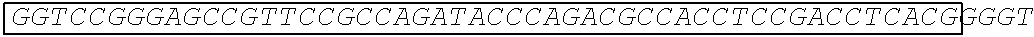 |
| | 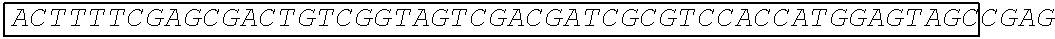 |
| | 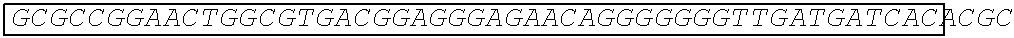 |
| | 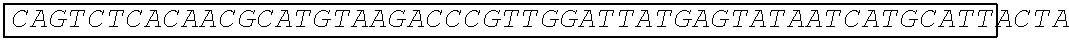 |
| | 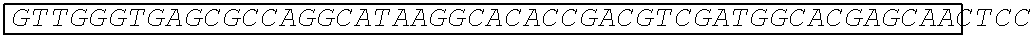 |
| | 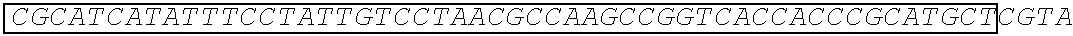 |
| | 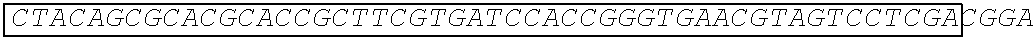 |
| | 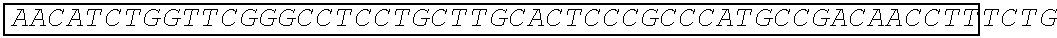 |
| | 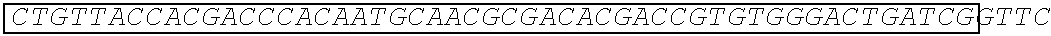 |
| | 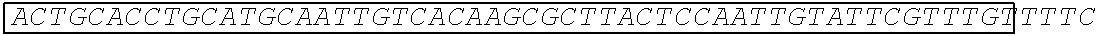 |
| | 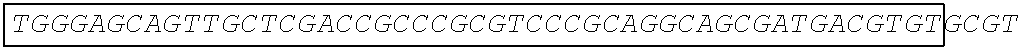 |
| | 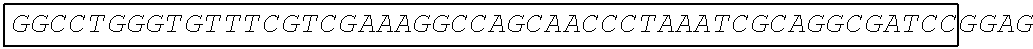 |
| | 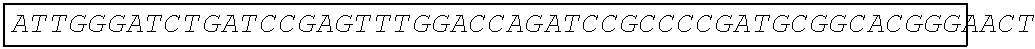 |
| | 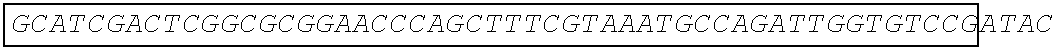 |
| | 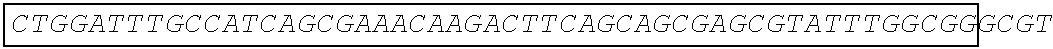 |
| | 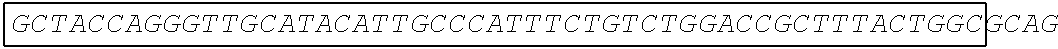 |
| | 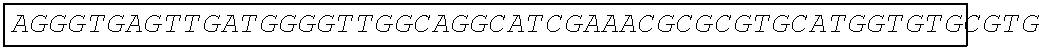 |
| | 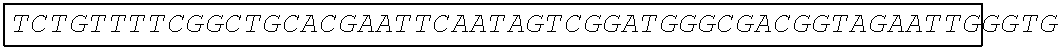 |
| | 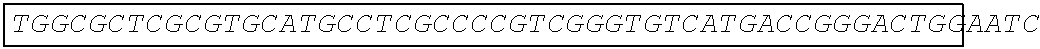 |
| | 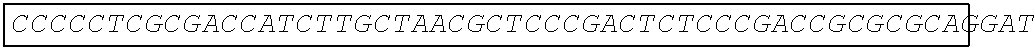 |
| | 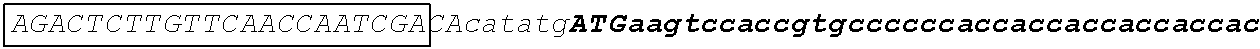 |
| | caccaccaccctgtacaagcgcaagaaggacatcaacctgacctccgtgaacgactccgtggacatggtgtccaacaag |
| | aacttcgccaacggcaacgtgaacggcggcggggctacaccgccttcaaccgcttcgacccctccttcatgaagtggac |
| | cacccacgacgtggtgaacgtggtgaagttccactggctgccctgcgtgttcggcctgggcctgctgttcttcatggccgtgg |
| | agtacaccctgcgcatggtgcccgcctcctccccccccttcgacctgggcttcctggtgacccgccacctgcacctgctgctgt |
| | cctcctggcccgccctgaacaccctgctggccttcctgaacaccgtgttcgtgctgatgcagaccgcctacatcctgtggacc |
| | tggctgatcgagggccgcccccgcgccaccatctccgccctgttcatgttcacctgccgcggcatcctgggctactccaccc |
| | agctccccctgcccgagggcttcctgggctccggcgtggacttccccgtgggcaacgtgtccttcttcctgttcttctccggcca |
| | cgtggcgggctccgtgatcgcctccctggacatgcgccgcatgcagcgctgggagctggcctggacctacgacgtgctga |
| | acgtgctccaggccgtgcgcctgctgggcacccgcggccactacacgatcgacctggccacgggcgtcggcgcgggcat |
| | cctgttcgactccctggcgggcaagtacgaggagtccaagcgcaagcaggccgtggtggccaaggagtcctccctgttctc |
| | cTAAttaattaagcagcagcagctcggatagtatcgacacactctggacgctggtcgtgtgatggactgttgccgccacacttg |
| | ctgccttgacctgtgaatatccctgccgcttttatcaaacagcctcagtgtgtttgatcttgtgtgtacgcgcttttgcgagttgctagc |
| | tgcttgtgctatttgcgaataccacccccagcatccccttccctcgtttcatatcgcttgcatcccaaccgcaacttatctacgctgtc |
| | ctgctatccctcagcgctgctcctgctcctgctcactgcccctcgcacagccttggtttgggctccgcctgtattctcctggtactgc |
| | aacctgtaaaccagcactgcaatgctgatgcacgggaagtagtgggatgggaacacaaatggaggccggcccgcgtctcga |
| | acagagcgcggcggccgcatcgatacctgcaggacagcgccatgccacgccctttgatggcttcaagtacgattacggtg |
| | ttggattgtgtgtttgttgcgtagtgtgcatggtttagaataatacacttgatttcttgctcacggcaatctcggcttgtccgc |
| | aggttcaaccccatttcggagtctcaggtcagccgcgcaatgaccagccgctacttcaaggacttgcacgacaacgccg |
| | aggtgagctatgtttaggacttgattggaaattgtcgtcgacgcatattegcgctccgcgacagcacccaagcaaaatgt |
| | caagtgcgttccgatttgcgtccgcaggtcgatgttgtgatcgtcggcgccggatccgccggtctgtcctgcgcttacgag |
| | ctgaccaagcaccctgacgtccgggtacgcgagctgagattcgattagacataaattgaagattaaacccgtagaaaa |
| | atttgatggtcgcgaaactgtgctcgattgcaagaaattgatcgtcctccactccgcaggtegccatcatcgagcagggc |
| | gttgctcccggcggcggcgcctggctggggggacagctgttctcggccatgtgtgtacgtagaaggatgaatttcagctg |
| | gttttcgttgcacagctgtttgtgcatgatttgtttcagactattgttgaatgtttttagatttcttaggatgcatgatttgtctg |
| | catgcgact |
| 15 | MKSTVPPTTTTTTTTTLYKRKKDINLTSVNDSVDMVSNKNFANGNVNGGGGYT |
| | AFNRFDPSFMKWTTHDVVNVVKFHWLPCVFGLGLLFFMAVEYTLRMVPASSPP |
| | FDLGFLVTRHLHLLLSSWPALNTLLAFLNTVFVLMQTAYILWTWLIEGRPRATIS |
| | ALFMFTCRGILGYSTQLPLPEGFLGSGVDFPVGNVSFFLFFSGHVAGSVIASLDM |
| | RRMQRWELAWTYDVLNVLQAVRLLGTRGHYTIDLATGVGAGILFDSLAGKYEE |
| | SKRKQAVVAKESSLFS |

TABLE 15 shows the fatty acid profiles of nine primary transformants of CHK83 transformed with PCHK351 and grown as described for oil production and resulting FAMEs analyzed by GC-FID as described. P. moriformis base strain CHK22 is shown as non-transgenic control and CHK83 is shown as the parental strain control. Strains were grown for 96 hours in 96-well blocks at 200 rpm at 28° C. Biomass was harvested, lyophilized to dryness, and subjected to direct transesterification to generate FAMEs for subsequent quantitation and characterization by GC/FID.

TABLE 15
Screen of primary transformants of CHK83 with PCHK351

| | | | | | | | C18:3 | | | 12-OH- |
|---|---|---|---|---|---|---|---|---|---|---|
| Sample Name | C12:0 | C14:0 | C16:0 | C18:0 | C18:1 | C18:2 | alpha | C20:0 | C20:1 | C18:1 |
| Sample 40 | 0.05 | 1.81 | 23.87 | 3.40 | 43.22 | 20.01 | 0.64 | 0.43 | 0.10 | 4.63 |
| Sample 41 | 0.06 | 1.76 | 23.47 | 3.36 | 44.11 | 19.83 | 0.62 | 0.43 | 0.12 | 4.62 |
| Sample 42 | 0.06 | 1.87 | 24.55 | 3.24 | 44.12 | 18.75 | 0.66 | 0.41 | 0.10 | 4.59 |
| Sample 43 | 0.05 | 1.77 | 24.79 | 3.42 | 47.75 | 14.95 | 0.59 | 0.41 | 0.10 | 4.55 |
| Sample 44 | 0.06 | 1.84 | 24.84 | 3.21 | 44.84 | 17.88 | 0.63 | 0.40 | 0.10 | 4.54 |
| Sample 45 | 0.06 | 1.87 | 24.37 | 3.27 | 42.32 | 20.80 | 0.64 | 0.41 | 0.11 | 4.45 |
| Sample 46 | 0.05 | 1.69 | 24.67 | 3.30 | 47.84 | 15.48 | 0.56 | 0.39 | 0.10 | 4.37 |
| Sample 47 | 0.05 | 1.77 | 25.06 | 3.33 | 44.27 | 18.76 | 0.60 | 0.40 | 0.10 | 4.07 |
| CHK83 | 0.05 | 1.62 | 24.16 | 3.19 | 56.08 | 8.31 | 0.58 | 0.35 | 0.09 | 3.97 |
| Control 1 | | | | | | | | | | |
| Sample 48 | 0.05 | 1.67 | 24.64 | 3.06 | 44.38 | 19.56 | 0.60 | 0.38 | 0.10 | 3.84 |
| CHK22 | 0.04 | 1.58 | 28.88 | 3.67 | 56.14 | 7.50 | 0.52 | 0.29 | 0.07 | 0.07 |
| Control 4 | | | | | | | | | | |

Expressing RcDGAT2 in P. moriformis Strain CHK83

The R. communis DGAT2 (RcDGAT2) gene was introduced into a derivative P. moriformis UTEX1533 strain, CHK83. The expression construct contained 5′ and 3′ homology arms to permit its targeted integration into the P. moriformis genome.

TABLE 16 shows sequences of constructs. SEQ ID NO: 16 shows integrative sequences for the transformation of P. moriformis with pCHK349 encoding the RcDGAT2. The construct can be written as 5′Thi4::PmHXT1v2:ScMEL1:PmPGK:CvNR:PmSAD2-2:RcDGAT2:CvNR::3′Thi4. Proceeding in the 5′ to 3′ direction, bold, lowercase sequences represent genomic DNA from strain CHK83 that permit targeted integration at the Thi4 locus via homologous recombination. Proceeding in the 5′ to 3′ direction, the P. moriformis hexose transporter 1 (HXT1 v2) promoter driving expression of the S. carlbergensis melibiase (ScMEL1) is indicated by boxed uppercase text. The initiator ATG and terminator TAA for ScMEL1 are indicated by uppercase, bold italics while the coding region is indicated in lowercase italics. The P. moriformis phosphoglucokinase (PGK) 3′-UTR is indicated by uppercase underlined text followed by a stearoyl ACP desaturase-2 (SAD2-2) promoter from P. moriformis, indicated by boxed italicized text, that drives the expression of RcDGAT2. The Initiator ATG and terminator TGA codons of the RcDGAT2 are indicated by uppercase, bold italics, while the remainder of the gene is indicated by bold italics. The C. vulgaris nitrate reductase 3′-UTR is indicated by lowercase underlined text followed by the CHK83 Thi4 genomic region indicated by bold, lowercase text. SEQ ID NO: 17 shows the amino acid sequence of RcDGAT2.

TABLE 16
Sequences

| SEQ | |
|---|---|
| ID NO | Sequence |
| 16 | ccctcaactgcgacgctgggaaccttctccgggcaggcgatgtgcgtgggtttgcctccttggcacggctctacaccgtc |
| | gagtacgccatgaggcggtgatggctgtgtcggttgccacttcgtccagagacggcaagtcgtccatcctctgcgtgtgt |
| | ggcgcgacgctgcagcagtccctctgcagcagatgagcgtgactttggccatttcacgcactegagtgtacacaatccat |
| | ttttcttaaagcaaatgactgctgattgaccagatactgtaacgctgatttcgctccagatcgcacagatagcgaccatgt |
| | tgctgcgtctgaaaatctggattccgaattcgaccctggcgctccatccatgcaacagatggcgacacttgttacaattcc |
| | tgtcacccatcggcatggagcaggtccacttagattcccgatcacccacgcacatctcgctaatagtcattcgttcgtgtct |
| | tcgatcaatctcaagtgagtgtgcatggatcttggttgacgatgcggtatgggtttgcgccgctggctgcagggtctgccc |
| | aaggcaagctaacccagctcctctccccgacaatactctcgcaggcaaagceggtcacttgccttccagattgccaata |
| | aactcaattatggcctctgtcatgccatccatgggtctgatgaatggtcacgctcgtgtcctgaccgttccccagcctctgg |
| | 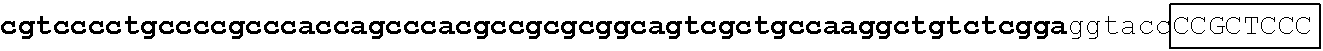 |
| | 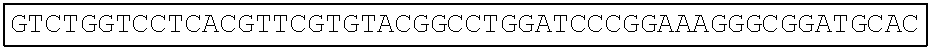 |
| | 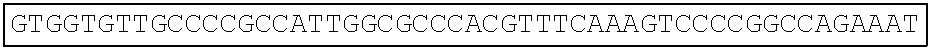 |
| | 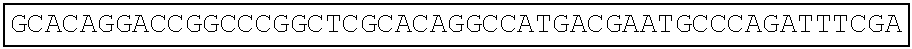 |
| | 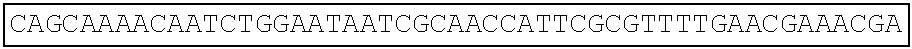 |
| | 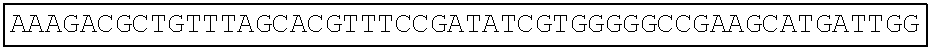 |
| | 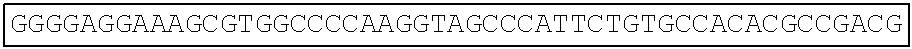 |
| | 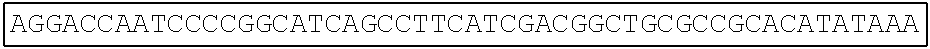 |
| | 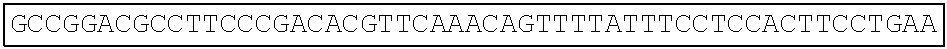 |
| | 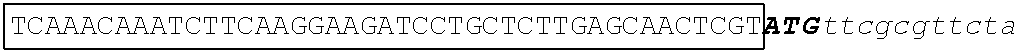 |
| | cttcctgacggcctgcatctccctgaagggcgtgtteggegtctccccctcctacaacggcctgggcctgacgccccagatg |
| | ggctgggacaactggaacacgttcgcctgcgacgtctccgagcagctgctgctggacacggccgaccgcatctccgacct |
| | gggcctgaaggacatgggctacaagtacatcatcctggacgactgctggtcctccggccgcgactccgacggcttcctggt |
| | cgccgacgagcagaagttccccaacggcatgggccacgtcgccgaccacctgcacaacaactccttcctgttcggcatgt |
| | actcctccgcgggcgagtacacgtgcgccggctaccccggctccctgggccgcgaggaggaggacgcccagttcttcgcg |
| | aacaaccgcgtggactacctgaagtacgacaactgctacaacaagggccagttcggcacgcccgagatctcctaccacc |
| | gctacaaggccatgtccgacgccctgaacaagacgggccgccccatcttctactccctgtgcaactggggccaggacctg |
| | accttctactggggctccggcatcgcgaactcctggcgcatgtccggcgacgtcacggeggagttcacgegccccgactcc |
| | cgctgcccctgcgacggcgacgagtacgactgcaagtacgccggcttccactgctccatcatgaacatcctgaacaaggc |
| | cgcccccatgggccagaacgcgggcgtcggcggctggaacgacctggacaacctggaggtcggcgtcggcaacctgac |
| | ggacgacgaggagaaggcgcacttctccatgtgggccatggtgaagtcccccctgatcatcggcgcgaacgtgaacaac |
| | ctgaaggcctcctcctactccatctactcccaggegtccgtcategccatcaaccaggactccaacggcatccccgccacgc |
| | gcgtctggcgctactacgtgtccgacacggacgagtacggccagggcgagatccagatgtggtccggccccctggacaa |
| | cggcgaccaggtcgtggcgctgctgaacggcggctccgtgtcccgccccatgaacacgaccctggaggagatcttcttcga |
| | ctccaacctgggctccaagaagctgacctccacctgggacatctacgacctgtgggcgaaccgcgtcgacaactccacgg |
| | cgtccgccatcctgggccgcaacaagaccgccaccggcatcctgtacaacgccaccgagcagtcctacaaggacggcct |
| | gtccaagaacgacacccgcctgttcggccagaagatcggctccctgtcccccaacgcgatcctgaacacgaccgtccccg |
| | cccacggcatcgcgttctaccgcctgcgcccctcctccTGATACAACTTATTACGTATTCTGACCGG |
| | CGCTGATGTGGCGCGGACGCCGTCGTACTCTTTCAGACTTTACTCTTGAGGA |
| | ATTGAACCTTTCTCGCTTGCTGGCATGTAAACATTGGCGCAATTAATTGTGTG |
| | ATGAAGAAAGGGTGGCACAAGATGGATCGCGAATGTACGAGATCGACAACG |
| | ATGGTGATTGTTATGAGGGGCCAAACCTGGCTCAATCTTGTCGCATGTCCGG |
| | CGCAATGTGATCCAGCGGCGTGACTCTCGCAACCTGGTAGTGTGTGCGCACC |
| | GGGTCGCTTTGATTAAAACTGATCGCATTGCCATCCCGTCAACTCACAAGCC |
| | TACTCTAGCTCCCATTGCGCACTCGGGCGCCCGGCTCGATCAATGTTCTGAGC |
| | GGAGGGCGAAGCGTCAGGAAATCGTCTCGGCAGCTGGAAGCGCATGGAATG |
| | 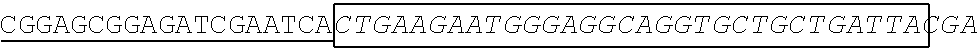 |
| | 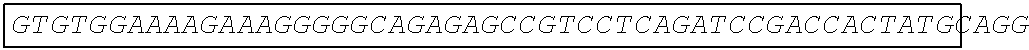 |
| | 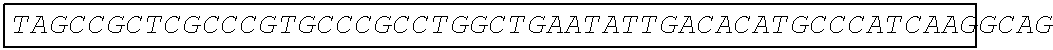 |
| | 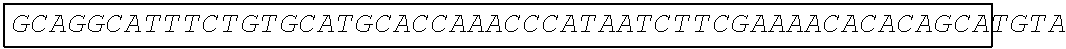 |
| | 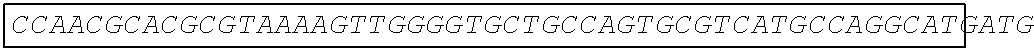 |
| | 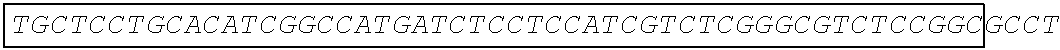 |
| | 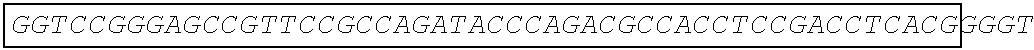 |
| | 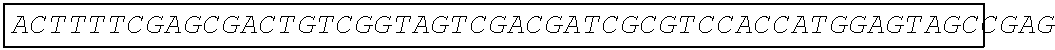 |
| | 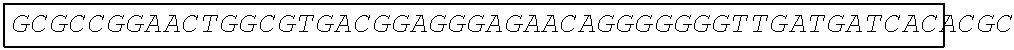 |
| | 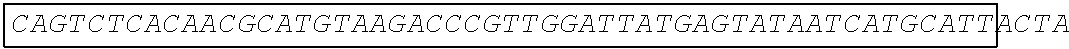 |
| | 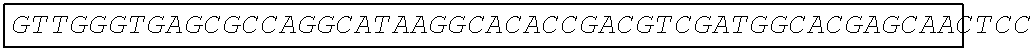 |
| | 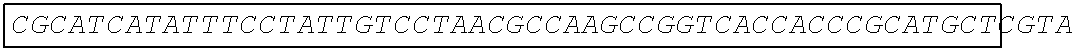 |
| | 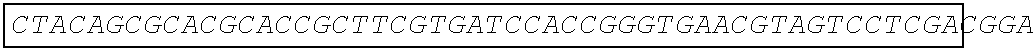 |
| | 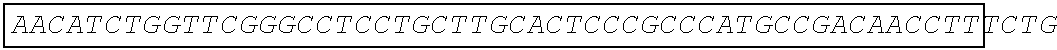 |
| | 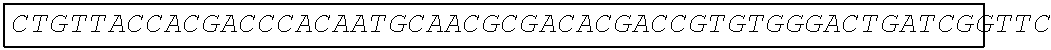 |
| | 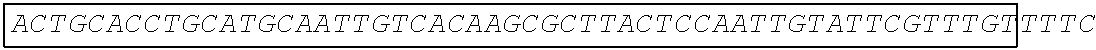 |
| | 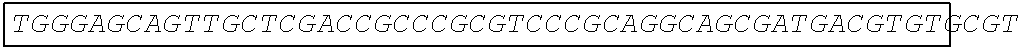 |
| | 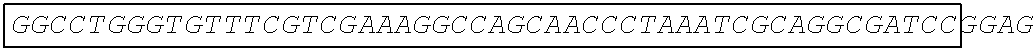 |
| | 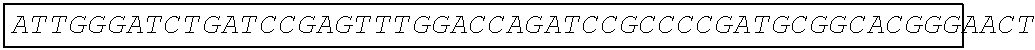 |
| | 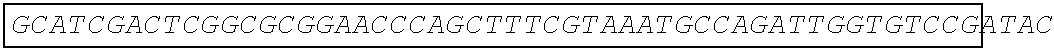 |
| | 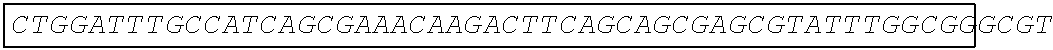 |
| | 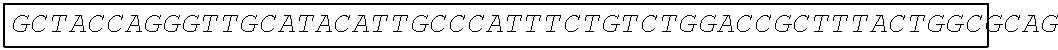 |
| | 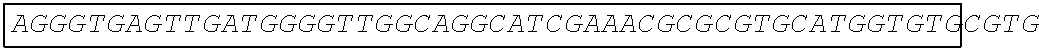 |
| | 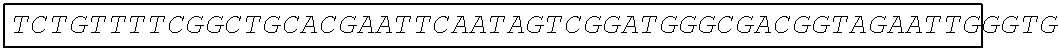 |
| | 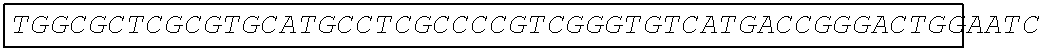 |
| | 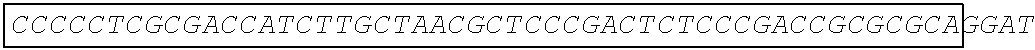 |
| | 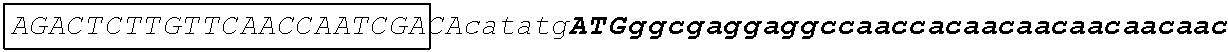 |
| | aacatcaactccaacgacgagaagaacgaggagaagtccaactacaccgtggtgaactcccgcgagctgtaccccacc |
| | aacatcttccacgccctgctggccctgtccatctggateggctccatccacttcaacctgttcctgctgttcatctcctacctgttc |
| | ctgtccttccccaccttcctgctgatcgtgggcttcttcgtggtgctgatgttcatccccatcgacgagcactccaagctgggcc |
| | gccgcctgtgccgctacgtgtgccgccacgcctgctcccacttccccgtgaccctgcacgtggaggacatgaacgcctttca |
| | ctccgaccgcgcctacgtgttcggctacgagccccactccgtgttccccctgggcgtgtccgtgctgtccgaccacttcgccgt |
| | gctgcccctgcccaagatgaaggtgctggcctccaacgccgtgttccgcacccccgtgctgcgccacatctggacctggtgc |
| | ggcctgacctccgccaccaagaagaactttaccgccctgctggcctccggctactcctgcatcgtgatccccggcggcgtgc |
| | aggagaccttctacatgaagcacggctccgagatcgccttcctgaaggcccgccgcggcttcgtgcgcgtggcgatggag |
| | atgggcaagcccctggtgcccgtgttctgcttcggccagtccaacgtgtacaagtggtggaagcccgacggcgagctgttc |
| | atgaagatcgcccgcgccatcaagttctcccccatcgtgttctggggcgtgctgggctcccacctgcccctccagcgccccat |
| | gcacgtcgtggtgggcaagcccatcgaggtgaagcagaacccccagcccaccgtggaggaggtgtccgaggtgcaggg |
| | ccagttcgtggccgccctgaaggacctgttcgagcgccacaaggcccgcgtgggctacgccgacctgaccctggagatcc |
| | tgTAAttaattaagcagcagcagctcggatagtatcgacacactctggacgctggtcgtgtgatggactgttgccgccacactt |
| | gctgccttgacctgtgaatatccctgccgcttttatcaaacagcctcagtgtgtttgatcttgtgtgtacgcgcttttgcgagttgcta |
| | gctgcttgtgctatttgcgaataccacccccagcatccccttccctcgtttcatatcgcttgcatcccaaccgcaacttatctacgct |
| | gtcctgctatccctcagcgctgctcctgctcctgctcactgcccctcgcacagccttggtttgggctccgcctgtattctcctggtac |
| | tgcaacctgtaaaccagcactgcaatgctgatgcacgggaagtagtgggatgggaacacaaatggaggccggcccgcgtctc |
| | gaacagagcgcggcggccgcatcgatacctgcaggacagcgccatgccacgccctttgatggcttcaagtacgattacgg |
| | tgttggattgtgtgtttgttgcgtagtgtgcatggtttagaataatacacttgatttcttgctcacggcaatctcggcttgtcc |
| | gcaggttcaaccccatttcggagtctcaggtcagccgcgcaatgaccagccgctacttcaaggacttgcacgacaacgc |
| | cgaggtgagctatgtttaggacttgattggaaattgtcgtcgacgcatattegcgctccgcgacagcacccaagcaaaat |
| | gtcaagtgcgttccgatttgcgtccgcaggtcgatgttgtgatcgtcggcgccggatccgccggtctgtcctgcgcttacg |
| | agctgaccaagcaccctgacgtccgggtacgcgagctgagattcgattagacataaattgaagattaaacccgtagaa |
| | aaatttgatggtcgcgaaactgtgctcgattgcaagaaattgatcgtcctccactccgcaggtcgccatcatcgagcagg |
| | gcgttgctcccggcggcggcgcctggctggggggacagctgttctcggccatgtgtgtacgtagaaggatgaatttcag |
| | ctggttttcgttgcacagctgtttgtgcatgatttgtttcagactattgttgaatgtttttagatttcttaggatgcatgatttgt |
| | ctgcatgcgact |
| 17 | MGEEANHNNNNNNINSNDEKNEEKSNYTVVNSRELYPTNIFHALLALSIWIGSIH |
| | FNLFLLFISYLFLSFPTFLLIVGFFVVLMFIPIDEHSKLGRRLCRYVCRHACSHFPV |
| | TLHVEDMNAFHSDRAYVFGYEPHSVFPLGVSVLSDHFAVLPLPKMKVLASNAV |
| | FRTPVLRHIWTWCGLTSATKKNFTALLASGYSCIVIPGGVQETFYMKHGSEIAFL |
| | KARRGFVRVAMEMGKPLVPVFCFGQSNVYKWWKPDGELFMKIARAIKFSPIVF |
| | WGVLGSHLPLQRPMHVVVGKPIEVKQNPQPTVEEVSEVQGQFVAALKDLFERH |
| | KARVGYADLTLEIL |

TABLE 17 shows the fatty acid profiles of 11 primary transformants of CHK83 transformed with pCHK349 and grown as described for oil production and resulting FAMEs analyzed by GC-FID as described. P. moriformis base strain CHK22 is shown as non-transgenic control and CHK83 is shown as the parental strain control. Strains were grown for 96 hours in 96-well blocks at 200 rpm at 28° C. Biomass was harvested, lyophilized to dryness, and subjected to direct transesterification to generate FAMEs for subsequent quantitation and characterization by GC/FID.

TABLE 17
Screen of primary transformants of CHK83 with pCHK349

| | | | | | | | | | | 12-OH, |
|---|---|---|---|---|---|---|---|---|---|---|
| | | | | | | | C18:3 | | | 9c- |
| Sample Name | C12:0 | C14:0 | C16:0 | C18:0 | C18:1 | C18:2 | alpha | C20:0 | C20:1 | C18:1 |
| Sample 49 | 0.07 | 2.09 | 25.54 | 2.39 | 50.04 | 8.70 | 0.69 | 0.19 | 0.04 | 8.46 |
| Sample 50 | 0.06 | 1.77 | 22.71 | 2.86 | 55.35 | 9.43 | 0.64 | 0.33 | 0.10 | 5.16 |
| Sample 51 | 0.05 | 1.73 | 25.54 | 3.00 | 54.18 | 8.09 | 0.61 | 0.30 | 0.07 | 4.96 |
| Sample 52 | 0.05 | 1.78 | 24.79 | 3.42 | 53.35 | 9.05 | 0.65 | 0.38 | 0.09 | 4.93 |
| Sample 53 | 0.05 | 1.77 | 25.85 | 3.34 | 53.20 | 8.30 | 0.59 | 0.37 | 0.09 | 4.92 |
| Sample 54 | 0.05 | 1.78 | 25.61 | 3.35 | 53.43 | 8.45 | 0.61 | 0.37 | 0.09 | 4.75 |
| Sample 55 | 0.05 | 1.62 | 24.68 | 3.20 | 55.02 | 8.29 | 0.60 | 0.35 | 0.09 | 4.60 |
| Sample 56 | 0.06 | 1.76 | 25.31 | 3.36 | 54.13 | 8.35 | 0.57 | 0.37 | 0.09 | 4.53 |
| Sample 57 | 0.05 | 1.62 | 25.47 | 3.25 | 54.83 | 7.93 | 0.57 | 0.35 | 0.09 | 4.33 |
| Sample 58 | 0.05 | 1.71 | 25.15 | 3.34 | 54.03 | 8.94 | 0.64 | 0.36 | 0.09 | 4.20 |
| Sample 59 | 0.05 | 1.76 | 25.59 | 3.03 | 53.74 | 8.99 | 0.64 | 0.34 | 0.08 | 4.20 |
| CHK83 | 0.05 | 1.62 | 24.16 | 3.19 | 56.08 | 8.31 | 0.58 | 0.35 | 0.09 | 3.97 |
| Control 1 | | | | | | | | | | |
| CHK22 | 0.04 | 1.58 | 28.88 | 3.67 | 56.14 | 7.50 | 0.52 | 0.29 | 0.07 | 0.07 |
| Control 4 | | | | | | | | | | |

Transformants of CHK83 were generated via particle bombardment using gold nanoparticles. Upon transformation of plasmid constructs (expressing R. communis acyltransferases) into strain CHK83, positive clones were selected on plates utilizing melibiose as the sole carbon source. Primary transformants were clonally purified and grown under standard lipid production conditions. Lipid samples were prepared from dried biomass from each transformant. Fatty acid profiles were determined using direct transesterification methods. Ricinoleic acid production (expressed as Area % of total fatty acid) of several positive transformants, compared to those of untransformed strain CHK22, and parental strain CHK83 controls.

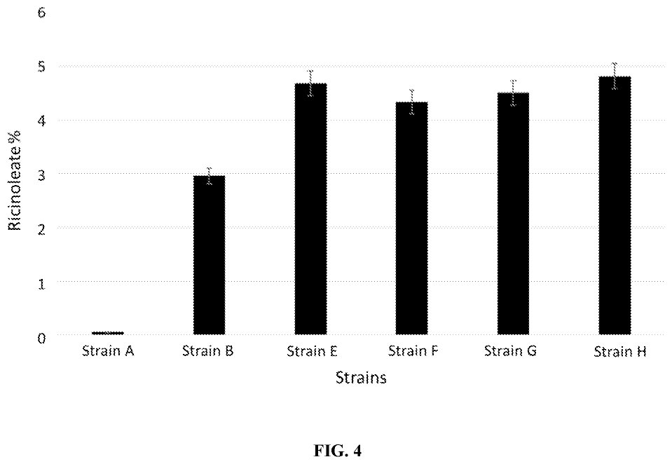

The untransformed P. moriformis (UTEX 1533) strain CHK22 exhibits a fatty acid profile comprising less than 0.1% ricinoleate, and the parental strain CHK83 produces 3.9% ricinoleate. Expression of R. communis acyltransferases in strain CHK83 resulted in increased ricinoleate production, particularly in transformants of CHK83 expressing pCHK349 and PCHK350. FIG. 4 shows a comparison of ricinoleate levels of P. moriformis strains.

### Example 4: Optimizing LFAH12 Expression by Utilizing Different 3′-UTRs

The 3′-untranslated regions (3′-UTRs) of messenger RNAs (mRNAs) are known to regulate mRNA localization, mRNA stability, and translation. As such, 3′-UTRs play a vital role in modulating gene expression. In this example, different 3′-UTRs were utilized for the expression of LFAH12, with the aim to enhance its expression in P. moriformis strains. The 3′-UTRs tested in this example include the C. vulgaris nitrate reductase 3′-UTR (CvNR), P. moriformis phosphoglycerate dehydrogenase (PGH) 3′-UTR, P. moriformis phosphoglucokinase (PGK) 3′-UTR, and the P. moriformis heat shock protein 90 (HSP90) 3′-UTR.

### Expression of PmAMT03:LFAH12:CvNR in CHK22

The sequence of the transforming DNA is shown in SEQ ID NO: 4 and fatty acid profile of the resulting transformants is shown in TABLE 5.

### Expression of PmAMT03:LFAH12:PmPGK in CHK22

The impact of the PmPGK 3′-UTR on expression of LFAH12 was assessed in P. moriformis strain CHK22. The expression construct contained 5′ and 3′ homology arms to permit its targeted integration into the P. moriformis genome and is shown in SEQ ID NO: 18.

TABLE 18 shows sequences of constructs. SEQ ID NO: 18 show integrative sequences for the transformation of P. moriformis with pCHK396 encoding the PmAMT03:LFAH12:PmPGK. The construct can be written as 5′DAO1b::CrTUB2:ScSUC2:PmPGH:CvNR:PmAMT03:LFAH12:PmPGK:CvNR::3′DAO1b. Proceeding in the 5′ to 3′ direction, bold, lowercase sequences represent genomic DNA from strain A that permit targeted integration at the DAO1b locus via homologous recombination. Proceeding in the 5′ to 3′ direction, the Chlamydomonas reinhardtii β-tubulin promoter driving expression of the yeast sucrose invertase gene (conferring the ability of strain A to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics. An AMT03 promoter from P. moriformis is indicated by boxed italicized text, and drives the expression of the LFAH12. The Initiator ATG and terminator TGA codons of the LFAH12 are indicated by uppercase, bold italics, while the remainder of the gene is indicated by bold italics. The PmPGK 3′-UTR is indicated by lowercase bold underlined text, followed by the C. vulgaris nitrate reductase 3′-UTR indicated by lowercase underlined text followed by the P. moriformis DAO1b genomic region indicated by bold, lowercase text.

TABLE 18
Sequences

| SEQ | |
|---|---|
| ID NO | Sequence |
| 18 | gctcttccgcttgcccgcaccctcgttgatctgggagccctgcgcagccccttaaatcatctcagtcaggtttctatgttca |
| | actgagcctaaagggctttcgtcacgcgcacgagcacacgtatatcggctacgcagtctctcagaagcggtagaacagt |
| | tcgcaagccctcgtcggtcgaaaacttgcgccagtactattgaattaaattaaatgatcgaatgagacgcgaaacttttg |
| | cagaatgccactgagtttgcccagagaatgggagtggcgccattcaccatccgcctgtgcacggcctgattcgccgaga |
| | cgatgaatggcgagaccagggagcggcttgcgagccccgagccggtagcaggaataacggtegacaatcttcctgtcc |
| | aattactggcaaccattagaaagagccggagcgcgttgaaattctgcaatcgagtaatttttcgatgcggcgggcctgct |
| | gaaccctaaggctccggcctatgtttaaggcgatccaagatgcacgggccccaggcacgtgtctcaagcacaaaccc |
| | cagccttagtttcgagactttgggagatagcggcagatatctagtttggcattttgtatattaattaagtctcgcaatggag |
| | cgctctgatgcggtgcagcgtcggctgcagcacctggcagtggcgctggggtcgccctatcgctcggcgcctggtcagct |
| | 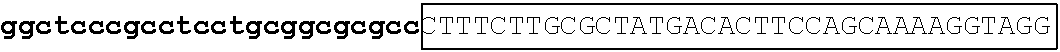 |
| | 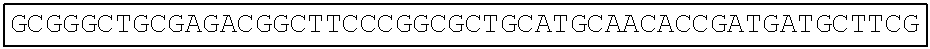 |
| | 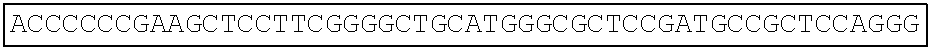 |
| | 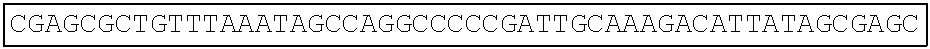 |
| | 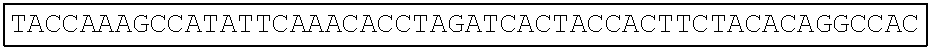 |
| | 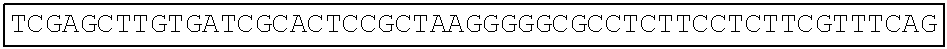 |
| | 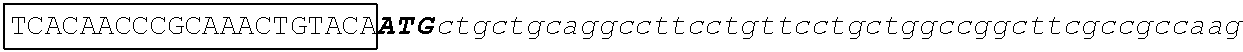 |
| | atcagcgcctccatgacgaacgagacgtccgaccgccccctggtgcacttcacccccaacaagggctggatgaacgacc |
| | ccaacggcctgtggtacgacgagaaggacgccaagtggcacctgtacttccagtacaacccgaacgacaccgtctgggg |
| | gacgcccttgttctggggccacgccacgtccgacgacctgaccaactgggaggaccagcccategccatcgccccgaag |
| | cgcaacgactccggcgccttctccggctccatggtggtggactacaacaacacctccggcttcttcaacgacaccatcgacc |
| | cgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctg |
| | gtacgagccctcccagaagtggatcatgaccgcggccaagtcccaggactacaagategagatctactcctccgacgacc |
| | tgaagtcctggaagctggagtccgcgttcgccaacgagggcttcctcggctaccagtacgagtgccccggcctgatcgagg |
| | tccccaccgagcaggaccccagcaagtcctactgggtgatgttcatctccatcaaccccggcgccccggccggeggctcct |
| | tcaaccagtacttcgtcggcagcttcaacggcacccacttcgaggccttcgacaaccagtcccgcgtggtggacttcggcaa |
| | ggactactacgccctgcagaccttcttcaacaccgacccgacctacgggagcgccctgggcatcgcgtgggcctccaactg |
| | ggagtactccgccttcgtgcccaccaacccctggcgctcctccatgtccctegtgegcaagttctccctcaacaccgagtacc |
| | aggccaacccggagacggagctgatcaacctgaaggccgagccgatcctgaacatcagcaacgccggcccctggagc |
| | cggttcgccaccaacaccacgttgacgaaggccaacagctacaacgtcgacctgtccaacagcaccggcaccctggagt |
| | tcgagctggtgtacgccgtcaacaccacccagacgatctccaagtccgtgttcgcggacctctccctctggttcaagggcctg |
| | gaggaccccgaggagtacctccgcatgggcttcgaggtgtccgcgtcctccttcttcctggaccgcgggaacagcaaggtg |
| | aagttcgtgaaggagaacccctacttcaccaaccgcatgagcgtgaacaaccagcccttcaagagcgagaacgacctgt |
| | cctactacaaggtgtacggcttgctggaccagaacatcctggagctgtacttcaacgacggcgacgtcgtgtccaccaaca |
| | cctacttcatgaccaccgggaacgccctgggctccgtgaacatgacgacgggggtggacaacctgttctacatcgacaagt |
| | tccaggtgcgcgaggtcaagtgactTAAGACGCCCGCGCGGCGCACCTGACCTGTTCTCTC |
| | GAGGGCGCCTGTTCTGCCTTGCGAAACAAGCCCCTGGAGCATGCGTGCATGA |
| | TCGTCTCTGGCGCCCCGCCGCGCGGTTTGTCGCCCTCGCGGGCGCCGCGGCC |
| | GCGGGGGCGCATTGAAATTGTTGCAAACCCCACCTGACAGATTGAGGGCCCA |
| | GGCAGGAAGGCGTTGAGATGGAGGTACAGGAGTCAAGTAACTGAAAGTTTT |
| | TATGATAACTAACAACAAAGGGTCGTTTCTGGCCAGCGAATGACAAGAACA |
| | AGATTCCACATTTCCGTGTAGAGGCTTGCCATCGAATGTGAGCGGGCGGGCC |
| | GCGGACCCGACAAAACCCTTACGACGTGGTAAGAAAAACGTGGCGGGCACT |
| | GTCCCTGTAGCCTGAAGACCAGCAGGAGACGATCGGAAGCATCACAGCACA |
| | ACCGGTgcagcagcagctcggatagtatcgacacactctggacgctggtcgtgtgatggactgttgccgccacacttg |
| | ctgccttgacctgtgaatatccctgccgcttttatcaaacagcctcagtgtgtttgatcttgtgtgtacgcgcttttgcgagttgcta |
| | gctgcttgtgctatttgcgaataccacccccagcatccccttccctcgtttcatatcgcttgcatcccaaccgcaacttatctacg |
| | ctgtcctgctatccctcagcgctgctcctgctcctgctcactgcccctcgcacagccttggtttgggctccgcctgtattctcctgg |
| | tactgcaacctgtaaaccagcactgcaatgctgatgcacgggaagtagtgggatgggaacacaaatggacctaggaata |
| | 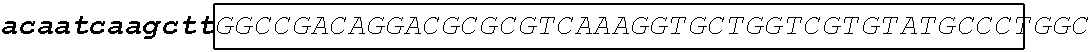 |
| | 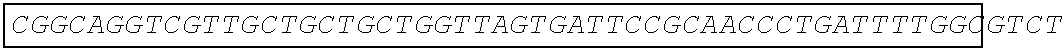 |
| | 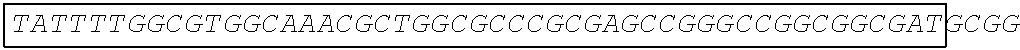 |
| | 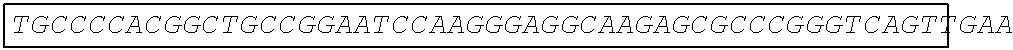 |
| | 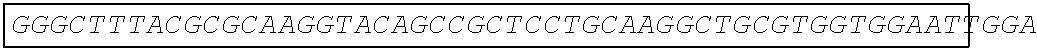 |
| | 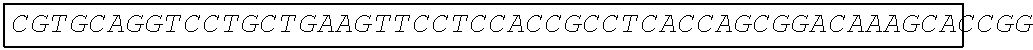 |
| | 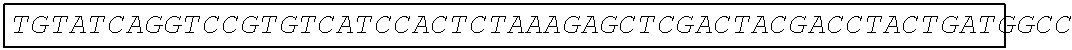 |
| | 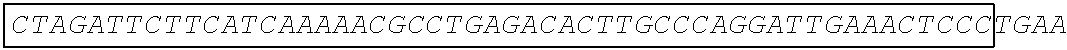 |
| | 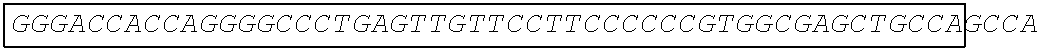 |
| | 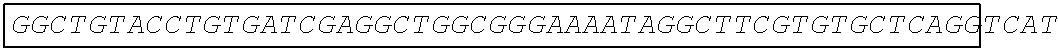 |
| | 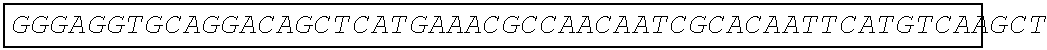 |
| | 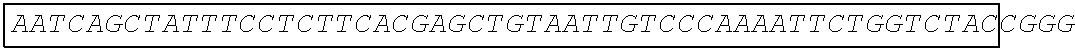 |
| | 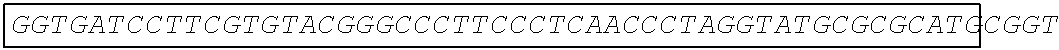 |
| | 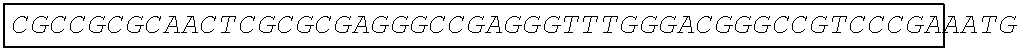 |
| | 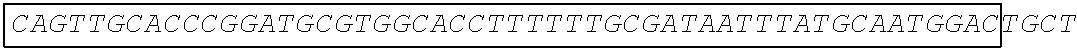 |
| | 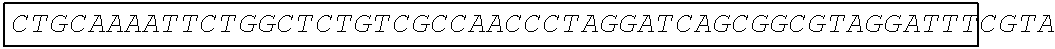 |
| | 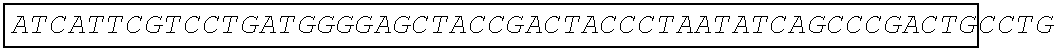 |
| | 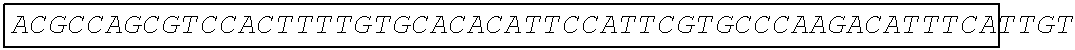 |
| | 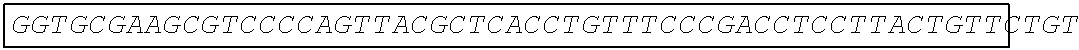 |
| | 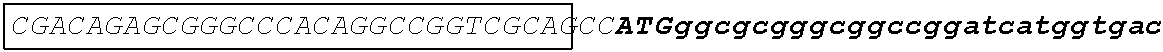 |
| | cccctcctccaagaagtccgagaccgaggccctgaagcgcggcccctgcgagaagccccccttcaccgtgaaggacctg |
| | aagaaggccatcccccagcactgcttcaagcgctccatcccccgctccttctcctacctgctgaccgacatcaccctggtgtc |
| | ctgcttctactacgtggccaccaactacttctccctgctgccccagcccctgtcgacctacctggcctggcccctgtactgggtg |
| | tgccagggctgcgtgctgacgggcatctgggtgctgggccacgagtgcggccaccacgccttctccgactaccagtggctg |
| | gacgacaccgtgggcttcatcttccactccctgctgctggtgccctacttctcctggaagtactcccaccgccgccaccactcc |
| | aacaacggctccctggagaaggacgaggtgttcgtgccccccaagaaggccgccgtgaagtggtacgtgaagtacctga |
| | acaaccccctgggccgcatcctggtgctgaccgtgcagttcatcctgggctggcccctgtacctggccttcaacgtgtccggc |
| | cgcccctacgacggcttcgcctcccacttcttcccccacgcccccatcttcaaggaccgcgagegcctccagatctacatctc |
| | cgacggggcatcctggccgtgtgctacggcctgtaccgctacgccgcctcccagggcctgaccgccatgatctgcgtgta |
| | cggcgtgcccctgctgatcgtcaacttcttcctggtgctggtgaccttcctccagcacacccacccctccctgccccactacga |
| | ctccaccgagtgggagtggatcaggggcgccctggtgaccgtggaccgcgactacggcatcctgaacaaggtgttccaca |
| | acatcaccgacacccacgtggcccaccacctgttcgccaccatcccccactacaacgccatggaggccaccgaggccat |
| | caagcccatcctgggcgactactaccacttcgacggcaccccctggtacgtggccatgtaccgcgaggccaaggagtgcc |
| | tgtacgtggagcccgacaccgagcgcggcaagaagggcgtgtactactacaacaacaagctgTGAttctgaccggcg |
| | ctgatgtggcgcggacgccgtcgtactctttcagactttactcttgaggaattgaacctttctcgcttgctggcatgtaaac |
| | attggcgcaattaattgtgtgatgaagaaagggtggcacaagatggatcgcgaatgtacgagatcgacaacgatggtg |
| | attgttatgaggggccaaacctggctcaatcttgtcgcatgtccggcgcaatgtgatccagcggcgtgactctcgcaacc |
| | tggtagtgtgtgcgcaccgggtcgctttgattaaaactgatcgcattgccatcccgtcaactcacaagcctactctagctc |
| | ccattgcgcactcgggcgcccggctcgatcaatgttctgagcggagggcgaagcgtcaggaaatcgtctcggcagctg |
| | gaagcgcatggaatgcggagcggagatcgaatcagcagcagcagctcggatagtatcgacacactctggacgctggtcgt |
| | gtgatggactgttgccgccacacttgctgccttgacctgtgaatatccctgccgcttttatcaaacagcctcagtgtgtttgatcttgt |
| | gtgtacgcgcttttgcgagttgctagctgcttgtgctatttgcgaataccacccccagcatccccttccctcgtttcatatcgcttgca |
| | tcccaaccgcaacttatctacgctgtcctgctatccctcagcgctgctcctgctcctgctcactgcccctcgcacagccttggtttg |
| | ggctccgcctgtattctcctggtactgcaacctgtaaaccagcactgcaatgctgatgcacgggaagtagtgggatgggaacac |
| | aaatggagagctcagcgtctgcgtgttgggagctggagtcgtgggcttgacgacggcgctgcagctcttgcaggatgtgc |
| | ctggcgtgcgcgttcacgtcgtggctgagaaatatggcgacgaaacgttgacggctggggccgggggctgtggatgc |
| | catacgcattgggtacgcggccattggatgggattggtaggcttatggagggataatagagtttttgtcggatccaacgc |
| | atgttgatgcagtagcccggtgtgctgaaagtatggaaggatggtgcattggctattcacatgcgctgcccaccccttttc |
| | gcaggaaatgtgccggcatcgttggtgcaccgatggggaaaatcgacgttcgaccactacatgaaaatgtatacctctg |
| | aagatgcagcaactgcgggtgcgaaacggataacggtttggtaggtgtatgtcgcagcatgtgctggattttgcgggca |
| | actccccctgccacggcccattgcaggtgtcatgttgactggaggatacgaactttcggccgtcaaattcccagaggagg |
| | accccctctgggccgacattgtgcccactgctcttccgcttgctgtttcctgtgtgaaattgt |

TABLE 19 shows the fatty acid profiles of 11 primary transformants of CHK22 transformed with pCHK396 and grown as described for oil production and resulting FAMEs analyzed by GC-FID as described. P. moriformis base strain CHK22 is shown as a non-transgenic control. Strains were grown for 120 hours in 96-well blocks at 200 rpm at 28° C. . . . Biomass was harvested, lyophilized to dryness, and subjected to direct transesterification to generate FAMEs for subsequent quantitation and characterization by GC/FID.

TABLE 19
Screen of primary transformants of CHK83 with pCHK396

| Sample | | | | | | | C18:3 | | | 12-OH, 9c- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 60 | 0.05 | 1.71 | 22.96 | 2.91 | 53.66 | 9.14 | 0.69 | 0.35 | 0.03 | 6.92 |
| Sample 61 | 0.05 | 1.59 | 23.94 | 2.92 | 54.87 | 10.26 | 0.70 | 0.30 | 0.03 | 3.80 |
| Sample 62 | 0.06 | 1.79 | 24.15 | 3.32 | 53.72 | 10.69 | 0.71 | 0.34 | 0.03 | 3.66 |
| Sample 63 | 0.06 | 1.61 | 24.01 | 2.93 | 55.04 | 10.36 | 0.71 | 0.30 | 0.03 | 3.46 |
| Sample 64 | 0.05 | 1.56 | 24.00 | 3.08 | 55.30 | 10.07 | 0.67 | 0.33 | 0.04 | 3.42 |
| Sample 65 | 0.05 | 1.56 | 24.09 | 2.81 | 54.61 | 10.96 | 0.72 | 0.32 | 0.03 | 3.37 |
| Sample 66 | 0.05 | 1.62 | 24.26 | 2.95 | 55.17 | 10.08 | 0.70 | 0.31 | 0.03 | 3.30 |
| Sample 67 | 0.05 | 1.56 | 25.18 | 3.03 | 55.90 | 9.45 | 0.62 | 0.30 | 0.03 | 2.42 |
| Sample 68 | 0.04 | 1.50 | 26.66 | 3.02 | 56.54 | 7.56 | 0.55 | 0.30 | 0.03 | 2.41 |
| Sample 69 | 0.05 | 1.78 | 28.89 | 2.94 | 55.40 | 8.66 | 0.58 | 0.20 | 0.03 | 0.11 |
| Sample 70 | 0.05 | 1.56 | 25.33 | 2.80 | 57.10 | 10.62 | 0.69 | 0.23 | 0.03 | 0.09 |
| CHK22 | 0.04 | 1.66 | 28.81 | 3.02 | 55.87 | 8.30 | 0.57 | 0.23 | 0.03 | 0.09 |
| Control 5 | | | | | | | | | | |

### Expressing PmAMT03:LFAH12:PmPGH in CHK22

The impact of the PmPGH 3′-UTR on expression of LFAH12 was assessed in P. moriformis strain CHK22. The expression construct contained 5′ and 3′ homology arms to permit its targeted integration into the P. moriformis genome and is shown in SEQ ID NO: 19.

TABLE 20 shows sequences of constructs. SEQ ID NO: 19 show integrative sequences for the transformation of P. moriformis with pCHK397 encoding the PmAMT03:LFAH12:PmPGH. The construct can be written as 5′DAO1b::CrTUB2:ScSUC2:PmPGH:CvNR:PmAMT03:LFAH12:PmPGH::3′DAO1b. Proceeding in the 5′ to 3′ direction, bold, lowercase sequences represent genomic DNA from CHK22 that permit targeted integration at the DAO1b locus via homologous recombination. Proceeding in the 5′ to 3′ direction, the Chlamydomonas reinhardtii β-tubulin promoter drives expression of the yeast sucrose invertase gene (conferring the ability of CHK22 to metabolize sucrose) and is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics. An AMT03 promoter from P. moriformis, indicated by boxed italicized text, drives the expression of the LFAH12 gene. The Initiator ATG and terminator TGA codons of LFAH12 are indicated by uppercase, bold italics, while the remainder of the gene is indicated by bold italics. The PmPGH 3′-UTR is indicated by lowercase underlined text, followed by the CHK22 DAO1b genomic region indicated by bold, lowercase text.

TABLE 20
Sequences

| SEQ | |
|---|---|
| ID NO | Sequence |
| 19 | gctcttccgcttgcccgcaccctcgttgatctgggagccctgcgcagccccttaaatcatctcagtcaggtttctatgttca |
| | actgagcctaaagggctttcgtcacgcgcacgagcacacgtatatcggctacgcagtctctcagaagcggtagaacagt |
| | tcgcaagccctcgtcggtcgaaaacttgcgccagtactattgaattaaattaaatgatcgaatgagacgcgaaacttttg |
| | cagaatgccactgagtttgcccagagaatgggagtggcgccattcaccatccgcctgtgcacggcctgattcgccgaga |
| | cgatgaatggcgagaccagggagcggcttgcgagccccgagccggtagcaggaataacggtcgacaatcttcctgtcc |
| | aattactggcaaccattagaaagagccggagcgcgttgaaattctgcaatcgagtaatttttcgatgcggcgggcctgct |
| | gaaccctaaggctccggcctatgtttaaggcgatccaagatgcacgcggccccaggcacgtgtctcaagcacaaaccc |
| | cagccttagtttcgagactttgggagatagcggcagatatctagtttggcattttgtatattaattaagtctcgcaatggag |
| | cgctctgatgcggtgcagcgtcggctgcagcacctggcagtggcgctggggtcgccctatcgctcggcgcctggtcagct |
| | 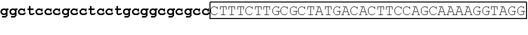 |
| | 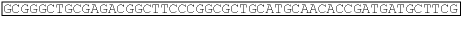 |
| | 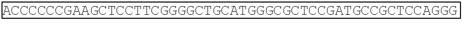 |
| | 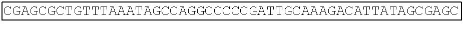 |
| | 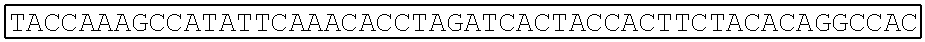 |
| | 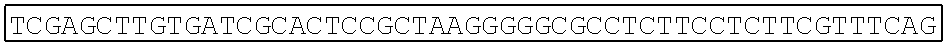 |
| | 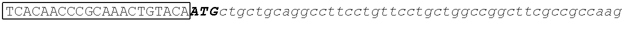 |
| | atcagcgcctccatgacgaacgagacgtccgaccgccccctggtgcacttcacccccaacaagggctggatgaacgacc |
| | ccaacggcctgtggtacgacgagaaggacgccaagtggcacctgtacttccagtacaacccgaacgacaccgtctgggg |
| | gacgcccttgttctggggccacgccacgtccgacgacctgaccaactgggaggaccagcccatcgccatcgccccgaag |
| | cgcaacgactccggcgccttctccggctccatggtggtggactacaacaacacctccggcttcttcaacgacaccatcgacc |
| | cgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctg |
| | gtacgagccctcccagaagtggatcatgaccgcggccaagtcccaggactacaagatcgagatctactcctccgacgacc |
| | tgaagtcctggaagctggagtccgcgttcgccaacgagggcttcctcggctaccagtacgagtgccccggcctgatcgagg |
| | tccccaccgagcaggaccccagcaagtcctactgggtgatgttcatctccatcaaccccggcgccccggccggcggctcct |
| | tcaaccagtacttcgtcggcagcttcaacggcacccacttcgaggccttcgacaaccagtcccgcgtggtggacttcggcaa |
| | ggactactacgccctgcagaccttcttcaacaccgacccgacctacgggagcgccctgggcatcgcgtgggcctccaactg |
| | ggagtactccgccttcgtgcccaccaacccctggcgctcctccatgtccctcgtgcgcaagttctccctcaacaccgagtacc |
| | aggccaacccggagacggagctgatcaacctgaaggccgagccgatcctgaacatcagcaacgccggcccctggagc |
| | cggttcgccaccaacaccacgttgacgaaggccaacagctacaacgtcgacctgtccaacagcaccggcaccctggagt |
| | tcgagctggtgtacgccgtcaacaccacccagacgatctccaagtccgtgttcgcggacctctccctctggttcaagggcctg |
| | gaggaccccgaggagtacctccgcatgggcttcgaggtgtccgcgtcctccttcttcctggaccgcgggaacagcaaggtg |
| | aagttcgtgaaggagaacccctacttcaccaaccgcatgagcgtgaacaaccagcccttcaagagcgagaacgacctgt |
| | cctactacaaggtgtacggcttgctggaccagaacatcctggagctgtacttcaacgacggcgacgtcgtgtccaccaaca |
| | cctacttcatgaccaccgggaacgccctgggctccgtgaacatgacgacgggggtggacaacctgttctacatcgacaagt |
| | tccaggtgcgcgaggtcaagtgactTAAGACGCCCGCGCGGCGCACCTGACCTGTTCTCTC |
| | GAGGGCGCCTGTTCTGCCTTGCGAAACAAGCCCCTGGAGCATGCGTGCATGA |
| | TCGTCTCTGGCGCCCCGCCGCGCGGTTTGTCGCCCTCGCGGGCGCCGCGGCC |
| | GCGGGGGCGCATTGAAATTGTTGCAAACCCCACCTGACAGATTGAGGGCCCA |
| | GGCAGGAAGGCGTTGAGATGGAGGTACAGGAGTCAAGTAACTGAAAGTTTT |
| | TATGATAACTAACAACAAAGGGTCGTTTCTGGCCAGCGAATGACAAGAACA |
| | AGATTCCACATTTCCGTGTAGAGGCTTGCCATCGAATGTGAGCGGGCGGGCC |
| | GCGGACCCGACAAAACCCTTACGACGTGGTAAGAAAAACGTGGCGGGCACT |
| | GTCCCTGTAGCCTGAAGACCAGCAGGAGACGATCGGAAGCATCACAGCACA |
| | ACCGGTgcagcagcagctcggatagtatcgacacactctggacgctggtcgtgtgatggactgttgccgccacacttg |
| | ctgccttgacctgtgaatatccctgccgcttttatcaaacagcctcagtgtgtttgatcttgtgtgtacgcgcttttgcgagttgcta |
| | gctgcttgtgctatttgcgaataccacccccagcatccccttccctcgtttcatatcgcttgcatcccaaccgcaacttatctacg |
| | ctgtcctgctatccctcagcgctgctcctgctcctgctcactgcccctcgcacagccttggtttgggctccgcctgtattctcctgg |
| | tactgcaacctgtaaaccagcactgcaatgctgatgcacgggaagtagtgggatgggaacacaaatggacctaggaaat |
| | 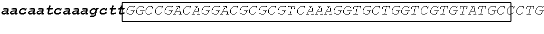 |
| | 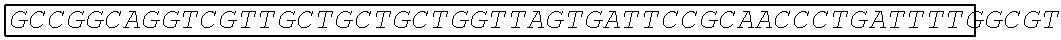 |
| | 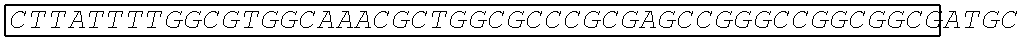 |
| | 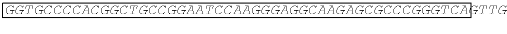 |
| | 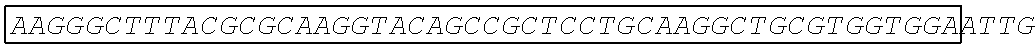 |
| | 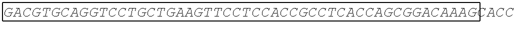 |
| | 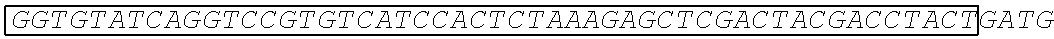 |
| | 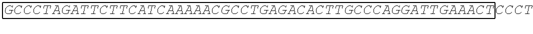 |
| | 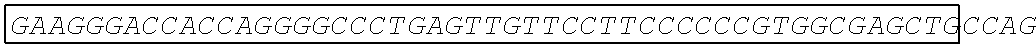 |
| | 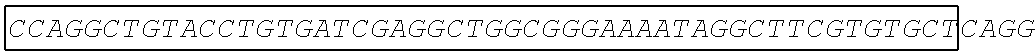 |
| | 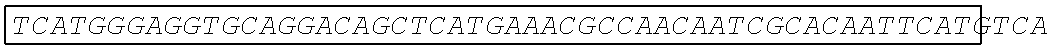 |
| | 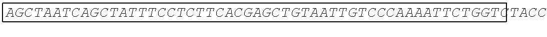 |
| | 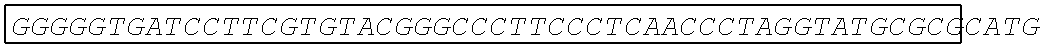 |
| | 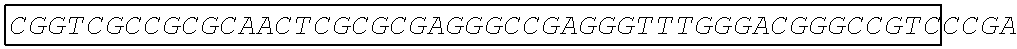 |
| | 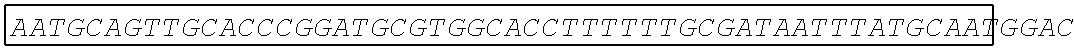 |
| | 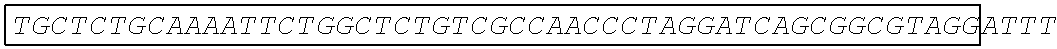 |
| | 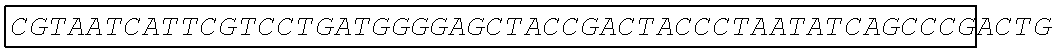 |
| | 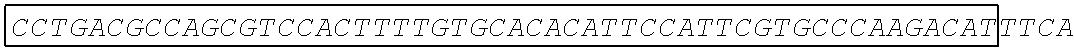 |
| | 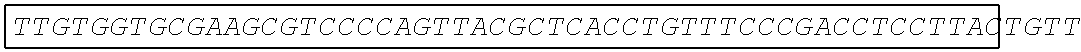 |
| | 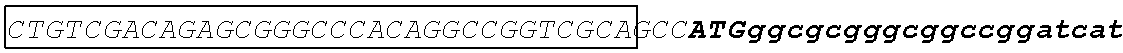 |
| | ggtgaccccctcctccaagaagtccgagaccgaggccctgaagcgcggcccctgcgagaagccccccttcaccgtgaag |
| | gacctgaagaaggccatcccccagcactgcttcaagcgctccatcccccgctccttctcctacctgctgaccgacatcaccct |
| | ggtgtcctgcttctactacgtggccaccaactacttctccctgctgccccagcccctgtcgacctacctggcctggcccctgtac |
| | tgggtgtgccagggctgcgtgctgacgggcatctgggtgctgggccacgagtgcggccaccacgccttctccgactaccag |
| | tggctggacgacaccgtgggcttcatcttccactccctgctgctggtgccctacttctcctggaagtactcccaccgccgccac |
| | cactccaacaacggctccctggagaaggacgaggtgttcgtgccccccaagaaggccgccgtgaagtggtacgtgaagt |
| | acctgaacaaccccctgggccgcatcctggtgctgaccgtgcagttcatcctgggctggcccctgtacctggccttcaacgtg |
| | tccggccgcccctacgacggcttcgcctcccacttcttcccccacgcccccatcttcaaggaccgcgagcgcctccagatct |
| | acatctccgacgcgggcatcctggccgtgtgctacggcctgtaccgctacgccgcctcccagggcctgaccgccatgatctg |
| | cgtgtacggcgtgcccctgctgatcgtcaacttcttcctggtgctggtgaccttcctccagcacacccacccctccctgcccca |
| | ctacgactccaccgagtgggagtggatcaggggcgccctggtgaccgtggaccgcgactacggcatcctgaacaaggtgt |
| | tccacaacatcaccgacacccacgtggcccaccacctgttcgccaccatcccccactacaacgccatggaggccaccga |
| | ggccatcaagcccatcctgggcgactactaccacttcgacggcaccccctggtacgtggccatgtaccgcgaggccaagg |
| | agtgcctgtacgtggagcccgacaccgagcgcggcaagaagggcgtgtactactacaacaacaagctgTGAacgccc |
| | gcgcggcgcacctgacctgttctctcgagggcgcctgttctgcgaaacaagcccctggagcatgcgtgcatcatcgtct |
| | ctggcgccccgccgcgcggtttgtcgccctcgcgggcgccgcggccgcgggggcgcattgaaattgttgcaaaccccacct |
| | gacagattgagggcccaggcaggaaggcgttgagatggaggtacaggagtcaagtaactgaaagtttttatgataactaacaa |
| | caaagggtcgtttctggccagcgaatgacaagaacaagattccacatttccgtgtagaggcttgccatcgaatgtgagcgggcg |
| | ggccgcggacccgacaaaacccttacgacgtggtaagaaaaacgtggcgggcactgtccctgtagcctgaagaccagcagg |
| | agacgatcggaagcatcacagcacaaccggtagcgtctgcgtgttgggagctggagtcgtgggcttgacgacggcgctgc |
| | agctcttgcaggatgtgcctggcgtgcgcgttcacgtcgtggctgagaaatatggcgacgaaacgttgacggctggggc |
| | cggcgggctgtggatgccatacgcattgggtacgcggccattggatgggattggtaggcttatggagggataatagagt |
| | ttttgtcggatccaacgcatgttgatgcagtagcccggtgtgctgaaagtatggaaggatggtgcattggctattcacatg |
| | cgctgcccaccccttttcgcaggaaatgtgccggcatcgttggtgcaccgatggggaaaatcgacgttcgaccactacat |
| | gaaaatgtatacctctgaagatgcagcaactgcgggtgcgaaacggataacggtttggtaggtgtatgtcgcagcatgt |
| | gctggattttgcgggcaactccccctgccacggcccattgcaggtgtcatgttgactggaggatacgaactttcggccgtc |
| | aaattcccagaggaggaccccctctgggccgacattgtgcccactgctcttccgcttgctgtttcctgtgtgaaattgt |

TABLE 21 shows the fatty acid profiles of 12 primary transformants of CHK22 transformed with pCHK397 and grown as described for oil production and resulting FAMEs analyzed by GC-FID as described. P. moriformis base strain CHK22 is shown as non-transgenic control. Strains were grown for 120 hours in 96-well blocks at 200 rpm at 28° C. Biomass was harvested, lyophilized to dryness, and subjected to direct transesterification to generate FAMEs for subsequent quantitation and characterization by GC/FID.

TABLE 21
Screen of primary transformants of CHK83 with pCHK397

| Sample | | | | | | | C18:3 | | | 12-OH, 9c- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 71 | 0.05 | 1.72 | 23.56 | 2.85 | 52.57 | 8.63 | 0.68 | 0.36 | 0.04 | 7.99 |
| Sample 72 | 0.06 | 1.86 | 23.23 | 3.22 | 52.34 | 9.18 | 0.68 | 0.42 | 0.03 | 7.45 |
| Sample 73 | 0.05 | 1.59 | 23.54 | 2.96 | 54.45 | 8.99 | 0.67 | 0.36 | 0.03 | 5.82 |
| Sample 74 | 0.05 | 1.53 | 21.37 | 4.03 | 57.31 | 9.78 | 0.59 | 0.32 | 0.02 | 3.60 |
| Sample 75 | 0.05 | 1.58 | 23.91 | 2.89 | 55.45 | 10.22 | 0.69 | 0.30 | 0.03 | 3.39 |
| Sample 76 | 0.05 | 1.66 | 25.25 | 2.85 | 55.10 | 9.38 | 0.67 | 0.29 | 0.03 | 3.24 |
| Sample 77 | 0.05 | 1.53 | 24.27 | 2.92 | 55.77 | 9.86 | 0.68 | 0.32 | 0.03 | 3.07 |
| Sample 78 | 0.05 | 1.54 | 24.29 | 2.99 | 55.86 | 9.72 | 0.67 | 0.33 | 0.03 | 2.99 |
| Sample 79 | 0.05 | 1.55 | 23.88 | 2.98 | 55.92 | 10.13 | 0.67 | 0.32 | 0.03 | 2.96 |
| Sample 80 | 0.07 | 1.91 | 25.68 | 2.98 | 54.60 | 9.78 | 0.68 | 0.30 | 0.04 | 2.56 |
| Sample 81 | 0.05 | 1.60 | 26.09 | 3.01 | 56.14 | 8.56 | 0.57 | 0.29 | 0.03 | 2.23 |
| Sample 82 | 0.04 | 1.50 | 25.12 | 2.93 | 57.59 | 10.37 | 0.66 | 0.24 | 0.03 | 0.09 |
| CHK22 | 0.04 | 1.66 | 28.81 | 3.02 | 55.87 | 8.30 | 0.57 | 0.23 | 0.03 | 0.09 |
| Control 5 | | | | | | | | | | |

### Expressing PmAMT03:LFAH12:PmHSP90 in CHK22

The impact of the PmHSP90 3′-UTR on expression of LFAH12 was assessed in P. moriformis strain CHK22. The expression construct contained 5′ and 3′ homology arms to permit its targeted integration into the P. moriformis genome and is shown in SEQ ID NO: 20

TABLE 22 shows sequences of constructs. SEQ ID NO: 20 shows integrative sequences for the transformation of P. moriformis with pCHK398 encoding the PmAMT03:LFAH12:PmHSP90. The construct can be written as 5′DAO1b::CrTUB2:ScSUC2:PmPGH:CvNR:PmAMT03:LFAH12:PmHSP90:CvNR::3′DAO1b. Proceeding in the 5′ to 3′ direction, bold, lowercase sequences represent genomic DNA from CHK22 that permits targeted integration at the DAO1b locus via homologous recombination. Proceeding in the 5′ to 3′ direction, the Chlamydomonas reinhardtii β-tubulin promoter drives expression of the yeast sucrose invertase gene (conferring the ability of CHK22 to metabolize sucrose) is indicated by boxed uppercase text. The initiator ATG and terminator TAA for invertase are indicated by uppercase, bold italics while the coding region is indicated in lowercase italics. The P. moriformis PmPGH 3′-UTR is indicated by uppercase underlined text followed by a linker, indicated by lowercase, bold italics. An AMT03 promoter from P. moriformis, indicated by boxed italicized text, drives the expression of the LFAH12. The Initiator ATG and terminator TGA codons of LFAH12 are indicated by uppercase, bold italics, while the remainder of the gene is indicated by bold italics. The PmHSP90 3′-UTR is indicated by lowercase bold underlined text, followed by the C. vulgaris nitrate reductase 3′-UTR, indicated by lowercase underlined text followed by the CHK22 DAO1b genomic region indicated by bold, lowercase text.

TABLE 22
Sequences

| SEQ | |
|---|---|
| ID NO | Sequence |
| 20 | gctcttccgcttgcccgcaccctcgttgatctgggagccctgcgcagccccttaaatcatctcagtcaggtttctatgttca |
| | actgagcctaaagggctttcgtcacgcgcacgagcacacgtatatcggctacgcagtctctcagaagcggtagaacagt |
| | tcgcaagccctcgtcggtcgaaaacttgcgccagtactattgaattaaattaaatgatcgaatgagacgcgaaacttttg |
| | cagaatgccactgagtttgcccagagaatgggagtggcgccattcaccatccgcctgtgcacggcctgattcgccgaga |
| | cgatgaatggcgagaccagggagcggcttgcgagccccgagccggtagcaggaataacggtcgacaatcttcctgtcc |
| | aattactggcaaccattagaaagagccggagcgcgttgaaattctgcaatcgagtaatttttcgatgcggcgggcctgct |
| | gaaccctaaggctccggcctatgtttaaggcgatccaagatgcacgcggccccaggcacgtgtctcaagcacaaaccc |
| | cagccttagtttcgagactttgggagatagcggcagatatctagtttggcattttgtatattaattaagtctcgcaatggag |
| | cgctctgatgcggtgcagcgtcggctgcagcacctggcagtggcgctggggtcgccctatcgctcggcgcctggtcagct |
| | 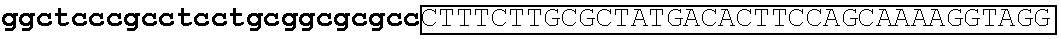 |
| | 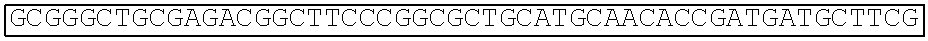 |
| | 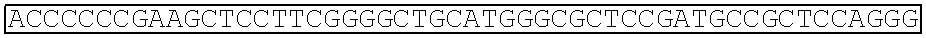 |
| | 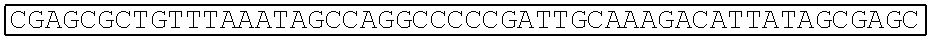 |
| | 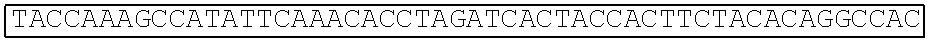 |
| | 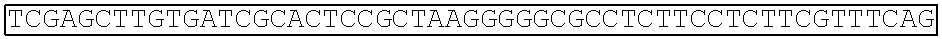 |
| | 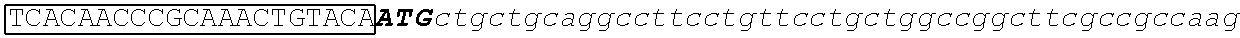 |
| | atcagcgcctccatgacgaacgagacgtccgaccgccccctggtgcacttcacccccaacaagggctggatgaacgacc |
| | ccaacggcctgtggtacgacgagaaggacgccaagtggcacctgtacttccagtacaacccgaacgacaccgtctgggg |
| | gacgcccttgttctggggccacgccacgtccgacgacctgaccaactgggaggaccagcccatcgccatcgccccgaag |
| | cgcaacgactccggcgccttctccggctccatggtggtggactacaacaacacctccggcttcttcaacgacaccatcgacc |
| | cgcgccagcgctgcgtggccatctggacctacaacaccccggagtccgaggagcagtacatctcctacagcctggacgg |
| | cggctacaccttcaccgagtaccagaagaaccccgtgctggccgccaactccacccagttccgcgacccgaaggtcttctg |
| | gtacgagccctcccagaagtggatcatgaccgcggccaagtcccaggactacaagatcgagatctactcctccgacgacc |
| | tgaagtcctggaagctggagtccgcgttcgccaacgagggcttcctcggctaccagtacgagtgccccggcctgatcgagg |
| | tccccaccgagcaggaccccagcaagtcctactgggtgatgttcatctccatcaaccccggcgccccggccggcggctcct |
| | tcaaccagtacttcgtcggcagcttcaacggcacccacttcgaggccttcgacaaccagtcccgcgtggtggacttcggcaa |
| | ggactactacgccctgcagaccttcttcaacaccgacccgacctacgggagcgccctgggcatcgcgtgggcctccaactg |
| | ggagtactccgccttcgtgcccaccaacccctggcgctcctccatgtccctcgtgcgcaagttctccctcaacaccgagtacc |
| | aggccaacccggagacggagctgatcaacctgaaggccgagccgatcctgaacatcagcaacgccggcccctggagc |
| | cggttcgccaccaacaccacgttgacgaaggccaacagctacaacgtcgacctgtccaacagcaccggcaccctggagt |
| | tcgagctggtgtacgccgtcaacaccacccagacgatctccaagtccgtgttcgcggacctctccctctggttcaagggcctg |
| | gaggaccccgaggagtacctccgcatgggcttcgaggtgtccgcgtcctccttcttcctggaccgcgggaacagcaaggtg |
| | aagttcgtgaaggagaacccctacttcaccaaccgcatgagcgtgaacaaccagcccttcaagagcgagaacgacctgt |
| | cctactacaaggtgtacggcttgctggaccagaacatcctggagctgtacttcaacgacggcgacgtcgtgtccaccaaca |
| | cctacttcatgaccaccgggaacgccctgggctccgtgaacatgacgacgggggtggacaacctgttctacatcgacaagt |
| | tccaggtgcgcgaggtcaagtgactTAAGACGCCCGCGCGGCGCACCTGACCTGTTCTCTC |
| | GAGGGCGCCTGTTCTGCCTTGCGAAACAAGCCCCTGGAGCATGCGTGCATGA |
| | TCGTCTCTGGCGCCCCGCCGCGCGGTTTGTCGCCCTCGCGGGCGCCGCGGCC |
| | GCGGGGGCGCATTGAAATTGTTGCAAACCCCACCTGACAGATTGAGGGCCCA |
| | GGCAGGAAGGCGTTGAGATGGAGGTACAGGAGTCAAGTAACTGAAAGTTTT |
| | TATGATAACTAACAACAAAGGGTCGTTTCTGGCCAGCGAATGACAAGAACA |
| | AGATTCCACATTTCCGTGTAGAGGCTTGCCATCGAATGTGAGCGGGCGGGCC |
| | GCGGACCCGACAAAACCCTTACGACGTGGTAAGAAAAACGTGGCGGGCACT |
| | GTCCCTGTAGCCTGAAGACCAGCAGGAGACGATCGGAAGCATCACAGCACA |
| | ACCGGTgcagcagcagctcggatagtatcgacacactctggacgctggtcgtgtgatggactgttgccgccacacttg |
| | ctgccttgacctgtgaatatccctgccgcttttatcaaacagcctcagtgtgtttgatcttgtgtgtacgcgcttttgcgagttgcta |
| | gctgcttgtgctatttgcgaataccacccccagcatccccttccctcgtttcatatcgcttgcatcccaaccgcaacttatctacg |
| | ctgtcctgctatccctcagcgctgctcctgctcctgctcactgcccctcgcacagccttggtttgggctccgcctgtattctcctgg |
| | tactgcaacctgtaaaccagcactgcaatgctgatgcacgggaagtagtgggatgggaacacaaatggacctaggaaat |
| | 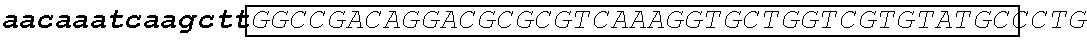 |
| | 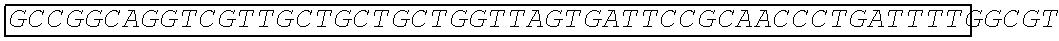 |
| | 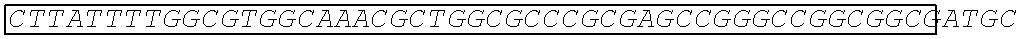 |
| | 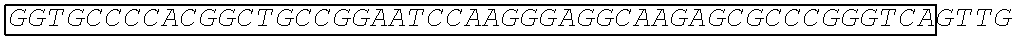 |
| | 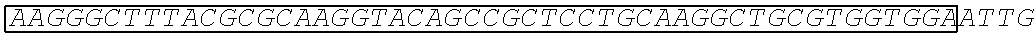 |
| | 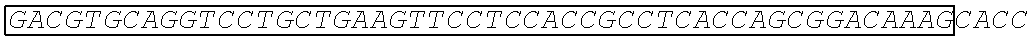 |
| | 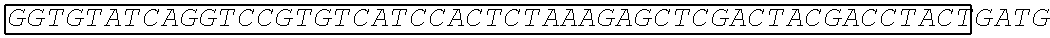 |
| | 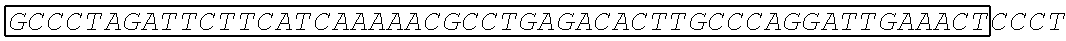 |
| | 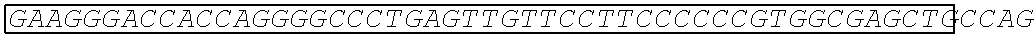 |
| | 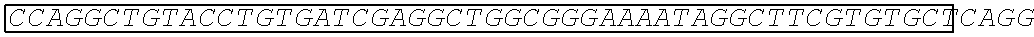 |
| | 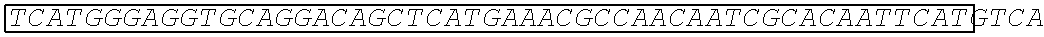 |
| | 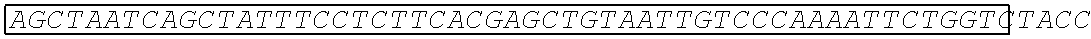 |
| | 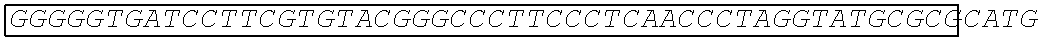 |
| | 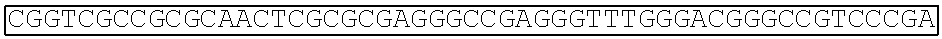 |
| | 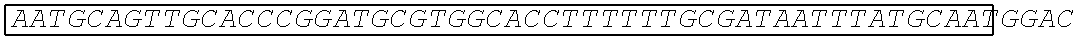 |
| | 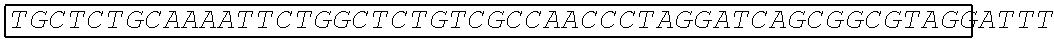 |
| | 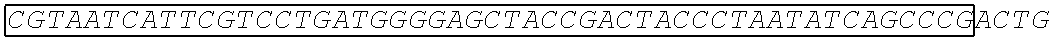 |
| | 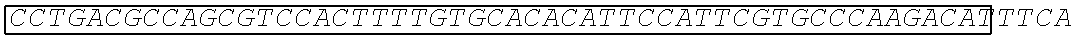 |
| | 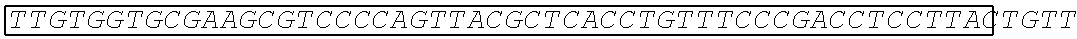 |
| | 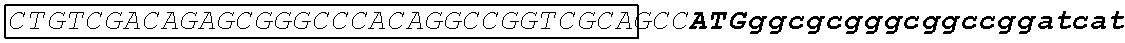 |
| | ggtgaccccctcctccaagaagtccgagaccgaggccctgaagcgcggcccctgcgagaagccccccttcaccgtgaag |
| | gacctgaagaaggccatcccccagcactgcttcaagcgctccatcccccgctccttctcctacctgctgaccgacatcaccct |
| | ggtgtcctgcttctactacgtggccaccaactacttctccctgctgccccagcccctgtcgacctacctggcctggcccctgtac |
| | tgggtgtgccagggctgcgtgctgacgggcatctgggtgctgggccacgagtgcggccaccacgccttctccgactaccag |
| | tggctggacgacaccgtgggcttcatcttccactccctgctgctggtgccctacttctcctggaagtactcccaccgccgccac |
| | cactccaacaacggctccctggagaaggacgaggtgttcgtgccccccaagaaggccgccgtgaagtggtacgtgaagt |
| | acctgaacaaccccctgggccgcatcctggtgctgaccgtgcagttcatcctgggctggcccctgtacctggccttcaacgtg |
| | tccggccgcccctacgacggcttcgcctcccacttcttcccccacgcccccatcttcaaggaccgcgagcgcctccagatct |
| | acatctccgacgcgggcatcctggccgtgtgctacggcctgtaccgctacgccgcctcccagggcctgaccgccatgatctg |
| | cgtgtacggcgtgcccctgctgatcgtcaacttcttcctggtgctggtgaccttcctccagcacacccacccctccctgcccca |
| | ctacgactccaccgagtgggagtggatcaggggcgccctggtgaccgtggaccgcgactacggcatcctgaacaaggtgt |
| | tccacaacatcaccgacacccacgtggcccaccacctgttcgccaccatcccccactacaacgccatggaggccaccga |
| | ggccatcaagcccatcctgggcgactactaccacttcgacggcaccccctggtacgtggccatgtaccgcgaggccaagg |
| | agtgcctgtacgtggagcccgacaccgagcgcggcaagaagggcgtgtactactacaacaacaagctgTGAcagacg |
| | accttggcaggcgtcgggtagggaggtggtggtgatggcgtctcgatgccatcgcacgcatccaacgaccgtatacgca |
| | tcgtccaatgaccgtcggtgtcctctctgcctccgttttgtgagatgtctcaggcttggtgcatcctcgggggccagccac |
| | gttgcgcgtcgtgctgcttgcctctcttgcgcctctgtggtactggaaaatatcatcgaggcccgtttttttgctcccatttcc |
| | tttccgctacatcttgaaagcaaacgacaaacgaagcagcaagcaaagagcacgaggacggtgaacaagtctgtcac |
| | ctgtatacatctatttccccgcgggtgcacctactctctctcctgccccggcagagtcagcgcagcagcagctcggatagta |
| | tcgacacactctggacgctggtcgtgtgatggactgttgccgccacacttgctgccttgacctgtgaatatccctgccgcttttatc |
| | aaacagcctcagtgtgtttgatcttgtgtgtacgcgcttttgcgagttgctagctgcttgtgctatttgcgaataccacccccagcat |
| | ccccttccctcgtttcatatcgcttgcatcccaaccgcaacttatctacgctgtcctgctatccctcagcgctgctcctgctcctgctc |
| | actgcccctcgcacagccttggtttgggctccgcctgtattctcctggtactgcaacctgtaaaccagcactgcaatgctgatgca |
| | cgggaagtagtgggatgggaacacaaatggagagctcagcgtctgcgtgttgggagctggagtcgtgggcttgacgacgg |
| | cgctgcagctcttgcaggatgtgcctggcgtgcgcgttcacgtcgtggctgagaaatatggcgacgaaacgttgacggc |
| | tggggccggcgggctgtggatgccatacgcattgggtacgcggccattggatgggattggtaggcttatggagggataa |
| | tagagtttttgtcggatccaacgcatgttgatgcagtagcccggtgtgctgaaagtatggaaggatggtgcattggctatt |
| | cacatgcgctgcccaccccttttcgcaggaaatgtgccggcatcgttggtgcaccgatggggaaaatcgacgttcgacc |
| | actacatgaaaatgtatacctctgaagatgcagcaactgcgggtgcgaaacggataacggtttggtaggtgtatgtcgc |
| | agcatgtgctggattttgcgggcaactccccctgccacggcccattgcaggtgtcatgttgactggaggatacgaactttc |
| | ggccgtcaaattcccagaggaggaccccctctgggccgacattgtgcccactgctcttccgcttgctgtttcctgtgtgaa |
| | attgt |

TABLE 23 shows the fatty acid profiles of 11 primary transformants of CHK22 transformed with pCHK398 and grown as described for oil production and resulting FAMEs analyzed by GC-FID as described. P. moriformis base strain CHK22 is shown as non-transgenic control. Strains were grown for 120 hours in 96-well blocks at 200 rpm at 28° C. Biomass was harvested, lyophilized to dryness, and subjected to direct transesterification to generate FAMEs for subsequent quantitation and characterization by GC/FID.

TABLE 23
Screen of primary transformants of CHK83 with pCHK398

| Sample | | | | | | | C18:3 | | | 12-OH, 9c- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 83 | 0.05 | 1.72 | 23.98 | 3.12 | 53.65 | 9.38 | 0.69 | 0.35 | 0.03 | 5.56 |
| Sample 84 | 0.06 | 1.73 | 24.60 | 3.40 | 54.26 | 9.99 | 0.68 | 0.37 | 0.03 | 3.39 |
| Sample 85 | 0.05 | 1.58 | 23.69 | 2.98 | 55.50 | 10.30 | 0.69 | 0.31 | 0.03 | 3.36 |
| Sample 86 | 0.05 | 1.52 | 23.94 | 3.11 | 55.97 | 9.62 | 0.68 | 0.34 | 0.03 | 3.28 |
| Sample 87 | 0.05 | 1.59 | 24.51 | 2.93 | 55.74 | 9.44 | 0.66 | 0.29 | 0.03 | 3.25 |
| Sample 88 | 0.05 | 1.57 | 25.01 | 2.96 | 55.82 | 10.03 | 0.70 | 0.27 | 0.03 | 2.08 |
| Sample 89 | 0.04 | 1.57 | 26.16 | 2.93 | 56.26 | 8.67 | 0.61 | 0.26 | 0.03 | 2.05 |
| Sample 90 | 0.05 | 1.51 | 24.55 | 3.03 | 56.60 | 10.07 | 0.67 | 0.28 | 0.03 | 1.78 |
| Sample 91 | 0.05 | 1.54 | 25.01 | 3.11 | 56.30 | 9.85 | 0.64 | 0.29 | 0.03 | 1.73 |
| Sample 92 | 0.05 | 1.48 | 25.03 | 3.20 | 56.75 | 9.68 | 0.63 | 0.31 | 0.03 | 1.42 |
| Sample 93 | 0.05 | 1.49 | 25.11 | 2.92 | 57.83 | 10.11 | 0.67 | 0.24 | 0.03 | 0.09 |
| CHK22 | 0.04 | 1.66 | 28.81 | 3.02 | 55.87 | 8.30 | 0.57 | 0.23 | 0.03 | 0.09 |
| Control 5 | | | | | | | | | | |

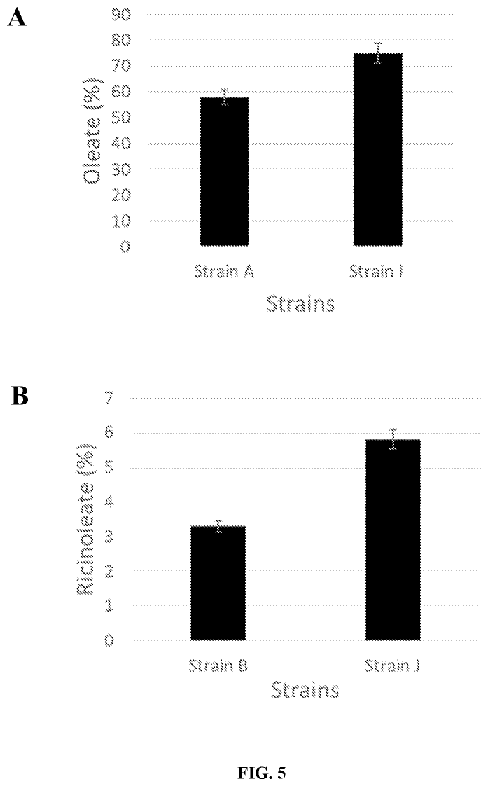

Transformants of CHK22 were generated via particle bombardment using gold nanoparticles. Upon transformation of plasmid constructs into strain CHK22, positive clones were selected on plates with sucrose as the sole carbon source. Primary transformants were clonally purified and grown under standard lipid production conditions. Lipid samples were prepared from dried biomass from each transformant. Fatty acid profiles were determined using direct transesterification methods. Ricinoleic acid production (expressed as Area % of total fatty acid) of several positive transformants, compared to those of untransformed strain CHK22 control, are summarized in TABLE 5, 19, 21, and 23. These results indicated that the PmPGH and PmPGK 3′-UTRs had a greater impact on LFAH12 expression than either the PmCvNR or HSP90 3′-UTRs. FIG. 5 shows a comparison of oleate and ricinoleate levels of P. moriformis strains CHK22 and CHK48 (FIG. 5, Panel A) and with strains CHK22 and CHK48 with expression of LFAH12 (FIG. 5, Panel B).

### Example 5: Expression of an Optimized LFAH12 Expression Cassette in a Higher Oleic Accumulating Strain, CHK64

The transforming DNA sequence of the optimized LFAH12 construct 5′DAO1b::CrTUB2:ScSUC2:PmPGH:CvNR:PmAMT03:LFAH12:PmPGH::3′DAO1b (pCHK397) was shown in SEQ ID NO: 19. To determine if expressing this optimized oleate hydroxylase construct in a higher oleate strain would result in higher ricinoleate production, pCHK397 was transformed into a higher oleate producing strain obtained through mutagenesis designated CHK64 producing 81% oleate.

Transformants of CHK64 were generated via particle bombardment using gold nanoparticles. Upon transformation of pCHK397 into CHK64, positive clones were selected on plates with sucrose as the sole carbon source. Primary transformants were clonally purified and grown under standard lipid production conditions. Lipid samples were prepared from dried biomass from each transformant. Fatty acid profiles were determined using direct transesterification methods. Fatty acid profiles (expressed as Area % of total fatty acid) of 14 positive transgenic lines, compared to the untransformed strain CHK64 control, are shown in TABLE 24.

TABLE 24
Screen of primary transformants of CHK64 with pCHK397

| Sample | | | | | | | C18:3 | | | 12-OH, 9c- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 94 | 0.02 | 0.40 | 5.24 | 2.80 | 73.19 | 7.94 | 0.40 | 0.26 | 0.77 | 8.32 |
| Sample 95 | 0.02 | 0.36 | 5.23 | 2.91 | 75.01 | 6.39 | 0.31 | 0.24 | 0.66 | 8.29 |
| Sample 96 | 0.02 | 0.35 | 5.27 | 2.64 | 74.78 | 7.65 | 0.33 | 0.20 | 0.69 | 7.41 |
| Sample 97 | 0.02 | 0.39 | 5.64 | 2.74 | 76.26 | 6.67 | 0.36 | 0.20 | 0.59 | 6.57 |
| Sample 98 | 0.02 | 0.39 | 5.68 | 2.79 | 75.68 | 7.12 | 0.37 | 0.21 | 0.62 | 6.53 |
| Sample 99 | 0.02 | 0.38 | 5.74 | 2.76 | 76.45 | 6.51 | 0.34 | 0.19 | 0.57 | 6.49 |
| Sample 100 | 0.03 | 0.45 | 5.39 | 2.83 | 73.58 | 9.18 | 0.41 | 0.22 | 0.83 | 6.40 |
| Sample 101 | 0.03 | 0.52 | 4.20 | 3.35 | 76.20 | 8.32 | 0.50 | 0.25 | 0.82 | 5.33 |
| Sample 102 | 0.03 | 0.42 | 5.47 | 2.65 | 74.64 | 9.37 | 0.48 | 0.22 | 0.00 | 5.30 |
| Sample 103 | 0.03 | 0.44 | 6.67 | 2.91 | 76.28 | 7.01 | 0.44 | 0.21 | 0.45 | 5.00 |
| Sample 104 | 0.03 | 0.37 | 6.14 | 2.76 | 77.76 | 6.90 | 0.33 | 0.19 | 0.00 | 4.41 |
| Sample 105 | 0.02 | 0.37 | 5.90 | 2.72 | 78.58 | 6.91 | 0.35 | 0.18 | 0.56 | 3.86 |
| Sample 106 | 0.03 | 0.38 | 5.90 | 2.44 | 78.06 | 8.04 | 0.44 | 0.13 | 0.49 | 3.55 |
| Sample 107 | 0.03 | 0.41 | 5.96 | 2.35 | 77.87 | 8.36 | 0.45 | 0.12 | 0.00 | 3.45 |
| CHK64 | 0.02 | 0.42 | 7.45 | 2.53 | 80.42 | 7.61 | 0.39 | 0.08 | 0.32 | 0.11 |
| Control 1 | | | | | | | | | | |

P. moriformis high oleic base strain CHK64 is shown as non-transgenic control. Strains were grown for 96 hours in 96-well blocks at 200 rpm at 28° C. Biomass was harvested, lyophilized to dryness, and subjected to direct transesterification to generate FAMEs for subsequent quantitation and characterization by GC/FID.

Three transgenic lines (Samples 108, 109, and 110) were further evaluated in 5 mL cultures grown in 50-mL conical tubes. The results are shown in TABLE 25. P. moriformis high oleic base strain CHK64 is shown as non-transgenic control. Strains were grown for 96 hours in a 50-mL bioreactor tube at 200 rpm at 28° C. Biomass was harvested, lyophilized to dryness, and subjected to direct transesterification to generate FAMEs for subsequent quantitation and characterization by GC/FID.

TABLE 25
Screen of primary transformants of CHK64 with pCHK397 in a 50-mL shake tube experiment

| Sample | | | | | | | C18:3 | | | 12-OH- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 108 | 0.00 | 0.37 | 3.96 | 3.02 | 67.30 | 7.83 | 0.38 | 0.32 | 0.99 | 15.26 |
| Sample 109 | 0.02 | 0.43 | 5.13 | 3.06 | 71.14 | 6.74 | 0.38 | 0.26 | 0.63 | 11.55 |
| Sample 110 | 0.02 | 0.40 | 4.96 | 3.01 | 71.26 | 6.61 | 0.34 | 0.27 | 0.74 | 11.78 |
| CHK64 | 0.02 | 0.36 | 6.63 | 2.58 | 81.73 | 7.09 | 0.34 | 0.11 | 0.39 | 0.10 |
| Control 2 | | | | | | | | | | |

Sample 108 was selected for additional rounds of stability to ensure that its fatty acid profile was reproducible. Two single colonies from Sample 108 were subsequently banked as CHK72 and CHK84, respectively. Strains CHK84 and CHK72 were then evaluated in a 50-mL shake tube experiment with an elongated incubation time (144 h). CHK84 and CHK72 produced 16.23% and 14.52% ricinoleate as shown in TABLE 26. P. moriformis high oleic base strain CHK64 is shown as non-transgenic control. Strains were grown for 144 hours in 50-mL bioreactor tube at 200 rpm at 28° C. Biomass was harvested, lyophilized to dryness, and subjected to direct transesterification to generate FAMEs for subsequent quantitation and characterization by GC/FID.

TABLE 26
Evaluation of CHK84 and CHK72 in a shake tube experiment

| Sample | | | | | | | C18:3 | | | 12-OH- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| CHK84 | 0.03 | 0.46 | 4.65 | 3.23 | 64.04 | 9.03 | 0.37 | 0.40 | 0.98 | 16.23 |
| CHK72 | 0.02 | 0.41 | 4.68 | 2.91 | 66.16 | 8.92 | 0.40 | 0.40 | 0.95 | 14.52 |
| CHK64 | 0.02 | 0.35 | 6.98 | 2.57 | 81.67 | 6.82 | 0.31 | 0.11 | 0.44 | 0.11 |

### Example 6: Allele-Specific Down-Regulation of Endogenous P. moriformis Thioesterases in Base Strain CHK27

FATAs are the sole fatty acyl-ACP thioesterases present in P. moriformis. As such, these enzymes play a key role in determining the fatty acyl chain length distribution of fatty acids in triglyceride oils. This experiment selectively downregulated each allele of FATA in P. moriformis to analyze individual effects on oleate accumulation, and by extension, these effects on elevating ricinoleate levels in an LFAH12 expressing strain.

### Down-Regulation of FATA-1 in CHK27

The expression construct contains 5′ and 3′ homology arms to permit its targeted integration into the P. moriformis genome and is depicted in SEQ ID NO: 21.

TABLE 27 shows sequences of constructs. SEQ ID NO: 21 shows integrative sequences for the transformation of P. moriformis with pCHK55. The construct can be written as FATA-1::PmHXT1:ScMEL1:PmPGK1::FATA-1. Proceeding in the 5′ to 3′ direction, bold, lowercase sequences represent genomic DNA from strain CHK27 that permit targeted integration at the FATA-1 locus via homologous recombination. The P. moriformis hexose transporter 1 (HXT1) promoter driving expression of the S. carlbergensis melibiase (ScMEL1) is indicated by boxed uppercase text. The initiator ATG and terminator TAA for ScMEL1 are indicated by uppercase, bold italics while the coding region is indicated in lowercase italics. P. moriformis phosphoglucokinase (PGK) 3′-UTR is indicated by uppercase underlined text followed by the CHK27 FATA-1 3′ flanking genomic region indicated by bold, lowercase text.

TABLE 27
Sequences

| SEQ | |
|---|---|
| ID NO | Sequence |
| 21 | gctcttccgcttggagtcactgtgccactgagttcgactggtagctgaatggagtcgctgctccactaaacgaattgtcag |
| | caccgccagccggccgaggacccgagtcatagcgagggtagtagcgcgccatggcaccgaccagcctgcttgccagt |
| | actggcgtctcttccgcttctctgtggtcctctgcgcgctccagcgcgtgcgcttttccggtggatcatgcggtccgtggcg |
| | caccgcagcggccgctgcccatgcagcgccgctgcttccgaacagtggcggtcagggccgcacccgcggtagccgtcc |
| | gtccggaacccgcccaagagttttgggagcagcttgagccctgcaagatggcggaggacaagcgcatcttcctggagg |
| | agcaccggtgcgtggaggtccggggctgaccggccgtcgcattcaacgtaatcaatcgcatgatgatcagaggacacg |
| | aagtcttggtggcggtggccagaaacactgtccattgcaagggcatagggatgcgttccttcacctctcatttctcatttct |
| | 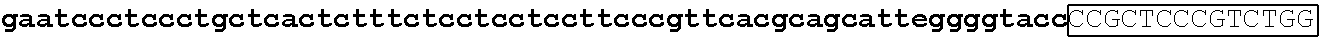 |
| | 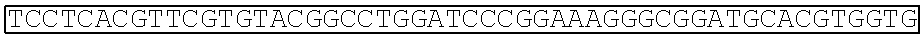 |
| | 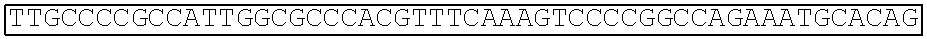 |
| | 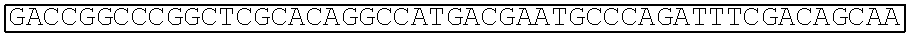 |
| | 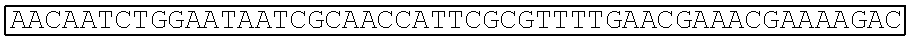 |
| | 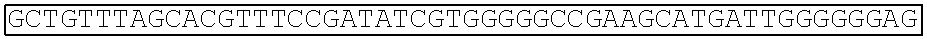 |
| | 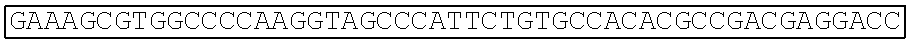 |
| | 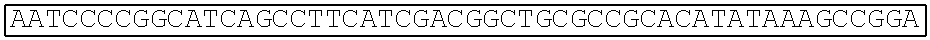 |
| | 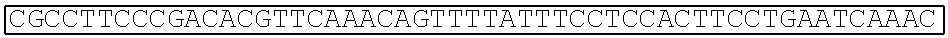 |
| | 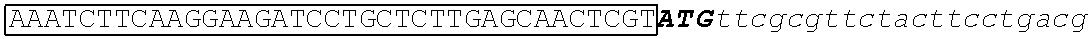 |
| | gcctgcatctccctgaagggcgtgttcggcgtctccccctcctacaacggcctgggcctgacgccccagatgggctgggac |
| | aactggaacacgttcgcctgcgacgtctccgagcagctgctgctggacacggccgaccgcatctccgacctgggcctgaa |
| | ggacatgggctacaagtacatcatcctggacgactgctggtcctccggccgcgactccgacggcttcctggtcgccgacga |
| | gcagaagttccccaacggcatgggccacgtcgccgaccacctgcacaacaactccttcctgttcggcatgtactcctccgcg |
| | ggcgagtacacgtgcgccggctaccccggctccctgggccgcgaggaggaggacgcccagttcttcgcgaacaaccgc |
| | gtggactacctgaagtacgacaactgctacaacaagggccagttcggcacgcccgagatctcctaccaccgctacaaggc |
| | catgtccgacgccctgaacaagacgggccgccccatcttctactccctgtgcaactggggccaggacctgaccttctactgg |
| | ggctccggcatcgcgaactcctggcgcatgtccggcgacgtcacggcggagttcacgcgccccgactcccgctgcccctg |
| | cgacggcgacgagtacgactgcaagtacgccggcttccactgctccatcatgaacatcctgaacaaggccgcccccatgg |
| | gccagaacgcgggcgtcggcggctggaacgacctggacaacctggaggtcggcgtcggcaacctgacggacgacgag |
| | gagaaggcgcacttctccatgtgggccatggtgaagtcccccctgatcatcggcgcgaacgtgaacaacctgaaggcctc |
| | ctcctactccatctactcccaggcgtccgtcatcgccatcaaccaggactccaacggcatccccgccacgcgcgtctggcgc |
| | tactacgtgtccgacacggacgagtacggccagggcgagatccagatgtggtccggccccctggacaacggcgaccag |
| | gtcgtggcgctgctgaacggcggctccgtgtcccgccccatgaacacgaccctggaggagatcttcttcgactccaacctgg |
| | gctccaagaagctgacctccacctgggacatctacgacctgtgggcgaaccgcgtcgacaactccacggcgtccgccatc |
| | ctgggccgcaacaagaccgccaccggcatcctgtacaacgccaccgagcagtcctacaaggacggcctgtccaagaac |
| | gacacccgcctgttcggccagaagatcggctccctgtcccccaacgcgatcctgaacacgaccgtccccgcccacggcat |
| | cgcgttctaccgcctgcgcccctcctccTGAtacaacttattacgtattctgaccggcgctgatgtggcgcggacgccgtcgta |
| | ctctttcagactttactcttgaggaattgaacctttctcgcttgctggcatgtaaacattggcgcaattaattgtgtgatgaagaaagg |
| | gtggcacaagatggatcgcgaatgtacgagatcgacaacgatggtgattgttatgaggggccaaacctggctcaatcttgtcgc |
| | atgtccggcgcaatgtgatccagcggcgtgactctcgcaacctggtagtgtgtgcgcaccgggtcgctttgattaaaactgatcg |
| | cattgccatcccgtcaactcacaagcctactctagctcccattgcgcactcgggcgcccggctcgatcaatgttctgagcggag |
| | ggcgaagcgtcaggaaatcgtctcggcagctggaagcgcatggaatgcggagcggagatcgaatcagcggccgcgacag |
| | ggtggttggctggatggggaaacgctggtcgcgggattcgatcctgctgcttatatcctccctggaagcacacccacgac |
| | tctgaagaagaaaacgtgcacacacacaacccaaccggccgaatatttgcttccttatcccgggtccaagagagactgc |
| | gatgcccccctcaatcagcatcctcctccctgccgcttcaatcttccctgcttgcctgcgcccgcggtgcgccgtctgcccg |
| | cccagtcagtcactcctgcacaggccccttgtgcgcagtgctcctgtaccctttaccgctccttccattctgcgaggccccc |
| | tattgaatgtattcgttgcctgtgtggccaagcgggctgctgggcgcgccgccgtcgggcagtgctcggcgactttggcg |
| | gaagccgattgttcttctgtaagccacgcgcttgctgctttgggaagagaagggggggggtactgaatggatgaggagg |
| | agaaggaggggtattggtattatctgagttgggtgctcttccgct |

Transformants of CHK27 were generated via particle bombardment using gold nanoparticles. Upon transformation of pCHK55 into CHK27, positive clones were selected on plates with melibiose as the sole carbon source. Primary transformants were clonally purified and grown under standard lipid production conditions. Lipid samples were prepared from dried biomass from each transformant. Fatty acid profiles were determined using direct transesterification methods. Fatty acid profiles (expressed as Area % of total fatty acid) of 15 positive transgenic lines, compared to the untransformed strain CHK27 control, are shown in TABLE 28. P. moriformis high oleic base strain CHK27 is shown as a non-transgenic control. Strains were grown for 72 hours in 96-well blocks at 200 rpm at 28° C. Biomass was harvested, lyophilized to dryness, and subjected to direct transesterification to generate FAMEs for subsequent quantitation and characterization by GC/FID.

TABLE 28
Screen of primary transformants of CHK27 with pCHK55

| Sample | | | | | | | C18:3 | | | | |
|---|---|---|---|---|---|---|---|---|---|---|---|
| Name | C12:0 | C14:0 | C16:0 | C18:0 | C18:1 | C18:2 | alpha | C20:0 | C20:1 | C22:0 | C24:0 |
| Sample 111 | 0.06 | 1.53 | 18.43 | 2.48 | 67.10 | 8.85 | 0.43 | 0.13 | 0.14 | 0.00 | 0.00 |
| Sample 112 | 0.00 | 1.48 | 17.23 | 3.41 | 67.87 | 8.64 | 0.43 | 0.27 | 0.00 | 0.00 | 0.00 |
| Sample 113 | 0.00 | 1.43 | 16.94 | 3.22 | 68.24 | 8.88 | 0.44 | 0.23 | 0.00 | 0.00 | 0.00 |
| Sample 114 | 0.00 | 1.37 | 16.83 | 3.23 | 68.41 | 8.80 | 0.46 | 0.21 | 0.09 | 0.00 | 0.00 |
| Sample 115 | 0.00 | 1.44 | 16.76 | 3.33 | 68.61 | 8.53 | 0.48 | 0.22 | 0.00 | 0.00 | 0.00 |
| Sample 116 | 0.00 | 1.40 | 16.57 | 3.39 | 68.79 | 8.62 | 0.46 | 0.20 | 0.00 | 0.00 | 0.00 |
| Sample 117 | 0.00 | 1.38 | 16.48 | 3.16 | 68.46 | 9.14 | 0.48 | 0.23 | 0.00 | 0.00 | 0.00 |
| Sample 118 | 0.00 | 1.36 | 16.47 | 3.42 | 68.44 | 8.83 | 0.45 | 0.25 | 0.20 | 0.00 | 0.00 |
| Sample 119 | 0.06 | 1.42 | 16.44 | 3.15 | 69.15 | 8.21 | 0.37 | 0.18 | 0.17 | 0.00 | 0.00 |
| Sample 120 | 0.00 | 1.30 | 16.36 | 3.38 | 68.85 | 8.56 | 0.46 | 0.21 | 0.19 | 0.00 | 0.00 |
| Sample 121 | 0.00 | 1.38 | 16.22 | 3.25 | 68.89 | 8.82 | 0.44 | 0.23 | 0.19 | 0.00 | 0.00 |
| Sample 122 | 0.00 | 1.31 | 16.15 | 3.43 | 68.33 | 9.27 | 0.52 | 0.25 | 0.12 | 0.00 | 0.00 |
| Sample 123 | 0.00 | 1.33 | 16.15 | 3.31 | 69.43 | 8.36 | 0.41 | 0.22 | 0.21 | 0.00 | 0.00 |
| Sample 124 | 0.00 | 1.32 | 15.88 | 3.61 | 69.34 | 8.52 | 0.42 | 0.22 | 0.06 | 0.00 | 0.00 |
| Sample 125 | 0.06 | 1.38 | 15.68 | 3.85 | 69.21 | 8.31 | 0.40 | 0.26 | 0.17 | 0.00 | 0.00 |
| CHK27 | 0.06 | 1.08 | 13.96 | 2.95 | 72.99 | 7.57 | 0.34 | 0.13 | 0.17 | 0.00 | 0.00 |
| Control 1 | | | | | | | | | | | |

### Down-Regulation of FATA-2 in CHK27

TABLE 29 shows sequences of constructs. The expression construct contains 5′ and 3′ homology arms to permit targeted integration into the P. moriformis genome and is depicted in SEQ ID NO: 22. SEQ ID NO: 22 shows integrative sequences for the transformation of P. moriformis with pCHK58. The construct can be written as FATA-2::CrTUB2:ScSUC2:CvNR::FATA-2. Bold, lowercase sequences represent genomic DNA from CHK27 that permits targeted integration at the FATA-2 locus via homologous recombination. Proceeding in the 5′ to 3′ direction, the Chlamydomonas reinhardtii β-tubulin promoter drives expression of the yeast sucrose invertase gene, conferring the ability of CHK27 to metabolize sucrose, is indicated by boxed uppercase text. The initiator ATG and terminator TAA for invertase are indicated by uppercase, bold italics while the coding region is indicated in lowercase italics. The Chlorella vulgaris nitrate reductase 3′-UTR is indicated by lower case underlined text followed by the strain CHK27 FATA-2 3′ flanking genomic region indicated by bold, lowercase text.

TABLE 29
Sequences

| SEQ | |
|---|---|
| ID NO | Sequence |
| 22 | cttaatggagtcgctgctccactaatcgaattgtcagcaccgccagccggccgaggacccgagtcatagcgagggtagt |
| | agcgcgccatggcaccgaccagcctgcttgcccgtactggcgtctcttccgcttctctgtgctcctctacgcgctccggcg |
| | cgtgcgcttttccggtggatcatgcggtccgtggcgcaccgcagcggccgctgcccatgcagcgccgctgcttccgaaca |
| | gtggctgtcagggccgcacccgcagtagccgtccgtccggaacccgcccaagagttttgggagcagcttgagccctgca |
| | agatggcggaggacaagcgcatcttcctggaggagcaccggtgcgcggaggtccggggctgaccggccgtcgcattc |
| | aacgtaatcaatcgcatgatgatcacaggacgcgacgtcttggtggcggtggccagggacactgcccattgcacaggc |
| | ataggaatgcgttccttctcatttctcagttttctgagcccctccctcttcactctttctcctcctcctcccctctcacgcagca |
| | 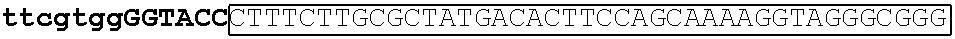 |
| | 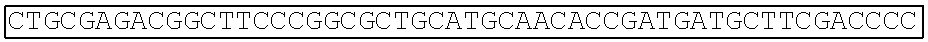 |
| | 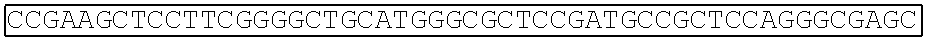 |
| | 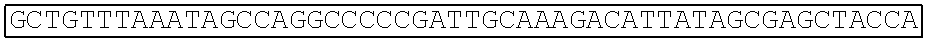 |
| | 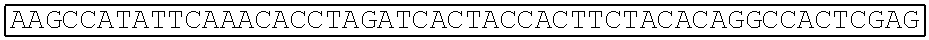 |
| | 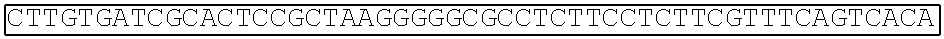 |
| | 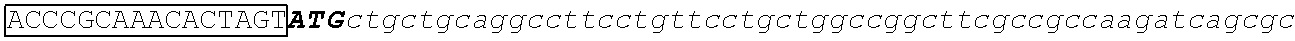 |
| | ctccatgacgaacgagacgtccgaccgccccctggtgcacttcacccccaacaagggctggatgaacgaccccaacggc |
| | ctgtggtacgacgagaaggacgccaagtggcacctgtacttccagtacaacccgaacgacaccgtctgggggacgccctt |
| | gttctggggccacgccacgtccgacgacctgaccaactgggaggaccagcccatcgccatcgccccgaagcgcaacga |
| | ctccggcgccttctccggctccatggtggtggactacaacaacacctccggcttcttcaacgacaccatcgacccgcgccag |
| | cgctgcgtggccatctggacctacaacaccccggagtccgaggagcagtacatctcctacagcctggacggcggctacac |
| | cttcaccgagtaccagaagaaccccgtgctggccgccaactccacccagttccgcgacccgaaggtcttctggtacgagcc |
| | ctcccagaagtggatcatgaccgcggccaagtcccaggactacaagatcgagatctactcctccgacgacctgaagtcct |
| | ggaagctggagtccgcgttcgccaacgagggcttcctcggctaccagtacgagtgccccggcctgatcgaggtccccacc |
| | gagcaggaccccagcaagtcctactgggtgatgttcatctccatcaaccccggcgccccggccggcggctccttcaaccag |
| | tacttcgtcggcagcttcaacggcacccacttcgaggccttcgacaaccagtcccgcgtggtggacttcggcaaggactact |
| | acgccctgcagaccttcttcaacaccgacccgacctacgggagcgccctgggcatcgcgtgggcctccaactgggagtac |
| | tccgccttcgtgcccaccaacccctggcgctcctccatgtccctcgtgcgcaagttctccctcaacaccgagtaccaggccaa |
| | cccggagacggagctgatcaacctgaaggccgagccgatcctgaacatcagcaacgccggcccctggagccggttcgc |
| | caccaacaccacgttgacgaaggccaacagctacaacgtcgacctgtccaacagcaccggcaccctggagttcgagctg |
| | gtgtacgccgtcaacaccacccagacgatctccaagtccgtgttcgcggacctctccctctggttcaagggcctggaggacc |
| | ccgaggagtacctccgcatgggcttcgaggtgtccgcgtcctccttcttcctggaccgcgggaacagcaaggtgaagttcgt |
| | gaaggagaacccctacttcaccaaccgcatgagcgtgaacaaccagcccttcaagagcgagaacgacctgtcctactac |
| | aaggtgtacggcttgctggaccagaacatcctggagctgtacttcaacgacggcgacgtcgtgtccaccaacacctacttca |
| | tgaccaccgggaacgccctgggctccgtgaacatgacgacgggggtggacaacctgttctacatcgacaagttccaggtg |
| | cgcgaggtcaagtgattaatTAAgcagcagcagctcggatagtatcgacacactctggacgctggtcgtgtgatggactgtt |
| | gccgccacacttgctgccttgacctgtgaatatccctgccgcttttatcaaacagcctcagtgtgtttgatcttgtgtgtacgcgcttt |
| | tgcgagttgctagctgcttgtgctatttgcgaataccacccccagcatccccttccctcgtttcatatcgcttgcatcccaaccgcaa |
| | cttatctacgctgtcctgctatccctcagcgctgctcctgctcctgctcactgcccctcgcacagccttggtttgggctccgcctgta |
| | ttctcctggtactgcaacctgtaaaccagcactgcaatgctgatgcacgggaagtagtgggatgggaacacaaatggacctgca |
| | gggcggccgcagaggagagggtggctggtagttgcgggatggctggtcgcccgtcgatcctgctgctgctattgtctcct |
| | cctgcacaagcccacccacgactccgaagaagaagaagaaaacgcgcacacacacaacccaaccggccgaatattt |
| | gcttccttatcccgggtccaagagagacggcgatgcccccctcaatcagcctcctcctccctgccgctccaatcttccctg |
| | cttgcatgcgcccgcgagaggctgtctgcgcgccccgtcagtcactccccgtgcagacgcctcgtgctcggtgctcctgt |
| | atcctttaccgctcctttcattctgcgaggccccctgttgaatgtattcgttgcctgtgtggccaagcgcgctgctgggcgc |
| | gccgccgtcgggcggtgctcggcgactctggcggaagccggttgttct |

Transformants of CHK27 were generated via particle bombardment using gold nanoparticles. Upon transformation of pCHK58 into CHK27, positive clones were selected on plates with sucrose as the sole carbon source. Primary transformants were clonally purified and grown under standard lipid production conditions. Lipid samples were prepared from dried biomass from each transformant. Fatty acid profiles were determined using direct transesterification methods. Fatty acid profiles (expressed as Area % of total fatty acid) of 16 positive transgenic lines, compared to the untransformed strain CHK27 control, are shown in TABLE 30.

P. moriformis base strain CHK27 is shown as non-transgenic control. Strains were grown for 72 hours in 96-well blocks at 200 rpm at 28° C. Biomass was harvested, lyophilized to dryness, and subjected to direct transesterification to generate FAMEs for subsequent quantitation and characterization by GC/FID.

TABLE 30
Screen of primary transformants of CHK27 with pCHK58

| Sample | | | | | | | C18:3 | | | 12-OH- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 126 | 0.0 | 0.56 | 4.33 | 3.10 | 78.32 | 11.74 | 0.66 | 0.17 | 0.80 | 0.00 |
| Sample 127 | 0.00 | 0.82 | 7.63 | 2.79 | 78.42 | 8.92 | 0.42 | 0.13 | 0.35 | 0.00 |
| Sample 128 | 0.00 | 0.93 | 8.13 | 2.85 | 77.47 | 9.26 | 0.39 | 0.15 | 0.29 | 0.00 |
| Sample 129 | 0.06 | 0.86 | 8.15 | 2.89 | 77.74 | 8.95 | 0.41 | 0.13 | 0.31 | 0.00 |
| Sample 130 | 0.00 | 0.90 | 8.49 | 3.14 | 77.14 | 8.98 | 0.41 | 0.14 | 0.30 | 0.00 |
| Sample 131 | 0.00 | 0.87 | 8.62 | 3.06 | 77.31 | 8.74 | 0.42 | 0.15 | 0.33 | 0.00 |
| Sample 132 | 0.00 | 0.81 | 8.63 | 2.96 | 77.71 | 8.50 | 0.41 | 0.14 | 0.31 | 0.00 |
| Sample 133 | 0.00 | 0.85 | 8.63 | 2.93 | 77.54 | 8.73 | 0.44 | 0.14 | 0.32 | 0.00 |
| Sample 134 | 0.00 | 0.89 | 8.68 | 3.09 | 76.99 | 8.95 | 0.41 | 0.16 | 0.33 | 0.00 |
| Sample 135 | 0.00 | 0.80 | 8.69 | 2.99 | 77.92 | 8.24 | 0.41 | 0.12 | 0.28 | 0.00 |
| Sample 136 | 0.00 | 0.86 | 8.71 | 3.10 | 77.39 | 8.54 | 0.37 | 0.13 | 0.21 | 0.00 |
| Sample 137 | 0.00 | 0.90 | 8.76 | 3.13 | 77.15 | 8.72 | 0.38 | 0.13 | 0.29 | 0.00 |
| Sample 138 | 0.00 | 0.87 | 9.06 | 3.10 | 76.28 | 9.23 | 0.48 | 0.18 | 0.30 | 0.00 |
| Sample 139 | 0.06 | 0.93 | 9.10 | 3.10 | 76.72 | 8.70 | 0.40 | 0.12 | 0.26 | 0.00 |
| Sample 140 | 0.00 | 0.87 | 9.68 | 3.12 | 75.75 | 9.08 | 0.50 | 0.23 | 0.29 | 0.00 |
| Sample 141 | 0.00 | 0.97 | 9.74 | 3.28 | 75.07 | 9.60 | 0.46 | 0.20 | 0.27 | 0.00 |
| CHK27 | 0.00 | 1.20 | 13.89 | 3.07 | 72.32 | 8.12 | 0.36 | 0.15 | 0.21 | 0.00 |
| Control 2 | | | | | | | | | | |

Transformants of CHK27 were generated via particle bombardment using gold nanoparticles. Upon transformation of pCHK55 (FATA-1 allele knockout cassette) or pCHK58 (FATA-2 allele knockout cassette) into CHK27, positive clones were selected on plates with melibiose or sucrose, respectively as the sole carbon source. Primary transformants were clonally purified and grown under standard lipid production conditions. Lipid samples were prepared from dried biomass from each transformant. Fatty acid profiles were determined using direct transesterification methods. Fatty acid profiles (expressed as Area % of total fatty acid) of positive transgenic lines, compared to the untransformed strain CHK27 control are shown in TABLE 28 and TABLE 30. These results indicate that gene deletion of P. moriformis thioesterases (PmFATA-1 and PmFATA-2) results in distinct shifts in fatty acid profiles, with deletion of the FATA-1 allele resulting in decreased oleate and increased levels of palmitate (TABLE 28) while deletion of the FATA-2 allele results in decreased levels of saturates and increased levels of oleate (TABLE 30) in base strain CHK27.

### Example 7: Allele-Specific Down-Regulation of P. moriformis FATA Thioesterases in Ricinoleate Producing Strain CHK72

Because oleate hydroxylases benefit from higher substrate levels, namely oleic acid, this experiment sought to determine the effect of allele-specific knockouts of FATA-1 and FATA-2 in a strain elaborating high levels of ricinoleic acid, namely CHK72.

### Down-Regulation of FATA-1 in CHK72

The expression construct FATA-1::PmHXT1:ScMEL1:PmPGK1::FATA-1 (PM12) was utilized in the transformation of strain CHK72. Transformants of CHK72 were generated via particle bombardment using gold nanoparticles. Upon transformation of pCHK55 into CHK72, positive clones were selected on plates with melibiose as the sole carbon source. Primary transformants were clonally purified and grown under standard lipid production conditions. Lipid samples were prepared from dried biomass from each transformant. Fatty acid profiles were determined using direct transesterification methods. Fatty acid profiles (expressed as Area % of total fatty acid) of 24 positive transgenic lines, compared to the untransformed strain CHK64 and the ricinoleate expressing strain, CHK72, are shown in TABLE 31.

P. moriformis ricinoleate producing strain CHK72 and high oleic base strain CHK64 are shown as controls. Strains were grown for 72 hours in 96-well blocks at 200 rpm at 28° C. Biomass was harvested, lyophilized to dryness, and subjected to direct transesterification to generate FAMEs for subsequent quantitation and characterization by GC/FID.

TABLE 31
Screen of primary transformants of CHK72 with pCHK55

| Sample | | | | | | | C18:3 | | | 12-OH- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 142 | 0.02 | 0.53 | 6.89 | 2.45 | 72.01 | 9.71 | 0.37 | 0.27 | 0.84 | 6.20 |
| Sample 143 | 0.02 | 0.55 | 6.89 | 2.46 | 70.96 | 10.14 | 0.38 | 0.28 | 0.89 | 6.83 |
| Sample 144 | 0.02 | 0.51 | 6.81 | 2.33 | 72.12 | 9.14 | 0.34 | 0.25 | 0.80 | 7.02 |
| Sample 145 | 0.02 | 0.50 | 6.79 | 2.32 | 72.14 | 8.86 | 0.36 | 0.24 | 0.73 | 7.41 |
| Sample 146 | 0.02 | 0.82 | 10.95 | 2.36 | 67.46 | 8.88 | 0.30 | 0.24 | 0.45 | 7.59 |
| Sample 147 | 0.02 | 0.54 | 6.66 | 2.37 | 70.75 | 9.71 | 0.36 | 0.27 | 0.80 | 7.82 |
| Sample 148 | 0.02 | 0.53 | 6.64 | 2.34 | 70.82 | 9.76 | 0.35 | 0.24 | 0.83 | 7.82 |
| Sample 149 | 0.02 | 0.53 | 6.78 | 2.34 | 70.78 | 9.31 | 0.35 | 0.25 | 0.76 | 8.23 |
| Sample 150 | 0.02 | 0.51 | 6.65 | 2.32 | 70.88 | 9.22 | 0.35 | 0.24 | 0.77 | 8.26 |
| Sample 151 | 0.02 | 0.52 | 6.62 | 2.40 | 70.16 | 9.73 | 0.38 | 0.28 | 0.81 | 8.34 |
| Sample 152 | 0.02 | 0.57 | 6.84 | 2.36 | 68.41 | 11.07 | 0.48 | 0.30 | 0.77 | 8.51 |
| Sample 153 | 0.02 | 0.53 | 6.67 | 2.35 | 70.30 | 9.52 | 0.35 | 0.27 | 0.80 | 8.53 |
| Sample 154 | 0.02 | 0.54 | 6.62 | 2.42 | 68.88 | 10.06 | 0.39 | 0.28 | 0.81 | 9.35 |
| Sample 155 | 0.02 | 0.55 | 6.70 | 2.36 | 69.26 | 9.57 | 0.37 | 0.27 | 0.76 | 9.46 |
| Sample 156 | 0.02 | 0.67 | 8.40 | 2.11 | 67.39 | 9.75 | 0.38 | 0.24 | 0.67 | 9.56 |
| Sample 157 | 0.02 | 0.53 | 6.59 | 2.33 | 69.38 | 9.54 | 0.36 | 0.26 | 0.78 | 9.56 |
| Sample 158 | 0.02 | 0.58 | 6.70 | 2.39 | 67.28 | 10.43 | 0.39 | 0.29 | 0.79 | 10.39 |
| Sample 159 | 0.02 | 0.61 | 6.74 | 2.42 | 66.33 | 10.76 | 0.40 | 0.28 | 0.76 | 10.96 |
| Sample 160 | 0.02 | 0.71 | 7.19 | 2.07 | 63.98 | 12.00 | 0.50 | 0.34 | 0.55 | 11.55 |
| Sample 161 | 0.02 | 0.61 | 6.80 | 2.38 | 65.62 | 10.69 | 0.40 | 0.29 | 0.77 | 11.69 |
| Sample 162 | 0.02 | 0.64 | 6.96 | 2.36 | 65.13 | 10.93 | 0.41 | 0.30 | 0.76 | 11.75 |
| Sample 163 | 0.02 | 0.70 | 6.96 | 2.32 | 64.31 | 11.67 | 0.41 | 0.29 | 0.76 | 11.81 |
| Sample 164 | 0.02 | 0.83 | 7.76 | 2.42 | 62.61 | 11.66 | 0.49 | 0.34 | 0.72 | 12.33 |
| Sample 165 | 0.02 | 0.63 | 6.76 | 2.37 | 64.18 | 11.31 | 0.43 | 0.28 | 0.80 | 12.46 |
| CHK72 | 0.02 | 0.57 | 5.97 | 2.59 | 65.16 | 10.13 | 0.37 | 0.29 | 0.72 | 13.50 |
| Control 1 | | | | | | | | | | |
| CHK64 | 0.02 | 0.58 | 7.79 | 2.69 | 78.18 | 9.02 | 0.37 | 0.17 | 0.46 | 0.10 |
| Control 3 | | | | | | | | | | |

### Down-Regulation of FATA-2 in Strain CHK72

TABLE 32 shows sequences of constructs. The expression construct contains 5′ and 3′ homology arms to permit targeted integration of the transforming DNA. SEQ ID NO: 23 shows the integrative sequences for the transformation of P. moriformis with pCHK697. The construct can be written as FATA-2::PmHXT1:ScMEL1:PmPGK1::FATA-2. Bold, lowercase sequences represent genomic DNA from CHK72 that permit targeted integration at the FATA-2 locus via homologous recombination. Proceeding in the 5′ to 3′ direction, the P. moriformis hexose transporter 1 (HXT1) promoter driving expression of the S. carlbergensis melibiase (ScMEL1) is indicated by boxed uppercase text. The initiator ATG and terminator TAA for ScMEL1 are indicated by uppercase, bold italics while the coding region is indicated in lowercase italics. P. moriformis phosphoglucokinase (PGK) 3′-UTR is indicated by uppercase underlined text followed by the CHK72 FATA-2 3′ flanking genomic region indicated by bold, lowercase text.

TABLE 32
Sequences

| SEQ | |
|---|---|
| ID NO | Sequence |
| 23 | cttaatggagtcgctgctccactaatcgaattgtcagcaccgccagccggccgaggacccgagtcatagcgagggtagt |
| | agcgcgccatggcaccgaccagcctgcttgcccgtactggcgtctcttccgcttctctgtgctcctctacgcgctccggcg |
| | cgtgcgcttttccggtggatcatgcggtccgtggcgcaccgcagcggccgctgcccatgcagcgccgctgcttccgaaca |
| | gtggctgtcagggccgcacccgcagtagccgtccgtccggaacccgcccaagagttttgggagcagcttgagccctgca |
| | agatggcggaggacaagcgcatcttcctggaggagcaccggtgcgcggaggtccggggctgaccggccgtcgcattc |
| | aacgtaatcaatcgcatgatgatcacaggacgcgacgtcttggtggcggtggccagggacactgcccattgcacaggc |
| | ataggaatgcgttccttctcatttctcagttttctgagcccctccctcttcactctttctcctcctcctcccctctcacgcagca |
| | 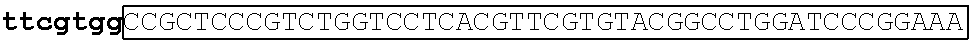 |
| | 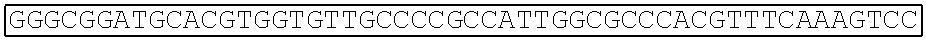 |
| | 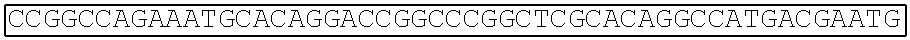 |
| | 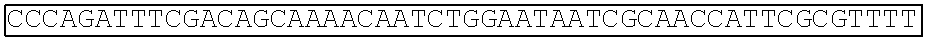 |
| | 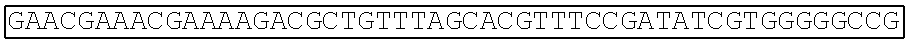 |
| | 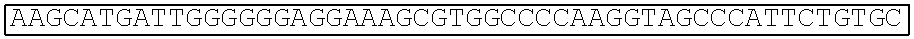 |
| | 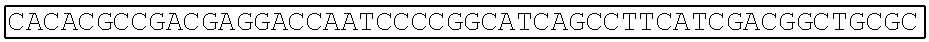 |
| | 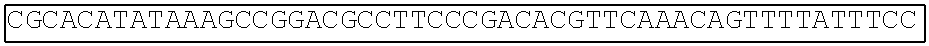 |
| | 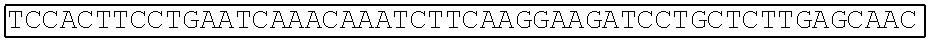 |
| | 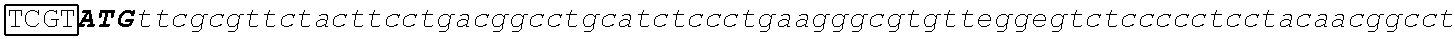 |
| | gggcctgacgccccagatgggctgggacaactggaacacgttcgcctgcgacgtctccgagcagctgctgctggacacg |
| | gccgaccgcatctccgacctgggcctgaaggacatgggctacaagtacatcatcctggacgactgctggtcctccggccgc |
| | gactccgacggcttcctggtcgccgacgagcagaagttccccaacggcatgggccacgtcgccgaccacctgcacaaca |
| | actccttcctgttcggcatgtactcctccgcgggcgagtacacgtgcgccggctaccccggctccctgggccgcgaggagg |
| | aggacgcccagttcttcgcgaacaaccgcgtggactacctgaagtacgacaactgctacaacaagggccagttcggcac |
| | gcccgagatctcctaccaccgctacaaggccatgtccgacgccctgaacaagacgggccgccccatcttctactccctgtg |
| | caactggggccaggacctgaccttctactggggctccggcatcgcgaactcctggcgcatgtccggcgacgtcacggcgg |
| | agttcacgcgccccgactcccgctgcccctgcgacggcgacgagtacgactgcaagtacgccggcttccactgctccatca |
| | tgaacatcctgaacaaggccgcccccatgggccagaacgcgggcgtcggcggctggaacgacctggacaacctggagg |
| | tcggcgtcggcaacctgacggacgacgaggagaaggcgcacttctccatgtgggccatggtgaagtcccccctgatcatc |
| | ggcgcgaacgtgaacaacctgaaggcctcctcctactccatctactcccaggcgtccgtcatcgccatcaaccaggactcc |
| | aacggcatccccgccacgcgcgtctggcgctactacgtgtccgacacggacgagtacggccagggcgagatccagatgt |
| | ggtccggccccctggacaacggcgaccaggtcgtggcgctgctgaacggcggctccgtgtcccgccccatgaacacgac |
| | cctggaggagatcttcttcgactccaacctgggctccaagaagctgacctccacctgggacatctacgacctgtgggcgaa |
| | ccgcgtcgacaactccacggcgtccgccatcctgggccgcaacaagaccgccaccggcatcctgtacaacgccaccgag |
| | cagtcctacaaggacggcctgtccaagaacgacacccgcctgttcggccagaagatcggctccctgtcccccaacgcgat |
| | cctgaacacgaccgtccccgcccacggcatcgcgttctaccgcctgcgcccctcctccTGAtacaacttattacgtattctga |
| | ccggcgctgatgtggcgcggacgccgtcgtactctttcagactttactcttgaggaattgaacctttctcgcttgctggcatgtaaa |
| | cattggcgcaattaattgtgtgatgaagaaagggtggcacaagatggatcgcgaatgtacgagatcgacaacgatggtgattgtt |
| | atgaggggccaaacctggctcaatcttgtcgcatgtccggcgcaatgtgatccagcggcgtgactctcgcaacctggtagtgtg |
| | tgcgcaccgggtcgctttgattaaaactgatcgcattgccatcccgtcaactcacaagcctactctagctcccattgcgcactcgg |
| | gcgcccggctcgatcaatgttctgagcggagggcgaagcgtcaggaaatcgtctcggcagctggaagcgcatggaatgcgg |
| | agcggagatcgaatcagaggagagggtggctggtagttgcgggatggctggtcgcccgtcgatcctgctgctgctattgt |
| | ctcctcctgcacaagcccacccacgactccgaagaagaagaagaaaacgcgcacacacacaacccaaccggccgaa |
| | tatttgcttccttatcccgggtccaagagagacggcgatgcccccctcaatcagcctcctcctccctgccgctccaatcttc |
| | cctgcttgcatgcgcccgcgagaggctgtctgcgcgccccgtcagtcactccccgtgcagacgcctcgtgctcggtgctc |
| | ctgtatcctttaccgctcctttcattctgcgaggccccctgttgaatgtattcgttgcctgtgtggccaagcgcgctgctggg |
| | cgcgccgccgtcgggcggtgctcggcgactctggcggaagccggttgttct |

The expression construct FATA-2::PmHXT1:ScMEL1:PmPGK1::FATA-2 (pCHK697) was utilized in the transformation of strain CHK72. Transformants of CHK72 were generated via particle bombardment using gold nanoparticles. Upon transformation of pCHK55 into CHK72, positive clones were selected on plates with melibiose as the sole carbon source. Primary transformants were clonally purified and grown under standard lipid production conditions. Lipid samples were prepared from dried biomass from each transformant. Fatty acid profiles were determined using direct transesterification methods. Fatty acid profiles (expressed as Area % of total fatty acid) of 24 positive transgenic lines, compared to the untransformed strain CHK64 and the ricinoleate expressing strain, CHK72, are shown in TABLE 33

P. moriformis ricinoleate producing strain CHK72 and high oleic base strain CHK64 are shown as controls. Strains were grown for 72 hours in 96-well blocks at 200 rpm at 28° C. Biomass was harvested, lyophilized to dryness, and subjected to direct transesterification to generate FAMEs for subsequent quantitation and characterization by GC/FID.

TABLE 33
Screen of primary transformants of CHK72 with pCHK697

| Sample | | | | | | | C18:3 | | | 12-OH- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 166 | 0.05 | 0.35 | 3.35 | 2.73 | 47.88 | 16.43 | 1.00 | 0.38 | 4.01 | 22.72 |
| Sample 167 | 0.04 | 0.32 | 3.24 | 2.79 | 49.32 | 16.16 | 0.94 | 0.45 | 3.83 | 21.97 |
| Sample 168 | 0.04 | 0.41 | 3.98 | 3.09 | 49.77 | 15.43 | 1.03 | 0.48 | 3.09 | 21.97 |
| Sample 169 | 0.04 | 0.46 | 4.12 | 3.02 | 50.34 | 15.50 | 0.97 | 0.43 | 2.97 | 21.39 |
| Sample 170 | 0.04 | 0.26 | 3.06 | 2.70 | 50.84 | 15.81 | 0.82 | 0.36 | 3.81 | 21.35 |
| Sample 171 | 0.03 | 0.33 | 3.36 | 2.88 | 48.46 | 17.27 | 0.99 | 0.40 | 3.92 | 21.30 |
| Sample 172 | 0.04 | 0.35 | 3.50 | 3.03 | 52.65 | 14.01 | 0.92 | 0.47 | 3.20 | 21.09 |
| Sample 173 | 0.04 | 0.30 | 3.11 | 2.77 | 50.83 | 15.88 | 0.89 | 0.41 | 3.91 | 20.80 |
| Sample 174 | 0.04 | 0.30 | 3.19 | 2.76 | 50.99 | 15.97 | 0.88 | 0.38 | 3.85 | 20.77 |
| Sample 175 | 0.04 | 0.31 | 3.20 | 2.77 | 50.86 | 16.15 | 0.91 | 0.38 | 3.94 | 20.58 |
| Sample 176 | 0.03 | 0.37 | 3.70 | 3.00 | 52.89 | 15.09 | 0.94 | 0.45 | 3.35 | 19.42 |
| Sample 177 | 0.03 | 0.29 | 3.15 | 2.73 | 53.03 | 15.43 | 0.86 | 0.36 | 3.89 | 19.39 |
| Sample 178 | 0.03 | 0.65 | 5.76 | 1.98 | 58.52 | 11.58 | 0.48 | 0.20 | 0.78 | 19.23 |
| Sample 179 | 0.04 | 0.33 | 3.38 | 2.98 | 55.25 | 13.87 | 0.83 | 0.43 | 3.27 | 18.81 |
| Sample 180 | 0.04 | 0.33 | 3.62 | 2.42 | 52.97 | 16.42 | 1.15 | 0.44 | 3.44 | 18.34 |
| Sample 181 | 0.03 | 0.32 | 3.36 | 3.01 | 55.05 | 14.55 | 0.90 | 0.47 | 3.46 | 17.86 |
| Sample 182 | 0.03 | 0.30 | 3.01 | 2.64 | 58.62 | 15.23 | 0.92 | 0.40 | 3.22 | 15.01 |
| Sample 183 | 0.02 | 0.24 | 3.94 | 2.60 | 61.30 | 14.03 | 0.45 | 0.32 | 2.38 | 14.13 |
| CHK72 | 0.02 | 0.57 | 5.97 | 2.59 | 65.16 | 10.13 | 0.37 | 0.29 | 0.72 | 13.50 |
| Control 1 | | | | | | | | | | |
| Sample 184 | 0.02 | 0.60 | 6.06 | 2.73 | 64.64 | 10.98 | 0.44 | 0.34 | 0.88 | 12.60 |
| Sample 185 | 0.03 | 0.76 | 6.66 | 2.65 | 64.65 | 10.94 | 0.45 | 0.35 | 0.81 | 11.95 |
| Sample 186 | 0.03 | 0.81 | 9.23 | 2.21 | 63.59 | 10.87 | 0.47 | 0.25 | 0.62 | 11.04 |
| Sample 187 | 0.02 | 0.51 | 5.78 | 2.70 | 67.57 | 10.19 | 0.41 | 0.30 | 0.86 | 10.99 |
| Sample 188 | 0.02 | 0.53 | 5.86 | 2.69 | 67.66 | 10.12 | 0.38 | 0.29 | 0.87 | 10.90 |
| Sample 189 | 0.02 | 0.47 | 5.81 | 2.61 | 70.38 | 9.08 | 0.36 | 0.27 | 0.85 | 9.53 |
| CHK64 | 0.02 | 0.58 | 7.79 | 2.69 | 78.18 | 9.02 | 0.37 | 0.17 | 0.46 | 0.10 |
| Control 3 | | | | | | | | | | |

Transformants of CHK72 were generated via particle bombardment using gold nanoparticles, upon transformation of pCH55 (FATA-1 knockout) or pCHK697 (FATA-2 knockout) into CHK72, positive clones were selected on plates with melibiose as the sole carbon source. Primary transformants were clonally purified and grown under standard lipid production conditions. Lipid samples were prepared from dried biomass from each transformant. Fatty acid profiles were determined using direct transesterification methods. Fatty acid profiles (expressed as Area % of total fatty acid) of some positive transgenic lines, compared to the untransformed strain CHK72 control are shown in TABLE 31 and 33.

P. moriformis strain CHK72 exhibits a fatty acid profile comprising 13.5% ricinoleate. When transformed with a FATA-1 deletion construct (pCHK55), all transformants produced slightly lower ricinoleate compared to the parental strain CHK72 under same conditions, indicating that deletion of FATA-1 negatively impacts ricinoleate production. On the other hand, when CHK72 is transformed with a FATA-2 deletion construct (pCHK697), most of transformants produce significantly higher ricinoleate levels compared to the parental strain CHK72 under same conditions, indicating that deletion of FATA-2 positively impacts ricinoleate production (TABLE 33).

### Example 8: Down-Regulation of P. moriformis Delta-12 Fatty Acid Desaturase FAD2 in Strain CHK72

This experiment determined whether down-regulation of PmFAD2 can positively impact ricinoleate production through selective down-regulation of a single allele and elevation of oleate substrate. Toward this end, a PmFAD2 hairpin (PmFAD2 hp) construct was introduced into the P. moriformis CHK72 background as shown in SEQ ID NO: 24. The expression construct contains 5′ and 3′ homology arms to permit targeted integration of the transforming DNA.

FIG. 6 shows a diagram of ricinoleate TAG formation. Delta-12 fatty acid desaturase (PmFAD2) and hydroxylase (LFAH12) both use oleic acid as a substrate, while FATA-1 and FATA-2 cleave fatty acyl groups form acyl carrier protein (ACP), terminating fatty acid elongation at 18 carbons.

TABLE 34 shows sequences of constructs. SEQ ID NO: 24 shows integrative sequences for the transformation of P. moriformis strain CHK72 with pCHK698. The construct can be written as 5′Thi4::PmHXT1:ScMEL1:PmPGK1:CvNR:CrTUB2:PmFAD2 hp:CvNR::3′Thi4. Bold, lowercase sequences represent genomic DNA from CHK72 that permit targeted integration at the Thi4 locus via homologous recombination. Proceeding in the 5′ to 3′ direction, the P. moriformis hexose transporter 1 (HXT1) promoter driving expression of the S. carlbergensis melibiase (ScMEL1) is indicated by boxed uppercase text. The initiator ATG and terminator TAA for ScMEL1 are indicated by uppercase, bold italics while the coding region is indicated in lowercase italics. P. moriformis phosphoglucokinase (PGK) 3′-UTR is indicated by uppercase underlined text followed by a C. vulgaris nitrate reductase 3′-UTR that is indicated by lowercase, underlined italics.

The Chlamydomonas reinhardtii β-tubulin promoter driving expression of the PmFAD2 hairpin cassette is indicated by boxed uppercase text. The initiator ATG of the PmFAD2 exon 1, followed by the PmFAD2 intron 1 and reverse sequence of the PmFAD2 exon 1 are indicated by lowercase, bold italics. The C. vulgaris nitrate reductase 3′-UTR is indicated by lowercase underlined text followed by the CHK72 Thi4 genomic region indicated by bold, lowercase text.

TABLE 34
Sequences

| SEQ | |
|---|---|
| ID NO | Sequence |
| 24 | ccctcaactgcgacgctgggaaccttctccgggcaggcgatgtgcgtgggtttgcctccttggcacggctctacaccgtc |
| | gagtacgccatgaggcggtgatggctgtgtcggttgccacttcgtccagagacggcaagtcgtccatcctctgcgtgtgt |
| | ggcgcgacgctgcagcagtccctctgcagcagatgagcgtgactttggccatttcacgcactcgagtgtacacaatccat |
| | ttttcttaaagcaaatgactgctgattgaccagatactgtaacgctgatttcgctccagatcgcacagatagcgaccatgt |
| | tgctgcgtctgaaaatctggattccgaattcgaccctggcgctccatccatgcaacagatggcgacacttgttacaattcc |
| | tgtcacccatcggcatggagcaggtccacttagattcccgatcacccacgcacatctcgctaatagtcattcgttcgtgtct |
| | tcgatcaatctcaagtgagtgtgcatggatcttggttgacgatgcggtatgggtttgcgccgctggctgcagggtctgccc |
| | aaggcaagctaacccagctcctctccccgacaatactctcgcaggcaaagccggtcacttgccttccagattgccaata |
| | aactcaattatggcctctgtcatgccatccatgggtctgatgaatggtcacgctcgtgtcctgaccgttccccagcctctgg |
| | 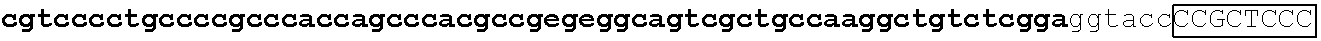 |
| | 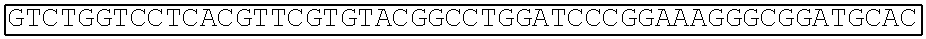 |
| | 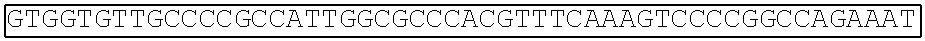 |
| | 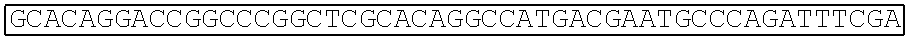 |
| | 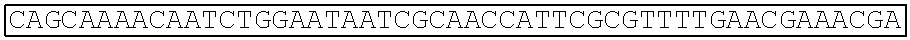 |
| | 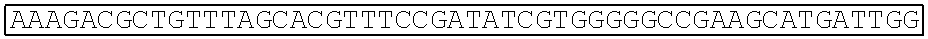 |
| | 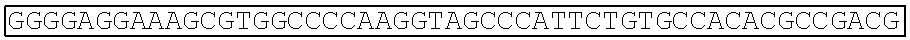 |
| | 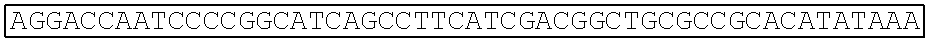 |
| | 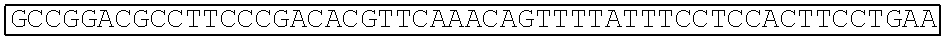 |
| | 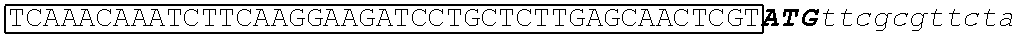 |
| | cttcctgacggcctgcatctccctgaagggcgtgttcggcgtctccccctcctacaacggcctgggcctgacgccccagatg |
| | ggctgggacaactggaacacgttcgcctgcgacgtctccgagcagctgctgctggacacggccgaccgcatgtccgacct |
| | gggcctgaaggacatgggctacaagtacatcatcctggacgactgcaggtcctccggccgcgactccgacggcttcctggt |
| | cgccgacgagcagaagttccccaacggcatgggccacgtcgccgaccacctgcacaacaactcctcctgttcggcatgt |
| | actcctccggggcgagtacacgtgcgccggctaccccggctccctgggccgcgaggaggaggacgccagttcttcgcg |
| | aacaaccgcgtggactacctgaagtacgacaactgctacaacaagggccagttcggcacgcccgagatctcctaccacc |
| | gctacaaggccatgtccgacgccctgaacaagacgggccgccccatcttctactccctgtgcaactggggccaggacctg |
| | accttctactggggctccggcatcgcgaatcctggagcatgtccggcgacgtcacggcggagttcacgcgccccgactcc |
| | cgctgcccctgcgacggcgacgagtacgactgcaagtacgccggcttccactgctccatcatgaacatcctgaacaaggc |
| | cgcccccatgggccagaacgcgggcgtcggcggctggaacgacctggacaacctggaggtcggcgtcggcaacctgac |
| | ggacgacgaggagaaggcgcacttctccatgtgggccatggtgaagtcccccctgatcggcgcgaacgtgaacaac |
| | ctgaaggcctcctcctactccatctactcccaggcgtccgtcatcgccatcaaccaggactccaacggcatccccgccacgc |
| | gcgtctggcgctactacgtgtccgacacggacgagtacggccagggcgagatccagatgtggtccggccccctggacaa |
| | cggcgaccaggtcgtggcgctgctgaacggcggctccgtgtcccgccccatgaacacgaccctggaggagatcttcttcga |
| | ctccaacctgggctccaagaagctgacctccacctgggacatctacgacctgtgggcgaaccgcgtcgacaactccacgg |
| | cgtccgccatcctgggccgcaacaagaccgccaccggcatcctgtacaacgccaccgagcagtcctacaaggacggcct |
| | gtccaagaacgacacccgcctgttcggccagaagatcggctccctgtcccccaacgcgatcctgaacacgaccgtccccg |
| | cccacggcatcgcgttctaccgcctgcgcccctcctccTGATACAACTTATTACGTATTCTGACCGG |
| | CGCTGATGTGGCGCGGACGCCGTCGTACTCTTTCAGACTTTACTCTTGAGGA |
| | ATTGAACCTTTCTCGCTTGCTGGCATGTAAACATTGGCGCAATTAATTGTGTG |
| | ATGAAGAAAGGGTGGCACAAGATGGATCGCGAATGTACGAGATCGACAACG |
| | ATGGTGATTGTTATGAGGGGCCAAACCTGGCTCAATCTTGTCGCATGTCCGG |
| | CGCAATGTGATCCAGCGGCGTGACTCTCGCAACCTGGTAGTGTGTGCGCACC |
| | GGGTCGCTTTGATTAAAACTGATCGCATTGCCATCCCGTCAACTCACAAGCC |
| | TACTCTAGCTCCCATTGCGCACTCGGGCGCCCGGCTCGATCAATGTTCTGAGC |
| | GGAGGGCGAAGCGTCAGGAAATCGTCTCGGCAGCTGGAAGCGCATGGAATG |
| | CGGAGCGGAGATCGAATCAATCGATgcagcagcagctcggatagtatcgacacactctggacgctg |
| | gtcgtgtgatggactgttgccgccacacttgctgccttgacctgtgaatatccctgccgcttttatcaaacagcctcagtgtgttt |
| | gatcttgtgtgtacgcgcttttgcgagttgctagctgcttgtgctatttgcgaataccacccccagcatccccttccctcgtttcata |
| | tcgcttgcatcccaaccgcaacttatctacgctgtcctgctatccctcagcgctgctcctgctcctgctcactgcccctcgcaca |
| | gccttggtttgggctccgcctgtattctcctggtactgcaacctgtaaaccagcactgcaatgctgatgcacgggaagtagtg |
| | 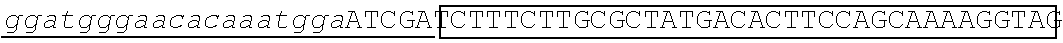 |
| | 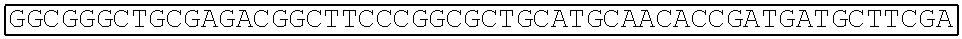 |
| | 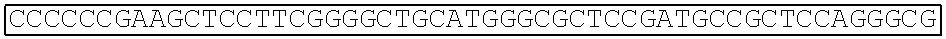 |
| | 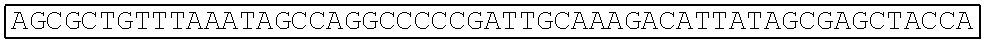 |
| | 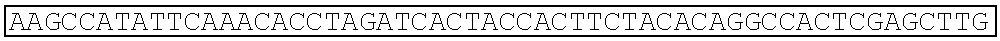 |
| | 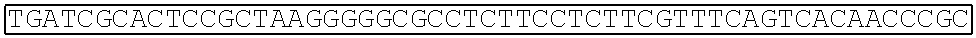 |
| | 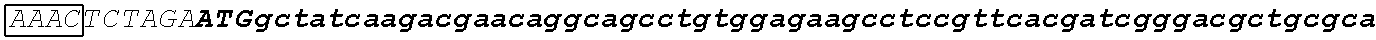 |
| | aggccatccccgcgcactgtttcgagcgctcggcgcttcgtagcagcatgtacctggcctttgacatcgcggtcatgtccctg |
| | ctctacgtcgcgtcgacgtacatcgaccctgcaccggtgcctacgtgggtcaagtacggcatcatgtggccgctctactggtt |
| | cttccaggtgtgtttgagggttttggttgcccgtattgaggtcctggtggcgcgcatggaggagaaggcgcctgtcccgctgac |
| | ccccccggctaccctcccggcaccttccagggcgcgtacgggaagaaccagtagagcggccacatgatgccgtacttgac |
| | ccacgtaggcaccggtgcagggtcgatgtacgtcgacgcgacgtagagcagggacatgaccgcgatgtcaaaggccag |
| | gtacatgctgctacgaagcgccgagcgctcgaaacagtgcgcggggatggccttgcgcagcgtcccgatcgtgaacgga |
| | ggcttctccacaggctgcctgttcgtcttgatagccatcctagggcagcagcagctcggatagtatcgacacactctggacgct |
| | ggtcgtgtgatggactgttgccgccacacttgctgccttgacctgtgaatatccctgccgcttttatcaaacagcctcagtgtgtttg |
| | atcttgtgtgtacgcgcttttgcgagttgctagctgcttgtgctatttgcgaataccacccccagcatccccttccctcgtttcatatcg |
| | cttgcatcccaaccgcaacttatctacgctgtcctgctatccctcagcgctgctcctgctcctgctcactgcccctcgcacagcctt |
| | ggtttgggctccgcctgtattctcctggtactgcaacctgtaaaccagcactgcaatgctgatgcacgggaagtagtgggatggg |
| | aacacaaatggacctgcagggcggccgccagcgccatgccacgccctttgatggcttcaagtacgattacggtgttggat |
| | tgtgtgtttgttgcgtagtgtgcatggtttagaataatacacttgatttcttgctcacggcaatctcggcttgtccgcaggttc |
| | aaccccatttcggagtctcaggtcagccgcgcaatgaccagccgctacttcaaggacttgcacgacaacgccgaggtg |
| | agctatgtttaggacttgattggaaattgtcgtcgacgcatattcgcgctccgcgacagcacccaagcaaaatgtcaagt |
| | gcgttccgatttgcgtccgcaggtcgatgttgtgatcgtcggcgccggatccgccggtctgtcctgcgcttacgagctgac |
| | caagcaccctgacgtccgggtacgcgagctgagattcgattagacataaattgaagattaaacccgtagaaaaatttga |
| | tggtcgcgaaactgtgctcgattgcaagaaattgatcgtcctccactccgcaggtcgccatcatcgagcagggcgttgct |
| | cccggcggcggcgcctggctggggggacagctgttctcggccatgtgtgtacgtagaaggatgaatttcagctggttttc |
| | gttgcacagctgtttgtgcatgatttgtttcagactattgttgaatgtttttagatttcttaggatgcatgatttgtctgcatgc |
| | gact |

The expression construct 5′Thi4::PmHXT1:ScMEL1:PmPGK1:CvNR:CrTUB2:PmFAD2 hp:CvNR::3′Thi4 (PM15) was utilized in the transformation of strain CHK72 via particle bombardment using gold nanoparticles. Upon transformation of pCHK698 into CHK72, positive clones were selected on plates with melibiose as the sole carbon source. Primary transformants were clonally purified and grown under standard lipid production conditions. Lipid samples were prepared from dried biomass from each transformant. Fatty acid profiles were determined using direct transesterification methods. Fatty acid profiles (expressed as Area % of total fatty acid) of 39 positive transgenic lines, compared to the untransformed strain CHK64 and the ricinoleate expressing strain, CHK72, are shown in TABLE 35.

TABLE 35 shows a screen of primary transformants of CHK72 with pCHK698. P. moriformis ricinoleate producing strain CHK72 and high oleic base strain CHK64 are shown as controls. Strains were grown for 72 hours in 96-well blocks at 200 rpm at 28° C. Biomass was harvested, lyophilized to dryness, and subjected to direct transesterification to generate FAMEs for subsequent quantitation and characterization by GC/FID.

TABLE 35
Screen of primary transformants of CHK72 with pCHK698

| Sample | | | | | | | C18:3 | | | 12-OH, 9c-- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 190 | 0.03 | 0.87 | 7.92 | 2.63 | 59.22 | 8.82 | 0.44 | 0.31 | 0.64 | 18.29 |
| Sample 191 | 0.02 | 0.41 | 4.70 | 3.36 | 61.18 | 10.45 | 0.48 | 0.41 | 1.16 | 17.21 |
| Sample 192 | 0.03 | 0.77 | 6.25 | 2.64 | 60.33 | 10.91 | 0.52 | 0.34 | 0.81 | 16.60 |
| Sample 193 | 0.03 | 0.83 | 6.95 | 2.84 | 60.42 | 10.24 | 0.52 | 0.38 | 0.74 | 16.21 |
| Sample 194 | 0.03 | 0.78 | 6.83 | 2.79 | 60.91 | 10.21 | 0.51 | 0.36 | 0.67 | 16.13 |
| Sample 195 | 0.02 | 1.12 | 6.54 | 3.14 | 58.68 | 11.94 | 0.71 | 0.40 | 0.72 | 15.90 |
| Sample 196 | 0.03 | 0.85 | 6.85 | 2.59 | 61.20 | 10.58 | 0.48 | 0.33 | 0.67 | 15.69 |
| Sample 197 | 0.03 | 0.63 | 6.01 | 2.64 | 63.32 | 10.12 | 0.41 | 0.32 | 0.74 | 15.06 |
| Sample 198 | 0.03 | 0.70 | 7.56 | 2.41 | 63.04 | 9.57 | 0.40 | 0.27 | 0.61 | 14.60 |
| Sample 199 | 0.03 | 0.80 | 6.8 | 2.73 | 61.95 | 10.86 | 0.47 | 0.36 | 0.69 | 14.53 |
| Sample 200 | 0.03 | 0.58 | 5.87 | 2.66 | 64.69 | 9.81 | 0.40 | 0.31 | 0.76 | 14.24 |
| Sample 201 | 0.02 | 0.59 | 5.96 | 2.67 | 64.33 | 10.07 | 0.41 | 0.32 | 0.77 | 14.23 |
| Sample 202 | 0.02 | 0.54 | 5.83 | 2.64 | 65.21 | 9.49 | 0.38 | 0.30 | 0.76 | 14.19 |
| Sample 203 | 0.02 | 0.54 | 5.79 | 2.68 | 65.67 | 9.25 | 0.40 | 0.30 | 0.75 | 13.95 |
| Sample 204 | 0.02 | 0.69 | 6.73 | 2.66 | 62.91 | 10.88 | 0.48 | 0.33 | 0.70 | 13.93 |
| Sample 205 | 0.03 | 0.64 | 6.00 | 2.53 | 63.82 | 10.74 | 0.43 | 0.32 | 0.87 | 13.90 |
| Sample 206 | 0.02 | 0.53 | 5.75 | 2.62 | 65.60 | 9.49 | 0.38 | 0.30 | 0.77 | 13.88 |
| Sample 207 | 0.02 | 0.57 | 6.04 | 2.69 | 64.52 | 10.23 | 0.42 | 0.32 | 0.74 | 13.78 |
| Sample 208 | 0.02 | 0.53 | 5.8 | 2.64 | 66.09 | 9.21 | 0.38 | 0.30 | 0.76 | 13.68 |
| Sample 209 | 0.03 | 0.65 | 6.21 | 2.73 | 63.37 | 11.00 | 0.45 | 0.33 | 0.85 | 13.62 |
| CHK72 | 0.02 | 0.57 | 5.97 | 2.59 | 65.16 | 10.13 | 0.37 | 0.29 | 0.72 | 13.50 |
| Control 1 | | | | | | | | | | |
| Sample 210 | 0.02 | 0.53 | 5.81 | 2.65 | 66.20 | 9.29 | 0.39 | 0.30 | 0.76 | 13.44 |
| Sample 211 | 0.03 | 0.63 | 6.14 | 2.71 | 63.60 | 11.30 | 0.43 | 0.33 | 0.83 | 13.32 |
| Sample 212 | 0.02 | 0.56 | 6.94 | 2.43 | 64.23 | 10.52 | 0.31 | 0.26 | 0.68 | 13.32 |
| Sample 213 | 0.02 | 0.55 | 5.94 | 2.70 | 65.57 | 9.85 | 0.40 | 0.31 | 0.74 | 13.32 |
| Sample 214 | 0.02 | 0.54 | 5.88 | 2.63 | 66.11 | 9.48 | 0.39 | 0.30 | 0.75 | 13.27 |
| Sample 215 | 0.02 | 0.54 | 6.17 | 2.76 | 65.80 | 9.52 | 0.41 | 0.31 | 0.73 | 13.10 |
| Sample 216 | 0.02 | 0.57 | 6.02 | 2.79 | 65.99 | 9.50 | 0.40 | 0.31 | 0.75 | 13.00 |
| Sample 217 | 0.02 | 0.49 | 5.85 | 2.85 | 70.23 | 5.96 | 0.30 | 0.28 | 0.76 | 12.63 |
| Sample 218 | 0.02 | 0.51 | 5.77 | 2.69 | 66.95 | 9.58 | 0.36 | 0.30 | 0.76 | 12.46 |
| Sample 219 | 0.02 | 0.54 | 6.21 | 2.85 | 67.31 | 8.71 | 0.34 | 0.30 | 0.72 | 12.41 |
| Sample 220 | 0.03 | 0.56 | 5.89 | 2.55 | 68.07 | 8.31 | 0.37 | 0.29 | 0.68 | 12.37 |
| Sample 221 | 0.02 | 0.54 | 5.87 | 2.71 | 67.32 | 9.15 | 0.38 | 0.31 | 0.84 | 12.15 |
| Sample 222 | 0.02 | 0.51 | 5.86 | 2.65 | 67.84 | 9.00 | 0.37 | 0.29 | 0.76 | 12.11 |
| Sample 223 | 0.02 | 0.47 | 5.72 | 2.62 | 69.12 | 8.23 | 0.35 | 0.26 | 0.75 | 11.90 |
| Sample 224 | 0.02 | 0.49 | 5.83 | 2.65 | 68.51 | 8.86 | 0.36 | 0.28 | 0.74 | 11.68 |
| Sample 225 | 0.02 | 0.50 | 5.54 | 2.90 | 68.81 | 8.51 | 0.43 | 0.33 | 0.87 | 11.53 |
| Sample 226 | 0.02 | 0.49 | 6.57 | 2.80 | 68.05 | 8.65 | 0.39 | 0.29 | 0.68 | 11.43 |
| Sample 227 | 0.02 | 0.55 | 5.97 | 2.46 | 69.27 | 8.45 | 0.34 | 0.25 | 0.75 | 11.31 |
| Sample 228 | 0.03 | 0.55 | 6.09 | 1.85 | 64.57 | 14.66 | 0.29 | 0.26 | 0.79 | 9.84 |
| CHK64 | 0.02 | 0.58 | 7.79 | 2.69 | 78.18 | 9.02 | 0.37 | 0.17 | 0.46 | 0.10 |
| Control 3 | | | | | | | | | | |

Transformants of CHK72 were generated via particle bombardment using gold nanoparticles utilizing a transforming DNA, pCHK698, designed to down regulate endogenous FAD2 activity. Positive clones were selected on plates with melibiose as the sole carbon source. Primary transformants were clonally purified and grown under standard lipid production conditions. Lipid samples were prepared from dried biomass from each transformant. Fatty acid profiles were determined using direct transesterification methods. Fatty acid profiles (expressed as Area % of total fatty acid) of some positive transgenic lines, compared to the untransformed strain CHK72 control.

The P. moriformis strain CHK72 exhibits a fatty acid profile comprising 13.5% ricinoleate. When transformed with a PmFAD2 hairpin construct, pCHK698, a significant number of transformants produce elevated ricinoleate levels compared to the parental strain CHK72 under same conditions, indicating that down-regulation of PmFAD2 by RNAi positively impacts ricinoleate accumulation.

### Example 9: Simultaneous Down-Regulation of Both P. moriformis FATA-2 and FAD2 in Strains CHK72 and CHK84

To determine if simultaneously down-regulating FATA-2 and delta-12 fatty acid desaturase (PmFAD2) activity would significantly increase ricinoleate production, a PmFAD2 hairpin construct, pCHK636, was introduced into the PmFATA-2 locus of the P. moriformis ricinoleate producing strains CHK72 or CHK84. The expression construct contains 5′ and 3′ homology arms to permit targeted integration of the transforming DNA (SEQ ID NO: 25).

TABLE 36 shows sequences of constructs. SEQ ID NO: 25 shows integrative sequences for the transformation of P. moriformis strains CHK72 or CHK84 with pCHK636. The construct can be written as FATA-2::PmHXT1:ScMEL1:PmPGK1:CvNR:CrTUB2:PmFAD2 hp:CvNR::FATA-2. Bold, lowercase sequences represent genomic DNA from strains CHK72 or CHK84 that permit targeted integration at the FATA-2 locus via homologous recombination. Proceeding in the 5′ to 3′ direction, the P. moriformis hexose transporter 1 (HXT1 v2) promoter driving expression of the S. carlbergensis melibiase (ScMEL1) is indicated by boxed uppercase text. The initiator ATG and terminator TAA for ScMEL1 are indicated by uppercase, bold italics while the coding region is indicated in lowercase italics. P. moriformis phosphoglucokinase (PGK) 3′-UTR is indicated by uppercase underlined text followed by a C. vulgaris nitrate reductase 3′-UTR that is indicated by lowercase, underlined italics. The C. reinhardtii β-tubulin promoter drives expression of the PmFAD2 hairpin cassette and is indicated by boxed uppercase text. The initiator ATG of the PmFAD2 exon 1, followed by the PmFAD2 intron 1 and reverse sequence of the PmFAD2 exon 1 are indicated by lowercase, bold italics. The C. vulgaris nitrate reductase 3′-UTR is indicated by lowercase underlined text followed by the CHK72 or CHK84 FATA-2 3′-flanking genomic region indicated by bold, lowercase text.

TABLE 36

| SEQ | |
|---|---|
| ID NO | Sequence |
| 25 | cttaatggagtcgctgctccactaatcgaattgtcagcaccgccagccggccgaggacccgagtcatagcgagggtagt |
| | agcgcgccatggcaccgaccagcctgcttgcccgtactggcgtctcttccgcttctctgtgctcctctacgcgctccggcg |
| | cgtgcgcttttccggtggatcatgcggtccgtggcgcaccgcagcggccgctgcccatgcagcgccgctgcttccgaaca |
| | gtggctgtcagggccgcacccgcagtagccgtccgtccggaacccgcccaagagttttgggagcagcttgagccctgca |
| | agatggcggaggacaagcgcatcttcctggaggagcaccggtgcgcggaggtccggggctgaccggccgtcgcattc |
| | aacgtaatcaatcgcatgatgatcacaggacgcgacgtcttggtggcggtggccagggacactgcccattgcacaggc |
| | ataggaatgcgttccttctcatttctcagttttctgagcccctccctcttcactctttctcctcctcctcccctctcacgcagca |
| | 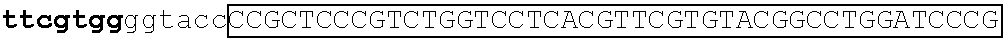 |
| | 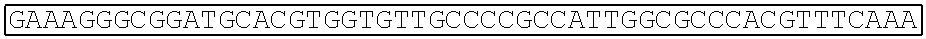 |
| | 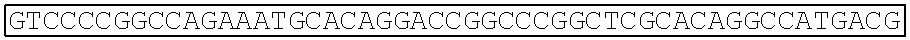 |
| | 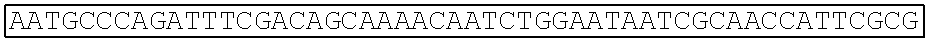 |
| | 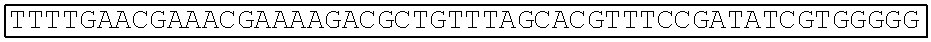 |
| | 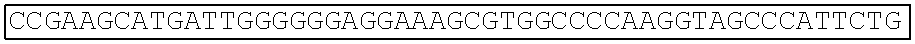 |
| | 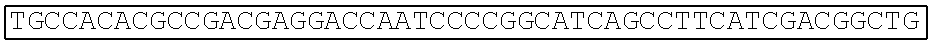 |
| | 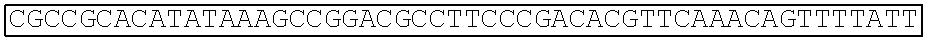 |
| | 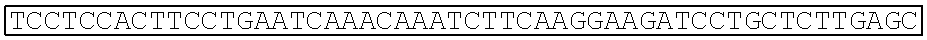 |
| | 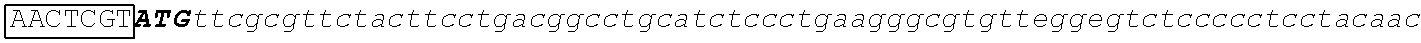 |
| | ggcctgggcctgacgccccagatgggctgggacaactggaacacgttcgcctgcgacgtctccgagcagctgctgctgga |
| | cacggccgaccgcatctccgacctgggcctgaaggacatgggctacaagtacatcatcctggacgactgctggtcctccgg |
| | ccgcgactccgacggcttcctggtcgccgacgagcagaagttccccaacggcatgggccacgtcgccgaccacctgcac |
| | aacaactccttcctgttcggcatgtactcctccgcgggcgagtacacgtgcgccggctaccccggctccctgggccgcgag |
| | gaggaggacgcccagttcttcgcgaacaaccgcgtggactacctgaagtacgacaactgctacaacaagggccagttcg |
| | gcacgcccgagatctcctaccaccgctacaaggccatgtccgacgccctgaacaagacgggccgccccatcttctactccc |
| | tgtgcaactggggccaggacctgaccttctactggggctccggcatcgcgaactcctggcgcatgtccggcgacgtcacgg |
| | cggagttcacgcgccccgactcccgctgcccctgcgacggcgacgagtacgactgcaagtacgccggcttccactgctcc |
| | atcatgaacatcctgaacaaggccgcccccatgggccagaacgcgggcgtcggcggctggaacgacctggacaacctg |
| | gaggtcggcgtcggcaacctgacggacgacgaggagaaggcgcacttctccatgtgggccatggtgaagtcccccctgat |
| | catcggcgcgaacgtgaacaacctgaaggcctcctcctactccatctactcccaggcgtccgtcatcgccatcaaccagga |
| | ctccaacggcatccccgccacgcgcgtctggcgctactacgtgtccgacacggacgagtacggccagggcgagatccag |
| | atgtggtccggccccctggacaacggcgaccaggtcgtggcgctgctgaacggcggctccgtgtcccgccccatgaacac |
| | gaccctggaggagatcttcttcgactccaacctgggctccaagaagctgacctccacctgggacatctacgacctgtgggc |
| | gaaccgcgtcgacaactccacggcgtccgccatcctgggccgcaacaagaccgccaccggcatcctgtacaacgccacc |
| | gagcagtcctacaaggacggcctgtccaagaacgacacccgcctgttcggccagaagatcggctccctgtcccccaacgc |
| | gatcctgaacacgaccgtccccgcccacggcatcgcgttctaccgcctgcgcccctcctccTGATACAACTTATT |
| | ACGTATTCTGACCGGCGCTGATGTGGCGCGGACGCCGTCGTACTCTTTCAGA |
| | CTTTACTCTTGAGGAATTGAACCTTTCTCGCTTGCTGGCATGTAAACATTGGC |
| | GCAATTAATTGTGTGATGAAGAAAGGGTGGCACAAGATGGATCGCGAATGT |
| | ACGAGATCGACAACGATGGTGATTGTTATGAGGGGCCAAACCTGGCTCAATC |
| | TTGTCGCATGTCCGGCGCAATGTGATCCAGCGGCGTGACTCTCGCAACCTGG |
| | TAGTGTGTGCGCACCGGGTCGCTTTGATTAAAACTGATCGCATTGCCATCCC |
| | GTCAACTCACAAGCCTACTCTAGCTCCCATTGCGCACTCGGGCGCCCGGCTC |
| | GATCAATGTTCTGAGCGGAGGGCGAAGCGTCAGGAAATCGTCTCGGCAGCT |
| | GGAAGCGCATGGAATGCGGAGCGGAGATCGAATCAATCGATgcagcagcagctcgg |
| | atagtatcgacacactctggacgctggtcgatgactgttgccgccacacttgctgccttgacctgtgaatatccctgcc |
| | gcttttatcaaacagcctcagtgtgtttgatcttgtgtgtacgcgcttttgcgagttgctagctgcttgtgctatttgcgaataccac |
| | ccccagcatccccttccctcgtttcatatcgcttgcatcccaaccgcaacttatctacgctgtcctgctatccctcagcgctgctc |
| | ctgctcctgctcactgcccctcgcacagccttggtttgggctccgcctgtattctcctggtactgcaacctgtaaaccagcactg |
| | 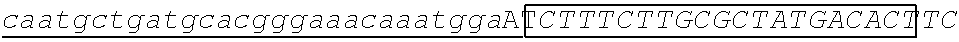 |
| | 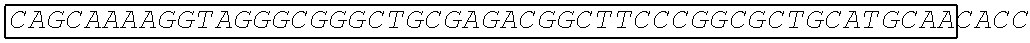 |
| | 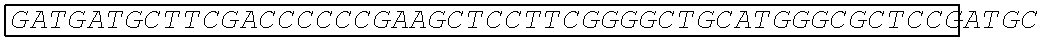 |
| | 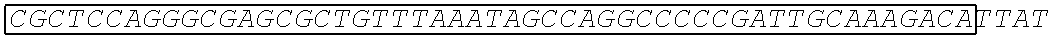 |
| | 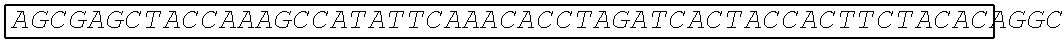 |
| | 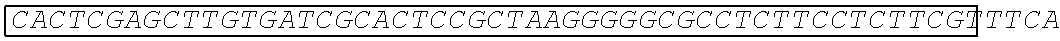 |
| | 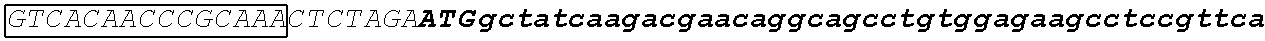 |
| | cgatcgggacgctgcgcaaggccatccccgcgcactgtttcgagcgctcggcgcttcgtagcagcatgtacctggcctttga |
| | catcgcggtcatgtccctgctctacgtcgcgtcgacgtacatcgaccctgcaccggtgcctacgtgggtcaagtacggcatc |
| | atgtggccgctctactggttcttccaggtgtgtttgagggttttggttgcccgtattgaggtcctggtggcgcgcatggaggaga |
| | aggcgcctgtcccgctgacccccccggctaccctcccggcaccttccagggcgcgtacgggaagaaccagtagagcggc |
| | cacatgatgccgtacttgacccacgtaggcaccggtgcagggtcgatgtacgtcgacgcgacgtagagcagggacatga |
| | ccgcgatgtcaaaggccaggtacatgctgctacgaagcgccgagcgctcgaaacagtgcgcggggatggccttgcgcag |
| | cgtcccgatcgtgaacggaggcttctccacaggctgcctgttcgtcttgatagccatcctagggcagcagcagctcggatagt |
| | atcgacacactctggacgctggtcgtgtgatggactgttgccgccacacttgctgccttgacctgtgaatatccctgccgcttttat |
| | caaacagcctcagtgtgtttgatcttgtgtgtacgcgcttttgcgagttgctagctgcttgtgctatttgcgaataccacccccagca |
| | tccccttccctcgtttcatatcgcttgcatcccaaccgcaacttatctacgctgtcctgctatccctcagcgctgctcctgctcctgct |
| | cactgcccctcgcacagccttggtttgggctccgcctgtattctcctggtactgcaacctgtaaaccagcactgcaatgctgatgc |
| | acgggaagtagtgggatgggaacacaaatggacctgcagggcggccgcagaggagagggtggctggtagttgcgggat |
| | ggctggtcgcccgtcgatcctgctgctgctattgtctcctcctgcacaagcccacccacgactccgaagaagaagaaga |
| | aaacgcgcacacacacaacccaaccggccgaatatttgcttccttatcccgggtccaagagagacggcgatgcccccct |
| | caatcagcctcctcctccctgccgctccaatcttccctgcttgcatgcgcccgcgagaggctgtctgcgcgccccgtcagt |
| | cactccccgtgcagacgcctcgtgctcggtgctcctgtatcctttaccgctcctttcattctgcgaggccccctgttgaatgt |
| | attcgttgcctgtgtggccaagcgcgctgctgggcgcgccgccgtcgggcggtgctcggcgactctggcggaagccggt |
| | tgttct |

The expression construct pCHK636 targeting the FATA-2 locus with a cassette designed to simultaneously down-regulate the endogenous FAD2 activity, was utilized in the transformation of strains CHK72 or CHK84 via particle bombardment using gold nanoparticles. Positive clones were selected on plates with melibiose as the sole carbon source. Primary transformants were clonally purified and grown under standard lipid production conditions. Lipid samples were prepared from dried biomass from each transformant. Fatty acid profiles were determined using direct transesterification methods as shown in TABLE 37. Fatty acid profiles (expressed as Area % of total fatty acid) of 36 and 30 positive transgenic lines were isolated in the CHK72 and CHK84 backgrounds. In all cases, ricinoleate levels were compared to the untransformed strain CHK64 and the ricinoleate expressing strains CHK72 or CHK84.

P. moriformis ricinoleate producing strain CHK72 and high oleic base strain CHK64 are shown as controls. Strains were grown for 72 hours in 96-well blocks at 200 rpm at 28° C. Biomass was harvested, lyophilized to dryness, and subjected to direct transesterification to generate FAMEs for subsequent quantitation and characterization by GC/FID.

TABLE 37
Screen of primary transformants of CHK72 with pCHK636

| Sample | | | | | | | C18:3 | | | 12-OH- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 229 | 0.05 | 0.34 | 2.79 | 2.29 | 52.81 | 11.89 | 0.99 | 0.28 | 3.53 | 23.87 |
| Sample 230 | 0.07 | 0.33 | 2.72 | 2.27 | 53.32 | 11.48 | 0.97 | 0.27 | 3.58 | 23.80 |
| Sample 231 | 0.06 | 0.37 | 2.84 | 1.99 | 51.28 | 14.09 | 0.83 | 0.24 | 3.68 | 23.34 |
| Sample 232 | 0.06 | 0.33 | 2.73 | 2.25 | 53.49 | 12.55 | 1.00 | 0.26 | 3.42 | 22.88 |
| Sample 233 | 0.07 | 0.31 | 2.67 | 2.23 | 54.52 | 11.98 | 0.93 | 0.26 | 3.52 | 22.46 |
| Sample 234 | 0.08 | 0.30 | 2.67 | 2.23 | 53.96 | 12.59 | 0.97 | 0.26 | 3.49 | 22.27 |
| Sample 235 | 0.06 | 0.30 | 2.71 | 2.23 | 54.56 | 12.04 | 0.97 | 0.27 | 3.53 | 22.20 |
| Sample 236 | 0.06 | 0.29 | 2.73 | 2.25 | 54.87 | 12.53 | 0.96 | 0.27 | 3.45 | 21.40 |
| Sample 237 | 0.06 | 0.40 | 3.95 | 2.39 | 55.37 | 11.95 | 0.99 | 0.30 | 2.45 | 21.12 |
| Sample 238 | 0.06 | 0.33 | 3.32 | 2.29 | 56.80 | 11.18 | 0.82 | 0.26 | 2.85 | 21.04 |
| Sample 239 | 0.06 | 0.34 | 2.92 | 2.44 | 57.51 | 10.92 | 0.94 | 0.30 | 2.91 | 20.57 |
| Sample 240 | 0.06 | 0.30 | 2.79 | 2.28 | 56.10 | 12.68 | 1.24 | 0.31 | 3.28 | 19.88 |
| Sample 241 | 0.05 | 0.39 | 3.7 | 2.57 | 56.49 | 11.89 | 0.91 | 0.32 | 2.79 | 19.85 |
| Sample 242 | 0.07 | 0.41 | 4.52 | 1.92 | 56.53 | 13.09 | 0.95 | 0.23 | 2.11 | 19.06 |
| Sample 243 | 0.08 | 1.48 | 6.15 | 2.49 | 52.64 | 17.29 | 0.96 | 0.40 | 0.86 | 16.38 |
| Sample 244 | 0.05 | 0.45 | 4.66 | 2.35 | 61.78 | 11.63 | 0.85 | 0.37 | 1.66 | 15.29 |
| Sample 245 | 0.03 | 0.48 | 5.38 | 2.47 | 66.34 | 9.47 | 0.46 | 0.31 | 0.72 | 13.53 |
| Sample 246 | 0.02 | 0.43 | 5.32 | 2.69 | 69.90 | 6.50 | 0.33 | 0.28 | 0.63 | 13.20 |
| Sample 247 | 0.03 | 0.35 | 4.93 | 2.06 | 67.54 | 9.84 | 0.26 | 0.27 | 0.80 | 13.09 |
| Sample 248 | 0.03 | 0.60 | 5.98 | 2.1 | 63.34 | 12.84 | 0.52 | 0.34 | 0.70 | 12.55 |
| Sample 249 | 0.03 | 0.57 | 6.99 | 2.7 | 65.63 | 9.33 | 0.52 | 0.31 | 0.54 | 12.53 |
| Sample 250 | 0.02 | 0.44 | 5.47 | 2.6 | 70.28 | 7.42 | 0.39 | 0.26 | 0.61 | 11.83 |
| Sample 251 | 0.03 | 0.50 | 6.33 | 2.38 | 68.08 | 8.80 | 0.43 | 0.27 | 0.56 | 11.80 |
| Sample 252 | 0.02 | 0.44 | 5.56 | 2.65 | 69.29 | 8.20 | 0.40 | 0.29 | 0.64 | 11.79 |
| Sample 253 | 0.02 | 0.44 | 5.46 | 2.62 | 70.01 | 7.76 | 0.40 | 0.26 | 0.61 | 11.66 |
| Sample 254 | 0.03 | 0.52 | 6.41 | 2.92 | 69.24 | 7.17 | 0.43 | 0.32 | 0.59 | 11.61 |
| Sample 255 | 0.02 | 0.56 | 5.86 | 2.32 | 67.55 | 9.73 | 0.48 | 0.32 | 0.62 | 11.58 |
| Sample 256 | 0.02 | 0.43 | 5.42 | 2.73 | 70.31 | 7.68 | 0.39 | 0.27 | 0.63 | 11.45 |
| Sample 257 | 0.02 | 0.44 | 5.7 | 2.81 | 69.48 | 8.11 | 0.50 | 0.33 | 0.59 | 11.38 |
| Sample 258 | 0.03 | 0.43 | 5.56 | 2.64 | 70.59 | 7.50 | 0.39 | 0.27 | 0.62 | 11.20 |
| Sample 259 | 0.03 | 0.40 | 5.3 | 2.41 | 70.71 | 8.07 | 0.32 | 0.24 | 0.65 | 11.19 |
| Sample 260 | 0.03 | 0.42 | 5.43 | 2.67 | 70.86 | 7.48 | 0.37 | 0.27 | 0.62 | 11.12 |
| CHK72 | 0.03 | 0.42 | 5.54 | 2.47 | 71.69 | 7.56 | 0.37 | 0.07 | 0.00 | 10.71 |
| Control 2 | | | | | | | | | | |
| Sample 261 | 0.02 | 0.41 | 5.17 | 2.47 | 71.82 | 7.48 | 0.36 | 0.27 | 0.67 | 10.61 |
| Sample 262 | 0.02 | 0.41 | 6.09 | 2.79 | 71.11 | 7.20 | 0.36 | 0.28 | 0.58 | 10.41 |
| Sample 263 | 0.02 | 0.47 | 6.7 | 2.38 | 70.97 | 7.70 | 0.39 | 0.25 | 0.45 | 9.85 |
| Sample 264 | 0.02 | 0.26 | 5.15 | 2.33 | 73.91 | 7.83 | 0.25 | 0.22 | 0.70 | 8.67 |
| CHK64 | 0.03 | 0.50 | 8.05 | 2.46 | 78.66 | 8.65 | 0.49 | 0.49 | 0.00 | 0.11 |
| Control 4 | | | | | | | | | | |

P. moriformis ricinoleate producing strain CHK84 and high oleic base strain CHK64 are shown as controls. Strains were grown for 72 hours in 96-well blocks at 200 rpm at 28° C. Biomass was harvested, lyophilized to dryness, and subjected to direct transesterification to generate FAMEs for subsequent quantitation and characterization by GC/FID. The fatty acid profiles are shown in TABLE 38.

TABLE 38
Screen of primary transformants of CHK84 with pCHK636

| Sample | | | | | | | C18:3 | | | 12-OH- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 266 | 0.06 | 0.42 | 3.44 | 2.96 | 45.03 | 14.74 | 1.01 | 0.51 | 0.00 | 27.19 |
| Sample 267 | 0.06 | 0.43 | 3.45 | 3.03 | 45.44 | 14.50 | 1.01 | 0.51 | 0.00 | 27.08 |
| Sample 268 | 0.06 | 0.41 | 3.43 | 2.98 | 46.06 | 14.24 | 1.00 | 0.52 | 3.61 | 26.62 |
| Sample 269 | 0.06 | 0.41 | 3.33 | 2.91 | 46.61 | 14.13 | 0.94 | 0.51 | 3.49 | 26.51 |
| Sample 270 | 0.06 | 0.42 | 3.49 | 2.95 | 46.16 | 14.50 | 1.03 | 0.50 | 0.00 | 26.43 |
| Sample 271 | 0.06 | 0.42 | 3.57 | 2.99 | 46.03 | 14.90 | 1.11 | 0.51 | 0.00 | 25.93 |
| Sample 272 | 0.06 | 0.41 | 3.4 | 3.01 | 46.93 | 14.61 | 0.94 | 0.51 | 0.00 | 25.60 |
| Sample 276 | 0.06 | 0.42 | 3.46 | 3.01 | 48.06 | 14.90 | 1.01 | 0.52 | 3.56 | 23.95 |
| Sample 281 | 0.05 | 0.62 | 3.9 | 3.53 | 56.69 | 12.75 | 0.78 | 0.56 | 0.00 | 17.65 |
| Sample 283 | 0.04 | 0.58 | 5.33 | 3.08 | 60.93 | 11.68 | 0.64 | 0.41 | 0.00 | 15.06 |
| Sample 284 | 0.03 | 0.66 | 5.79 | 3.04 | 63.56 | 9.80 | 0.44 | 0.38 | 0.69 | 14.83 |
| Sample 285 | 0.03 | 0.64 | 5.72 | 2.9 | 64.32 | 9.59 | 0.42 | 0.37 | 0.00 | 14.55 |
| CHK84 | 0.03 | 0.67 | 6.1 | 2.87 | 64.37 | 9.50 | 0.45 | 0.38 | 0.62 | 14.28 |
| Control 1 | | | | | | | | | | |
| Sample 286 | 0.03 | 0.69 | 6.43 | 2.7 | 64.68 | 9.56 | 0.43 | 0.34 | 0.00 | 13.83 |
| Sample 287 | 0.04 | 0.80 | 4.54 | 3.79 | 62.91 | 11.18 | 0.63 | 0.48 | 0.00 | 13.82 |
| Sample 288 | 0.03 | 0.64 | 6.08 | 2.91 | 66.64 | 8.34 | 0.46 | 0.40 | 0.66 | 13.09 |
| Sample 289 | 0.04 | 0.76 | 4.22 | 3.81 | 64.83 | 10.54 | 0.61 | 0.47 | 1.02 | 12.94 |
| Sample 290 | 0.03 | 0.58 | 6.06 | 3.35 | 66.25 | 8.64 | 0.42 | 0.40 | 0.00 | 12.90 |
| Sample 291 | 0.03 | 0.56 | 5.79 | 3.03 | 68.25 | 7.29 | 0.40 | 0.36 | 0.66 | 12.87 |
| Sample 292 | 0.03 | 0.60 | 6.59 | 2.5 | 66.72 | 9.43 | 0.50 | 0.35 | 0.00 | 11.88 |
| Sample 293 | 0.03 | 0.57 | 6.03 | 2.94 | 69.96 | 7.23 | 0.41 | 0.33 | 0.60 | 11.16 |
| Sample 294 | 0.02 | 0.57 | 6.48 | 2.69 | 74.26 | 7.24 | 0.49 | 0.32 | 0.00 | 6.71 |
| CHK64 | 0.02 | 0.55 | 7.66 | 2.71 | 78.51 | 8.72 | 0.41 | 0.24 | 0.00 | 0.09 |
| Control 5 | | | | | | | | | | |

Nine transgenic lines resulting from CHK72's transformation with pCHK636 were further evaluated in a 50-mL conical tube experiment. The results are shown in TABLE 39. One of the transformants, Sample 295, produced over 30% ricinoleate.

P. moriformis high oleic base strain CHK64 and parental strain CHK72 are shown as controls. Strains were grown for 96 hours in 50-mL bioreactor tube at 200 rpm at 28° C. Biomass was harvested, lyophilized to dryness, and subjected to direct transesterification to generate FAMEs for subsequent quantitation and characterization by GC/FID.

TABLE 39
Evaluation of top transgenic lines of CHK72 with pCHK636 in a shake tube experiment

| Sample | | | | | | | C18:3 | | | 12-OH- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 295 | 0.08 | 0.35 | 2.35 | 2.23 | 44.11 | 13.94 | 1.11 | 0.26 | 4.60 | 30.07 |
| Sample 296 | 0.08 | 0.34 | 2.5 | 2.51 | 44.30 | 14.16 | 1.04 | 0.35 | 4.33 | 29.49 |
| Sample 297 | 0.08 | 0.33 | 2.47 | 2.46 | 45.72 | 13.21 | 0.89 | 0.35 | 4.55 | 29.05 |
| Sample 298 | 0.08 | 0.33 | 2.51 | 2.39 | 46.63 | 13.32 | 0.93 | 0.33 | 4.65 | 27.84 |
| Sample 299 | 0.07 | 0.41 | 3.08 | 3.23 | 44.46 | 15.65 | 0.94 | 0.51 | 3.54 | 27.08 |
| Sample 300 | 0.08 | 0.38 | 2.63 | 2.33 | 45.80 | 15.75 | 1.03 | 0.29 | 4.28 | 26.45 |
| Sample 301 | 0.08 | 0.31 | 2.43 | 2.4 | 47.55 | 14.10 | 0.91 | 0.32 | 4.59 | 26.21 |
| Sample 302 | 0.08 | 0.31 | 2.44 | 2.49 | 47.22 | 14.82 | 0.98 | 0.33 | 4.47 | 25.92 |
| Sample 303 | 0.08 | 0.31 | 2.47 | 2.46 | 48.23 | 14.51 | 0.92 | 0.32 | 4.34 | 25.57 |
| CHK72 | 0.02 | 0.31 | 4.43 | 2.74 | 71.90 | 7.07 | 0.34 | 0.25 | 0.89 | 11.53 |
| Control 3 | | | | | | | | | | |
| CHK64 | 0.02 | 0.36 | 7.11 | 2.63 | 81.31 | 7.06 | 0.36 | 0.11 | 0.00 | 0.10 |
| Control 6 | | | | | | | | | | |

Ten transgenic lines resulting from CHK84's transformation with pCHK636 were further evaluated in a 50-ml conical tube experiment. The results are shown in TABLE 40. One of the transformants, Sample 304, produced over 34% ricinoleate.

P. moriformis high oleic base strain CHK64 and parental strain CHK84 are shown as controls. Strains were grown for 120 hours in 50-mL bioreactor tube at 200 rpm at 28° C. Biomass was harvested, lyophilized to dryness, and subjected to direct transesterification to generate FAMEs for subsequent quantitation and characterization by GC/FID.

TABLE 40
Evaluation of top transgenic lines of CHK84 with pCHK636 in a shake tube experiment

| Sample | | | | | | | C18:3 | | | 12-OH- |
|---|---|---|---|---|---|---|---|---|---|---|
| Name | C12:0 | C14:0 | C16:0 | C18:0 | C18:1 | C18:2 | alpha | C20:0 | C20:1 | C18:1 |
| Sample 304 | 0.11 | 0.44 | 2.81 | 2.77 | 39.52 | 13.05 | 0.98 | 0.38 | 4.73 | 34.04 |
| Sample 305 | 0.10 | 0.49 | 2.85 | 2.86 | 37.42 | 15.47 | 1.19 | 0.38 | 4.51 | 33.49 |
| Sample 306 | 0.10 | 0.43 | 2.82 | 2.8 | 40.62 | 12.97 | 0.96 | 0.39 | 4.70 | 33.07 |
| Sample 307 | 0.10 | 0.47 | 2.89 | 2.81 | 37.79 | 15.83 | 1.13 | 0.37 | 4.57 | 32.80 |
| Sample 308 | 0.11 | 0.47 | 2.89 | 2.71 | 38.59 | 15.45 | 1.12 | 0.33 | 4.84 | 32.19 |
| Sample 309 | 0.13 | 0.48 | 2.91 | 2.73 | 38.38 | 15.59 | 1.18 | 0.34 | 4.93 | 32.16 |
| Sample 310 | 0.12 | 0.46 | 2.83 | 2.73 | 39.11 | 15.45 | 1.11 | 0.35 | 4.92 | 31.60 |
| Sample 311 | 0.11 | 0.47 | 2.91 | 2.77 | 39.17 | 15.86 | 1.20 | 0.35 | 4.67 | 31.20 |
| Sample 312 | 0.11 | 0.46 | 2.89 | 2.76 | 39.89 | 15.85 | 1.14 | 0.34 | 4.58 | 30.71 |
| Sample 313 | 0.11 | 0.37 | 2.49 | 2.42 | 45.59 | 12.75 | 0.84 | 0.31 | 5.11 | 29.00 |
| CHK84 | 0.03 | 0.45 | 4.81 | 3.2 | 66.31 | 8.53 | 0.36 | 0.34 | 0.95 | 14.40 |
| Control 2 | | | | | | | | | | |
| CHK64 | 0.02 | 0.40 | 7.04 | 2.69 | 81.03 | 7.27 | 0.34 | 0.11 | 0.48 | 0.11 |
| Control 7 | | | | | | | | | | |

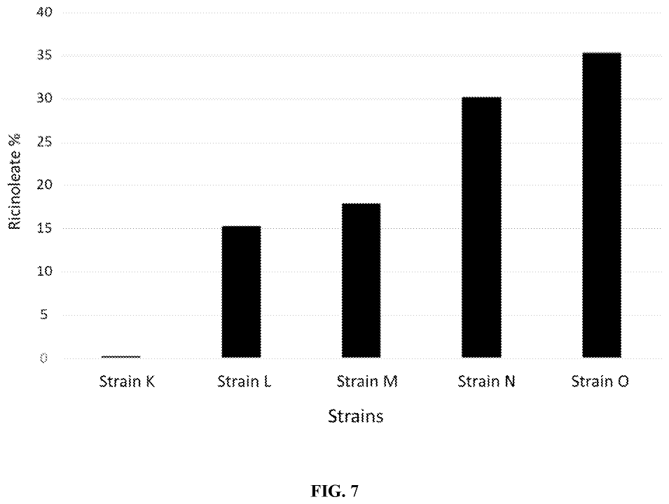

FIG. 7 shows a comparison of ricinoleate levels of P. moriformis strains.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A non-naturally occurring oil having a fatty acid profile comprising:
at least 10% C18:1; and
at least 1% of a hydroxylated C18:1.

2. The oil of claim 1, wherein the fatty acid profile is determined by gas chromatography-flame ionization detection (GC/FID).

3. The oil of claim 1, wherein the fatty acid profile comprises at least 5%, at least 10%, at least 15%, at least 20%, or at least 30% of the hydroxylated C18:1.

4-8. (canceled)

9. The oil of claim 1, wherein the hydroxylated C18:1 is 12-OH-C18:1.

10. The oil of claim 1, wherein the hydroxylated C18:1 is ricinoleic acid.

11-14. (canceled)

15. The oil of claim 1, wherein the C18:1 is oleic acid.

16-21. (canceled)

22. The oil of claim 1, wherein the fatty acid profile further comprises less than 20% C18:2.

23-27. (canceled)

28. The oil of claim 1, wherein the fatty acid profile further comprises less than 20% of saturated fatty acids.

29-33. (canceled)

34. The oil of claim 1, wherein the saturated fatty acids are midchain saturated fatty acids.

35. The oil of claim 34, wherein the midchain fatty acids comprise C10 and C12 fatty acids.

36. The oil of claim 1, wherein the fatty acid profile comprises at least 20% of one or more midchain fatty acids.

37-39. (canceled)

40. The oil of claim 1, wherein the fatty acid profile further comprises 0.1-5% C10:0.

41. The oil of claim 1, wherein the fatty acid profile further comprises 10-30% C12:0.

42. The oil of claim 1, wherein the non-naturally occurring oil is a microalgal oil.

43. A non-naturally occurring oil having a fatty acid profile comprising:
at least 5% of saturated fatty acids; and
at least 1% of a hydroxylated C18:1.

44-45. (canceled)

46. The oil of claim 43, wherein the fatty acid profile comprises at least 20% of one or more midchain fatty acids.

47-49. (canceled)

50. The oil of claim 43, wherein the midchain fatty acids comprise C10 and C12 fatty acids.

51-52. (canceled)

53. The oil of claim 43, wherein the fatty acid profile comprises at least 5%, at least 10%, at least 15%, at least 20%, or at least 30% of the hydroxylated C18:1.

54-58. (canceled)

59. The oil of claim 43, wherein the hydroxylated C18:1 is 12-OH-C18:1.

60. The oil of claim 43, wherein the hydroxylated C18:1 is ricinoleic acid.

61. The oil of claim 43, wherein the non-naturally occurring oil is a microalgal oil.

62. A composition comprising the oil of claim 1.

63-64. (canceled)

65. A microalgal cell comprising an exogenous oleate 12-hydroxylase, wherein the cell produces an oil having a fatty acid profile comprising at least 1% of a hydroxylated C18:1.

66. The cell of claim 65, wherein the cell produces the oil having a fatty acid profile comprising: at least 10% C18:1; and at least 1% of a hydroxylated C18:1.

67. (canceled)

68. The cell of claim 65, wherein the exogenous oleate 12-hydroxylase is codon-optimized for expression in Prototheca sp.

69. (canceled)

70. The cell of claim 65, wherein the cell is from a non-genetically modified Prototheca strain that produces an oil having a fatty acid profile of at least 60%, or at least 70% oleic acid.

71-72. (canceled)

73. The cell of claim 65, wherein the cell produces at least 60% lipid by dry cell weight.

74. The cell of claim 65, wherein the exogenous oleate 12-hydroxylase is a Lesquerella fendleri oleate 12-hydroxylase (LFAH12).

75-76. (canceled)

77. The cell of claim 65, wherein the exogenous oleate 12-hydroxylase is a Ricinus communis oleate 12-hydroxylase (RcFAH12).

78. (canceled)

79. The cell of claim 65, wherein the exogenous oleate 12-hydroxylase is Claviceps purpurea oleate 12-hydroxylase (CpFAH12).

80. (canceled)

81. The cell of claim 65, wherein the cell further comprises an exogenous acyl-ACP thioesterase.

82. The cell of claim 81, wherein the exogenous acyl-ACP thioesterase is a Lindera benzoin acyl-ACP thioesterase (FATB).

83. The cell of claim 81, wherein the exogenous acyl-ACP thioesterase is L benzoin FATB1.

84-85. (canceled)

86. The cell of claim 65, wherein the cell further comprises an exogenous acyltransferase.

87. The cell of claim 86, wherein the exogenous acyltransferase is a phospholipid:diacylglycerol acyltransferase (PDAT).

88-91. (canceled)

92. The cell of claim 86, wherein the exogenous acyltransferase is R. communis PDAT2.

93-94. (canceled)

95. The cell of claim 86, wherein the exogenous acyltransferase is a phosphatidylcholine:diacylglycerol phosphocholine acyltransferase (PDCT).

96. The cell of claim 86, wherein the exogenous acyltransferase is a R. communis PDCT.

97. The cell of claim 86, wherein the exogenous acyltransferase is R. communis PDCT1.

98-99. (canceled)

100. The cell of claim 86, wherein the exogenous acyltransferase is an acyl-CoA:diacylglycerol acyltransferase (DGAT).

101. The cell of claim 86, wherein the exogenous acyltransferase is a R. communis DGAT.

102. The cell of claim 86, wherein the exogenous acyltransferase is R. communis DGAT2.

103-104. (canceled)

105. The cell of claim 65, wherein the cell further comprises an exogenous three prime untranslated region (3′-UTR) downstream of the exogenous oleate 12-hydroxylase.

106. The cell of claim 105, wherein the exogenous 3′-UTR is Chlorella vulgaris nitrate reductase (CvNR) 3′-UTR.

107-113. (canceled)

114. The cell of claim 65, wherein the cell is genetically modified to decrease expression of an endogenous acyl-ACP thioesterase.

115. (canceled)

116. The cell of claim 65, wherein the endogenous acyl-ACP thioesterase is FATA-1.

117-120. (canceled)

121. The cell of claim 65, wherein the cell is genetically modified to decrease expression of an endogenous delta-12 fatty acid desaturase.

122-123. (canceled)

124. The cell of claim 65, wherein the cell further comprises an exogenous PmFAD2 hairpin sequence.

125-126. (canceled)

127. A method of producing an oil composition, the method comprising cultivating a microalgal cell in a culture medium, wherein the oil composition has a fatty acid profile comprising at least 1% of a hydroxylated C18:1.

128-130. (canceled)

131. A method of producing an oil composition, the method comprising cultivating a microalgal cell in a culture medium, wherein the oil composition has a fatty acid profile comprising:
at least 5% of saturated fatty acids; and
at least 1% of a hydroxylated C18:1.

132-134. (canceled)
